SEQUENCE LISTING (332 sequences)

SEQ ID NO: 1 - protein, 742 aa, Yarrowia lipolytica
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 2 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 3 - protein, 742 aa, Artificial Sequence
  Synthetic
MGIHESVSKQFAKNGHSKYRSDRYGLPKTDLRRWTFHASDLGAQWWKYDGTTPLEELEKRATDYVRYSLELPGYAPVTLDSKPVKNAYEAALKSWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCSFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALVTQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRLYFVMKALGLYFKGYCQ

SEQ ID NO: 4 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacggtaccacaccgctggaagagctggaaaagagggctaccgactacgtcagatactcgctggagctgccgggatacgcgcccgtgactctggactccaagcccgtgaaaaatgcctacgaagcggctctcaaaagctggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtagctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccatcggatacattattcgagagcagcagcctgacggtggcttctttggctcctggggcgtgtgctactgctacgctcacatgtttgccatggaggctctggtgacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttatattttgtcatgaaggctttggggttgtatttcaagggatattgccagtga

SEQ ID NO: 5 - protein, 5 aa, Artificial Sequence
  Synthetic
DCTAE

SEQ ID NO: 6 - protein, 329 aa, Unknown
  Streptomyces sp. (strain CL190)
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 7 - DNA, 990 bp, Artificial Sequence
  Synthetic
atgaccgacgtccgattccgaattatcggtactggtgcctacgttcccgaacgaatcgtttccaacgatgaagtcggtgctcctgccggtgttgacgacgactggatcacccgaaagaccggtattcgacagcgacgatgggctgccgatgaccaggccacctctgatctggccactgctgccggtcgagctgccctgaaggccgctggtatcactcccgagcagctgaccgttattgctgttgccacctccactcccgatcgaccccagcctcccactgctgcctatgttcagcaccacctcggagccaccggtactgctgccttcgacgtcaacgctgtctgctccggtaccgttttcgccctgtcctctgttgctggcaccctcgtttaccgaggtggttacgctctggtcattggcgctgacctgtactctcgaatcctcaaccctgccgaccgaaagaccgtcgttctgttcggtgatggtgccggtgccatggttctcggtcctacctccaccggtactggtcccattgttcgacgagttgccctgcacaccttcggtggtctgaccgacctgattcgagtccccgctggtggttctcgacagcccctggacactgatggcctcgatgctggactgcagtacttcgctatggacggtcgtgaggtccgacgattcgtcactgagcacctcccccagctgatcaagggtttcctgcacgaggccggtgtcgacgctgccgacatctctcacttcgtccctcatcaggccaacggtgtcatgctcgacgaggtcttcggcgagctgcatctgcctcgagctaccatgcaccgaactgtcgagacttacggcaacaccggagctgcctccattcccatcaccatggacgctgccgttcgagccggttccttccgacctggtgagctggtcctgctggccggtttcggtggcggtatggccgcttccttcgccctgatcgagtggtag

SEQ ID NO: 8 - DNA, 2196 bp, Unknown
  Saccharomyces cerevisiae S288
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacacatacgctttaa

SEQ ID NO: 9 - protein, 489 aa, Yarrowia lipolytica
MVTQQSAAETSATQTNEYDVVIVGAGIAGPALAVALGNQGRKVLVVERDLSEPDRIVGELLQPGGVAALKTLGLGSCIEDIDAIPCQGYNVIYSGEECVLKYPKVPRDIQQDYNELYRSGKSADISNEAPRGVSFHHGRFVMNLRRAARDTPNVTLLEATVTEVVKNPYTGHIIGVKTFSKTGGAKIYKHFFAPLTVVCDGTFSKFRKDFSTNKTSVRSHFAGLILKDAVLPSPQHGHVILSPNSCPVLVYQVGARETRILCDIQGPVPSNATGALKEHMEKNVMPHLPKSIQPSFQAALKEQTIRVMPNSFLSASKNDHHGLILLGDALNMRHPLTGGGMTVALNDALLLSRLLTGVNLEDTYAVSSVMSSQFHWQRKHLDSIVNILSMALYSLFAADSDYLRILQLGCFNYFKLGGICVDHPVMLLAGVLPRPMYLFTHFFVVAIYGGICNMQANGIAKLPASLLQFVASLVTACIVIFPYIWSELT

SEQ ID NO: 10 - DNA, 1470 bp, Yarrowia lipolytica
ctaagtcagctcgctccaaatgtaagggaagatgacgatgcaagcggtgaccagagaggcgacaaattgcagtagcgacgcgggcagcttggcaatgccgttggcctgcatgttgcagattccgccgtagatggccactacgaagaaatgcgtaaacaggtacatgggtcgggggagaactccagccaacagcatgacggggtggtccacacagatgcctcccagcttgaagtagttgaagcatccgagctgcaggattcgcaagtagtccgagtcggcggcgaagagcgagtagagggccatggagagaatgttgacgatggagtcgaggtgttttcgctgccagtggaactgcgagctcatgacggaggacacggcataggtgtcttccaggttaacgccggtgagaagtctgctgagtagaagggcatcattgagagcaacggtcattcctcctccggtaagtggatgtcgcatgttgagtgcgtcacccagcagaatcaaaccgtggtgatcgttcttggaggccgacaggaaagagttgggcatgactcgaatggtctgctccttgagagcggcttggaaagacggctggatggacttaggcaggtggggcatgacgttcttctccatgtgttccttgagggctccggttgcattagaggggacgggtccctgaatgtcacacagaattcgggtctctcgagctccaacctggtagacaagaacgggacacgagttgggcgacagaatcacgtggccatgctggggggagggcagaacagcgtccttgagaatcagaccggcgaaatgcgaacgcacagacgtcttgttggtgctaaagtcctttcggaacttggaaaaagttccatcacagacgacggtgagaggagcaaagaagtgcttgtagattttggcgcctccagttttagagaaggtcttgactccaataatgtggccggtgtaagggttcttgaccacctcggtgactgtggcctccagcagagtcacattgggtgtgtctcgtgcggcccttcgcaagttcatgacaaatcggccgtggtggaaggatactcctcggggagcctcgttggagatgtcggcagactttccgcttctgtacagctcgttgtagtcctgctggatgtctcgggggaccttggggtatttgagaacgcactcttctccagagtagatcacgttgtatccctggcaggggatcgcgtcgatatcctcgatacaagagccgagacccagagtcttgagagcagcgactcctccgggctgaagcagctctcccacgattcggtccggttcggagagatctcgttccacaacaagaacctttctgccctgatttccaagagccacggccagagcgggcccggcaataccagctccgacaatgaccacgtcgtactcgttggtctgggtggcgctggtctctgctgcagactgttgggtgaccat

SEQ ID NO: 11-54 - skipped (44 entries)

SEQ ID NO: 55 - protein, 9 aa, Artificial Sequence
  Synthetic
  misc_feature (3)..(4): Xaa can be any naturally occurring amino acid
  misc_feature (6)..(8): Xaa can be any naturally occurring amino acid
  SITE (9)..(9): Xaa may be Asp or Glu
  misc_feature (9)..(9): Xaa may be Asp or Glu
NDXXSXXXX

SEQ ID NO: 56-60 - skipped (5 entries)

SEQ ID NO: 61 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcgtggcagagcgaatacgacggaccgcagttcatgagcatcggctatgtgacggcgtgctactttggcggcaacgagatccccacgccggtcaaaaccgaaatgatcagatacattgtcaacacagcccacccagttgacggaggctggggccttcacaaagaagacaagagcacctgcttcggtaccagcatcaactacgtggtcctgcgactactgggcctgtcacgggaccatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctccaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccggtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctctcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 62 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagttggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcgcgggatcatccggtctgcgtcaaggcgtgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattggtttctccaaacattgcatcaccatctccggagtcgacctctactatccccacaccggccttttgaagtttggcaacgcgcttctccgacgataccgcaagttcagaccgcagtggatcaaagaaaaggtcaaggaggaaatttataacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagagtctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtttttcaagggatattgccagtga

SEQ ID NO: 63 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcaacaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgagtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttatgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtagtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 64 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtctgattttctcgatcggtcccagatcaacgagccgacggaggaaaattcctatcgagacggccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagttcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggtggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 65 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaataatgcctacgaagcggctctcaaaaactggcatctgtttgcgtcgctgcaagaccccgactccggcgcatggcagtcggaatacgacggaccgcagttcatgtcgatcggttatgtgacggcatgctactttggcggcaacgagatccccacgccggtcaaaaccgaaatgatcagatacattgtcaacacagcccacccagttgacggaggctggagccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctccaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccggtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctctcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 66 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctggtcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacataaagccctactcggagattgatttctccaaacattgcaactccatctccggagtcgacctctactatccccacaccggccttttgaagtttggcagcgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggctcgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatatattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 67 - skipped

SEQ ID NO: 68 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcaacaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgagtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttatgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 69 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcgtggcagagcgaatacgacggaccgcagttcatgagcatcggctatgtgacggcgtgctactttggcggcaacgagatccccacgccggtcaaaaccgaaatgatcagatacattgtcaacacagcccacccagttgacggaggctggggccttcacaaagaagacaagagcacctgcttcggtaccagcatcaactacgtggtcctgcgactactgggcctgtcacgggaccatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgtttcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatatttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacgatgagtggctcaaggaggagatggagggggtgtttaaccatacttgtgccattgagtatcccaactaccggttttattttgtcatgaaggctttggggttgtatttcaagggatattgccagtga

SEQ ID NO: 70 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcgtggcagagcgaatacgacggaccgcagttcatgagcatcggctatgtgacggcgtgctactttggcggcaacgagatccccacgccggtcaaaaccgaaatgatcagatacattgtcaacacagcccacccagttgacggaggctggggccttcacaaagaagacaagagcacctgcttcggtaccagcatcaactacgtggtcctgcgactactgggcctgtcacgggaccatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgtttcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaatcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatatttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacgatgagtggctcaaggaggagatggagggggtgtttaaccatacttgtgccattgagtatcccaactaccggttttattttgtcatgaaggctttggggttgtatttcaagggatattgccagtga

SEQ ID NO: 71 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctgggctccaagctcgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtgccagcatcaactacgtggtcctgcgactactgggcctgtcacgggatcatccggtctgcgtcaaggcgcgcaaaacgctgctcatcaagtttggcggcgccatcaacaacccccattggggcaagacctggctgtcgattctcaatctctacaaatgggagggtgtgaatccgacccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 72 - skipped

SEQ ID NO: 73 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcgtggcagagcgaatacgacggaccgcagttcatgagcatcggctatgtgacggcgtgctactttggcggcaacgagatccccacgccggtcaaaaccgaaatgatcagatacattgtcaacacagcccacccagttgacggaggctggggccttcacaaagaagacaagagcacctgcttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggtcgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgtcctccattgtcatgtatctccatgaggggcccgatccggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgatggaggaaaattcctatcgagacgaccgcgtcggaggatggccctttagtaccaagacccaggggtatccagtctccgactgtactgccgaggctctcaaggccatcatcatggtccagaatacacctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctctcctactttcgaaagtaccacgattaccgcaacgaagacgtggacccagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagacccagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 74 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacggtaccacaccgctggaagagctggaaaagagggctaccgactacgtcagatactcgctggagctgccgggatacgcgcccgtgactctggactccaagcccgtgaaaaatgcctacgaagcggctctcaaaagctggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtagctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccatcggatacattattcgagagcagcagcctgacggtggcttctttggctcctggggcgtgtgctactgctacgctcacatgtttgccatggaggctctggtgacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 75-77 - skipped (3 entries)

SEQ ID NO: 78 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagatgaaatctacaaaaagccctactcggagattgatttctccaaacattgcaactccatctccggagtcgacctctactacccccacaccggctttttgaagtttggcaacgcgcttctccgacgataccgcaagttcagaccgcagtggatcaaagaaaaggtcaaggaggaaatttacaacttgtgccttcgagaggcttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcatcaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtatctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgtaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcaggttaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacgagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 79 - skipped

SEQ ID NO: 80 - DNA, 2178 bp, Artificial Sequence
  Synthetic
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaatttctgagcatgagagaatagaattaattagatacatcgtcaatacagtacatccggttgatggtggctggggtctacattctgttgacaaatccaccgtgtttggtacagtattaaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacggccttgtagtgttttacgaaaaatacctaagaaaccggttcatttactctctatccaagaagaagggttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggatgagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatctttagaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaactaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatggaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatag

SEQ ID NO: 81 - DNA, 2196 bp, Artificial Sequence
  Synthetic
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagctgtaaactatatcgccggtattgaaattcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactagtccgtttgacaagattaacttctccaagaacaggaatgccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatctcttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaacgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccacatgggcgctaattgcacttcttttcgctgaatatcctaataaagaagtcatcgaccgcggtattgaccttttaaaaaatagacaagaagaatccggggaatggaaatttggaagtgtagaaggtgttttcaaccactcttgtgcaattgaatacccaagttatcgattcttattccctattaaggcattaggtatgtacagcagggcatatgaaacacatacgctttaa

SEQ ID NO: 82 - DNA, 2196 bp, Artificial Sequence
  Synthetic
atgacagaattttattctgacacaatcggtctaccaaagacagatccacgtctttggagactgagaactgatgagctaggccgagaaagctgggaatatttaacccctcagcaagccgcaaacgacccaccatccactttcacgcagtggcttcttcaagatcccaaatttcctcaacctcatccagaaggaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaagttcctgagcatgagagaatagaattaattagatacatcgtcaatacagcacatccggttgatggtggctggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccattcatccggggagatggtgggttcatactagaggtgtttacattccggtcagttacctgtcattggtcaaattttcttgcccaatgactcctcttcttgaagaactgaggaatgaaatttacactaaaccgtttgacaagattaacatctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatgctgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggtagcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacacatacgctttaa

SEQ ID NO: 83 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKACKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIGFSKHCITISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQSLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLFFKGYCQ

SEQ ID NO: 84 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSNNYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYASVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVMGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSSRYPQEDKIHAAARFLMSKQMSNGEWLKEEMEGVFNHTCAIEYPNYRFYFVMKALGLYFKGYCQ

SEQ ID NO: 85 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAASDFLDRSQINEPTEENSYRDGRVGGWPFSTKTQGYPVSDCTAEALKAIIMVQNTPGYEDLKKQVSDKRKHTAIDLLLGMQNVGSFEPGSFASYEPIRASSMLEKFNPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 86 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVNNAYEAALKNWHLFASLQDPDSGAWQSEYDGPQFMSIGYVTACYFGGNEIPTPVKTEMIRYIVNTAHPVDGGWSLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 87 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLVTKFGGAINNPHWGKTWLSILNLYKWEGVNPAPGELWLLPYFVPVHPGRWWVHTRWIYLAMGYLEAAEAQCELTPLLEELRDEIYIKPYSEIDFSKHCNSISGVDLYYPHTGLLKFGSALLRRYRKFRPQWIKEKVKEEIYNLCLREVSNTRHLCLAPVNNAMTSIVMYLHEGLDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 88 - skipped

SEQ ID NO: 89 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSNNYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYASVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVMGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 90 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPVSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHIFAMEALETQNLNYNNCSTVQKACDFLAGYQEADGGWAEDFKSCETQMYVRGPHSLVVPTAMALLSLMSGRYPQEDKIHAAARFLMSKQMSNDEWLKEEMEGVFNHTCAIEYPNYRFYFVMKALGLYFKGYCQ

SEQ ID NO: 91 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPVSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDNRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHIFAMEALETQNLNYNNCSTVQKACDFLAGYQEADGGWAEDFKSCETQMYVRGPHSLVVPTAMALLSLMSGRYPQEDKIHAAARFLMSKQMSNDEWLKEEMEGVFNHTCAIEYPNYRFYFVMKALGLYFKGYCQ

SEQ ID NO: 92 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLGSKLVKNAYEAALKNWHLFASLQDPDSGAWQSEYDGPQFMSIGYVTACYFGGNEIPTPVKTEMIRYIVNTAHPVDGGWGLHKEDKSTCFGASINYVVLRLLGLSRDHPVCVKARKTLLIKFGGAINNPHWGKTWLSILNLYKWEGVNPTPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 93 - skipped

SEQ ID NO: 94 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMSSIVMYLHEGPDPANYKKIAARWPEFLSLNPSGMFMNGTNGLQVWDTAFAVQYACVCGFAELPQYQKTIRAAFDFLDRSQINEPMEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDPAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 95 - protein, 742 aa, Artificial Sequence
  Synthetic
MGIHESVSKQFAKNGHSKYRSDRYGLPKTDLRRWTFHASDLGAQWWKYDGTTPLEELEKRATDYVRYSLELPGYAPVTLDSKPVKNAYEAALKSWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCSFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALVTQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 96-98 - skipped (3 entries)

SEQ ID NO: 99 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGFLKFGNALLRRYRKFRPQWIKEKVKEEIYNLCLREASNTRHLCLAPVNNAMTSIVMYLHEGPDSANYKKIAARWPEFLSLNPSGMFMNGINGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDEIHAAARFLMSKQMSNGEWLKEEMEGVFNHTCAIEYPNYRFYFVMKALGLYFKGYCQ

SEQ ID NO: 100 - protein, 725 aa, Artificial Sequence
  Synthetic
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEISEHERIELIRYIVNTVHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANGLVVFYEKYLRNRFIYSLSKKKGYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDERKGAWGFSTKTQGYTVADCTAEAIKAIIMVKNSPVFSEVHHMISSERLFEGIDVLLNLQNIGSLEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMEDGGWGESMKSSELHSYVDSEKSLVVQTAWALIALLFAEYPNKEVIDRGIDLLKNRQEESGEWKFESVEGVFNHSCAIEYPSYRFLFPIKALGMYSRA

SEQ ID NO: 101 - protein, 731 aa, Artificial Sequence
  Synthetic
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTSPFDKINFSKNRNAVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSLEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHNVGETYENSSTVRKGCDFLVSKQMKDGGWGESMKSSELHSYVDSEKSLVVQTTWALIALLFAEYPNKEVIDRGIDLLKNRQEESGEWKFGSVEGVFNHSCAIEYPSYRFLFPIKALGMYSRAYETHTL

SEQ ID NO: 102 - protein, 731 aa, Artificial Sequence
  Synthetic
MTEFYSDTIGLPKTDPRLWRLRTDELGRESWEYLTPQQAANDPPSTFTQWLLQDPKFPQPHPEGNKHSPDFSAFDACHNGASFFKLLQEPDSGIFPCQYKGPMFMTIGYVAVNYIAGIEVPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPIHPGRWWVHTRGVYIPVSYLSLVKFSCPMTPLLEELRNEIYTKPFDKINISKNRNTVCGVDLYYPHSTTLNIANSLVVFYEKYLRNRFIYSLSKKKVYDLIKTELQNADSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGSDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETHTL

SEQ ID NO: 103 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtggttccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgtttcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacgatgagtggctcaaggaggagatggagggggtgtttaaccatacttgtgccattgagtatcccaactaccggttttattttgtcatgaaggctttggggttgtatttcaagggatattgccagtga

SEQ ID NO: 104 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcatgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgatgccctactttgttcctgttcatccgggccgatggtgggtccatacccggtggatctaccttgccatgggctatcgggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatgcagggggtgtttaaccatacttgtgccattgagtatcccaactaccggttttattttgtcatgaaggctttggggttgtatttcaagggatattgccagtga

SEQ ID NO: 105 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccacacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctcagacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatcagaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggattcattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcacggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 106 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctactgactacgtcaaatactcgctggagctgccgggatacgcgcccgtgactctggactccaagcccgtgaagaatgcctacgaagcggctctcaaaaactggcatctgtttgcgtcgctgcaagaccccgactccggcgcatggcagtcggaatacgacggaccgcagttcatgtcgatctgttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccttacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcatgggatattgccagtga

SEQ ID NO: 107 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctactgactacgtcaaatactcgctggagctgccgggatacgcgcccgtgactctggactccaagcccgtgaagaatgcctacgaagcggctctcaaaaactggcatctgtttgcgtcgctgcaagaccccgactccggcgcatggcagtcggaatacgacggaccgcagttcatgtcgatctgttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccttacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatctgtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgttgaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcatgggatattgccagtga

SEQ ID NO: 108 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgctatcaacaacccccattggggcaagacctggctgtcgattctcaatctctacaaatgggagggtgtgaatctggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaagaaaattcctatcgagacgaccgcgtcggaggatggccctttagtaccaagacccaggggtatccagtctccgactgtactgccgtggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgctctgggtctgtccaactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 109 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccgacgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaagaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 110 - skipped

SEQ ID NO: 111 - DNA, 2226 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaataatgcctacgaagcggctctcaaaaactggcatctgtttgcgtcgctgcaagaccccgactccggcgcatggcagtcggaatacgacggaccgcagttcatgtcgatcggttatgtgacggcatgctactttggcggcaacgagatccccacgccggtcaaaaccgaaatgatcagatacattgtcaacacagcccacccagttgacggaggctggagccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggcttgtttgaaccgggctctttcgcctcctatgagactatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgctgaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgctag

SEQ ID NO: 112 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcatgctactttggcggcaacgagatccccacgccggtcaaaactgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagatcttgctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggatacatcatggtggagtatccgtacgaggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacagttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 113 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgtgctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgtgtttgccgtgcaatacgcgtgtgtttgtggctttgccgaacttcccctgtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccggacgtcgtccatgctggagaagatcaatccggccgaggtgtttggaaacatcatggtggagtatccgtacgtggaatgcactgattctgttgttctgggtctgtcctgctttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 114 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactcaggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggggattgatttctccaaacattgcaactccatctccggagtcgacctctactatccccacaccggccttttgaagtttggcaacgcgcttctccgacgataccgcaagttcagaccgcagtggatcaatgaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggtgacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccgggtttattttgtcatgaaggctttggggttgtatttcaagggatattgccagtga

SEQ ID NO: 115 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtgggagtatgacgataccacaccgctggaagagctggaaaagagggctaccgactacgtcaaatactcgctggagctgccgggatacgcgcccgtgactctggactccaagcccgtgaaaaatgcctacgaagcggctatcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggtcgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgtcctccattgtcatgtatctccatgaggggcccgatccggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgatggaggaaaattcctatcgagacgaccgcgtcggaggatggccctttagtaccaagacccaggggtatccagtctccgactgtactgccgaggctctcaaggccatcatcatggtccagaatacacctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggacccagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 116 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtctgattttctcgatcggtcccagatcaacgagccgacggaggaaaattcctatcgagacggccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagttcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggtggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 117 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctggtcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacataaagccctactcggagattgatttctccaaacattgcaactccatctccggagtcgacctctactatccccacaccggccttttgaagtttggcagcgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggctcgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatatattattcgagagcagcagcctgacggcggcttctttggctcctggggcgtgtgctactgctacactcacatgtttgccatggaggctctggagacgcagaatctcaactataacaactgttccacggttcaaaaggcgtgcgactttctggcggactaccaggaagcagatggaggctgggccgaggaccttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

SEQ ID NO: 118 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWFHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPVSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNDEWLKEEMEGVFNHTCAIEYPNYRFYFVMKALGLYFKGYCQ

SEQ ID NO: 119 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSWDHPVCVKARKTLLTKFGGAINNPHWGKTWLSILNLYKWEGVNPAPGELWLMPYFVPVHPGRWWVHTRWIYLAMGYREAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMQGVFNHTCAIEYPNYRFYFVMKALGLYFKGYCQ

SEQ ID NO: 120 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDQKKQVSDKRKHTAIDLLLGMQNVGSFEPGSFASYEPIRASSMLEKINPAEVFGNIMVEYPYVECTDSVVLGLSYFRKYHDYRNEDVDRAISAAIGFIIREQQPDGGFFGSWGVCYCYAHMFAMEALETQNLNYNNCSTVQKACDFLAGYQEADGGWAEDFKSCETQMYVHGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 121-225 - skipped (105 entries)

SEQ ID NO: 226 - protein, 761 aa, Unknown
  Aquilaria agallocha
MWRLKTGSETVGDNGRWLRSTNNHVGRQVWEFFPEMGSPEELVAIEAAHREFHLNRFHKQHSSDLLMRLQYEREKPCVQKEGAVRLDATETPTEAAVETTLRRALTFYSTMQSDDGHWANDLGGPMFLLPGLVITLTITGTINVVLSKEHQREIRRYLYNHQNQDGGWGLHIEGPSTMFGSALNYVTLRLLGEGPDDGEGAMERARQWILSRGGAVAVTSWGKLWLSVLGVYEWDGNNPLPPELWLLPYSLPLHPGRMWCHCRMVYLPMSYLYGKRFVGPITPTVLSLREELYPIPYHHVDWNKARNTCAQDDLYYPHPFVQDLLWGSLYHVYEPLVMRWPGKRLRERALQHVMKHIHYEDENTEYICLGPVNKALNMLCCWVEDPHSEAFKMHIPRIYDYLWIAEDGMKMQGYNGSQLWDTAFAVQAIVATKLTDEFSETLAKANKYILDAQILKNCPGDPNVWYRHITKGAWSFSTADQGWLVSDCTAEGLKALLLYSMLPHQKAPSSIEKNRLYDAVNVLLSMQNADGGFASFELTRSYPWLEMINPAETFGDIVIDYTYVECTSAVIQALALFKRLHPGHRKKEIERCMANAAKFLEMRQEADGSWYGCWGVCYTYAGWFGIKGLTSCGRTYNNCANIRRACDFLLSKQLPNGGWGESYLSCQNKLYTNLNNDRMHTVNTAWAMMALIEAGQAKTDPMPLHHAARTLINAQMETGDFPQQEIMGVFNKNCMISYAGYRNVFPVWALGEYHHRVLNGC

SEQ ID NO: 227-255 - skipped (29 entries)

SEQ ID NO: 256 - protein, 759 aa, Unknown
  Siraitia grosvenorii
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 257-276 - skipped (20 entries)

SEQ ID NO: 277 - DNA, 1587 bp, Artificial Sequence
  Synthetic
atggtggaccaatgtgcacttgggtggatcctagcgtcagctctgggcttagtaatagccctctgcttctttgtcgctcctcgaagaaaccaccgcggagttgattcgaaagagagagatgaatgtgttcagtctgcggcaaccactaagggtgaatgtaggtttaatgaccgtgatgtagatgttattgtcgtgggtgctggtgttgccggatccgcattggcacatacgttgggcaaagacggtagaagggtgcatgtaattgaaagagacctcacagaaccagatcggatagttggggagttacttcaaccgggtggttacttaaagctaatcgagttaggattgcaagattgcgtggaagaaattgatgctcagagagtatatggctatgccttgttcaaagatggaaaaaatacacgtttgagctacccattagagaactttcacagtgacgtttctggtcgatcattccataatggtagatttattcaacgtatgagagaaaaggctgcgtccctacccaacgtcaggctggaacaaggaactgttacctcgctcttggaggaaaaaggcactatcaagggtgtccaatataaatcaaagaatggggaagaaaaaacagcatacgctccgctcactatagtgtgtgacggttgtttctctaacttacgccgaagtctgtgcaatcctatggtcgatgttccaagctattttgtaggcttggtgttggaaaattgcgagctgccattcgctaaccacggacatgtaattttaggcgatccttctcccattcttttttaccagatttccaggaccgaaataagatgtttggttgatgtccctggtcaaaaagttccatcaatagcaaatggcgagatggaaaagtatctgaaaacagtggtagctcctcaggttcctccacaaatctatgatagttttattgcggccatagacaagggtaacatcaggacgatgcccaatagatctatgccagctgccccacatcctacgccgggtgcccttctaatgggggatgcatttaacatgagacatcccctgacaggaggtggtatgaccgtggcattgagcgatattgtagttttacgtaatcttttaaaacctctcaaggacctgtcagatgcaagtactctgtgcaagtatttagaaagtttctacacccttagaaaaccagttgcttcaactattaacacgttggccggggctctatataaagtattttgtgcctctccggaccaggctaggaaagaaatgcgtcaagcttgtttcgattatttatccttgggaggcatattttcaaatggccctgtatcgctattaagcggactaaacccaagaccactatctctagtcctccacttctttgctgtggcaatatacggtgttggtcgcttgctacttccatttccttctgtcaaggggatctggattggagcgcgtttaatctatagcgcgagtggtattatttttcccattataagagctgagggtgttagacagatgtttttccctgcaacagttcctgcctactataggtccccacccgtgttcaaacccatagtttaa

SEQ ID NO: 278 - DNA, 1575 bp, Artificial Sequence
  Synthetic
atggtggaccaatgtgcacttgggtggatcctagcgtcagttctgggcgctgccgcactctactttttgttcggacgaaaaaatggtggtgtatcgaacgaaaggcgccatgagtccataaagaatattgctactaccaacggtgaatataaaagctctaattccgatggggatattataatcgtcggcgcaggagttgctggtagtgccttagcttatacgttgggtaaggacggaagaagagtgcacgtcattgagcgtgatttgacagaaccggatagaatcgtgggggaattattgcagcccggcggttacttaaaactaactgagttaggtcttgaagactgcgttgatgatattgacgcacaaagggtatatggttacgctttatttaaggatggtaaagatacacggctatcttatccattagaaaagttccattcagacgtagccggaaggtcttttcacaacggcagattcatccaaagaatgcgggaaaaagcggctagtttgccaaaagtttcacttgagcagggtaccgtaacttcgctgcttgaagaaaatggcattataaagggcgtccaatacaaaacaaaaacaggtcaagaaatgaccgcctatgcacctctcactattgtttgtgacggatgcttttctaacctgcgtagatccttgtgtaatcctaaggttgatgtaccatcatgctttgtgggcttagttcttgagaactgtgatctaccctacgctaatcatgggcatgtgatactggctgaccctagtccaatattgttctatcgaatttcttcaacggagatcagatgtttagtcgacgttcccggacaaaaagttccttcgatctctaatggtgaaatggcgaattacctgaagaacgtagtcgccccacagataccaagtcagttgtatgatagctttgttgcagcaattgataaaggaaatattaggactatgccgaaccgtagcatgccggccgatccttatcccactccgggtgctttattgatgggtgacgcgtttaatatgagacacccattgacaggcggaggtatgaccgtcgcgctgagtgatgttgtcgtgctaagagacctattaaaaccattacgcgatttaaacgatgctcctacactgtcaaagtaccttgaagcattctacacgctacgaaaaccagtagccagtaccatcaacacattggctggtgcattgtataaggtgttttgcgcatctccagatcaagcacgtaaagaaatgagacaggcatgtttcgattatttgtctcttggtggtattttttccaatggacctgtatcactactctcagggttgaatccaaggccaattagcctagtactacatttctttgccgttgccatctacggcgtaggtcgtctattaataccgtttccttctcctaagagagtctggatcggcgctagaattataagcggcgcttcggcaattatcttccctataataaaggctgaaggagttagacaaatgttctttccggctactgtggcggcttattatcgtgcaccaagagttgtcaagggtaggtaa

SEQ ID NO: 279 - DNA, 1536 bp, Artificial Sequence
  Synthetic
atggacggggtaattgatatgcagaccatacccctacgtacggccatcgcaattggaggcactgcggttgctcttgtcgtggctttgtacttctggtttctccgtagctatgcatctccttcccaccattcaaatcatttaccgcctgttccagaagtaccaggtgtgccagttttaggtaacctgttgcaattgaaagagaagaaaccctatatgacatttactaagtgggccgaaatgtacggtcctatctattcgattcgcacaggagctacatcaatggtcgttgtaagtagtaatgagatagcgaaagaagtggttgtgactaggttcccatctatctcaactagaaaactgtcctatgccttgaaggtcttaaccgaagataaatctatggtcgcaatgagcgattaccatgactaccacaaaacagttaagcgacatattttaacggctgttctaggcccaaacgcacaaaagaaatttagagctcacagagataccatgatggagaatgtaagcaacgaattgcatgctttctttgaaaagaatcctaatcaggaagtcaaccttagaaaaatatttcaatcccaacttttcggtttagctatgaaacaggcattagggaaggatgtggaatctatttatgtaaaggacctggaaactaccatgaaaagggaagagatattcgaagttcttgttgttgaccccatgatgggagccattgaagtcgattggcgagattttttcccgtatctcaaatgggtgccaaataaatcttttgaaaatataatccataggatgtacacgagaagagaggccgttatgaaagcgttgattcaagagcacaagaaaaggatcgcttcaggtgagaacctaaattcctacatagattatctcttgagtgaagctcaaacattaactgacaaacagttattaatgtcgttgtgggaacctattatagaatcgtctgatactaccatggtaacaacagaatgggcaatgtatgaacttgcgaagaaccctaatatgcaagatcgtctgtacgaagagattcaatcggtttgtggaagcgagaagattacagaagaaaacttatcacaactcccatatctatatgccgttttccaggaaactctgagaaagcattgccctgtgccgatcatgccacttagatacgtacatgagaacactgttttgggtggttaccacgtaccagcaggcaccgaagtggcaatcaatatttatggctgtaatatggacaagaaagtgtgggaaaatcccgaagagtggaatccagaacgttttctgtctgaaaaagagagtatggatttgtataaaacgatggcatttggtggaggtaaaagagtttgtgcgggatctctacaagctatggtaattagttgcatcggcatcgggaggttagttcaagattttgaatggaaactaaaagatgacgctgaagaagatgtgaatacattaggtttgactacgcaaaagctacaccctttgttagcactaattaacccgcggaaa

SEQ ID NO: 280-292 - skipped (13 entries)

SEQ ID NO: 293 - protein, 528 aa, Siraitia grosvenorii
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 294 - protein, 524 aa, Siraitia grosvenorii
MVDQCALGWILASVLGAAALYFLFGRKNGGVSNERRHESIKNIATTNGEYKSSNSDGDIIIVGAGVAGSALAYTLGKDGRRVHVIERDLTEPDRIVGELLQPGGYLKLTELGLEDCVDDIDAQRVYGYALFKDGKDTRLSYPLEKFHSDVAGRSFHNGRFIQRMREKAASLPKVSLEQGTVTSLLEENGIIKGVQYKTKTGQEMTAYAPLTIVCDGCFSNLRRSLCNPKVDVPSCFVGLVLENCDLPYANHGHVILADPSPILFYRISSTEIRCLVDVPGQKVPSISNGEMANYLKNVVAPQIPSQLYDSFVAAIDKGNIRTMPNRSMPADPYPTPGALLMGDAFNMRHPLTGGGMTVALSDVVVLRDLLKPLRDLNDAPTLSKYLEAFYTLRKPVASTINTLAGALYKVFCASPDQARKEMRQACFDYLSLGGIFSNGPVSLLSGLNPRPISLVLHFFAVAIYGVGRLLIPFPSPKRVWIGARIISGASAIIFPIIKAEGVRQMFFPATVAAYYRAPRVVKGR

SEQ ID NO: 295 - protein, 512 aa, Lactuca sativa
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYHDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLSQLPYLYAVFQETLRKHCPVPIMPLRYVHENTVLGGYHVPAGTEVAINIYGCNMDKKVWENPEEWNPERFLSEKESMDLYKTMAFGGGKRVCAGSLQAMVISCIGIGRLVQDFEWKLKDDAEEDVNTLGLTTQKLHPLLALINPRK

SEQ ID NO: 296-301 - skipped (6 entries)

SEQ ID NO: 302 - DNA, 1335 bp, Artificial Sequence
  Synthetic
atgggaaagctattacaattggcattgcatccggtcgagatgaaggcagctttgaagctgaagttttgcagaacaccgctattctccatctatgatcagtccacgtctccatatctcttgcactgtttcgaactgttgaacttgacctccagatcgtttgctgctgtgatcagagagctgcatccagaattgagaaactgtgttactctcttttatttgattttaagggctttggataccatcgaagacgatatgtccatcgaacacgatttgaaaattgacttgttgcgtcacttccacgagaaattgttgttaactaaatggagtttcgacggaaatgcccccgatgtgaaggacagagccgttttgacagatttcgaatcgattcttattgaattccacaaattgaaaccagaatatcaagaagtcatcaaggagatcaccgagaaaatgggtaatggtatggccgactacatcttggatgaaaattacaacttgaatgggttgcaaaccgtccacgactacgacgtgtactgtcactacgtagctggtttggtcggtgatggtttgacccgtttgattgtcattgccaagtttgccaacgaatctttgtattctaatgagcaattgtatgaaagcatgggtcttttcctacaaaaaaccaacatcatcagagactacaatgaagatttggtcgatggtagatccttctggcccaaggaaatctggtcacaatacgctcctcagttgaaggacttcatgaaacctgaaaacgaacaactggggttggactgtataaaccacctcgtcttaaacgcattgagtcatgttatcgatgtgttgacttatttggccagtatccacgagcaatccactttccaattttgtgccattccccaagttatggccattgcaaccttggctttggtattcaacaaccgtgaagtgctacatggcaatgtaaagattcgtaagggtactacctgctatttaattttgaaatcaaggactttgcgtggctgtgtcgagatttttgactattacttacgtgatatcaaatctaaattggctgtgcaagatccaaatttcttaaaattgaacattcaaatctccaagatcgaacaattcatggaagaaatgtaccaggataaattacctcctaacgtgaagccaaatgaaactccaattttcttgaaagttaaagaaagatccagatacgatgatgaattggtcccaacccaacaagaagaagagtacaagttcaatatggttttatctatcatcttgtccgttcttcttgggttttattatatatacactttacacagagcgtga

SEQ ID NO: 303 - DNA, 1491 bp, Artificial Sequence
  Synthetic
atgtctgctgttaacgttgcacctgaattgattaatgccgacaacacaattacctacgatgcgattgtcatcggtgctggtgttatcggtccatgtgttgctactggtctagcaagaaagggtaagaaagttcttatcgtagaacgtgactgggctatgcctgatagaattgttggtgaattgatgcaaccaggtggtgttagagcattgagaagtctgggtatgattcaatctatcaacaacatcgaagcatatcctgttaccggttataccgtctttttcaacggcgaacaagttgatattccatacccttacaaggccgatatccctaaagttgaaaaattgaaggacttggtcaaagatggtaatgacaaggtcttggaagacagcactattcacatcaaggattacgaagatgatgaaagagaaaggggtgttgcttttgttcatggtagattcttgaacaacttgagaaacattactgctcaagagccaaatgttactagagtgcaaggtaactgtattgagatattgaaggatgaaaagaatgaggttgttggtgccaaggttgacattgatggccgtggcaaggtggaattcaaagcccacttgacatttatctgtgacggtatcttttcacgtttcagaaaggaattgcacccagaccatgttccaactgtcggttcttcgtttgtcggtatgtctttgttcaatgctaagaatcctgctcctatgcacggtcacgttattcttggtagtgatcatatgccaatcttggtttaccaaatcagtccagaagaaacaagaatcctttgtgcttacaactctccaaaggtcccagctgatatcaagagttggatgattaaggatgtccaacctttcattccaaagagtctacgtccttcatttgatgaagccgtcagccaaggtaaatttagagctatgccaaactcctacttgccagctagacaaaacgacgtcactggtatgtgtgttatcggtgacgctctaaatatgagacatccattgactggtggtggtatgactgtcggtttgcatgatgttgtcttgttgattaagaaaataggtgacctagacttcagcgaccgtgaaaaggttttggatgaattactagactaccatttcgaaagaaagagttacgattccgttattaacgttttgtcagtggctttgtattctttgttcgctgctgacagcgataacttgaaggcattacaaaaaggttgtttcaaatatttccaaagaggtggcgattgtgtcaacaaacccgttgaatttctgtctggtgtcttgccaaagcctttgcaattgaccagggttttcttcgctgtcgctttttacaccatttacttgaacatggaagaacgtggtttcttgggattaccaatggctttattggaaggtattatgattttgatcacagctattagagtattcaccccatttttgtttggtgagttgattggttaa

SEQ ID NO: 304 - DNA, 2196 bp, Artificial Sequence
  Synthetic
atgacagaattttattctgacacaatcggtctaccaaagacagatccacgtctttggagactgagaactgatgagctaggccgagaaagctgggaatatttaacccctcagcaagccgcaaacgacccaccatctacctttacacaatggctactgcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatactgctcaccctgtcgacggaggttggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacacatacgctttaa

SEQ ID NO: 305-310 - skipped (6 entries)

SEQ ID NO: 311 - protein, 444 aa, Saccharomyces cerevisiae
MGKLLQLALHPVEMKAALKLKFCRTPLFSIYDQSTSPYLLHCFELLNLTSRSFAAVIRELHPELRNCVTLFYLILRALDTIEDDMSIEHDLKIDLLRHFHEKLLLTKWSFDGNAPDVKDRAVLTDFESILIEFHKLKPEYQEVIKEITEKMGNGMADYILDENYNLNGLQTVHDYDVYCHYVAGLVGDGLTRLIVIAKFANESLYSNEQLYESMGLFLQKTNIIRDYNEDLVDGRSFWPKEIWSQYAPQLKDFMKPENEQLGLDCINHLVLNALSHVIDVLTYLASIHEQSTFQFCAIPQVMAIATLALVFNNREVLHGNVKIRKGTTCYLILKSRTLRGCVEIFDYYLRDIKSKLAVQDPNFLKLNIQISKIEQFMEEMYQDKLPPNVKPNETPIFLKVKERSRYDDELVPTQQEEEYKFNMVLSIILSVLLGFYYIYTLHRA

SEQ ID NO: 312 - protein, 496 aa, Saccharomyces cerevisiae
MSAVNVAPELINADNTITYDAIVIGAGVIGPCVATGLARKGKKVLIVERDWAMPDRIVGELMQPGGVRALRSLGMIQSINNIEAYPVTGYTVFFNGEQVDIPYPYKADIPKVEKLKDLVKDGNDKVLEDSTIHIKDYEDDERERGVAFVHGRFLNNLRNITAQEPNVTRVQGNCIEILKDEKNEVVGAKVDIDGRGKVEFKAHLTFICDGIFSRFRKELHPDHVPTVGSSFVGMSLFNAKNPAPMHGHVILGSDHMPILVYQISPEETRILCAYNSPKVPADIKSWMIKDVQPFIPKSLRPSFDEAVSQGKFRAMPNSYLPARQNDVTGMCVIGDALNMRHPLTGGGMTVGLHDVVLLIKKIGDLDFSDREKVLDELLDYHFERKSYDSVINVLSVALYSLFAADSDNLKALQKGCFKYFQRGGDCVNKPVEFLSGVLPKPLQLTRVFFAVAFYTIYLNMEERGFLGLPMALLEGIMILITAIRVFTPFLFGELIG

SEQ ID NO: 313 - protein, 731 aa, Saccharomyces cerevisiae
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETHTL

SEQ ID NO: 314-315 - skipped (2 entries)

SEQ ID NO: 316 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSICYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVLTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFMGYCQ

SEQ ID NO: 317 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSICYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVLTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDLSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFMGYCQ

SEQ ID NO: 318 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNLAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAVALKAIIMVQNTPGYEDLKKQVSDKRKHTAIDLLLGMQNVGSFEPGSFASYEPIRASSMLEKINPAEVFGNIMVEYPYVECTDSVALGLSNFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 319 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSDAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQEACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 320 - skipped

SEQ ID NO: 321 - protein, 741 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVNNAYEAALKNWHLFASLQDPDSGAWQSEYDGPQFMSIGYVTACYFGGNEIPTPVKTEMIRYIVNTAHPVDGGWSLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGLFEPGSFASYETIRASSMLEKINPAEVFGNIMVEYPYVECTDSVVLGLSYFRKYHDYRNEDVDRAISAAIGYIIREQQPDGGFFGSWGVCYCYAHMFAMEALETLNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYC

SEQ ID NO: 322 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKILLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGYIMVEYPYEECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 323 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGVLWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTVFAVQYACVCGFAELPLYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRTSSMLEKINPAEVFGNIMVEYPYVECTDSVVLGLSCFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 324 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSGIDFSKHCNSISGVDLYYPHTGLLKFGNALLRRYRKFRPQWINEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALVTQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRVYFVMKALGLYFKGYCQ

SEQ ID NO: 325 - protein, 742 aa, Artificial Sequence
  Synthetic
MGIHESVSKQFAKNGHSKYRSDRYGLPKTDLRRWTFHASDLGAQWWEYDDTTPLEELEKRATDYVKYSLELPGYAPVTLDSKPVKNAYEAAIKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMSSIVMYLHEGPDPANYKKIAARWPEFLSLNPSGMFMNGTNGLQVWDTAFAVQYACVCGFAELPQYQKTIRAAFDFLDRSQINEPMEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDPAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 326 - protein, 742 aa, Artificial Sequence
  Synthetic
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLVTKFGGAINNPHWGKTWLSILNLYKWEGVNPAPGELWLLPYFVPVHPGRWWVHTRWIYLAMGYLEAAEAQCELTPLLEELRDEIYIKPYSEIDFSKHCNSISGVDLYYPHTGLLKFGSALLRRYRKFRPQWIKEKVKEEIYNLCLREVSNTRHLCLAPVNNAMTSIVMYLHEGLDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYTHMFAMEALETQNLNYNNCSTVQKACDFLADYQEADGGWAEDLKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 327 - DNA, 2286 bp, Artificial Sequence
  Synthetic
atgtggagacttaagaccggttccgagactgtcggcgacaacggccgatggcttcgaagcaccaacaaccatgtgggtcgacaggtctgggagttcttccccgagatgggctcccccgaggagctggtcgccattgaggccgcccaccgagagttccacctcaatcgattccacaagcagcattcttccgacctgctgatgcgacttcagtacgagcgagagaagccatgtgttcagaaggagggcgccgttcgacttgacgcaaccgaaacccctaccgaggcggccgttgagactaccctgcgaagggcccttacattctactctactatgcagtccgacgacgggcattgggccaacgacctgggtggacccatgttcctcctacccggactggttattactctgaccatcaccggcaccatcaacgttgttctgagcaaggagcatcagcgagagattcgacgatacctctacaaccatcagaatcaggatggcggatggggcctgcatattgagggccctagcactatgttcggctctgcccttaactacgtcacgctacgacttcttggggagggccccgacgacggcgaaggtgccatggagcgtgcacgacagtggattttgagccgaggtggcgcggttgcagtcacttcttggggcaagctctggctttcggtcctgggcgtttacgagtgggatggcaacaaccccctgcctcccgaactctggttattgccctactcccttcctctccaccccggccggatgtggtgccactgccgaatggtgtaccttcccatgtcgtacttgtacggtaagcgattcgtgggtcccatcacacccacggtactcagtcttcgagaggagctctaccccatcccctaccatcatgtggactggaacaaggcccgaaacacttgtgcccaggacgatttgtactaccctcatccgttcgttcaggacctgctttggggttccctctaccacgtctacgagccccttgttatgcgatggcccggaaagcgattgcgagagagagcgcttcagcacgtcatgaagcacatacactatgaggatgagaacactgagtacatctgcctcggccccgtgaacaaggccctcaacatgctgtgttgttgggtcgaggacccccactcagaggccttcaagatgcacatcccacgcatttacgactacctctggattgcagaggatgggatgaagatgcagggttacaacggcagccagctctgggacaccgcctttgccgttcaagccattgtcgccaccaagctcactgatgagttttccgaaaccctcgccaaggcgaataagtacattctcgacgctcaaatcctgaagaactgtccaggcgaccccaacgtttggtaccgacacatcacaaagggcgcctggtccttctccactgctgaccagggctggctggtttctgactgtactgctgagggtctgaaagcccttctgctgtactccatgctgcctcaccagaaggccccctcctctatcgaaaagaaccgactgtacgacgctgtgaacgtccttctgtctatgcagaacgcggacggtggcttcgcttctttcgagttgacccgtagctacccctggctggagatgatcaaccccgctgagacatttggcgatatcgttatcgattacacctacgtcgagtgtacatccgccgttatccaggccctcgccctcttcaagcgactccatcccggtcaccgaaagaaggagatcgagcgctgcatggccaacgcggctaagtttcttgagatgcgacaggaggctgacggctcttggtacggttgctggggcgtgtgctacacctacgcaggttggttcggcatcaagggcctcacatcctgtggccgaacatacaacaactgtgccaacatcagacgagcatgcgatttcctcctctctaagcagctgcctaacggaggctggggcgaatcatacttatcctgccaaaacaagctgtacacaaacctcaataacgaccgaatgcacactgtcaacaccgcttgggcaatgatggctctgatcgaggctggccaggctaagaccgaccctatgcccttgcatcacgccgcgcgaaccctcattaacgcccagatggaaacaggagacttcccccagcaggagatcatgggcgttttcaataagaactgcatgatttcttacgcgggctaccgaaacgttttccctgtgtgggctttgggtgagtaccaccaccgagttcttaacggttgctaa

SEQ ID NO: 328 - DNA, 2229 bp, Artificial Sequence
  Synthetic
atgggaatccacgaaagtgtgtcgaaacagtttgcgaaatacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcagcgagatccccacgccggtcaaaaccgaaatgatcagatgcattgtcaacacagcccacccagttgacggaggctggggccttcataaagaagacaagagcacctgtttcggtaccagcatcaactacgtggtcctgcgactactgggcctgtcacgggatcatccggtctgcgtcaaggcgcacaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagtttgatttctccaaacattgcaactccatctccggagtcgacctctactatccccacaccggccttttgaagtttggcaacgcgcgtctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgattgattttctcgatcggtcccagatcaacgtgccgtcggaggaaaattcctatcgagacgaccgcgtcggaggatggccctttagtaccaagacccaggggtatccagtctccgactgtactgccgaggctctcaaggccagcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaagggcgtgcgactttctggcgggctaccaggaagcagatggaggctgggccgaggactttaagtcgtgcgaggctcagatgtacgtgcgcggaccccattcgctggtcgtgcctactgccatggccctgttgagtttgatgagtggtcggtatccccaggaggacaagattcatgctgcggcccggtttctcatgagcaagcagatgagcaatggtgagtggctcaaggaggagatggagggggtgtttaaccatacttgtgccattgagtatcccaactaccggttttattttgtcatgaaggctttggggttgtatttcaagggatattgccagtga

SEQ ID NO: 329 - protein, 742 aa, Artificial Sequence
  Synthetic
MGIHESVSKQFAKYGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGSEIPTPVKTEMIRCIVNTAHPVDGGWGLHKEDKSTCFGTSINYVVLRLLGLSRDHPVCVKAHKTLLTKFGGAINNPHWGKTWLSILNLYKWEGVNPAPGELWLLPYFVPVHPGRWWVHTRWIYLAMGYLEAAEAQCELTPLLEELRDEIYKKPYSEFDFSKHCNSISGVDLYYPHTGLLKFGNARLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAIDFLDRSQINVPSEENSYRDDRVGGWPFSTKTQGYPVSDCTAEALKASIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQRACDFLAGYQEADGGWAEDFKSCEAQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 330 - DNA, 2229 bp, Artificial Sequence
  Synthetic
atgggaatccacgaaagtgtgtcgaaacagtttgcgaaaaacggacattccaagtaccgcagcgaccgatacggcttacctaagacggatctgcgacaatggacgttccacgcgtccgatctgggggcgcaatggtggaagtatgacgataccacaccgctggaagagttggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcgcgggatcatccggtctgcgtcaaggcgtgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattggtttctccaaacattgcatcaccatctccggagtcgacctctactatccccacaccggccttttgaagtttggcaacgcgcttctccgacgataccgcaagttcagaccgcagtggatcaaagaaaaggtcaaggaggaaatttataacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagagtctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtttttcaagggatattgccagtga

SEQ ID NO: 331 - protein, 742 aa, Artificial Sequence
  Synthetic
MGIHESVSKQFAKNGHSKYRSDRYGLPKTDLRQWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKACKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIGFSKHCITISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQSLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLFFKGYCQ

SEQ ID NO: 332 - DNA, 2280 bp, Artificial Sequence
  Synthetic
atgtggcgactgaaggttggtgctgagtccgtaggcgaaaatgacgagaagtggctcaaatctatcagtaaccatcttggaagacaagtgtgggagttttgccctgatgccgggactcagcagcagctgttgcaggtccacaaggcccgtaaggcattccacgacgaccgattccaccgaaagcagtcgtctgaccttttcattaccatccagtatggtaaggaggttgagaacggtggcaagaccgctggtgtcaagctgaaggagggtgaggaggtccgcaaggaggccgttgagtcctctctcgaacgagcgctctccttttactcctctattcagacctccgatggcaactgggccagcgatctgggtggtcccatgttcttactgcccggattggtcatcgccctctacgttacgggtgtgctaaactctgttctgtccaagcaccatcgacaggagatgtgtcggtacgtctacaaccaccaaaacgaggacggtggctggggtcttcacattgagggaccatctaccatgttcggttcagctctaaactacgtcgccctccgactgcttggggaggacgctaacgccggtgcaatgcccaaggctcgagcctggatcctcgaccacggcggtgctactggtatcacctcctggggtaagctctggctgagtgtgcttggcgtctacgagtggtccggcaacaaccccctccctcccgagttctggctgtttccctacttcctgcctttccatcccggaaggatgtggtgtcactgccgaatggtctacttgcccatgtcttatctctacggtaagcgattcgttggtcccatcacccctatcgtcctgtcccttcgaaaggagctttacgccgtcccgtaccacgagattgattggaacaagtcccgaaacacttgtgccaaggaggacctctactaccctcaccccaagatgcaggacattctgtggggctcccttcatcacgtgtacgagcccctgttcacccgatggcccgctaagcgacttcgagagaaggccttgcagacagccatgcagcacatccactacgaagacgaaaatacccgatacatctgcctgggtcccgtcaacaaggttctgaacctcttgtgttgttgggtcgaggatccctactctgatgctttcaaactccacctccagcgagttcacgactacctgtgggttgccgaggacggaatgaagatgcagggatacaacggttctcagctctgggatactgcattttcgattcaggccatcgtcagcaccaagctggtagacaactacggaccgacactccgaaaggctcacgactttgttaagtcttcccagatccaacaggactgccccggtgatcccaacgtctggtacagacacattcataaaggtgcctggcctttctccacccgtgaccacggctggctcatttctgattgtaccgctgagggccttaaggccgccctgatgctgtccaagctcccctctgagactgtgggtgagtcgctcgagcgaaaccgactttgcgacgccgtgaacgttctccttagtctccagaacgacaacggtggtttcgcttcctatgagctgacccgttcctacccctggcttgaactgattaaccctgcggagacattcggtgatatcgtcatcgactacccctacgttgagtgtacgtctgccaccatggaggctcttaccctgtttaagaagctccatcctggtcaccgaaccaaggagattgacaccgccatcgtccgagccgctaatttcctggagaacatgcagcgaaccgacgggtcatggtacggttgctggggagtctgttttacctacgccggatggttcggtattaagggtcttgtcgccgctggccgaacttacaacaactgtttggccatcagaaaggcctgcgacttcctcctgtctaaggagctgcccggaggtggctggggcgaatcctatctctcctgtcagaataaggtttacaccaacttagagggtaacaggccccacttggtgaatactgcttgggttcttatggcgctgatcgaggccggtcaggcggagcgagatcccacccccctacaccgagctgcccgactgcttatcaactcccagctcgagaacggtgacttccctcaacaggagattatgggtgtttttaacaagaactgcatgatcacgtacgccgcctaccgaaacatcttccctatctgggctcttggtgaatactgtcaccgagtcctgaccgagtaa

(19) United States
(12) Patent Application Publication
(10) Pub. No.: US 2024/0218403 A1
(43) Pub. Date: Jul. 4, 2024

(54) BIOSYNTHESIS OF ISOPRENOIDS AND PRECURSORS THEREOF

(71) Applicant: Ginkgo Bioworks, Inc., Boston, MA (US)

(72) Inventors: Guillaume Beaudoin, Cambridge, MA (US); Elena Brevnova, Winchester, MA (US); Alkiviadis Orfefs Chatzivasileiou, Boston, MA (US); Alexandra Exner, Boston, MA (US); Annapuma Kamineni, Watertown, MA (US); Matthew McMahon, Melrose, MA (US); Joshua Trueheart, Walttam, MA (US)

(73) Assignee: Ginkgo Bioworks, Inc., Boston, MA (US)

(21) Appl. No.: 18/285,138

(22) PCT Filed: Apr. 1, 2022
(86) PCT No.: PCT/US2022/023165

§ 371 Date: Sep. 29, 2023

Related U.S. Application Data
(60) Provisional application No. 63/170,347, filed on Apr. 2, 2021.

(51) Int. Cl. C12P 7/42; C12N 1/16; C12N 1/20; C12N 9/04; C12N 9/10; C12N 9/12; C12N 9/88; C12N 9/90; C12P 17/02; C12P 33/00; C12R 1/19; C12R 1/865

(52) U.S. Cl. CPC C12P 7/42; C12N 1/165; C12N 1/205; C12N 9/0006; C12N 9/1029; C12N 9/1205; C12N 9/1229; C12N 9/88; C12N 9/90; C12P 17/02; C12P 33/00; C12R 2001/19; C12R 2001/865; C12Y 101/01034; C12Y 101/01088; C12Y 203/01009; C12Y 203/0301; C12Y 207/01036; C12Y 207/04002; C12Y 207/04026; C12Y 401/01033; C12Y 503/03002; C12Y 504/99007

(57) ABSTRACT

Described in this application are proteins and host cells involved in methods of producing isoprenoid precursors and/or isoprenoids.

### CROSS-REFERENCE TO RELATED APPLICATIONS

This application claims the benefit under 35 U.S.C. § 119(e) of U.S. Provisional Application No. 63/170,347, filed Apr. 2, 2021, entitled “BIOSYNTHESIS OF ISOPRENOIDS AND PRECURSORS THEREOF,” the entire disclosure of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Apr. 1, 2022, is named G091970078W000-SEQ-FL.TXT and is 392,553 bytes in size.

### FIELD OF THE INVENTION

The present disclosure relates to the production of isoprenoid precursors and isoprenoids in recombinant cells.

### BACKGROUND

Isoprenoids are a diverse class of organic compounds derived from five carbon building blocks and encompass at least 50,000 compounds. Given their structural diversity, isoprenoids have numerous uses as flavoring agents, fragrance compounds, antioxidants, and medicinal compounds. Although the mevalonate biosynthesis pathway has been characterized and is used by eukaryotes, archaea, and some bacteria to produce isoprenoids, the wide array of isoprenoid isomers often hinder high yield extractions from naturally occurring sources. Furthermore, the structural complexity of isoprenoids often limits de novo chemical synthesis.

### SUMMARY

Aspects of the disclosure relate to host cells for producing an isoprenoid precursor or isoprenoid. In some embodiments, the host cell comprises a heterologous polynucleotide encoding a lanosterol synthase with reduced activity as compared to a wild-type lanosterol synthase, wherein the host cell is capable of producing more of an isoprenoid or isoprenoid precursor as compared to a control host cell that does not comprise the heterologous polynucleotide.

In some embodiments, the wild-type lanosterol synthase comprises SEQ ID NO: 1 or SEQ ID NO: 313.

In some embodiments, the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

In some embodiments, the lanosterol synthase comprises 2, 3, 4, 5, 6, 7, 8, 9, 10, 11, or 12 amino acid substitutions and/or deletions relative to SEQ ID NO: 1.

In some embodiments, the lanosterol synthase comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO:1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO:1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO:1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO:1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO:1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO:1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1.

In some embodiments, the lanosterol synthase comprises the amino acid substitution E617V, G107D, and/or K631E relative to SEQ ID NO: 1.

In some embodiments, relative to SEQ ID NO: 1, the lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

In some embodiments, the lanosterol synthase comprises relative to SEQ ID NO: 1: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, lanosterol synthase comprises a sequence that is at least 90% identical to SEQ ID NO: 3, 83-87, 89-92, 94-95, 99, 118-120, 316-319, 321-326, 329, or 331.

In some embodiments, the lanosterol synthase comprises SEQ ID NO: 3, 83-87, 89-92, 94-95, 99, 118-120, 316-319, 321-326, 329, or 331.

In some embodiments, the heterologous polynucleotide comprises a sequence that is at least 90% identical to SEQ ID NO: 4, 62-66, 68-71, 73-74, 78, 103-109, 111-117, 328, or 330.

In some embodiments, the heterologous polynucleotide comprises the sequence of SEQ ID NO: 4, 62-66, 68-71, 73-74, 78, 103-109, 111-117, 328, or 330.

Further aspects of the disclosure relate to host cells that comprise a heterologous polynucleotide encoding a lanosterol synthase, wherein the lanosterol synthase comprises a sequence that is at least 90% identical to SEQ ID NO: 3, 83-87, 89-92, 94-95, 99, 100-102, 118-120, 316-319, 321-326, 329, or 331.

In some embodiments, the lanosterol synthase comprises SEQ ID NO: 3, 83-87, 89-92, 94-95, 99, 100-102, 118-120, 316-319, 321-326, 329, or 331.

Further aspects of the disclosure relate to host cells that comprise a heterologous polynucleotide encoding a lanosterol synthase, wherein the lanosterol synthase comprises relative to SEQ ID NO: 1: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, 1479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

Further aspects of the disclosure relate to host cells that comprise a heterologous polynucleotide encoding a lanosterol synthase, wherein the heterologous polynucleotide comprises a sequence that is at least 90% identical to SEQ ID NO: 4, 62-66, 68-71, 73-74, 78, 80-82, 103-109, 111-117, 328, or 330.

In some embodiments, the heterologous polynucleotide comprises SEQ ID NO: 4, 62-66, 68-71, 73-74, 78, 80-82, 103-109, 111-117, 328, or 330.

In some embodiments, the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

In some embodiments, the lanosterol synthase comprises a sequence that is at least 90% identical to any one of SEQ ID NOs: 100-102.

In some embodiments, the lanosterol synthase comprises a sequence selected from SEQ ID NOs: 100-102.

In some embodiments, the heterologous polynucleotide encoding the lanosterol synthase comprises a sequence that is at least 90% identical to a sequence selected from SEQ ID NOs: 80-82.

In some embodiments, the heterologous polynucleotide encoding the lanosterol synthase comprises a sequence selected from SEQ ID NOs: 80-82.

In some embodiments, the host cell is capable of producing mevalonate.

In some embodiments, the host cell is capable of producing at least 0.2 g/L mevalonate.

In some embodiments, the host cell is capable of producing at least 0.7 g/L mevalonate.

In some embodiments, the host cell is capable of producing at least 9 mg/L of an isoprenoid.

In some embodiments, the host cell is capable of producing at least 1.1 fold more of an isoprenoid than a control host cell comprising SEQ ID NO: 1 and/or a control host cell comprising SEQ ID NO: 313.

In some embodiments, the host cell is capable of producing at least 3 fold more of an isoprenoid than a control host cell comprising SEQ ID NO: 1 and/or a control host cell comprising SEQ ID NO: 313.

In some embodiments, the host cell is capable of producing at most 200 mg/L lanosterol.

In some embodiments, the host cell is capable of producing at least 5 mg/L oxidosqualene.

In some embodiments, the host cell is capable of producing more mevalonate than a control host cell that does not comprise the heterologous polynucleotide encoding the lanosterol synthase with reduced activity as compared to the wild-type lanosterol synthase.

In some embodiments, the host cell is capable of producing more 2-3-oxidosqualene as compared to a host cell that does not comprise the heterologous polynucleotide encoding the lanosterol synthase with reduced activity as compared to the wild-type lanosterol synthase.

In some embodiments, the host cell further comprises: (a) a heterologous polynucleotide encoding a squalene epoxidase with reduced activity as compared to a wild-type squalene epoxidase; or (b) a heterologous polynucleotide that reduces squalene epoxidase activity, wherein the host cell is capable of producing more of an isoprenoid or isoprenoid precursor as compared to a control host cell that does not comprise the heterologous polynucleotide of (a) and/or (b).

In some embodiments, the wild-type squalene epoxidase comprises SEQ ID NO: 9 or 312.

Further aspects of the disclosure relate to host cells for producing an isoprenoid precursor or isoprenoid, wherein the host cell comprises: (a) a heterologous polynucleotide encoding a squalene epoxidase with reduced activity as compared to a wild-type squalene epoxidase; or (b) a heterologous polynucleotide that reduces squalene epoxidase activity, wherein the host cell is capable of producing more of an isoprenoid or isoprenoid precursor as compared to a control host cell that does not comprise the heterologous polynucleotide of (a) and/or (b).

In some embodiments, the wild-type squalene epoxidase comprises SEQ ID NO: 9 or 312.

In some embodiments, the heterologous polynucleotide encodes a squalene epoxidase that comprises 1, 2, 3, 4, 5, 6, 7, 8, 9, or 10 amino acid substitutions and/or deletions relative to SEQ ID NO: 9 or 312.

In some embodiments, the host cell is capable of producing mevalonate.

In some embodiments, the host cell is capable of producing at least 0.2 g/L mevalonate.

In some embodiments, the host cell is capable of producing at least 0.7 g/L mevalonate.

In some embodiments, the host cell is capable of producing more mevalonate than a control host cell that does not comprise (a) the heterologous polynucleotide encoding the squalene epoxidase with reduced activity as compared to the wild-type squalene epoxidase; and/or (b) the heterologous polynucleotide that reduces squalene epoxidase activity.

In some embodiments, the host cell is capable of producing more 2-3-oxidosqualene as compared to a host cell that does not comprise (a) the heterologous polynucleotide encoding the squalene epoxidase with reduced activity as compared to the wild-type squalene epoxidase; and/or (b) the heterologous polynucleotide that reduces squalene epoxidase activity.

In some embodiments, the host cell further comprises: (a) a heterologous polynucleotide encoding a lanosterol synthase with reduced activity as compared to a lanosterol synthase; or (b) a heterologous polynucleotide that reduces lanosterol synthase activity, wherein the host cell is capable of producing more of an isoprenoid or isoprenoid precursor as compared to a control host cell that does not comprise the heterologous polynucleotide of (a) and/or (b).

In some embodiments, the wild-type lanosterol synthase comprises SEQ ID NO: 1 or 313.

Further aspects of the disclosure relates to host cells comprising: (a) one or more enzymes in the yeast mevalonate pathway; and (b) a heterologous polynucleotide encoding a lanosterol synthase with reduced activity as compared to a wild-type lanosterol synthase; and/or (c) a heterologous polynucleotide encoding a squalene epoxidase with reduced activity as compared to a wild-type squalene epoxidase; or (d) a heterologous polynucleotide that reduces squalene epoxidase activity.

In some embodiments, the one or more enzymes in the yeast mevalonate pathway is selected from an enzyme with one of the following enzyme classification numbers: EC 2.3.1.9, EC 2.3.3.10, EC 1.1.1.88, EC 1.1.1.34, EC 2.7.1.36, EC 2.7.4.2, EC 4.1.1.33, and/or EC 5.3.3.2,

Further aspects of the disclosure provide host cells comprising: (a) one or more enzymes in the Archaea I mevalonate pathway; and (b) a heterologous polynucleotide encoding a lanosterol synthase with reduced activity as compared to a wild-type lanosterol synthase; or (c) a heterologous polynucleotide that reduces lanosterol synthase activity; and/or (d) a heterologous polynucleotide encoding a squalene epoxidase with reduced activity as compared to a wild-type squalene epoxidase; or (e) a heterologous polynucleotide that reduces squalene epoxidase activity.

In some embodiments, the one or more enzymes in the archaea I mevalonate pathway is selected from an enzyme with one of the following enzyme classification numbers: EC 4.1.1.99, EC 2.7.4.26, EC 2.3.1.9, EC 2.3.3.10, EC 1.1.1.88, EC 1.1.1.34, EC 2.7.1.36, and/or EC 5.3.3.2.

Further aspects of the disclosure provide host cells comprising: (a) one or more enzymes in the Archaea II mevalonate pathway; and (b) a heterologous polynucleotide encoding a lanosterol synthase with reduced activity as compared to a wild-type lanosterol synthase; or (c) a heterologous polynucleotide that reduces lanosterol synthase activity; and/or (d) a heterologous polynucleotide encoding a squalene epoxidase with reduced activity as compared to a wild-type squalene epoxidase; or (e) a heterologous polynucleotide that reduces squalene epoxidase activity.

In some embodiments, the one or more enzymes in the Archaea II mevalonate pathway is selected from an enzyme with one of the following enzyme classification numbers: EC 2.7.1.185, EC 2.7.1.186, EC 2.7.4.26, EC 4.1.1.99, EC 2.3.1.9, EC 2.3.3.10, EC 1.1.1.88, EC 1.1.1.34, EC 2.7.1.36, and/or EC 5.3.3.2.

Further aspects of the disclosure provide host cell comprising: (a) one or more enzymes in the MEP pathway; and (b) a heterologous polynucleotide encoding a lanosterol synthase with reduced activity as compared to a wild-type lanosterol synthase; or (c) a heterologous polynucleotide that reduces lanosterol synthase activity; and/or (d) a heterologous polynucleotide encoding a squalene epoxidase with reduced activity as compared to a wild-type squalene epoxidase; or (e) a heterologous polynucleotide that reduces squalene epoxidase activity.

In some embodiments, the one or more enzymes in the MEP pathway is selected from an enzyme with one of the following enzyme classification numbers: EC 2.2.1.7, EC 1.1.1.267, EC 2.7.7.60, EC 2.7.1.148, EC 4.6.1.12, EC 1.17.7.1, and/or EC 1.17.1.2.

In some embodiments, the host cell is a yeast cell, a plant cell, or a bacterial cell.

In some embodiments, the host cell is a yeast cell.

In some embodiments, the yeast cell is a Saccharomyces cerevisiae cell.

In some embodiments, the yeast cell is a Yarrowia lipolytica cell.

In some embodiments, the host cell is a bacterial cell.

In some embodiments, the bacterial cell is an E. coli cell.

Further aspects of the present disclosure provide methods of producing mevalonate comprising culturing any of the host cells associated with the disclosure.

Further aspects of the present disclosure provide methods of producing an isoprenoid precursor or isoprenoid comprising culturing any of the host cells associated with the disclosure.

Further aspects of the present disclosure relate to methods of producing 2-C-Methyl-d-erythritol-2,4-cyclopyrophosphate (MEcPP) comprising culturing any of the host cells associated with the disclosure.

Further aspects of the present disclosure relates to method of producing an isoprenoid precursor or isoprenoid comprising culturing a host that comprises: (a) a heterologous polynucleotide encoding a lanosterol synthase with reduced activity as compared to a wild-type lanosterol synthase; and/or (b) a heterologous polynucleotide encoding a squalene epoxidase with reduced activity as compared to a wild-type squalene epoxidase; or (c) a heterologous polynucleotide that reduces squalene epoxidase activity, wherein the host cell is capable of producing more of an isoprenoid or isoprenoid precursor as compared to a control host cell that does not comprise one or more of (a)-(c).

In some embodiments, the wild-type lanosterol synthase comprises SEQ ID NO: 1 or 313.

In some embodiments, the heterologous polynucleotide in (a) encodes a lanosterol synthase that comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

In some embodiments, the heterologous polynucleotide in (a) encodes a lanosterol synthase that comprises 2, 3, 4, 5, 6, 7, 8, 9, 10, 11, or 12 amino acid substitutions and/or deletions relative to SEQ ID NO: 1.

In some embodiments, the heterologous polynucleotide encoding the lanosterol synthase with reduced activity encodes a lanosterol synthase that comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO:1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO:1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO:1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO:1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO:1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO:1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1.

In some embodiments, the heterologous polynucleotide encoding the lanosterol synthase with reduced activity encodes a lanosterol synthase that comprises the amino acid substitution E617V, G107D, and/or K631E relative to SEQ ID NO: 1.

In some embodiments, the heterologous polynucleotide encoding the lanosterol synthase with reduced activity encodes a lanosterol synthase that comprises relative to SEQ ID NO: 1, the lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

In some embodiments, the heterologous polynucleotide encodes a lanosterol synthase that comprises relative to SEQ ID NO: 1: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the heterologous polynucleotide encodes a lanosterol synthase that comprises a sequence that is at least 90% identical to SEQ ID NO: 3, 83-87, 89-92, 94-95, 99, 118-120, 316-319, 321-326, 329, or 331.

In some embodiments, the lanosterol synthase comprises SEQ ID NO: 3, 83-87, 89-92, 94-95, 99, 118-120, 316-319, 321-326, 329, or 331.

In some embodiments, the heterologous polynucleotide encoding the lanosterol synthase comprises a sequence that is at least 90% identical to SEQ ID NO: 4, 62-66, 68-71, 73-74, 78, 103-109, 111-117, 328, or 330.

In some embodiments, the heterologous polynucleotide comprises the sequence of SEQ ID NO: 4, 62-66, 68-71, 73-74, 78, 103-109, 111-117, 328, or 330.

In some embodiments, the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

In some embodiments, the lanosterol synthase comprises a sequence that is at least 90% identical to any one of SEQ ID NOs: 100-102.

In some embodiments, the lanosterol synthase comprises a sequence selected from SEQ ID NOs: 100-102.

In some embodiments, the heterologous polynucleotide encoding the lanosterol synthase comprises a sequence that is at least 90% identical to a sequence selected from SEQ ID NOs: 80-82.

In some embodiments, the heterologous polynucleotide encoding the lanosterol synthase comprises a sequence selected from SEQ ID NOs: 80-82.

In some embodiments, the host cell is capable of producing mevalonate.

In some embodiments, the host cell is capable of producing at least 0.2 g/L mevalonate.

In some embodiments, the host cell is capable of producing at least 0.7 g/L mevalonate.

In some embodiments, the host cell is capable of producing at least 9 mg/L of an isoprenoid.

In some embodiments, the host cell is capable of producing at least 1.1 fold more of an isoprenoid than a control host cell comprising SEQ ID NO: 1 and/or a control host cell comprising SEQ ID NO: 313.

In some embodiments, the host cell is capable of producing at least 3 fold more of an isoprenoid than a control host cell comprising SEQ ID NO: 1 and/or a control host cell comprising SEQ ID NO: 313.

In some embodiments, the host cell is capable of producing at most 200 mg/L lanosterol.

In some embodiments, the host cell is capable of producing at least 5 mg/L oxidosqualene.

In some embodiments, the host cell is capable of producing more mevalonate than a control host cell that does not comprise: (a) the heterologous polynucleotide encoding the lanosterol synthase with reduced activity as compared to a wild-type lanosterol synthase; and/or (b) the heterologous polynucleotide encoding the squalene epoxidase with reduced activity as compared to a wild-type squalene epoxidase; or (c) the heterologous polynucleotide that reduces squalene epoxidase activity.

In some embodiments, the host cell is capable of producing more 2-3-oxidosqualene as compared to a host cell that does not comprise: (a) the heterologous polynucleotide encoding the lanosterol synthase with reduced activity as compared to a wild-type lanosterol synthase; and/or (b) the heterologous polynucleotide encoding the squalene epoxidase with reduced activity as compared to a wild-type squalene epoxidase; or (c) the heterologous polynucleotide that reduces squalene epoxidase activity.

In some embodiments, the wild-type squalene epoxidase comprises SEQ ID NO: 9 or 312.

In some embodiments, the heterologous polynucleotide encodes a squalene epoxidase comprises 1, 2, 3, 4, 5, 6, 7, 8, 9, or 10 amino acid substitutions and/or deletions relative to SEQ ID NO: 9 or 312.

In some embodiments, the host cell is a yeast cell, a plant cell, or a bacterial cell.

In some embodiments, the host cell is a yeast cell.

In some embodiments, the yeast cell is a Saccharomyces cerevisiae cell.

In some embodiments, the yeast cell is a Yarrowia lipolytica cell.

In some embodiments, the host cell is a bacterial cell.

In some embodiments, the bacterial cell is an E. coli cell.

In some embodiments, the isoprenoid precursor is mevalonate, 2-C-Methyl-d-erythritol-2,4-cyclopyrophosphate (MEcPP), and/or 2-3-oxidosqualene.

In some embodiments, the host cell is capable of producing more of an isoprenoid or isoprenoid precursor as compared to a control host cell that does not comprise: the heterologous polynucleotide encoding the lanosterol synthase with reduced activity as compared to the control wild-type lanosterol synthase; and/or the heterologous polynucleotide encoding the squalene epoxidase with reduced activity as compared to the control wild-type squalene epoxidase; or the heterologous polynucleotide that reduces squalene epoxidase activity.

In some embodiments, the host cell is capable of producing more of an isoprenoid or isoprenoid precursor as compared to a control host cell that does not comprise: the heterologous polynucleotide encoding the lanosterol synthase with reduced activity as compared to the control wild-type lanosterol synthase; the heterologous polynucleotide that reduces lanosterol synthase activity; and/or the heterologous polynucleotide encoding the squalene epoxidase with reduced activity as compared to the control wild-type squalene epoxidase; or the heterologous polynucleotide that reduces squalene epoxidase activity.

In some embodiments, the wild-type lanosterol synthase comprises SEQ ID NO: 1 or 313.

In some embodiments, the wild-type squalene epoxidase comprises SEQ ID NO: 9 or 312.

In some embodiments, the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

In some embodiments, the heterologous polynucleotide encoding the lanosterol synthase with reduced activity encodes a lanosterol synthase that comprises 2, 3, 4, 5, 6, 7, 8, 9, 10, 11, or 12 amino acid substitutions and/or deletions relative to SEQ ID NO: 1.

In some embodiments, the heterologous polynucleotide encoding the lanosterol synthase with reduced activity encodes a lanosterol synthase that comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO:1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO:1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO:1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO:1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO:1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO:1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1.

In some embodiments, the heterologous polynucleotide encoding the lanosterol synthase with reduced activity encodes a lanosterol synthase that comprises the amino acid substitution E617V, G107D, and/or K631E relative to SEQ ID NO: 1.

In some embodiments, the heterologous polynucleotide encoding the lanosterol synthase with reduced activity encodes a lanosterol synthase that comprises relative to SEQ ID NO: 1, the lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO: 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E. In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

In some embodiments, the heterologous polynucleotide encodes a lanosterol synthase that comprises relative to SEQ ID NO: 1: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the heterologous polynucleotide encodes a lanosterol synthase that comprises a sequence that is at least 90% identical to SEQ ID NO: 33, 83-87, 89-92, 94-95, 99, 118-120, 316-319, 321-326, 329, or 331.

In some embodiments, the lanosterol synthase comprises SEQ ID NO: 33, 83-87, 89-92, 94-95, 99, 118-120, 316-319, 321-326, 329, or 331.

In some embodiments, the heterologous polynucleotide encoding the lanosterol synthase comprises a sequence that is at least 90% identical to SEQ ID NO: 4, 62-66, 68-71, 73-74, 78, 103-109, 111-117, 328, or 330.

In some embodiments, the heterologous polynucleotide comprises the sequence of SEQ ID NO: 4, 62-66, 68-71, 73-74, 78, 103-109, 111-117, 328, or 330.

In some embodiments, the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

In some embodiments, the lanosterol synthase comprises a sequence that is at least 90% identical to any one of SEQ ID NOs: 100-102.

In some embodiments, the lanosterol synthase comprises a sequence selected from SEQ ID NOs: 100-102.

In some embodiments, the heterologous polynucleotide encoding the lanosterol synthase comprises a sequence that is at least 90% identical to a sequence selected from SEQ ID NOs: 80-82.

In some embodiments, the heterologous polynucleotide encoding the lanosterol synthase comprises a sequence selected from SEQ ID NOs: 80-82.

In some embodiments, the host cell is capable of producing mevalonate.

In some embodiments, the host cell is capable of producing at least 0.2 g/L mevalonate.

In some embodiments, the host cell is capable of producing at least 0.7 g/L mevalonate.

In some embodiments, the host cell is capable of producing more mevalonate than a control host cell that does not comprise: (a) the heterologous polynucleotide encoding the lanosterol synthase with reduced activity as compared to a wild-type lanosterol synthase; and/or (b) the heterologous polynucleotide encoding the squalene epoxidase with reduced activity as compared to a wild-type squalene epoxidase; or (c) the heterologous polynucleotide that reduces squalene epoxidase activity.

In some embodiments, the host cell is capable of producing more mevalonate than a control host cell that does not comprise: (a) the heterologous polynucleotide encoding the lanosterol synthase with reduced activity as compared to a wild-type lanosterol synthase; or (b) the heterologous polynucleotide that reduces lanosterol synthase activity; and/or (c) the heterologous polynucleotide encoding the squalene epoxidase with reduced activity as compared to a wild-type squalene epoxidase; or (d) the heterologous polynucleotide that reduces squalene epoxidase activity.

In some embodiments, the host cell is capable of producing more 2-3-oxidosqualene as compared to a host cell that does not comprise: the heterologous polynucleotide encoding the lanosterol synthase with reduced activity as compared to a wild-type lanosterol synthase; and/or the heterologous polynucleotide encoding the squalene epoxidase with reduced activity as compared to a wild-type squalene epoxidase; or the heterologous polynucleotide that reduces squalene epoxidase activity.

In some embodiments, the host cell is capable of producing more 2-3-oxidosqualene as compared to a host cell that does not comprise: the heterologous polynucleotide encoding the lanosterol synthase with reduced activity as compared to a wild-type lanosterol synthase; or the heterologous polynucleotide that reduces lanosterol synthase activity; and/or the heterologous polynucleotide encoding the squalene epoxidase with reduced activity as compared to a wild-type squalene epoxidase; or the heterologous polynucleotide that reduces squalene epoxidase activity.

In some embodiments, the heterologous polynucleotide encoding the squalene epoxidase with reduced activity encodes a squalene epoxidase comprises 1, 2, 3, 4, 5, 6, 7, 8, 9, or 10 amino acid substitutions and/or deletions relative to SEQ ID NO: 9 or 312.

In some embodiments, the host cell is a yeast cell, a plant cell, or a bacterial cell.

In some embodiments, the host cell is a yeast cell.

In some embodiments, the yeast cell is a Saccharomyces cerevisiae cell.

In some embodiments, the yeast cell is a Yarrowia lipolytica cell.

In some embodiments, the host cell is a bacterial cell.

In some embodiments, the bacterial cell is an E. coli cell.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1A-1D provide four biosynthetic pathways for forming the isoprenoid precursors isopentenyl pyrophosphate (IPP) and dimethylallyl pyrophosphate (DMPP) from acetyl-CoA including: the mevalonate (MEV) pathway from Saccharomyces cerevisiae (FIG. 1A), Archaea I (FIG. 1B) and Archaea II (FIG. 1C), as well as the non-mevalonate or methylerithritol phosphate (MEP) pathway (FIG. 1D) found in eubacteria, algae, and plant plastids. Structures of intermediates and pathway enzymes are shown.

FIG. 2 shows a sterol biosynthesis pathway in which IPP and DMPP are converted various multiple enzymatic steps to lanosterol. ERG7 is shown as a non-limiting example of a lanosterol synthase.

FIG. 3 is a graph depicting mevalonate production by Yarrowia strains comprising a lanosterol synthase.

FIG. 4 is a graph depicting cucurbitadienol production by strains comprising a lanosterol synthase (erg7 allele). Strain 870688 comprising SEQ ID NO: 1 was used as a control.

FIG. 5 is a graph depicting cucurbitadienol, ergosterol, lanosterol, and mevalonate production by strains comprising a lanosterol synthase (erg7 allele). Strain 887779 comprising SEQ ID NO: 1 was used as a control.

FIG. 6 is a graph depicting oxidosqualene production in lanosterol synthase temperature sensitive mutant (erg7 mutant) strains at 30° C. and 35° C. Three lanosterol synthase mutant strains 756247, 756248 and 756249 comprising SEQ ID NOs: 100-102, respectively, were tested and the parent BY4742 Saccharomyces cerevisiae strain was included as the negative control.

FIG. 7 is a graph depicting production of ergosterol, ethanol, and mevalonate and consumption of glucose in lanosterol synthase temperature sensitive mutant (erg7 mutant) strains at 30° C. Three lanosterol synthase mutant strains 756247, 756248 and 756249 comprising SEQ ID NOs: 100-102, respectively, were tested and the parent BY4742 Saccharomyces cerevisiae strain was included as the negative control.

FIG. 8 is a graph depicting production of ergosterol, ethanol, and mevalonate and consumption of glucose in lanosterol synthase temperature sensitive mutant (erg7 mutant) strains at 35° C. Three lanosterol synthase mutant strains 756247, 756248 and 756249 comprising SEQ ID NOs: 100-102, respectively, were tested and the parent BY4742 Saccharomyces cerevisiae strain was included as the negative control.

### DETAILED DESCRIPTION

The structural diversity of isoprenoids renders these compounds suitable for numerous applications, including use as flavoring agents, production of pharmaceutical drugs, and use as fragrance compounds. However, purification of isoprenoids from natural sources and de novo chemical synthesis often have high production costs and low yield. Furthermore, while the mevalonate pathway is used by eukaryotes and other natural sources to produce building blocks for isoprenoids, bottlenecks and production of off-target compounds limit flux through the mevalonate pathway.

This disclosure is premised, in part, on the unexpected finding that hypomorphic lanosterol synthases and/or squalene epoxidases (SQE) can be leveraged to increase production of isoprenoids and isoprenoid precursors. In some embodiments, the lanosterol synthase variant is encoded by a variant of the ERG7 coding sequence. In some embodiments, the squalene epoxidase is encoded by the ERG1 gene. Accordingly, provided herein, in some embodiments, are host cells that are engineered to efficiently produce isoprenoids and precursors thereof. Methods include heterologous expression of lanosterol synthases and/or squalene epoxidases. Examples 1 and 3-4 describe the identification and functional characterization of lanosterol synthases that can be used to increase isoprenoids and isoprenoid production. Proteins and host cells described in this disclosure can be used for making isoprenoids and precursors thereof.

### Synthesis of Isoprenoids and Precursors Thereof

Isoprenoids and precursors thereof can be synthesized from acetyl-CoA through a mevalonate intermediate (mevalonate (“MEV” or “MVA”) pathway) or from pyruvate and glyceraldehyde-3-phosphate through a 1-deoxyxylulose-5-phosphate (DXP) intermediate (non-mevalonate or methylerythritol phosphate (MEP) pathway).

Synthesis of isoprenoids in many eukaryotes such as yeasts, archaea, and some bacteria begins with the MEV pathway, which converts acetyl-CoA to isopentenyl pyrophosphate (IPP). In the mevalonate pathway, two acetyl-COA molecules are condensed to form acetoacetyl-CoA, which is in turn condensed to form 3-hydroxy-3-methyl-glutaryl-CoA (HMG-CoA). Then, HMG-CoA is reduced to form mevalonate.

From mevalonate, the isoprenoid precursor IPP can be formed in three ways.

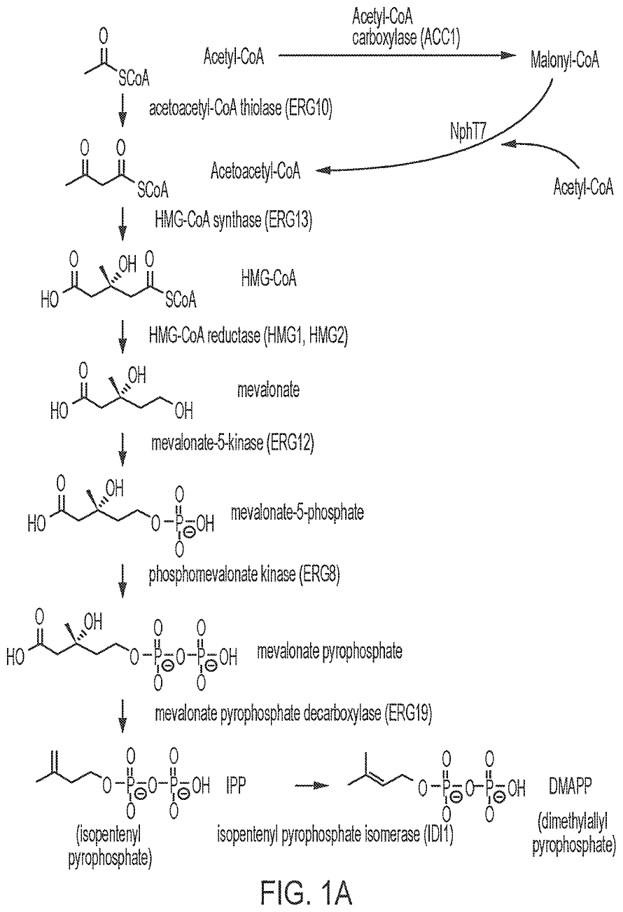

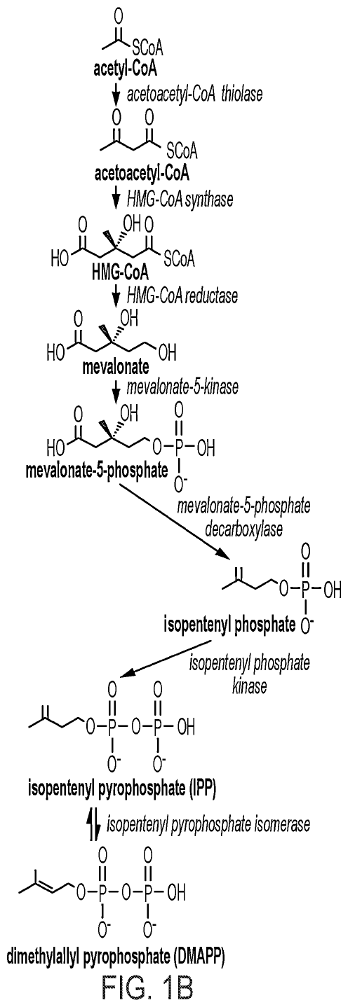

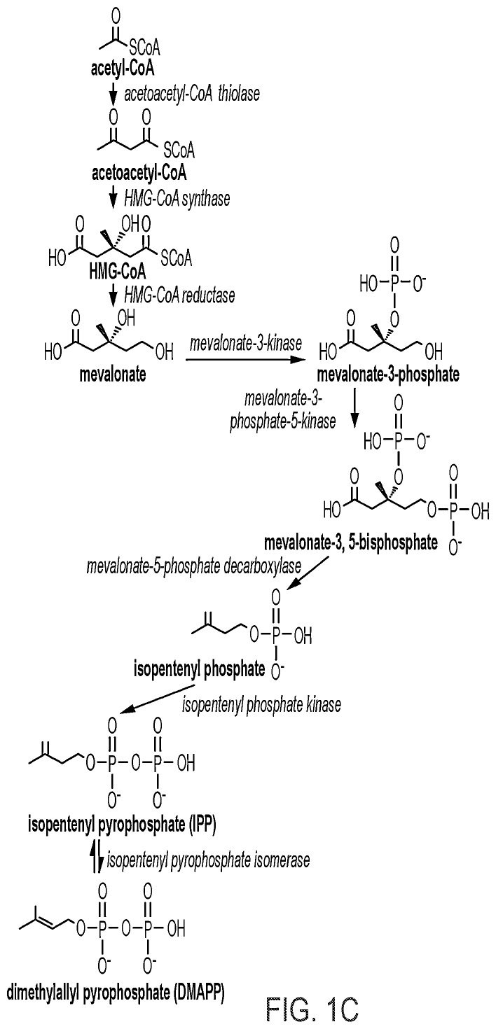

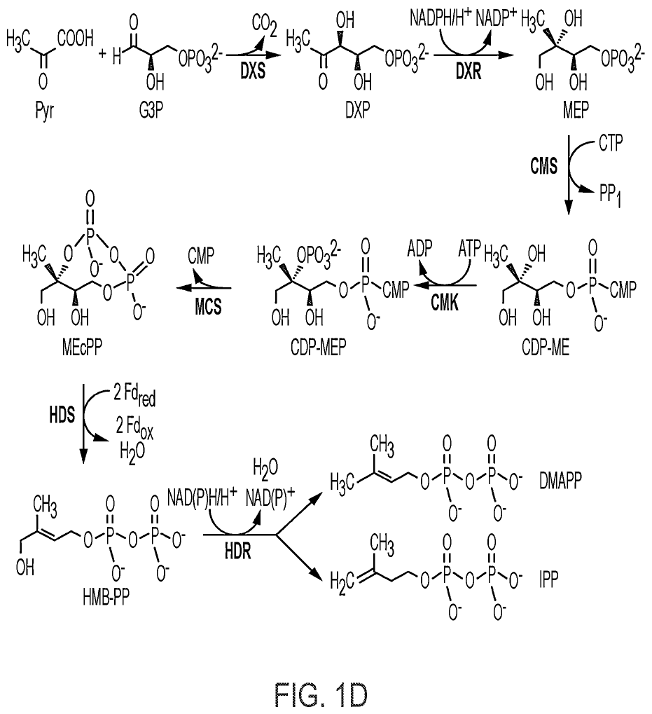

As shown in FIG. 1A, mevalonate can be phosphorylated to form mevalonate-5-phosphate, which can be phosphorylated to form mevalonate pyrophosphate. Mevalonate pyrophosphate can be decarboxylated to form IPP. IPP can then be isomerized to form dimethylallyl pyrophosphate (DMAPP). Exemplary enzymes useful form forming IPP from acetyl-CoA as shown in FIG. 1A (yeast MEV pathway) are within the classes summarized in the following table.

TABLE 1
Non-limiting Examples of Enzymes in the Yeast MEV Pathway

| Gene | Enzyme | EC |
|---|---|---|
| ERG10, ACAT | Acetoacetyl-CoA thiolase; Acetyl-CoA C-acetyltransferase; thiolase II | EC 2.3.1.9 |
| ERG13 | hydroxymethylglutaryl-CoA synthase or HMG-CoA synthase | EC 2.3.3.10 |
| HMGCR | NAD+ HMG-CoA reductase (3-hydroxy-3-methyl-glutaryl-coenzyme A | EC 1.1.1.88 |
| | reductase) | |
| HMG1, HMG2, | NADP+ HMG-CoA reductase | EC 1.1.1.34 |
| HMGCR | | |
| ERG12 | Mevalonate kinase, mevalonate-5-kinase | EC 2.7.1.36 |
| ERG8 | Phosphomevalonate kinase | EC 2.7.4.2 |
| MVD1 | Diphosphomevalonate decarboxylase, mevalonate pyrophosphate decarboxylase | EC 4.1.1.33 |
| IDI1 | Diphosphomevalonate decarboxylase, isopentenylpyrophosphate isomerase | EC 5.3.3.2 |

Alternatively, mevalonate can be phosphorylated to form mevalonate-5-phosphate, as shown in FIG. 1B, which depicts a mevalonate pathway from Archaea I bacteria. Mevalonate-5-phosphate can be decarboxylated to form isopentenyl phosphate, which can be further phosphorylated to form isopentenyl pyrophosphate (IPP). Exemplary enzymes that can be used to form IPP from acetyl-COA using the Archaea I mevalonate (MEV-A1) pathway are within the classes summarized in the following table.

TABLE 2
Non-limiting Examples of Enzymes in the Archaea I Mevalonate Pathway

| Gene | Enzyme | EC |
|---|---|---|
| PMD | phosphomevalonate decarboxylase, mevalonate-5-phosphate decarboxylase | EC 4.1.1.99 |
| | (Uniprot Accession: D4GXZ3 & Q18K00) | |
| IPK | isopentenyl phosphate kinase (Uniprot Accession: Q60352 & Q56187) | EC 2.7.4.26 |
| ERG10, ACAT | Acetoacetyl-CoA thiolase; Acetyl-CoA C-acetyltransferase; thiolase II | EC 2.3.1.9 |
| ERG13 | hydroxymethylglutaryl-CoA synthase or HMG-CoA synthase | EC 2.3.3.10 |
| HMGCR | NAD+ HMG-CoA reductase (3-hydroxy-3-methyl-glutaryl-coenzyme A | EC 1.1.1.88 |
| | reductase) | |
| HMG1, HMG2, | NADP+ HMG-CoA reductase | EC 1.1.1.34 |
| HMGCR | | |
| ERG12 | Mevalonate kinase, mevalonate-5-kinase | EC 2.7.1.36 |
| IDI1 | Diphosphomevalonate decarboxylase, isopentenylpyrophosphate isomerase | EC 5.3.3.2 |

Mevalonate also can be converted to IPP in four steps as shown in FIG. 1C, which depicts a mevalonate pathway from Archaea II bacteria (MEV-AII). Mevalonate can be phosphorylated to form mevalonate-3-phosphate, which can be phosphorylated to form mevalonate-3,5-bisphosphate. Mevalonate-3,5-bisphosphate can be decarboxylated to form isopentenyl phosphate, which can be phosphorylated to IPP. Exemplary enzymes that can be used to form IPP from acetyl-COA using the Archaea II mevalonate pathway are within the classes summarized in the following table.

TABLE 3
Non-limiting Examples of Enzymes in the Archaea II Mevalonate Pathway

| Gene | Enzyme | EC |
|---|---|---|
| M3K | mevalonate-3-kinase (Uniprot accession: Q9HIN1 & Q6KZB1) | EC 2.7.1.185 |
| MVA3P | Mevalonate-3-phosphate-5-kinase (Uniprot accession: Q9HK44 & | EC 2.7.1.186 |
| | A0A5J5FDZ3) | |
| IPK | isopentenyl phosphate kinase (Uniprot accession: Q60352 & Q56187) | EC 2.7.4.26 |
| PMD | phosphomevalonate decarboxylase, mevalonate-5-phosphate decarboxylase | EC 4.1.1.99 |
| | (Uniprot Accession: D4GXZ3 & Q18K00) | |
| ERG10, ACAT | Acetoacetyl-CoA thiolase; Acetyl-CoA C-acetyltransferase; thiolase II | EC 2.3.1.9 |
| ERG13 | hydroxymethylglutaryl-CoA synthase or HMG-CoA synthase | EC 2.3.3.10 |
| HMGCR | NAD+ HMG-CoA reductase (3-hydroxy-3-methyl-glutaryl-coenzyme A | EC 1.1.1.88 |
| | reductase) | |
| HMG1, HMG2, | NADP+ HMG-CoA reductase | EC 1.1.1.34 |
| HMGCR | | |
| ERG12 | Mevalonate kinase, mevaolnate-5-kinase | EC 2.7.1.36 |
| IDI1 | Diphosphomevalonate decarboxylase, isopentenylpyrophosphate isomerase | EC 5.3.3.2 |

IPP and DMPP can also be formed in a non-mevalonate or methylerithritol phosphate (MEP) pathway as illustrated in FIG. 1D. In the MEP pathway (from eubacteria, algae and higher plants), pyruvate and glyceraldehyde-3-phosphate can be condensed to form 1-deoxyxylulose-5-phosphate (DXP). Then follows the NADPH-dependent reduction and isomerization of DXP into 2C-methyl-D-erythritol 4-phosphate (MEP), which is catalyzed by DXP reductoisomerase (DXR). MEP then reacts with CTP and is converted into 4-diphosphocytidyl-2C-methyl D-erythritol (CDP-ME) through the enzymatic action of 2-C-methyl-D-erythritol 4-phosphate cytidylyltransferase (CMS). CDP-ME undergoes a phosphorylation by the ATP-dependent 4-diphosphocytidyl-2-C-methyl-D-erythrito kinase (CME) to produce 4-diphosphocytidyl-2C-methyl D-erythritol 2-phosphate (CDP-MEP). Then, CDP-MEP is cyclized by 2-C-methyl-Derythritol 2,4-cyclodiphosphate synthase (MCS), with simultaneous elimination of CMP, to form 2C-methyl-D-erythritol 2,4-cyclodiphosphate (2-C-Methyl-D-erythritol-2,4-cyclopyrophosphate, MEC, or MEcPP). Then, MEC undergoes a reductive ring opening, catalyzed by the NADPH-dependent 4-hydroxy-3-methylbut-2-en-1-yl diphosphate synthase (HDS), which produces 4-hydroxy-3-methylbut-2-en-1-yl diphosphate (HMB-PP). Finally, HMB-PP is reduced by 4-hydroxy-3-methylbut-2-en-1-yl diphosphate reductase (HDR) to produce a mixture of IPP and DMAPP (12, 18). Exemplary enzymes that can be used for forming IPP from pyruvate and glyceraldehyde-3-phosphate using the non-mevalonate pathway are within the classes summarized in the following table.

TABLE 4
Non-limiting Examples of Enzymes in the Methylerithritol Phosphate (MEP) Pathway

| Gene | Enzyme | EC |
|---|---|---|
| Dxs; DXS | 1-deoxy-D-xylulose-5-phosphate synthase (Uniprot | EC 2.2.1.7 |
| | Accession:) | |
| Dxr, IspC; DXR | DXP reductoisomerase (1-deoxy-D-xylulose 5- | EC 1.1.1.267 |
| | phosphate reductoisomerase or DXR) (Uniprot | |
| | Accession:) | |
| YgbP, IspD; CMS | 2-C-methyl-D-erythritol 4-phosphate | EC 2.7.7.60 |
| | cytidylyltransferase (Uniprot Accession:) | |
| YchB, IspE; CMK | 4-(cytidine 5′-diphospho)-2-C-methyl-D-erythritol | EC 2.7.1.148 |
| | kinase (Uniprot Accession:) | |
| MEcPP synthase, IspF; MCS | 2-C-methyl-D-erythritol 2,4-cyclodiphosphate synthase | EC 4.6.1.12 |
| | (Uniprot Accession:) | |
| HMB-PP synthase, IspG, GcpE, | 4-hydroxy-3-methylbut-2-en-1-yl diphosphate synthase | EC 1.17.7.1 |
| HDS | (Uniprot Accession:) | |
| LytB, IspH; HDR | 4-Hydroxy-3-methylbut-2-enyl diphosphate reductase | EC 1.17.1.2 |
| | (Uniprot Accession:) | |

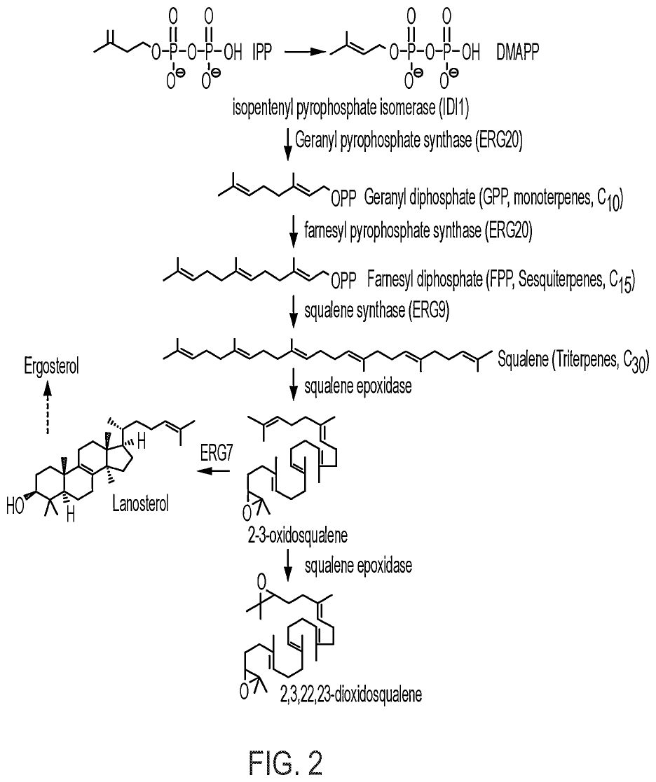

The isoprenoid precursors IPP and/or DMAPP produced from the MEV or MEP pathway can be used to produce a variety of isoprenoids, for example, as shown in FIG. 2, which illustrates the prenyltransferase-catalyzes elongation of isoprenoid chains to generate prenyl diphosphates of different lengths. For example, geranyl pyrophosphate synthase catalyzes the formation of GPP, farnesyl pyrophosphate synthase catalyzes the production of FPP, and geranylgeranyl pyrophosphate synthase catalyzes the formation of GGPP. As used herein, the term “prenyl diphosphate” encompasses monoprenyl diphosphates that have only one prenyl group and polyprenyl diphosphates that comprise at least two prenyl groups. IPP and DMAPP are non-limiting examples of monoprenyl diphosphates. Geranylgeranyl diphosphate (GGPP) is a non-limiting example of a polyprenyl diphosphate. Exemplary prenyltransferases useful for producing iosprenoids are within the classes summarized in the following table.

TABLE 5
Non-limiting Examples of Prenyltransferases

| Enzyme | EC |
|---|---|
| geranylgeranyl diphosphate synthase | EC 2.5.1.29 |
| hexaprenyl diphosphate synthase | EC:2.5.1.82; |
| | 2.5.1.83 |
| heptaprenyl diphosphate synthase | EC 2.5.1.30; |
| octaprenyl diphosphate synthase | EC 2.5.1.90 |
| solanesyl diphosphate synthase | EC 2.5.1.84; |
| | EC 2.5.1.85 |
| decaprenyl diphosphate (DPP) synthase | EC 2.5.1.86; |
| | EC 2.5.1.91 |
| nonaprenyl diphosphate synthase | EC 2.5.1.84; |
| | EC 2.5.1.85 |
| undecaprenyl diphosphate synthase | EC 2.5.1.89; |
| | EC 2.5.1.31 |
| dehydrodolichyl diphosphate synthase | EC 2.5.1.87 |
| chicle synthase | |
| gutta-percha synthase | |
| eicosaprenyl diphosphate synthase | |

Prenyl diphosphates serve as the substrate for numerous isoprenoid synthesis pathways. As a non-limiting example, FIG. 2 shows how IPP and DMAPP are incorporated into a sterol biosynthesis pathway from Saccharomyces cerevisiae. Squalene synthase catalyzes the formation of squalene from FPP. A squalene synthase may be encoded by the ERG9 gene. A non-limiting example of a squalene synthase is provided by UniProtKB Accession No. P29704. Then, a squalene epoxidase (SQE) oxidizes squalene to form 2-3-oxidosqualene, which serves as a substrate for a lanosterol synthase to produce lanosterol. Lanosterol may then be converted to the sterol ergosterol through a series of steps as known in the art. See, e.g., Klug and Daum, FEMS Yeast Res. 2014 May; 14(3):369-88. Prenyl diphosphate substrates can also be used by terpene synthases as substrates to produce isoprenoid.

### Isoprenoid and Isoprenoid Precursors

Isoprenoid and isoprenoid precursors that can be produced as described herein include the following non-limiting examples.

Isoprenoid precursors include but are not limited to acetyl-CoA, acetoacetyl-CoA, HMG-CoA, mevalonate, mevalonate-5-phosphate, mevalonate pyrophosphate, isopentenyl pyrophosphate (IPP), dimethylallyl pyrophosphate (DMAPP), geranyl pyrophosphate (GPP), farnesyl diphosphate (FPP), squalene, and 2-3-oxidosqualene. In some embodiments, an isoprenoid precursor is a compound shown in FIGS. 1A-1D and/or FIG. 2.

As used herein, unless otherwise indicated, isoprenoids are organic compounds comprising isoprene (i.e., C₅H₈) units and derivatives thereof. The terms “isoprenoid,” “terpene,” and “terpenoid” are used interchangeably in this application. For example, isoprenoids include pure hydrocarbons with the molecular formula (C₅H₈)ₙ, in which n represents the number of isoprene subunits. An isoprenoid may include carbon atoms in multiples of five. As a non-limiting example, an isoprenoid may comprise 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1,000, or more than 1,000 carbons. In some embodiments, an isoprenoid is an irregular isoprenoid. Isoprenoids also include oxygenated compounds. Isoprenoids are structurally diverse compounds and, for example, may be cyclic (e.g., monocyclic, multi-cyclic, homocyclic and heterocyclic compounds) or acyclic (e.g., linear and branched compounds). In some embodiments, an isoprenoid may have a flavor and/or odor. As used herein, an aroma compound refers to a compound that has an odor.

Non-limiting examples of isoprenoids include monoterpenes, sesquiterpenes, diterpenes, sesterterpenes, triterpenes, and tetraterpenes. Monoterpenes comprise ten carbons. Non-limiting examples of monoterpenes include, but are not limited to, myrcene, methanol, carvone, hinokitiol, linalool, limonene, sabinene, thujene, carene, borneol, eucalyptol and camphene. Sesquiterpenes comprise 15 carbons. As used herein, sesquiterpenes include sesquiterpene hydrocarbons and sesquiterpene alcohols (sesquiterpenols). Non-limiting examples of sesquiterpenes include but are not limited to, delta-cadinene, epi-cubenol, tau-cadinol, alpha-cadinol, gamma-selinene, 10-epi-gamma-eudesmol, gamma-eudesmol, alpha/beta-eudesmol, juniper camphor, 7-epi-alpha-eudesmol, cryptomeridiol isomer 1, cryptomeridiol isomer 2, cryptomeridiol isomer 3, humulene, alpha-guaiene, delta-guaiene, zingiberene, beta-bisabolene, beta-farnesene, beta-sesquiphellandrene, cubenol, alpha-bisabolol, alpha-curcumene, trans-nerolidol, gamma, bisabolene, beta-caryophyllene, trans-Sesquisabinene hydrate, delta-elemene, cis-eudesm-6-en-11-ol, daucene, isodaucene, trans-bergamotene, alpha-zingiberene, sesquisabinene hydrate, and 8-Isopropenyl-1,5-dimethyl-1,5-cyclodecadiene. Diterpenes comprise 20 carbons. Non-limiting examples of diterpenes include, but are not limited to, cembrene and sclareol. Sesterterpenes comprise 25 carbons. A non-limiting example of a sesterterpene is geranylfarnesol. Triterpenes comprise 30 carbons. Non-limiting examples of triterpenes include squalene, polypodatetraene, malabaricane, lanostane, cucurbitacin, hopane, oleanane, and ursolic acid. Tetraterpenes comprise 40 carbons. Non-limiting examples of tetraterpenes include carotenoids, e.g., xanthophylls and carotenes. See also, e.g., WO 2019/161141. In some embodiments, an isoprenoid is a cannabinoid. See, e.g., WO 2020/176547.

Any methods known in the art, including mass spectrometry (e.g., gas chromatography-mass spectrometry), may be used to identify an isoprenoid precursor or isoprenoid of interest.

In some embodiments, an isoprenoid is a mogrol (11, 24, 25-trihydroxy cucurbitadienol), mogrol precursor, or mogroside.

In some embodiments, a decrease in ERG7 expression, level or activity decreases the amount of 2-3-oxido-squalene converted into lanosterol, and increases the amount of 2-3-oxido-squalene available to be converted, via one or more enzymatic steps, into a mogrol precursor, mogrol, and/or mogroside.

In some embodiments, mogrol precursors include but are not limited to: 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol.

In some embodiments, precursors to a mogroside include mogrol precursors, mogrol, and other mogrosides.

In some embodiments, mogrosides include, but are not limited to: mogroside I-A1 (MIA1), mogroside IE (MIE or M1E), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), mogroside VIA (MVIA), mogroside VIB (MVIB), isomogroside V, mogroside VIa1 (MVIa1 or MVIa1), and mogroside VI (MVI or M6). In some embodiments, the mogroside is siamenoside I, which may be referred to as siamenoside or Siam. In some embodiments, the mogroside is MIIIE.

### Enzymes for Increasing Production of Isoprenoid or Isoprenoid Precursors

In various aspects, the present disclosure pertains to methods of increasing production of an isoprenoid or isoprenoid precursor in a host cell, wherein the host cell expresses (1) a reduced level of one or more enzymes of the MEV, MEV-A1, MEV-All or MEP pathway; (2) a reduced level of one or more enzymes involved in the conversion of IPP or DMAPP to a sterol such as lanosterol or ergosterol; (3) one or more attenuated forms of the foregoing enzymes; or (4) any combination thereof. For example, a host cell with increased production of an isoprenoid can comprise a variant of a lanosterol synthase and/or squalene epoxidase (SQE) with reduced (e.g., decreased but not abolished) activity. In some embodiments, the lanosterol synthase variant is encoded by a variant of the ERG7 coding sequence. In some embodiments, the squalene epoxidase is encoded by an ERG1 gene.

In some embodiments, a decrease in lanosterol synthase or squalene epoxidase activity is associated with a surprising increase in the abundance of mevalonate (which is neither a substrate nor a product of lanosterol synthase or squalene epoxidase), and the increase in mevalonate can facilitate an increase in the synthesis of compounds which are directly or indirectly (e.g., via one or more enzymatic steps) derived from mevalonate, including various isoprenoids and isoprenoid precursors. In some embodiments, the lanosterol synthase variant is encoded by a variant of the ERG7 coding sequence. In some embodiments, the squalene epoxidase is encoded by an ERG1 gene.

In some embodiments, the decrease in lanosterol synthase or squalene epoxidase activity can also decrease the amount of 2-3-oxido-squalene being converted into lanosterol, and can increase the amount of 2-3-oxido-squalene available to be shunted into another pathway, for example, into a mogrol precursor, mogrol, and/or mogroside. In some embodiments, the lanosterol synthase variant is encoded by a variant of the ERG7 coding sequence. In some embodiments, the squalene epoxidase is encoded by an ERG1 gene.

### 1. Lanosterol Synthases

Isoprenoid and isoprenoid production can be augmented by upregulating or downregulating the expression of one or more genes or the activity of their gene product or encoded enzymes including, for example, a lanosterol synthase.

As used in this disclosure, a lanosterol synthase is an enzyme that is capable of catalyzing cyclization of 2-3-oxidosqualene to produce lanosterol. In some embodiments, a lanosterol synthase disclosed herein is a hypomorph of lanosterol synthase (e.g., a variant that has reduced but not abolished lanosterol synthase activity). Without being bound by a particular theory, complete inactivation of lanosterol synthase is lethal in yeast, as lanosterol synthase may be needed to produce a hydrophobic component of the cell membrane important for maintaining the integrity of the cell. In some embodiments, a lanosterol synthase disclosed herein is useful for isoprenoid precursor and/or isoprenoid production as reduction in lanosterol synthase activity increases flux through a terpene synthesis pathway. In some embodiments, a lanosterol synthase disclosed herein increases flux through a terpene synthesis pathway and/or reduces competition for oxidosqualene. In some embodiments, a terpene synthesis pathway comprises one or more enzymes shown in FIGS. 1A-1D, FIG. 2, Tables 1-5, and/or an enzyme disclosed herein. Structurally, a lanosterol synthase may comprise the catalytic motif DCTAE (SEQ ID NO: 5). See e.g., Corey et al. PNAS 1994 Mar. 15; 91(6):2211-5 and Shi et al. 1994 Jul. 19; 91(15):7370-4. In some embodiments, a lanosterol synthase corresponds to the enzyme classification number EC 5.4.99.7.

As a non-limiting example, a lanosterol synthase may comprise the amino acid sequence:

(SEQ ID NO: 1)
MGIHESVSKQFAKNGHSKYRSDRYGLPKTDLRRWTFHASDLGAQWWKYDD
TTPLEELEKRATDYVKYSLELPGYAPVTLDSKPVKNAYEAALKNWHLFAS
LQDPDSGAWQSEYDGPQFMSIGYVTACYFGGNEIPTPVKTEMIRYIVNTA
HPVDGGWGLHKEDKSTCFGTSINYVVLRLLGLSRDHPVCVKARKTLLTKF
GGAINNPHWGKTWLSILNLYKWEGVNPAPGELWLLPYFVPVHPGRWWVHT
RWIYLAMGYLEAAEAQCELTPLLEELRDEIYKKPYSEIDFSKHCNSISGV
DLYYPHTGLLKFGNALLRRYRKFRPQWIKEKVKEEIYNLCLREVSNTRHL
CLAPVNNAMTSIVMYLHEGPDSANYKKIAARWPEFLSLNPSGMFMNGTNG
LQVWDTAFAVQYACVCGFAELPQYQKTIRAAFDFLDRSQINEPTEENSYR
DDRVGGWPFSTKTQGYPVSDCTAEALKAIIMVQNTPGYEDLKKQVSDKRK
HTAIDLLLGMQNVGSFEPGSFASYEPIRASSMLEKINPAEVFGNIMVEYP
YVECTDSVVLGLSYFRKYHDYRNEDVDRAISAAIGYIIREQQPDGGFFGS
WGVCYCYAHMFAMEALETQNLNYNNCSTVQKACDFLAGYQEADGGWAEDF
KSCETQMYVRGPHSLVVPTAMALLSLMSGRYPQEDKIHAAARFLMSKQMS
NGEWLKEEMEGVFNHTCAIEYPNYRFYFVMKALGLYFKGYCQ.

SEQ ID NO: 1 may be encoded by an ERG7 gene. In some embodiments, SEQ ID NO: 1 is encoded by the nucleotide sequence:

(SEQ ID NO: 2)
ATGGGAATCCACGAAAGTGTGTCGAAACAGTTTGCGAAAAACGGACATTC
CAAGTACCGCAGCGACCGATACGGCTTACCTAAGACGGATCTGCGACGAT
GGACGTTCCACGCGTCCGATCTGGGGGCGCAATGGTGGAAGTATGACGAT
ACCACACCGCTGGAAGAGCTGGAAAAGAGGGCTACCGACTACGTCAAATA
CTCGCTGGAGCTGCCGGGATACGCGCCCGTGACTCTGGACTCCAAGCCCG
TGAAAAATGCCTACGAAGCGGCTCTCAAAAACTGGCATCTGTTTGCGTCG
CTGCAAGACCCCGACTCCGGCGCATGGCAGTCGGAATACGACGGACCGCA
GTTCATGTCGATCGGTTATGTGACGGCGTGCTACTTTGGCGGCAACGAGA
TCCCCACGCCGGTCAAAACCGAAATGATCAGATACATTGTCAACACAGCC
CACCCAGTTGACGGAGGCTGGGGCCTTCACAAAGAAGACAAGAGCACCTG
TTTCGGTACCAGCATCAACTACGTGGTCCTGCGACTACTGGGCCTGTCAC
GGGATCATCCGGTCTGCGTCAAGGCGCGCAAAACGCTGCTCACCAAGTTT
GGCGGCGCCATCAACAACCCCCATTGGGGCAAGACCTGGCTGTCGATTCT
CAATCTCTACAAATGGGAGGGTGTGAATCCGGCCCCTGGCGAGCTCTGGC
TGTTGCCCTACTTTGTTCCTGTTCATCCGGGCCGATGGTGGGTCCATACC
CGGTGGATCTACCTTGCCATGGGCTATCTGGAGGCTGCGGAGGCCCAATG
CGAACTCACTCCGTTGCTGGAGGAGCTCCGAGACGAAATCTACAAAAAGC
CCTACTCGGAGATTGATTTCTCCAAACATTGCAACTCCATCTCCGGAGTC
GACCTCTACTATCCCCACACCGGCCTTTTGAAGTTTGGCAACGCGCTTCT
CCGACGATACCGCAAGTTCAGACCGCAGTGGATCAAAGAAAAGGTCAAGG
AGGAAATTTACAACTTGTGCCTTCGAGAGGTTTCCAACACACGACACTTG
TGTCTCGCTCCCGTCAACAATGCCATGACCTCCATTGTCATGTATCTCCA
TGAGGGGCCCGATTCGGCGAATTACAAAAAGATTGCGGCCCGATGGCCCG
AATTTCTGTCTCTGAATCCGTCGGGAATGTTTATGAACGGCACCAACGGT
CTGCAGGTCTGGGATACTGCGTTTGCCGTGCAATACGCGTGTGTTTGTGG
CTTTGCCGAACTTCCCCAGTACCAGAAGACGATCCGAGCGGCGTTTGATT
TTCTCGATCGGTCCCAGATCAACGAGCCGACGGAGGAAAATTCCTATCGA
GACGACCGCGTCGGAGGATGGCCCTTTAGTACCAAGACCCAGGGGTATCC
AGTCTCCGACTGTACTGCCGAGGCTCTCAAGGCCATCATCATGGTCCAGA
ATACGCCTGGATACGAGGATCTGAAGAAACAAGTGTCTGACAAGCGGAAA
CACACTGCCATCGATCTACTTTTGGGAATGCAGAACGTGGGCTCGTTTGA
ACCGGGCTCTTTCGCCTCCTATGAGCCTATCCGGGCGTCGTCCATGCTGG
AGAAGATCAATCCGGCCGAGGTGTTTGGAAACATCATGGTGGAGTATCCG
TACGTGGAATGCACTGATTCTGTTGTTCTGGGTCTGTCCTACTTTCGAAA
GTACCACGATTACCGCAACGAAGACGTGGACCGAGCCATCTCTGCTGCCA
TTGGATACATTATTCGAGAGCAGCAGCCTGACGGCGGCTTCTTTGGCTCC
TGGGGCGTGTGCTACTGCTACGCTCACATGTTTGCCATGGAGGCTCTGGA
GACGCAGAATCTCAACTATAACAACTGTTCCACGGTTCAAAAGGCGTGCG
ACTTTCTGGCGGGCTACCAGGAAGCAGATGGAGGCTGGGCCGAGGACTTT
AAGTCGTGCGAGACTCAGATGTACGTGCGCGGACCCCATTCGCTGGTCGT
GCCTACTGCCATGGCCCTGTTGAGTTTGATGAGTGGTCGGTATCCCCAGG
AGGACAAGATTCATGCTGCGGCCCGGTTTCTCATGAGCAAGCAGATGAGC
AACGGTGAGTGGCTCAAGGAGGAGATGGAGGGGGTGTTTAACCATACTTG
TGCCATTGAGTATCCCAACTACCGGTTTTATTTTGTCATGAAGGCTTTGG
GGTTGTATTTCAAGGGATATTGCCAGTGA.

In some embodiments, a lanosterol synthase comprises the amino acid sequence set forth in UniProtKB Accession No. P38604 (SEQ ID NO: 313, Tables 15-16).

In some embodiments, a lanosterol synthase comprising SEQ ID NO: 313 is encoded by the polynucleotide:

(SEQ ID NO: 8)
ATGACAGAATTTTATTCTGACACAATCGGTCTACCAAAGACAGATCCACG
TCTTTGGAGACTGAGAACTGATGAGCTAGGCCGAGAAAGCTGGGAATATT
TAACCCCTCAGCAAGCCGCAAACGACCCACCATCCACTTTCACGCAGTGG
CTTCTTCAAGATCCCAAATTTCCTCAACCTCATCCAGAAAGAAATAAGCA
TTCACCAGATTTTTCAGCCTTCGATGCGTGTCATAATGGTGCATCTTTTT
TCAAACTGCTTCAAGAGCCTGACTCAGGTATTTTTCCGTGTCAATATAAA
GGACCCATGTTCATGACAATCGGTTACGTAGCCGTAAACTATATCGCCGG
TATTGAAATTCCTGAGCATGAGAGAATAGAATTAATTAGATACATCGTCA
ATACAGCACATCCGGTTGATGGTGGCTGGGGTCTACATTCTGTTGACAAA
TCCACCGTGTTTGGTACAGTATTGAACTATGTAATCTTACGTTTATTGGG
TCTACCCAAGGACCACCCGGTTTGCGCCAAGGCAAGAAGCACATTGTTAA
GGTTAGGCGGTGCTATTGGATCCCCTCACTGGGGAAAAATTTGGCTAAGT
GCACTAAACTTGTATAAATGGGAAGGTGTGAACCCTGCCCCTCCTGAAAC
TTGGTTACTTCCATATTCACTGCCCATGCATCCGGGGAGATGGTGGGTTC
ATACTAGAGGTGTTTACATTCCGGTCAGTTACCTGTCATTGGTCAAATTT
TCTTGCCCAATGACTCCTCTTCTTGAAGAACTGAGGAATGAAATTTACAC
TAAACCGTTTGACAAGATTAACTTCTCCAAGAACAGGAATACCGTATGTG
GAGTAGACCTATATTACCCCCATTCTACTACTTTGAATATTGCGAACAGC
CTTGTAGTATTTTACGAAAAATACCTAAGAAACCGGTTCATTTACTCTCT
ATCCAAGAAGAAGGTTTATGATCTAATCAAAACGGAGTTACAGAATACTG
ATTCCTTGTGTATAGCACCTGTTAACCAGGCGTTTTGCGCACTTGTCACT
CTTATTGAAGAAGGGGTAGACTCGGAAGCGTTCCAGCGTCTCCAATATAG
GTTCAAGGATGCATTGTTCCATGGTCCACAGGGTATGACCATTATGGGAA
CAAATGGTGTGCAAACCTGGGATTGTGCGTTTGCCATTCAATACTTTTTC
GTCGCAGGCCTCGCAGAAAGACCTGAATTCTATAACACAATTGTCTCTGC
CTATAAATTCTTGTGTCATGCTCAATTTGACACCGAGTGCGTTCCAGGTA
GTTATAGGGATAAGAGAAAGGGGGCTTGGGGCTTCTCAACAAAAACACAG
GGCTATACAGTGGCAGATTGCACTGCAGAAGCAATTAAAGCCATCATCAT
GGTGAAAAACTCTCCCGTCTTTAGTGAAGTACACCATATGATTAGCAGTG
AACGTTTATTTGAAGGCATTGATGTGTTATTGAACCTACAAAACATCGGA
TCTTTTGAATATGGTTCCTTTGCAACCTATGAAAAAATCAAGGCCCCACT
AGCAATGGAAACCTTGAATCCTGCTGAAGTTTTTGGTAACATAATGGTAG
AATACCCATACGTGGAATGTACTGATTCATCCGTTCTGGGGTTGACATAT
TTTCACAAGTACTTCGACTATAGGAAAGAGGAAATACGTACACGCATCAG
AATCGCCATCGAATTCATAAAAAAATCTCAATTACCAGATGGAAGTTGGT
ATGGAAGCTGGGGTATTTGTTTTACATATGCCGGTATGTTTGCATTGGAG
GCATTACACACCGTGGGGGAGACCTATGAGAATTCCTCAACGGTAAGAAA
AGGTTGCGACTTCTTGGTCAGTAAACAGATGAAGGATGGCGGTTGGGGGG
AATCAATGAAGTCCAGTGAATTACATAGTTATGTGGATAGTGAAAAATCG
CTAGTCGTTCAAACCGCATGGGCGCTAATTGCACTTCTTTTCGCTGAATA
TCCTAATAAAGAAGTCATCGACCGCGGTATTGACCTTTTAAAAAATAGAC
AAGAAGAATCCGGGGAATGGAAATTTGAAAGTGTAGAAGGTGTTTTCAAC
CACTCTTGTGCAATTGAATACCCAAGTTATCGATTCTTATTCCCTATTAA
GGCATTAGGTATGTACAGCAGGGCATATGAAACACATACGCTTTAA.

In some embodiments, a lanosterol synthase comprises the amino acid sequence:

(SEQ ID NO: 3)
MGIHESVSKQFAKNGHSKYRSDRYGLPKTDLRRWTFHASDLGAQWWKYDG
TTPLEELEKRATDYVRYSLELPGYAPVTLDSKPVKNAYEAALKSWHLFAS
LQDPDSGAWQSEYDGPQFMSIGYVTACYFGGNEIPTPVKTEMIRYIVNTA
HPVDGGWGLHKEDKSTCFGTSINYVVLRLLGLSRDHPVCVKARKTLLTKF
GGAINNPHWGKTWLSILNLYKWEGVNPAPGELWLLPYFVPVHPGRWWVHT
RWIYLAMGYLEAAEAQCELTPLLEELRDEIYKKPYSEIDFSKHCNSISGV
DLYYPHTGLLKFGNALLRRYRKFRPQWIKEKVKEEIYNLCLREVSNTRHL
CLAPVNNAMTSIVMYLHEGPDSANYKKIAARWPEFLSLNPSGMFMNGTNG
LQVWDTAFAVQYACVCSFAELPQYQKTIRAAFDFLDRSQINEPTEENSYR
DDRVGGWPFSTKTQGYPVSDCTAEALKAIIMVQNTPGYEDLKKQVSDKRK
HTAIDLLLGMQNVGSFEPGSFASYEPIRASSMLEKINPAEVFGNIMVEYP
YVECTDSVVLGLSYFRKYHDYRNEDVDRAISAAIGYIIREQQPDGGFFGS
WGVCYCYAHMFAMEALVTQNLNYNNCSTVQKACDFLAGYQEADGGWAEDF
KSCETQMYVRGPHSLVVPTAMALLSLMSGRYPQEDKIHAAARFLMSKQMS
NGEWLKEEMEGVFNHTCAIEYPNYRLYFVMKALGLYFKGYCQ.

In some embodiments, a lanosterol synthase comprising SEQ ID NO: 3 is encoded by the nucleotide sequence:

(SEQ ID NO: 4)
ATGGGAATCCACGAAAGTGTGTCGAAACAGTTTGCGAAAAACGGACATTC
CAAGTACCGCAGCGACCGATACGGCTTACCTAAGACGGATCTGCGACGAT
GGACGTTCCACGCGTCCGATCTGGGGGCGCAATGGTGGAAGTATGACGGT
ACCACACCGCTGGAAGAGCTGGAAAAGAGGGCTACCGACTACGTCAGATA
CTCGCTGGAGCTGCCGGGATACGCGCCCGTGACTCTGGACTCCAAGCCCG
TGAAAAATGCCTACGAAGCGGCTCTCAAAAGCTGGCATCTGTTTGCGTCG
CTGCAAGACCCCGACTCCGGCGCATGGCAGTCGGAATACGACGGACCGCA
GTTCATGTCGATCGGTTATGTGACGGCGTGCTACTTTGGCGGCAACGAGA
TCCCCACGCCGGTCAAAACCGAAATGATCAGATACATTGTCAACACAGCC
CACCCAGTTGACGGAGGCTGGGGCCTTCACAAAGAAGACAAGAGCACCTG
TTTCGGTACCAGCATCAACTACGTGGTCCTGCGACTACTGGGCCTGTCAC
GGGATCATCCGGTCTGCGTCAAGGCGCGCAAAACGCTGCTCACCAAGTTT
GGCGGCGCCATCAACAACCCCCATTGGGGCAAGACCTGGCTGTCGATTCT
CAATCTCTACAAATGGGAGGGTGTGAATCCGGCCCCTGGCGAGCTCTGGC
TGTTGCCCTACTTTGTTCCTGTTCATCCGGGCCGATGGTGGGTCCATACC
CGGTGGATCTACCTTGCCATGGGCTATCTGGAGGCTGCGGAGGCCCAATG
CGAACTCACTCCGTTGCTGGAGGAGCTCCGAGACGAAATCTACAAAAAGC
CCTACTCGGAGATTGATTTCTCCAAACATTGCAACTCCATCTCCGGAGTC
GACCTCTACTATCCCCACACCGGCCTTTTGAAGTTTGGCAACGCGCTTCT
CCGACGATACCGCAAGTTCAGACCGCAGTGGATCAAAGAAAAGGTCAAGG
AGGAAATTTACAACTTGTGCCTTCGAGAGGTTTCCAACACACGACACTTG
TGTCTCGCTCCCGTCAACAATGCCATGACCTCCATTGTCATGTATCTCCA
TGAGGGGCCCGATTCGGCGAATTACAAAAAGATTGCGGCCCGATGGCCCG
AATTTCTGTCTCTGAATCCGTCGGGAATGTTTATGAACGGCACCAACGGT
CTGCAGGTCTGGGATACTGCGTTTGCCGTGCAATACGCGTGTGTTTGTAG
CTTTGCCGAACTTCCCCAGTACCAGAAGACGATCCGAGCGGCGTTTGATT
TTCTCGATCGGTCCCAGATCAACGAGCCGACGGAGGAAAATTCCTATCGA
GACGACCGCGTCGGAGGATGGCCCTTTAGTACCAAGACCCAGGGGTATCC
AGTCTCCGACTGTACTGCCGAGGCTCTCAAGGCCATCATCATGGTCCAGA
ATACGCCTGGATACGAGGATCTGAAGAAACAAGTGTCTGACAAGCGGAAA
CACACTGCCATCGATCTACTTTTGGGAATGCAGAACGTGGGCTCGTTTGA
ACCGGGCTCTTTCGCCTCCTATGAGCCTATCCGGGCGTCGTCCATGCTGG
AGAAGATCAATCCGGCCGAGGTGTTTGGAAACATCATGGTGGAGTATCCG
TACGTGGAATGCACTGATTCTGTTGTTCTGGGTCTGTCCTACTTTCGAAA
GTACCACGATTACCGCAACGAAGACGTGGACCGAGCCATCTCTGCTGCCA
TCGGATACATTATTCGAGAGCAGCAGCCTGACGGTGGCTTCTTTGGCTCC
TGGGGCGTGTGCTACTGCTACGCTCACATGTTTGCCATGGAGGCTCTGGT
GACGCAGAATCTCAACTATAACAACTGTTCCACGGTTCAAAAGGCGTGCG
ACTTTCTGGCGGGCTACCAGGAAGCAGATGGAGGCTGGGCCGAGGACTTT
AAGTCGTGCGAGACTCAGATGTACGTGCGCGGACCCCATTCGCTGGTCGT
GCCTACTGCCATGGCCCTGTTGAGTTTGATGAGTGGTCGGTATCCCCAGG
AGGACAAGATTCATGCTGCGGCCCGGTTTCTCATGAGCAAGCAGATGAGC
AACGGTGAGTGGCTCAAGGAGGAGATGGAGGGGGTGTTTAACCATACTTG
TGCCATTGAGTATCCCAACTACCGGTTATATTTTGTCATGAAGGCTTTGG
GGTTGTATTTCAAGGGATATTGCCAGTGA.

In some embodiments, a lanosterol synthase of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to any one of SEQ ID NOs: 1-4, 8, 61-66, 68-71, 73-74, 78, 80-87, 89-92, 94-95, 99-109, 111-120, 304, 313, 316-319, 321-326, and 328-331, any lanosterol synthase in Tables 15-16, or any lanosterol synthase sequence disclosed in this application or known in the art.

In some embodiments, a lanosterol synthase comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 amino acid changes relative to SEQ ID NO: 1 or 313.

In some embodiments, a lanosterol synthase comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, or at most 100 amino acid changes relative to SEQ ID NO: 1 or 313.

In some embodiments, a lanosterol synthase comprises between 1-5, between 1-10, between 1-15, between 1-20, between 1-25, between 1-30, between 1-35, between 1-40, between 1-45, between 1-50, between 5-10, between 5-20, between 5-30, between 5-40, between 5-50, between 5-60, between 5-70, between 5-80, between 5-90, between 5-100, between 10-20, between 10-30, between 10-40, between 10-50, between 10-60, between 10-70, between 10-80, between 10-90, or between 10-100 amino acid changes, including all values in between, relative to SEQ ID NO: 1 or 313.

In some embodiments, a lanosterol synthase comprises an amino acid change at one or more positions selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, or 742 of SEQ ID NO:1.

In some embodiments, a lanosterol synthase comprises an amino acid change at one or more positions selected from position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, or 731 of SEQ ID NO: 313.

In some embodiments, the amino acid change is a substitution, insertion, or a deletion. In some embodiments, the amino acid change results in a truncation of a lanosterol synthase relative to a control. In some embodiments, a control is a wild-type lanosterol synthase. In some embodiments, a control is a different lanosterol synthase. As a non-limiting example, a lanosterol synthase may comprise one or more changes indicated in Tables 7, 9, 10A-10B, 11-14, or relative to SEQ ID NO: 1 or 313.

In some embodiments, a lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1. In some embodiments, a lanosterol synthase comprises: the amino acid Y at the residue corresponding to position 14 in SEQ ID NO:1; the amino acid Q at the residue corresponding to position 33 in SEQ ID NO:1; the amino acid E at the residue corresponding to position 47 in SEQ ID NO:1; the amino acid G at the residue corresponding to position 50 in SEQ ID NO:1; the amino acid R at the residue corresponding to position 66 in SEQ ID NO:1; the amino acid G at the residue corresponding to position 80 in SEQ ID NO: 1; the amino acid L at the residue corresponding to position 83 in SEQ ID NO: 1; the amino acid N at the residue corresponding to position 85 in SEQ ID NO:1; the amino acid I at the residue corresponding to position 92 in SEQ ID NO:1; the amino acid S at the residue corresponding to position 94 in SEQ ID NO:1; the amino acid D at the residue corresponding to position 107 in SEQ ID NO:1; the amino acid C at the residue corresponding to position 122 in SEQ ID NO:1; the amino acid S at the residue corresponding to position 132 in SEQ ID NO:1; the amino acid C at the residue corresponding to position 145 in SEQ ID NO:1; the amino acid S at the residue corresponding to position 158 in SEQ ID NO:1; the amino acid A at the residue corresponding to position 170 in SEQ ID NO: 1; the amino acid N at the residue corresponding to position 172 in SEQ ID NO:1; the amino acid W at the residue corresponding to position 184 in SEQ ID NO:1; the amino acid C or H at the residue corresponding to position 193 in SEQ ID NO:1; the amino acid V at the residue corresponding to position 197 in SEQ ID NO:1; the amino acid I at the residue corresponding to position 198 in SEQ ID NO: 1; the amino acid I at the residue corresponding to position 212 in SEQ ID NO:1; the amino acid L at the residue corresponding to position 213 in SEQ ID NO:1; the amino acid L at the residue corresponding to position 227 in SEQ ID NO:1; the amino acid T at the residue corresponding to position 228 in SEQ ID NO: 1; the amino acid V at the residue corresponding to position 231 in SEQ ID NO:1; the amino acid M at the residue corresponding to position 235 in SEQ ID NO:1; the amino acid F at the residue corresponding to position 248 in SEQ ID NO:1; the amino acid L at the residue corresponding to position 249 in SEQ ID NO:1; the amino acid R at the residue corresponding to position 260 in SEQ ID NO:1; the amino acid I at the residue corresponding to position 282 in SEQ ID NO:1; the amino acid F at the residue corresponding to position 286 in SEQ ID NO: 1; the amino acid G at the residue corresponding to position 287 in SEQ ID NO:1; the amino acid G at the residue corresponding to position 289 in SEQ ID NO: 1; the amino acid I at the residue corresponding to position 295 in SEQ ID NO: 1; the amino acid T at the residue corresponding to position 296 in SEQ ID NO: 1; the amino acid F at the residue corresponding to position 309 in SEQ ID NO: 1; the amino acid S at the residue corresponding to position 314 in SEQ ID NO:1; the amino acid R at the residue corresponding to position 316 in SEQ ID NO:1; the amino acid N at the residue corresponding to position 329 in SEQ ID NO:1; the amino acid A at the residue corresponding to position 344 in SEQ ID NO: 1; the amino acid S at the residue corresponding to position 360 in SEQ ID NO:1; the amino acid L at the residue corresponding to position 370 in SEQ ID NO:1; the amino acid V at the residue corresponding to position 371 in SEQ ID NO:1; the amino acid P at the residue corresponding to position 372 in SEQ ID NO:1; the amino acid I at the residue corresponding to position 398 in SEQ ID NO: 1; the amino acid V at the residue corresponding to position 407 in SEQ ID NO:1; the amino acid S at the residue corresponding to position 414 in SEQ ID NO:1; the amino acid S at the residue corresponding to position 417 in SEQ ID NO:1; the amino acid L at the residue corresponding to position 423 in SEQ ID NO:1; the amino acid I or S at the residue corresponding to position 432 in SEQ ID NO:1; the amino acid L at the residue corresponding to position 437 in SEQ ID NO:1; the amino acid V at the residue corresponding to position 442 in SEQ ID NO:1; the amino acid M or S at the residue corresponding to position 444 in SEQ ID NO:1; the amino acid G at the residue corresponding to position 452 in SEQ ID NO:1; the amino acid V at the residue corresponding to position 474 in SEQ ID NO:1; the amino acid S at the residue corresponding to position 479 in SEQ ID NO:1; the amino acid Q at the residue corresponding to position 491 in SEQ ID NO:1; the amino acid N at the residue corresponding to position 498 in SEQ ID NO: 1; the amino acid L at the residue corresponding to position 515 in SEQ ID NO:1; the amino acid T at the residue corresponding to position 526 in SEQ ID NO:1; the amino acid T at the residue corresponding to position 529 in SEQ ID NO:1; the amino acid F at the residue corresponding to position 536 in SEQ ID NO:1; the amino acid Y at the residue corresponding to position 544 in SEQ ID NO:1; the amino acid E at the residue corresponding to position 552 in SEQ ID NO:1; the amino acid A at the residue corresponding to position 559 in SEQ ID NO:1; the amino acid M at the residue corresponding to position 560 in SEQ ID NO:1; the amino acid C or N at the residue corresponding to position 564 in SEQ ID NO:1; the amino acid P at the residue corresponding to position 578 in SEQ ID NO:1; the amino acid F at the residue corresponding to position 586 in SEQ ID NO:1; the amino acid T at the residue corresponding to position 608 in SEQ ID NO:1; the amino acid I at the residue corresponding to position 610 in SEQ ID NO: 1; the amino acid V at the residue corresponding to position 617 in SEQ ID NO:1; the amino acid L at the residue corresponding to position 619 in SEQ ID NO:1; the amino acid S at the residue corresponding to position 620 in SEQ ID NO:1; the amino acid E or R at the residue corresponding to position 631 in SEQ ID NO:1; the amino acid D at the residue corresponding to position 638 in SEQ ID NO:1; the amino acid L at the residue corresponding to position 650 in SEQ ID NO:1; the amino acid A at the residue corresponding to position 655 in SEQ ID NO:1; the amino acid H at the residue corresponding to position 660 in SEQ ID NO:1; the amino acid S at the residue corresponding to position 679 in SEQ ID NO:1; the amino acid E at the residue corresponding to position 686 in SEQ ID NO: 1; the amino acid D at the residue corresponding to position 702 in SEQ ID NO:1; the amino acid Q at the residue corresponding to position 710 in SEQ ID NO:1; the amino acid L or V at the residue corresponding to position 726 in SEQ ID NO:1; the amino acid F at the residue corresponding to position 736 in SEQ ID NO:1; the amino acid M at the residue corresponding to position 738 in SEQ ID NO:1; and/or a truncation that results in deletion of the residue corresponding to position 742 in SEQ ID NO: 1. In some embodiments, a lanosterol synthase comprises the amino acid substitution E617V, G107D, and/or K631E relative to SEQ ID NO: 1.

In some embodiments, relative to SEQ ID NO: 1, a lanosterol synthase comprises: R33Q, R193C, D289G, N295I, S296T, N620S, and Y736F; R184W, L235M, L260R, and E710Q; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; F432S, D452G, and I536F; E287G, K329N, E617V, and F726V; E231V, A407V, Q423L, A529T, and Y564C; V248F, D371V, and G702D; L197V, K282I, N314S, P370L, A608T, G638D, and F650L; L491Q, Y586F, and R660H; G122C, H249L, and K738M; P227L, E474V, V559A, and Y564N; K85N, G158S, S515L, P526T, Q619L, and a truncation resulting in a deletion of the residue corresponding to Q742 in SEQ ID NO 1; G107D and K631E; T212I, W213L, N544Y, and V552E; I172N, C414S, L560M, and G679S; R193C, D289G, N295I, S296T, N620S, and Y736F; K85N and G158S; L197V, K282I, N314S, and P370L; I172N, C414S, and L560M; D371V, M610I, and G702D; D371V, K498N, M610I, and G702D; D80G, P83L, T170A, T198I, and A228T; T360S, S372P, T444M, and R578P; D50G, K66R, N94S, G417S, and E617V; or L309F, V344A, T398I, and K686E.

In some embodiments, relative to SEQ ID NO: 1, the lanosterol synthase comprises the following amino acid substitutions: R193C, D289G, N295I, S296T, N620S, and Y736F; F432S, D452G, and I536F; K85N and G158S; L197V, K282I, N314S, and P370L; I172N, C414S, L560M, and G679S; I172N, C414S, and L560M; D371V, M610I, and G702D; D371V, K498N, M610I, and G702D; D80G, P83L, T170A, T198I, and A228T; D50G, K66R, N94S, G417S, E617V, and F726L; T360S, S372P, T444M, and R578P; D50G, K66R, N94S, G417S, and E617V; and L309F, V344A, T398I, and K686E.

In some embodiments, relative to SEQ ID NO: 1, the lanosterol synthase comprises the following amino acid substitutions: D50G, K66R, N94S, G417S, E617V, and F726L; K85N and G158S; K47E, L92I, T360S, S372P, T444M, and R578P; F432S, D452G, and I536F; T360S, S372P, T444M, and R578P; L491Q, Y586F, and R660H; K85N, G158S, S515L, P526T, Q619L, and a truncation that results in deletion of the residue corresponding to position 742 in SEQ ID NO: 1; or I172N, C414S, L560M, and G679S.

In some embodiments, a lanosterol comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1. In some embodiments, relative to SEQ ID NO: 1, a lanosterol synthase comprises: R33Q, R193C, D289G, N295I, S296T, N620S, and Y736F; K47E, L92I, T360S, S372P, T444M, and R578P; D50G, K66R, N94S, G417S, E617V, and F726L; N14Y, N132S, Y145C, R193H, I286F, L316R, F432I, E442V, T444S, I479S, K631R, and T655A; E287G, K329N, E617V, and F726V; E231V, A407V, Q423L, A529T, and Y564C; V248F, D371V, and G702D; G122C, H249L, and K738M; or K85N, G158S, S515L, P526T, and Q619L, and a truncation resulting in a deletion of the residue corresponding to Q742 in SEQ ID NO: 1.

In some embodiments, the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

In some embodiments, the lanosterol synthase comprises: the amino acid G at the residue corresponding to position 64 in SEQ ID NO: 313; the amino acid V at the residue corresponding to position 120 in SEQ ID NO: 313; the amino acid S at the residue corresponding to position 121 in SEQ ID NO: 313; the amino acid V at the residue corresponding to position 136 in SEQ ID NO: 313; the amino acid I at the residue corresponding to position 226 in SEQ ID NO: 313; the amino acid S at the residue corresponding to position 268 in SEQ ID NO: 313; the amino acid I at the residue corresponding to position 275 in SEQ ID NO: 313; the amino acid A at the residue corresponding to position 281 in SEQ ID NO: 313; the amino acid G at the residue corresponding to position 300 in SEQ ID NO: 313; the amino acid G at the residue corresponding to position 322 in SEQ ID NO: 313; the amino acid A at the residue corresponding to position 333 in SEQ ID NO: 313; the amino acid E at the residue corresponding to position 438 in SEQ ID NO: 313; the amino acid L at the residue corresponding to position 502 in SEQ ID NO: 313; the amino acid N at the residue corresponding to position 604 in SEQ ID NO: 313; the amino acid S at the residue corresponding to position 619 in SEQ ID NO: 313; the amino acid E at the residue corresponding to position 628 in SEQ ID NO: 313; the amino acid T at the residue corresponding to position 656 in SEQ ID NO: 313; the amino acid G at the residue corresponding to position 693 in SEQ ID NO: 313; and/or deletion of residues corresponding to positions 726-731 in SEQ ID NO: 313.

In some embodiments, the lanosterol synthase comprises relative to SEQ ID NO: 313: P121S, A136V, S300G, V322G, K438E, F502L, K628E, and deletion of residues corresponding to positions 726-731 in SEQ ID NO: 313; K268S, T281A, F502L, T604N, A656T, and E693G; or C619S, F275I, I120V, M226I, R64G, and T333A.

It should be appreciated that activity, such as specific activity, of a lanosterol synthase can be measured by any means known to one of ordinary skill in the art. In some embodiments, production of one or more isoprenoid precursors and/or isoprenoids can be used to determine lanosterol activity. As a non-limiting example, mevalonate production may be used as a readout of lanosterol synthase activity. For example, a lanosterol synthase with reduced activity (e.g., decreased but not abolished activity) may increase mevalonate production in a host cell relative to a control. In some embodiments, a control is a host cell with a different lanosterol synthase. In some embodiments, a control is a host cell with a wild-type lanosterol synthase.

The activity of a lanosterol synthase may be altered using any suitable method known in the art. In some embodiments, one or more amino acid changes reduces the activity of a lanosterol synthase as compared to a control lanosterol synthase. In some embodiments, a control lanosterol synthase is a wild-type lanosterol synthase. In some embodiments, the expression of a lanosterol synthase is altered to affect lanosterol synthase activity. In some embodiments, a host cell comprises a heterologous polynucleotide that is capable of reducing lanosterol synthase activity. In some embodiments, a reduction in lanosterol synthase expression in a host cell reduces lanosterol synthase activity. In some embodiments, the activity of a lanosterol synthase is reduced using: a weak promoter to drive expression of the lanosterol synthase, one or more codons that are not optimized for a particular host cell, use of an antisense nucleic acid, a genetic modification that alters gene expression and/or introduces one or more alterations, alteration of a promoter driving expression of a lanosterol synthase and/or altering the coding sequence of a lanosterol synthase.

In some embodiments, a lanosterol synthase is capable of increasing production of a isoprenoid precursor and/or isoprenoid by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of the isoprenoid precursor and/or isoprenoid by a host cell that does not comprise the lanosterol synthase. In some embodiments, a lanosterol synthase is capable of increasing production of a isoprenoid precursor and/or isoprenoid by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production of the isoprenoid precursor and/or isoprenoid by a host cell that does not comprise the lanosterol synthase. In some embodiments, a lanosterol synthase is capable of increasing production of a isoprenoid precursor and/or isoprenoid by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of the isoprenoid precursor and/or isoprenoid by a host cell that does not comprise the lanosterol synthase. In some embodiments, a lanosterol synthase is capable of increasing production of an isoprenoid precursor and/or isoprenoid by a host cell at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, at least 5 fold, at least 5.1 fold, at least 5.2 fold, at least 5.3 fold, at least 5.4 fold, at least 5.5 fold, at least 5.6 fold, at least 5.7 fold, at least 5.8 fold, at least 5.9 fold, at least 6 fold, at least 6.1 fold, at least 6.2 fold, at least 6.3 fold, at least 6.4 fold, at least 6.5 fold, at least 6.6 fold, at least 6.7 fold, at least 6.8 fold, at least 6.9 fold, at least 7 fold, at least 7.1 fold, at least 7.2 fold, at least 7.3 fold, at least 7.4 fold, at least 7.5 fold, at least 7.6 fold, at least 7.7 fold, at least 7.8 fold, at least 7.9 fold, at least 8 fold, at least 8.1 fold, at least 8.2 fold, at least 8.3 fold, at least 8.4 fold, at least 8.5 fold, at least 8.6 fold, at least 8.7 fold, at least 8.8 fold, at least 8.9 fold, at least 9 fold, at least 9.1 fold, at least 9.2 fold, at least 9.3 fold, at least 9.4 fold, at least 9.5 fold, at least 9.6 fold, at least 9.7 fold, at least 9.8 fold, at least 9.9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 21 fold, at least 22 fold, at least 23 fold, at least 24 fold, at least 25 fold, at least 26 fold, at least 27 fold, at least 28 fold, at least 29 fold, at least 30 fold, at least 31 fold, at least 32 fold, at least 33 fold, at least 34 fold, at least 35 fold, at least 36 fold, at least 37 fold, at least 38 fold, at least 39 fold, at least 40 fold, at least 41 fold, at least 42 fold, at least 43 fold, at least 44 fold, at least 45 fold, at least 46 fold, at least 47 fold, at least 48 fold, at least 49 fold, at least 50 fold, at least 51 fold, at least 52 fold, at least 53 fold, at least 54 fold, at least 55 fold, at least 56 fold, at least 57 fold, at least 58 fold, at least 59 fold, at least 60 fold, at least 61 fold, at least 62 fold, at least 63 fold, at least 64 fold, at least 65 fold, at least 66 fold, at least 67 fold, at least 68 fold, at least 69 fold, at least 70 fold, at least 71 fold, at least 72 fold, at least 73 fold, at least 74 fold, at least 75 fold, at least 76 fold, at least 77 fold, at least 78 fold, at least 79 fold, at least 80 fold, at least 81 fold, at least 82 fold, at least 83 fold, at least 84 fold, at least 85 fold, at least 86 fold, at least 87 fold, at least 88 fold, at least 89 fold, at least 90 fold, at least 91 fold, at least 92 fold, at least 93 fold, at least 94 fold, at least 95 fold, at least 96 fold, at least 97 fold, at least 98 fold, at least 99 fold, at least 100 fold, at least 200 fold, at least 300 fold, at least 400 fold, at least 500 fold, at least 600 fold, at least 700 fold, at least 800 fold, at least 900 fold, or at least 1000 fold, including all values in between as compared to production the isoprenoid precursor and/or isoprenoid by a host cell that does not comprise the lanosterol synthase. In some embodiments, the isoprenoid precursor is mevalonate. In some embodiments, the isoprenoid precursor is IPP, GPP, FPP. In some embodiments, the isoprenoid precursor is mevalonate or 2-3-oxidosqualene,

In some embodiments, a host cell comprising a lanosterol synthase is capable of producing at least 0.01 mg/L, at least 0.05 mg/L, at least 1 mg/L, at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 150 mg/L, at least 200 mg/L, at least 250 mg/L, at least 300 mg/L, at least 350 mg/L, at least 400 mg/L, at least 450 mg/L, at least 500 mg/L, at least 550 mg/L, at least 600 mg/L, at least 650 mg/L, at least 700 mg/L, at least 750 mg/L, at least 800 mg/L, at least 850 mg/L, at least 900 mg/L, at least 950 mg/L, at least 1 g/L, at least 1.1 g/L, at least 1.2 g/L, at least 1.3 g/L, at least 1.4 g/L, at least 1.5 g/L, at least 1.6 g/L, at least 1.7 g/L, at least 1.8 g/L, at least 1.9 g/L, at least 2 g/L, at least 2.1 g/L, at least 2.2 g/L, at least 2.3 g/L, at least 2.4 g/L, at least 2.5 g/L, at least 2.6 g/L, at least 2.7 g/L, at least 2.8 g/L, at least 2.9 g/L, at least 3 g/L, at least 3.1 g/L, at least 3.2 g/L, at least 3.3 g/L, at least 3.4 g/L, at least 3.5 g/L, at least 3.6 g/L, at least 3.7 g/L, at least 3.8 g/L, at least 3.9 g/L, at least 4 g/L, at least 4.1 g/L, at least 4.2 g/L, at least 4.3 g/L, at least 4.4 g/L, at least 4.5 g/L, at least 4.6 g/L, at least 4.7 g/L, at least 4.8 g/L, at least 4.9 g/L, at least 5 g/L, at least 5.1 g/L, at least 5.2 g/L, at least 5.3 g/L, at least 5.4 g/L, at least 5.5 g/L, at least 5.6 g/L, at least 5.7 g/L, at least 5.8 g/L, at least 5.9 g/L, at least 6 g/L, at least 6.1 g/L, at least 6.2 g/L, at least 6.3 g/L, at least 6.4 g/L, at least 6.5 g/L, at least 6.6 g/L, at least 6.7 g/L, at least 6.8 g/L, at least 6.9 g/L, at least 7 g/L, at least 7.1 g/L, at least 7.2 g/L, at least 7.3 g/L, at least 7.4 g/L, at least 7.5 g/L, at least 7.6 g/L, at least 7.7 g/L, at least 7.8 g/L, at least 7.9 g/L, at least 8 g/L, at least 8.1 g/L, at least 8.2 g/L, at least 8.3 g/L, at least 8.4 g/L, at least 8.5 g/L, at least 8.6 g/L, at least 8.7 g/L, at least 8.8 g/L, at least 8.9 g/L, at least 9 g/L, at least 9.1 g/L, at least 9.2 g/L, at least 9.3 g/L, at least 9.4 g/L, at least 9.5 g/L, at least 9.6 g/L, at least 9.7 g/L, at least 9.8 g/L, at least 9.9 g/L, at least 10 g/L, at least 20 g/L, at least 30 g/L, at least 40 g/L, at least 50 g/L, at least 60 g/L, at least 70 g/L, at least 80 g/L, at least 90 g/L, at least 100 g/L, at least 200 g/L, at least 300 g/L, at least 400 g/L, at least 500 g/L, at least 600 g/L, at least 700 g/L, at least 800 g/L, at least 900 g/L, or at least 1000 g/L including all values in between of a isoprenoid precursor and/or isoprenoid. In some embodiments, a host cell comprising a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of a isoprenoid precursor and/or isoprenoid. In some embodiments, a host cell comprising a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of a isoprenoid precursor and/or isoprenoid. In some embodiments, the isoprenoid precursor is mevalonate. In some embodiments, the isoprenoid precursor is IPP, GPP, FPP. In some embodiments, the isoprenoid precursor is mevalonate or 2-3-oxidosqualene,

In some embodiments, lanosterol is used as a readout of lanosterol synthase activity. For example, a lanosterol synthase with reduced activity may produce less lanosterol from 2-3-oxidosqualene relative to a control. In some embodiments, a control is a different lanosterol synthase. In some embodiments, a control is a wild-type lanosterol synthase. Lanosterol synthase activity may be determined using a cell lysate, a purified enzyme, or in a host cell.

In some embodiments, a lanosterol synthase is capable of decreasing production of lanosterol by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of lanosterol by a host cell that does not comprise the lanosterol synthase. In some embodiments, a lanosterol synthase is capable of decreasing production of lanosterol by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production of lanosterol by a host cell that does not comprise the lanosterol synthase. In some embodiments, a lanosterol synthase is capable of decreasing production of lanosterol by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of lanosterol by a host cell that does not comprise the lanosterol synthase.

In some embodiments, lanosterol synthase activity in a host cell is determined by the level of ergosterol produced by a cell. Ergosterol is a fungal cell membrane sterol that is produced from lanosterol. See, e.g., Klug and Daum, FEMS Yeast Res. 2014 May; 14(3):369-88. In some embodiments, a host cell comprising a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of ergosterol. In some embodiments, a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of ergosterol.

In some embodiments, a lanosterol synthase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of ergosterol.

In some embodiments, a lanosterol synthase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of ergosterol.

### 2. Squalene Epoxidases Enzymes (SQEs)

Isoprenoid and isoprenoid production can be augmented by upregulating or downregulating the expression of one or more genes or the activity of their gene product or encoded enzymes including, for example, squalene eposidase. In some embodiments, a squalene epoxidase corresponds to enzyme classification number EC 1.14.14.17.

Aspects of the present disclosure provide squalene epoxidases (SQEs), which are capable of oxidizing a squalene (e.g., squalene or 2-3-oxidosqualene) to produce a squalene epoxide (e.g., 2-3-oxidosqualene or 2-3, 22-23-diepoxysqualene). SQEs may also be referred to as squalene monooxygenases. In some embodiments, a squalene epoxidase is encoded by ERG1.

In some embodiments, an SQE comprises the sequence set forth in GenBank Accession No. AOW05469.1:

(SEQ ID NO: 9)
MVTQQSAAETSATQTNEYDVVIVGAGIAGPALAVALGNQGRKVLVVERDL
SEPDRIVGELLQPGGVAALKTLGLGSCIEDIDAIPCQGYNVIYSGEECVL
KYPKVPRDIQQDYNELYRSGKSADISNEAPRGVSFHHGRFVMNLRRAARD
TPNVTLLEATVTEVVKNPYTGHIIGVKTFSKTGGAKIYKHFFAPLTVVCD
GTFSKFRKDFSTNKTSVRSHFAGLILKDAVLPSPQHGHVILSPNSCPVLV
YQVGARETRILCDIQGPVPSNATGALKEHMEKNVMPHLPKSIQPSFQAAL
KEQTIRVMPNSFLSASKNDHHGLILLGDALNMRHPLTGGGMTVALNDALL
LSRLLTGVNLEDTYAVSSVMSSQFHWORKHLDSIVNILSMALYSLFAADS
DYLRILQLGCFNYFKLGGICVDHPVMLLAGVLPRPMYLFTHFFVVAIYGG
ICNMQANGIAKLPASLLQFVASLVTACIVIFPYIWSELT.

In some embodiments, SEQ ID NO: 9 is encoded by the nucleotide sequence:

(SEQ ID NO: 10)
CTAAGTCAGCTCGCTCCAAATGTAAGGGAAGATGACGATGCAAGCGGTGA
CCAGAGAGGCGACAAATTGCAGTAGCGACGCGGGCAGCTTGGCAATGCCG
TTGGCCTGCATGTTGCAGATTCCGCCGTAGATGGCCACTACGAAGAAATG
CGTAAACAGGTACATGGGTCGGGGGAGAACTCCAGCCAACAGCATGACGG
GGTGGTCCACACAGATGCCTCCCAGCTTGAAGTAGTTGAAGCATCCGAGC
TGCAGGATTCGCAAGTAGTCCGAGTCGGCGGCGAAGAGCGAGTAGAGGGC
CATGGAGAGAATGTTGACGATGGAGTCGAGGTGTTTTCGCTGCCAGTGGA
ACTGCGAGCTCATGACGGAGGACACGGCATAGGTGTCTTCCAGGTTAACG
CCGGTGAGAAGTCTGCTGAGTAGAAGGGCATCATTGAGAGCAACGGTCAT
TCCTCCTCCGGTAAGTGGATGTCGCATGTTGAGTGCGTCACCCAGCAGAA
TCAAACCGTGGTGATCGTTCTTGGAGGCCGACAGGAAAGAGTTGGGCATG
ACTCGAATGGTCTGCTCCTTGAGAGCGGCTTGGAAAGACGGCTGGATGGA
CTTAGGCAGGTGGGGCATGACGTTCTTCTCCATGTGTTCCTTGAGGGCTC
CGGTTGCATTAGAGGGGACGGGTCCCTGAATGTCACACAGAATTCGGGTC
TCTCGAGCTCCAACCTGGTAGACAAGAACGGGACACGAGTTGGGCGACAG
AATCACGTGGCCATGCTGGGGGGAGGGCAGAACAGCGTCCTTGAGAATCA
GACCGGCGAAATGCGAACGCACAGACGTCTTGTTGGTGCTAAAGTCCTTT
CGGAACTTGGAAAAAGTTCCATCACAGACGACGGTGAGAGGAGCAAAGAA
GTGCTTGTAGATTTTGGCGCCTCCAGTTTTAGAGAAGGTCTTGACTCCAA
TAATGTGGCCGGTGTAAGGGTTCTTGACCACCTCGGTGACTGTGGCCTCC
AGCAGAGTCACATTGGGTGTGTCTCGTGCGGCCCTTCGCAAGTTCATGAC
AAATCGGCCGTGGTGGAAGGATACTCCTCGGGGAGCCTCGTTGGAGATGT
CGGCAGACTTTCCGCTTCTGTACAGCTCGTTGTAGTCCTGCTGGATGTCT
CGGGGGACCTTGGGGTATTTGAGAACGCACTCTTCTCCAGAGTAGATCAC
GTTGTATCCCTGGCAGGGGATCGCGTCGATATCCTCGATACAAGAGCCGA
GACCCAGAGTCTTGAGAGCAGCGACTCCTCCGGGCTGAAGCAGCTCTCCC
ACGATTCGGTCCGGTTCGGAGAGATCTCGTTCCACAACAAGAACCTTTCT
GCCCTGATTTCCAAGAGCCACGGCCAGAGCGGGCCCGGCAATACCAGCTC
CGACAATGACCACGTCGTACTCGTTGGTCTGGGTGGCGCTGGTCTCTGCT
GCAGACTGTTGGGTGACCAT.

In some embodiments, an SQE comprises the amino acid sequence set forth in GenBank Accession No. CAA97201.1 (SEQ ID NO: 312).

In some embodiments, a nucleotide sequence encoding SEQ ID NO: 312 is set forth in SEQ ID NO: 303.

SQEs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to a SQE sequence (e.g., nucleic acid or amino acid sequence), to a sequence set forth as SEQ ID NO: 9-10, 277-279, 293-295, 303 or 312, or to any SQE sequence disclosed in this application or known in the art.

In some embodiments, an SQE of the present disclosure is capable of promoting formation of an epoxide in a squalene compound (e.g., epoxidation of squalene or 2,3-oxidosqualene). In some embodiments, an SQE of the present disclosure catalyzes the formation of a mogrol precursor (e.g., 2-3-oxidosqualene or 2-3, 22-23-diepoxysqualene).

Activity, such as specific activity, of a recombinant SQE may be measured as the concentration of an isoprenoid precursor (e.g., 2-3-oxidosqualene or 2-3, 22-23-diepoxysqualene) produced per unit of enzyme per unit of time. In some embodiments, an SQE of the present disclosure has an activity, such as specific activity, of at least 0.0000001 μmol/min/mg (e.g., at least 0.000001 μmol/min/mg, at least 0.00001 μmol/min/mg, at least 0.0001 μmol/min/mg, at least 0.001 μmol/min/mg, at least 0.01 μmol/min/mg, at least 0.1 pmol/min/mg, at least 1 μmol/min/mg, at least 10 μmol/min/mg, or at least 100 μmol/min/mg, including all values in between).

In some embodiments, the activity, such as specific activity, of a SQE is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control SQE.

The activity of a squalene epoxidase may be altered using any suitable method or method known in the art. In some embodiments, one or more amino acid changes alters the activity of a squalene epoxidase as compared to a control squalene epoxidase. In some embodiments, a control squalene epoxidase is a wild-type squalene epoxidase. In some embodiments, the expression of a squalene epoxidase is altered to affect squalene epoxidase activity. In some embodiments, a host cell comprises a heterologous polynucleotide that is capable of reducing squalene epoxidase activity. In some embodiments, a reduction in squalene epoxidase expression in a host cell reduces squalene epoxidase activity. In some embodiments, a host cell comprises a heterologous polynucleotide that is capable of increasing squalene epoxidase activity. In some embodiments, an increase in squalene epoxidase expression in a host cell increases squalene epoxidase activity.

In some embodiments, the activity of a squalene epoxidase is reduced using: a weak promoter to drive expression of the squalene epoxidase, one or more codons that are not optimized for a particular host cell, use of an antisense nucleic acid, genetic modification that alters gene expression and/or introduces one or more alterations, alteration of a promoter driving expression of a squalene epoxidase and/or altering the coding sequence of a squalene epoxidase.

In some embodiments, the activity of a squalene epoxidase is increased using: a strong promoter to drive expression of the squalene epoxidase, one or more codons that are optimized for a particular host cell, a nucleic acid encoding a squalene epoxidase, genetic modification that alters gene expression and/or introduces one or more alterations, alteration of a promoter driving expression of a squalene epoxidase and/or altering the coding sequence of a squalene epoxidase.

In some embodiments, a squalene epoxidase is capable of increasing production of an isoprenoid precursor and/or isoprenoid by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of the isoprenoid precursor and/or isoprenoid by a host cell that does not comprise the squalene epoxidase. In some embodiments, a squalene epoxidase is capable of increasing production of an isoprenoid precursor and/or isoprenoid by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production of the isoprenoid precursor and/or isoprenoid by a host cell that does not comprise the squalene epoxidase. In some embodiments, a squalene epoxidase is capable of increasing production of an isoprenoid precursor and/or isoprenoid by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of the isoprenoid precursor and/or isoprenoid by a host cell that does not comprise the squalene epoxidase.

In some embodiments, a host cell comprising a squalene epoxidase is capable of producing at least 0.01 mg/L, at least 0.05 mg/L, at least 1 mg/L, at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 150 mg/L, at least 200 mg/L, at least 250 mg/L, at least 300 mg/L, at least 350 mg/L, at least 400 mg/L, at least 450 mg/L, at least 500 mg/L, at least 550 mg/L, at least 600 mg/L, at least 650 mg/L, at least 700 mg/L, at least 750 mg/L, at least 800 mg/L, at least 850 mg/L, at least 900 mg/L, at least 950 mg/L, at least 1 g/L, at least 1.1 g/L, at least 1.2 g/L, at least 1.3 g/L, at least 1.4 g/L, at least 1.5 g/L, at least 1.6 g/L, at least 1.7 g/L, at least 1.8 g/L, at least 1.9 g/L, at least 2 g/L, at least 2.1 g/L, at least 2.2 g/L, at least 2.3 g/L, at least 2.4 g/L, at least 2.5 g/L, at least 2.6 g/L, at least 2.7 g/L, at least 2.8 g/L, at least 2.9 g/L, at least 3 g/L, at least 3.1 g/L, at least 3.2 g/L, at least 3.3 g/L, at least 3.4 g/L, at least 3.5 g/L, at least 3.6 g/L, at least 3.7 g/L, at least 3.8 g/L, at least 3.9 g/L, at least 4 g/L, at least 4.1 g/L, at least 4.2 g/L, at least 4.3 g/L, at least 4.4 g/L, at least 4.5 g/L, at least 4.6 g/L, at least 4.7 g/L, at least 4.8 g/L, at least 4.9 g/L, at least 5 g/L, at least 5.1 g/L, at least 5.2 g/L, at least 5.3 g/L, at least 5.4 g/L, at least 5.5 g/L, at least 5.6 g/L, at least 5.7 g/L, at least 5.8 g/L, at least 5.9 g/L, at least 6 g/L, at least 6.1 g/L, at least 6.2 g/L, at least 6.3 g/L, at least 6.4 g/L, at least 6.5 g/L, at least 6.6 g/L, at least 6.7 g/L, at least 6.8 g/L, at least 6.9 g/L, at least 7 g/L, at least 7.1 g/L, at least 7.2 g/L, at least 7.3 g/L, at least 7.4 g/L, at least 7.5 g/L, at least 7.6 g/L, at least 7.7 g/L, at least 7.8 g/L, at least 7.9 g/L, at least 8 g/L, at least 8.1 g/L, at least 8.2 g/L, at least 8.3 g/L, at least 8.4 g/L, at least 8.5 g/L, at least 8.6 g/L, at least 8.7 g/L, at least 8.8 g/L, at least 8.9 g/L, at least 9 g/L, at least 9.1 g/L, at least 9.2 g/L, at least 9.3 g/L, at least 9.4 g/L, at least 9.5 g/L, at least 9.6 g/L, at least 9.7 g/L, at least 9.8 g/L, at least 9.9 g/L, at least 10 g/L, at least 20 g/L, at least 30 g/L, at least 40 g/L, at least 50 g/L, at least 60 g/L, at least 70 g/L, at least 80 g/L, at least 90 g/L, at least 100 g/L, at least 200 g/L, at least 300 g/L, at least 400 g/L, at least 500 g/L, at least 600 g/L, at least 700 g/L, at least 800 g/L, at least 900 g/L, or at least 1000 g/L including all values in between of an isoprenoid precursor and/or isoprenoid. In some embodiments, a host cell comprising a squalene epoxidase is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, at most 1 g/L, at most 1.1 g/L, at most 1.2 g/L, at most 1.3 g/L, at most 1.4 g/L, at most 1.5 g/L, at most 1.6 g/L, at most 1.7 g/L, at most 1.8 g/L, at most 1.9 g/L, at most 2 g/L, at most 2.1 g/L, at most 2.2 g/L, at most 2.3 g/L, at most 2.4 g/L, at most 2.5 g/L, at most 2.6 g/L, at most 2.7 g/L, at most 2.8 g/L, at most 2.9 g/L, at most 3 g/L, at most 3.1 g/L, at most 3.2 g/L, at most 3.3 g/L, at most 3.4 g/L, at most 3.5 g/L, at most 3.6 g/L, at most 3.7 g/L, at most 3.8 g/L, at most 3.9 g/L, at most 4 g/L, at most 4.1 g/L, at most 4.2 g/L, at most 4.3 g/L, at most 4.4 g/L, at most 4.5 g/L, at most 4.6 g/L, at most 4.7 g/L, at most 4.8 g/L, at most 4.9 g/L, at most 5 g/L, at most 5.1 g/L, at most 5.2 g/L, at most 5.3 g/L, at most 5.4 g/L, at most 5.5 g/L, at most 5.6 g/L, at most 5.7 g/L, at most 5.8 g/L, at most 5.9 g/L, at most 6 g/L, at most 6.1 g/L, at most 6.2 g/L, at most 6.3 g/L, at most 6.4 g/L, at most 6.5 g/L, at most 6.6 g/L, at most 6.7 g/L, at most 6.8 g/L, at most 6.9 g/L, at most 7 g/L, at most 7.1 g/L, at most 7.2 g/L, at most 7.3 g/L, at most 7.4 g/L, at most 7.5 g/L, at most 7.6 g/L, at most 7.7 g/L, at most 7.8 g/L, at most 7.9 g/L, at most 8 g/L, at most 8.1 g/L, at most 8.2 g/L, at most 8.3 g/L, at most 8.4 g/L, at most 8.5 g/L, at most 8.6 g/L, at most 8.7 g/L, at most 8.8 g/L, at most 8.9 g/L, at most 9 g/L, at most 9.1 g/L, at most 9.2 g/L, at most 9.3 g/L, at most 9.4 g/L, at most 9.5 g/L, at most 9.6 g/L, at most 9.7 g/L, at most 9.8 g/L, at most 9.9 g/L, at most 10 g/L, at most 20 g/L, at most 30 g/L, at most 40 g/L, at most 50 g/L, at most 60 g/L, at most 70 g/L, at most 80 g/L, at most 90 g/L, at most 100 g/L, at most 200 g/L, at most 300 g/L, at most 400 g/L, at most 500 g/L, at most 600 g/L, at most 700 g/L, at most 800 g/L, at most 900 g/L, or at most 1000 g/L of an isoprenoid precursor and/or isoprenoid. In some embodiments, a host cell comprising a squalene epoxidase is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, between 1 mg/L and 1,000 mg/L, between 1 g/L and 10 g/L, between 10 g/L and 20 g/L, between 10 g/L and 50 g/L, between 50 g/L and 100 g/L, between 100 g/L and 200 g/L, between 200 g/L and 300 g/L, between 300 g/L and 400 g/L, between 400 g/L and 500 g/L, between 500 g/L and 600 g/L, between 600 g/L and 700 g/L, between 700 g/L and 800 g/L, between 800 g/L and 900 g/L, between 900 g/L and 1000 g/L, between 1 g/L and 50 g/L, between 1 g/L and 100 g/L, between 1 g/L and 500 g/L, or between 1 g/L and 1,000 g/L, including all values in between of an isoprenoid precursor and/or isoprenoid. In some embodiments, the isoprenoid precursor is mevalonate. In some embodiments, the isoprenoid precursor is IPP, GPP, FPP. In some embodiments, the isoprenoid precursor is mevalonate or 2-3-oxidosqualene.

In some embodiments, a squalene epoxidase is capable of decreasing production of lanosterol or an isoprenoid precursor by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between as compared to production of lanosterol or an isoprenoid precursor by a host cell that does not comprise the squalene epoxidase. In some embodiments, a squalene epoxidase is capable of decreasing production of lanosterol or an isoprenoid precursor by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between as compared to production lanosterol or an isoprenoid precursor by a host cell that does not comprise the squalene epoxidase. In some embodiments, a squalene epoxidase is capable of decreasing production of lanosterol or an isoprenoid precursor by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between as compared to production of lanosterol or an isoprenoid precursor by a host cell that does not comprise the squalene epoxidase.

In some embodiments, increasing the activity of squalene epoxidase promotes the production of 2-3-oxidosqualene, lanosterol, 2-3; 22,23-diepoxysqualene and/or isoprenoids derived from these compounds. In some embodiments, decreasing the activity of squalene epoxidase promotes the production of isoprenoids derived from farnesyl diphosphate except for 2-3-oxidosqualene and isoprenoids derived from it, promotes production of intermediate molecules in the mevalonate pathway, e.g. mevalonate, promotes production of intermediate molecules in the MEP pathway, e.g. 2C-methyl-D-erythritol 2,4-cyclodiphosphate, and/or reduces production of 2-3-oxidosqualene, lanosterol, 2-3; 22,23-diepoxysqualene or isoprenoids derived from them.

### 3. Mevalonate (MEV) Pathway Enzymes

Isoprenoid and isoprenoid production can be augmented by upregulating or downregulating the expression of one or more genes or the activity of their gene product or encoded enzymes including, for example, one or more enzymes in the MEV pathway as follows.

FIG. 1A provides non-limiting examples of the enzymes involved in the mevalonate (MEV) pathway. First, an acetoacetyl-CoA thiolase condenses two acetyl-CoA molecules to form acetoacetyl-CoA. An acetoacetyl-CoA thiolase may be encoded by an ERG10 gene. UniProtKB Accession Nos. P41338 and P10551 provide non-limiting examples of acetoacetyl-CoA thiolases. Increased expression of the ERG10 gene or increased activity of ERG10 enzyme can be used to increase production of isoprenoids or isoprenoid precursors.

Acetoacetyl CoA synthase synthesizes acetoacetyl-CoA by catalyzing the condensation of acetyl-CoA and malonyl-CoA to form acetoacetyl-CoA and CoA. Increased expression of the acetoacetyl CoA synthase gene or increased activity of the acetoacetyl CoA synthase enzyme can be used to increase production of isoprenoids or isoprenoid precursors.

HMG-CoA synthase condenses acetoacetyl-CoA to form 3-hydroxy-3-methyl-glutaryl-CoA (HMG-CoA). An HMG-CoA synthase may be encoded by an ERG13 gene. UniProtKB Accession Nos. P54839 and A0A1D8PTW6 provide non-limiting examples of HMG-CoA synthases. Increased expression of the ERG13 gene or increased activity of ERG13 enzyme can be used to increase production of isoprenoids or isoprenoid precursors.

HMG-CoA reductases subsequently reduce HMG-CoA to produce mevalonate. An HMG-CoA reductase may be encoded by an HMG1 gene. UniProtKB Accession No. P12683 provides a non-limiting example of an HMG-CoA reductase encoded by HMG1. An HMG-CoA reductase may be encoded by an HMG2 gene. UniProtKB Accession No. P12684 provides a non-limiting example of an HMG-CoA reductase encoded by HMG2. Increased expression of the HMG1 and/or HMG2 genes or increased activity of the HMG1 and/or HMG2 enzymes can be used to increase production of isoprenoids or isoprenoid precursors.

Mevalonate-5-kinase phosphorylates mevalonate to form mevalonate-5-phosphate. A mevalonate-5-kinase may be encoded by an ERG12 gene. UniProtKB Accession Nos. P07277 and A0A1D8PEL1 provide non-limiting examples of mevalonate-5-kinases. Increased expression of the ERG12 gene or increased activity of ERG12 enzyme can be used to increase production of isoprenoids or isoprenoid precursors.

Mevalonate-5-phosphate is phosphorylated by phosphomevalonate kinase to form mevalonate pyrophosphate. A phosphomevalonate kinase may be encoded by an ERG8 gene. UniProtKB Accession No. P24521 provides a non-limiting example of a phosphomevalonate kinase. Increased expression of the ERG8 gene or increased activity of ERG8 enzyme can be used to increase production of isoprenoids or isoprenoid precursors.

Mevalonate pyrophosphate decarboxylase converts mevalonate pyrophosphate into IPP. A mevalonate pyrophosphate decarboxylase may be encoded by an ERG19 gene. UniProtKB Accession No. P32377 provides a non-limiting example of a mevalonate pyrophosphate decarboxylase. Increased expression of the ERG19 gene or increased activity of ERG19 enzyme can be used to increase production of isoprenoids or isoprenoid precursors.

Isopentenyl pyrophosphate isomerase catalyzes the conversion of IPP to dimethylallyl pyrophosphate (DMAPP). IPP isomerization to DMAPP promotes isoprenoid biosynthesis as DMAPP is an electrophile and more reactive than IPP. An isopentenyl pyrophosphate isomerase may be encoded by an IDI1 gene. UniProtKB Accession No. P15496 provides a non-limiting example of an Isopentenyl pyrophosphate isomerase.

In some embodiments, increasing the activity of one or more of the mevalonate (MEV) pathway genes promotes the production of isoprenoids.

### Archaeal Mevalonate 1 (MEV-A1) Pathway Enzymes

Isoprenoid and isoprenoid production can be augmented by upregulating or downregulating the expression of one or more genes or the activity of their gene product or encoded enzymes including, for example, one or more enzymes in the MEV-A1 pathway as follows.

FIG. 1B provides non-limiting examples of the enzymes involved in the archaeal mevalonate 1 (MEV-A1) pathway. First, an acetoacetyl-CoA thiolase condenses two acetyl-CoA molecules to form acetoacetyl-CoA. An acetoacetyl-CoA thiolase may be encoded by an ERG10 gene. UniProtKB Accession Nos. P41338 and P10551 provide non-limiting examples of acetoacetyl-CoA thiolases.

Acetoacetyl CoA synthase also synthesizes acetoacetyl-CoA by catalyzing the condensation of acetyl-CoA and malonyl-CoA to form acetoacetyl-CoA and CoA.

Then, an HMG-CoA synthase condenses acetoacetyl-CoA to form 3-hydroxy-3-methyl-glutaryl-CoA (HMG-CoA). An HMG-CoA synthase may be encoded by an ERG13 gene. UniProtKB Accession Nos. P54839 and A0A1D8PTW6 provide non-limiting examples of HMG-CoA synthases.

HMG-CoA reductases subsequently reduce HMG-CoA to produce mevalonate. An HMG-CoA reductase may be encoded by an HMG1 gene. UniProtKB Accession No. P12683 provides a non-limiting example of an HMG-CoA reductase encoded by HMG1. An HMG-CoA reductase may be encoded by an HMG2 gene. UniProtKB Accession No. P12684 provides a non-limiting example of an HMG-CoA reductase encoded by HMG2.

Then, mevalonate-5-kinase phosphorylates mevalonate to form mevalonate-5-phosphate. A mevalonate-5-kinase may be encoded by an ERG12 gene. UniProtKB Accession Nos. P07277 and A0A1D8PEL1 provide non-limiting examples of mevalonate-5-kinases.

Mevalonate-5-phosphate is decarboxylated by mevalonate-5-phosphate decarboxylase to form isopentenyl pyrophosphate. A mevalonate-5-phosphate decarboxylase may be encoded by a PMD gene. UniProtKB Accession Nos. D4GXZ3 and Q18K00 provide non-limiting examples of mevalonate-5-phosphate decarboxylases.

Isopentenyl phosphate kinase converts isopentenyl pyrophosphate into IPP. An isopentenyl phosphate kinase may be encoded by an IPK gene. UniProtKB Accession Nos. Q60352 and Q56187 provide non-limiting examples of isopentenyl phosphate kinases.

Isopentenyl pyrophosphate isomerase catalyzes the conversion of IPP to DMAPP. IPP isomerization to DMAPP promotes isoprenoid biosynthesis as DMAPP is an electrophile and more reactive than IPP. An isopentenyl pyrophosphate isomerase may be encoded by an IDI1 gene. UniProtKB Accession No. P15496 provides a non-limiting example of an Isopentenyl pyrophosphate isomerase.

In some embodiments, increasing the activity of one or more of the Archaeal Mevalonate I (MEV-A1) pathway genes promotes the production of isoprenoids.

### Archaeal Mevalonate 2 (MEV-A2) Pathway Enzymes

Isoprenoid and isoprenoid production can be augmented by upregulating or downregulating the expression of one or more genes or the activity of their gene product or encoded enzymes including, for example, one or more enzymes in the MEV-A2 pathway as follows.

FIG. 1C provides non-limiting examples of the enzymes involved in the archaeal mevalonate 2 (MEV-A1) pathway. First, an acetoacetyl-CoA thiolase condenses two acetyl-CoA molecules to form acetoacetyl-CoA. An acetoacetyl-CoA thiolase may be encoded by an ERG10 gene. UniProtKB Accession Nos. P41338 and P10551 provide non-limiting examples of acetoacetyl-CoA thiolases.

Acetoacetyl CoA synthase also synthesizes acetoacetyl-CoA by catalyzing the condensation of acetyl-CoA and malonyl-CoA to form acetoacetyl-CoA and CoA.

Then, an HMG-CoA synthase condenses acetoacetyl-CoA to form 3-hydroxy-3-methyl-glutaryl-CoA (HMG-CoA). An HMG-CoA synthase may be encoded by an ERG13 gene. UniProtKB Accession Nos. P54839 and A0A1D8PTW6 provide non-limiting examples of HMG-CoA synthases.

HMG-CoA reductases subsequently reduce HMG-CoA to produce mevalonate. An HMG-CoA reductase may be encoded by an HMG1 gene. UniProtKB Accession No. P12683 provides a non-limiting example of an HMG-CoA reductase encoded by HMG1. An HMG-CoA reductase may be encoded by an HMG2 gene. UniProtKB Accession No. P12684 provides a non-limiting example of an HMG-CoA reductase encoded by HMG2.

Then, mevalonate-3-kinase phosphorylates mevalonate to form mevalonate-3-phosphate. A mevalonate-3-kinase may be encoded by an M3K gene. UniProtKB Accession Nos. Q9HIN1 and Q6KZB1 provide non-limiting examples of mevalonate-3-kinases.

Mevalonate-3-phosphate is phosphorylated by mevalonate-3-phosphate-5-kinase to form mevalonate-3,5-bisphosphate. A mevalonate-3-phosphate-5-kinase may be encoded by an M3K gene. UniProtKB Accession Nos. Q9HIN1 and Q6KZB1 provide non-limiting examples of mevalonate-3-kinases.

Then, mevalonate-3,5-phosphate is decarboxylated by mevalonate-5-phosphate decarboxylase to form isopentenyl pyrophosphate. A mevalonate-5-phosphate decarboxylase may be encoded by a PMD gene. UniProtKB Accession Nos. D4GXZ3 and Q18K00 provide non-limiting examples of mevalonate-5-phosphate decarboxylases.

Isopentenyl phosphate kinase converts isopentenyl pyrophosphate into IPP. An isopentenyl phosphate kinase may be encoded by an IPK gene. UniProtKB Accession Nos. Q60352 and Q56187 provide non-limiting examples of isopentenyl phosphate kinases.

Isopentenyl pyrophosphate isomerase catalyzes the conversion of IPP to DMAPP. IPP isomerization to DMAPP promotes isoprenoid biosynthesis as DMAPP is an electrophile and more reactive than IPP. An isopentenyl pyrophosphate isomerase may be encoded by an IDI1 gene. UniProtKB Accession No. P15496 provides a non-limiting example of an Isopentenyl pyrophosphate isomerase.

In some embodiments, increasing the activity of one or more of the Archaeal Mevalonate 2 (MEV-A2) pathway genes promotes the production of isoprenoids.

### Methylerithritol Phosphate (MEP) Pathway Enzymes

Isoprenoid and isoprenoid production can be augmented by upregulating or downregulating the expression of one or more genes or the activity of their gene product or encoded enzymes including, for example, one or more enzymes in the MEP pathway as follows.

FIG. 1D provides non-limiting examples of the enzymes involved in the methylerithritol phosphate (MEP) pathway. First, a 1-deoxy-D-xylulose-5-phosphate synthase condenses pyruvate and glyceraldehyde 3-phosphate to form 1-deoxy-D-xylulose 5-phosphate (DXP). An 1-deoxy-D-xylulose-5-phosphate synthase may be encoded by a DXS gene. UniProtKB Accession Nos. P77488 and A0A3D8XGB8 provide non-limiting examples of 1-deoxy-D-xylulose-5-phosphate synthases.

Then, a 1-deoxy-D-xylulose-5-phosphate reductoisomerase (DXR) reduces DXP to form 2C-methyl-D-erythritol 4-phosphate (MEP). A 1-deoxy-D-xylulose-5-phosphate reductoisomerase may be encoded by an IspC gene or a DXR gene. UniProtKB Accession Nos. P45568 and 096693 provide non-limiting examples of 1-deoxy-D-xylulose-5-phosphate reductoisomerases.

2-C-methyl-D-erythritol 4-phosphate cytidylyltransferase (CMS) subsequently converts DXP to 4-diphosphocytidyl-2C-methyl D-erythritol (CDP-ME). A 2-C-methyl-D-erythritol 4-phosphate cytidylyltransferase may be encoded by an YgpP gene or an IspD gene. UniProtKB Accession Nos. Q46893 and A0A5E7ZFQ6 provide non-limiting examples of 2-C-methyl-D-erythritol 4-phosphate cytidylyltransferases.

Then, CDP-ME undergoes a phosphorylation by the ATP-dependent 4-diphosphocytidyl-2-C-methyl-D-erythritol kinase (CMK) to produce 4-diphosphocytidyl-2C-methyl D-erythritol 2-phosphate (CDP-MEP). A 4-diphosphocytidyl-2-C-methyl-D-erythritol kinase may be encoded by an YchB gene or an IspE gene. UniProtKB Accession Nos. P62615 and A0A535X269 provide non-limiting examples of 4-diphosphocytidyl-2-C-methyl-D-erythritol kinases.

CDP-MEP is cyclized by 2-C-methyl-D-erythritol 2,4-cyclodiphosphate synthase (MCS) to form 2C-methyl-D-erythritol 2,4-cyclodiphosphate (MEC or MEcPP). A 2-C-methyl-D-erythritol 2,4-cyclodiphosphate synthase may be encoded by an IspF gene. UniProtKB Accession Nos. P62617 and Q8RQP5 provide non-limiting examples of 2-C-methyl-D-erythritol 2,4-cyclodiphosphate synthases.

4-hydroxy-3-methylbut-2-en-1-yl diphosphate synthase (HDS) converts MEC into 4-hydroxy-3-methylbut-2-en-1-yl diphosphate (HMB-PP or HMBPP). A 4-hydroxy-3-methylbut-2-en-1-yl diphosphate synthase may be encoded by a GcpE gene or an IspG gene. UniProtKB Accession Nos. P62620 and Q8DK70 provide non-limiting examples of 4-hydroxy-3-methylbut-2-en-1-yl diphosphate synthases.

4-hydroxy-3-methylbut-2-en-1-yl diphosphate reductase (HDR) converts mevalonate HMB-PP into a mixture of IPP and DMAPP. A 4-hydroxy-3-methylbut-2-en-1-yl diphosphate reductase may be encoded by an LytB gene or an IspH gene. UniProtKB Accession Nos. W1F471 and A0A113QNS4 provide non-limiting examples of 4-hydroxy-3-methylbut-2-en-1-yl diphosphate reductases.

Isopentenyl pyrophosphate isomerase catalyzes the conversion of IPP to DMAPP. IPP isomerization to DMAPP promotes isoprenoid biosynthesis as DMAPP is an electrophile and more reactive than IPP. An isopentenyl pyrophosphate isomerase may be encoded by an IDI1 gene. UniProtKB Accession No. P15496 provides a non-limiting example of an Isopentenyl pyrophosphate isomerase.

Increasing the activity of one or more of the methylerithritol phosphate (MEP) pathway genes promotes the production of isoprenoids.

### Prenyltransferases

As used herein, a “prenyltransferase” refers to a protein that promotes the transfer of a prenyl group onto a substrate. In some embodiments, a prenyltransferase promotes the condensation of IPP with an allylic substrate to generate prenyl diphosphates of different lengths. Geranyl pyrophosphate synthases catalyze the formation of GPP. A geranyl pyrophosphate synthase may be encoded by a ERG20 gene.

Farnesyl diphosphate synthase catalyzes conversion of GPP into FPP. A farnesyl diphosphate synthase may be encoded by the ERG20 gene. UniProtKB Accession Nos. P08524 and A0A1D8PH78 provide non-limiting examples of farnesyl diphosphate synthases.

Geranylgeranyl pyrophosphate synthase catalyzes the formation of GGPP. A geranylgeranyl pyrophosphate synthase may be encoded by a GGPPS gene. UniProtKB Accession No. Q64KQ5 provides a non-limiting example of a geranylgeranyl pyrophosphate synthase.

In some embodiments, increasing the activity of one or more of the prenyltransferases promotes the production of isoprenoids.

### Squalene Synthases

As used herein, a “squalene synthase” refers to a protein that catalyzes production of squalene from farnesyl diphosphate. A squalene synthase may be encoded by an ERG9 gene. UniProtKB Accession Nos. P36596, P29704, and Q9HGZ6 provide non-limiting examples of squalene synthases.

In some embodiments, increasing the activity of squalene synthase promotes the production of squalene, 2-3-oxidosqualene, lanosterol, 2-3; 22,23-diepoxysqualene and/or isoprenoids derived from them. In some embodiments, decreasing the activity of squalene synthase reduces production of isoprenoids derived from farnesyl diphosphate except for squalene and isoprenoids derived from it, promotes production of intermediate molecules in the mevalonate pathway, e.g. mevalonate, promotes production of intermediate molecules in the MEP pathway, e.g. 2C-methyl-D-erythritol 2,4-cyclodiphosphate, and/or decreases production of squalene, 2-3-oxidosqualene, lanosterol, 2-3; 22,23-diepoxysqualene or isoprenoids derived from them.

### Terpene Synthases

As used herein, a “terpene synthase” refers to a protein that is capable of producing an isoprenoid, optionally using a prenyl diphosphate as a substrate. At least two types of terpene synthases have been characterized: classic terpene synthases and isoprenyl diphosphate synthase-type terpene synthases. Classic terpene synthases are found in prokaryotes (e.g., bacteria) and in eukaryotes (e.g., plants, fungi and amoebae), while isoprenyl diphosphate synthase-type terpene synthases have been found in insects (see, e.g., Chen et al., Terpene synthase genes in eukaryotes beyond plants and fungi: Occurrence in social amoebae. Proc Nat Acad Sci USA. 2016; 113(43):12132-12137, which is hereby incorporated by reference in its entirety). Several highly conserved structural motifs have been reported in classic terpene synthases, including an aspartate-rich “DDxx(x)D/E” motif and a “NDxxSxxxD/E” (SEQ ID NO: 55) motif, which have both been implicated in coordinating substrate binding (see, e.g., Starks et al., Structural basis for cyclic terpene biosynthesis by tobacco 5-epi-aristolochene synthase. Science. 1997 Sep. 19; 277(5333):1815-20; and Christianson et al., Unearthing the roots of the terpenome. Curr Opin Chem Biol. 2008 April; 12(2):141-50, each of which is hereby incorporated by reference in its entirety for this purpose). See also, e.g., WO 2019/161141 and WO 2020/176547.

In some embodiments, increasing the activity of an isoprenoid-specific terpene synthase promotes the production of this isoprenoid.

### Acetoacetyl CoA Synthases

Aspects of the present invention provide acetoacetyl CoA synthases, which catalyze the condensation of acetyl-CoA and malonyl-CoA to form acetoacetyl-CoA and CoA, but do not accept malonyl-[acyl-carrier-protein] as a substrate. Acetoacetyl CoA synthases can also 5 convert mnaionyl-CoA into acetyl-CoA via decarboxylation of mnalonyl-CoA. Aspects of the present invention provide an acetoacetyl CoA synthase, which increases levels of acetoacetyl-CoA.

In some embodiments, the acetoacetyl CoA synthase is encoded by a NphT7 gene. NphT7 catalyzes an alternative path to acetoacetyl-CoA and is present in the MEV pathway but not the MEP pathway. See, e.g., FIG. 1A. In some embodiments, the acetoacetyl CoA synthase comprises the amino acid sequence:

(SEQ ID NO: 6)
MTDVRFRIIGTGAYVPERIVSNDEVGAPAGVDDDWITRKTGIRQRRWAAD
DQATSDLATAAGRAALKAAGITPEQLTVIAVATSTPDRPQPPTAAYVQHH
LGATGTAAFDVNAVCSGTVFALSSVAGTLVYRGGYALVIGADLYSRILNP
ADRKTVVLFGDGAGAMVLGPTSTGTGPIVRRVALHTFGGLTDLIRVPAGG
SRQPLDTDGLDAGLQYFAMDGREVRRFVTEHLPQLIKGFLHEAGVDAADI
SHFVPHQANGVMLDEVFGELHLPRATMHRTVETYGNTGAASIPITMDAAV
RAGSFRPGELVLLAGFGGGMAASFALIEW.

In some embodiments, the acetoacetyl CoA synthase is encoded by a polynucleotide having a sequence of:

(SEQ ID NO: 7)
ATGACCGACGTCCGATTCCGAATTATCGGTACTGGTGCCTACGTTCCCGA
ACGAATCGTTTCCAACGATGAAGTCGGTGCTCCTGCCGGTGTTGACGACG
ACTGGATCACCCGAAAGACCGGTATTCGACAGCGACGATGGGCTGCCGAT
GACCAGGCCACCTCTGATCTGGCCACTGCTGCCGGTCGAGCTGCCCTGAA
GGCCGCTGGTATCACTCCCGAGCAGCTGACCGTTATTGCTGTTGCCACCT
CCACTCCCGATCGACCCCAGCCTCCCACTGCTGCCTATGTTCAGCACCAC
CTCGGAGCCACCGGTACTGCTGCCTTCGACGTCAACGCTGTCTGCTCCGG
TACCGTTTTCGCCCTGTCCTCTGTTGCTGGCACCCTCGTTTACCGAGGTG
GTTACGCTCTGGTCATTGGCGCTGACCTGTACTCTCGAATCCTCAACCCT
GCCGACCGAAAGACCGTCGTTCTGTTCGGTGATGGTGCCGGTGCCATGGT
TCTCGGTCCTACCTCCACCGGTACTGGTCCCATTGTTCGACGAGTTGCCC
TGCACACCTTCGGTGGTCTGACCGACCTGATTCGAGTCCCCGCTGGTGGT
TCTCGACAGCCCCTGGACACTGATGGCCTCGATGCTGGACTGCAGTACTT
CGCTATGGACGGTCGTGAGGTCCGACGATTCGTCACTGAGCACCTCCCCC
AGCTGATCAAGGGTTTCCTGCACGAGGCCGGTGTCGACGCTGCCGACATC
TCTCACTTCGTCCCTCATCAGGCCAACGGTGTCATGCTCGACGAGGTCTT
CGGCGAGCTGCATCTGCCTCGAGCTACCATGCACCGAACTGTCGAGACTT
ACGGCAACACCGGAGCTGCCTCCATTCCCATCACCATGGACGCTGCCGTT
CGAGCCGGTTCCTTCCGACCTGGTGAGCTGGTCCTGCTGGCCGGTTTCGG
TGGCGGTATGGCCGCTTCCTTCGCCCTGATCGAGTGGTAG.

Acetoacetyl CoA synthases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with the acetoacetyl CoA synthase sequence set forth as SEQ ID NO: 6 or 7, or to any acetoacetyl CoA synthase disclosed in this application or known in the art. The present disclosure also pertains to a host cell comprising such an acetoacetyl CoA synthase, polynucleotides encoding such an acetoacetyl CoA synthase, and/or methods of use of such a host cell.

In some embodiments, an acetoacetyl CoA synthase of the present disclosure is capable of promoting formation of acetoacetyl-CoA.

Activity, such as specific activity, of a recombinant acetoacetyl CoA synthase may be measured as the concentration of acetoacetyl-CoA produced per unit of enzyme per unit of time. In some embodiments, an acetoacetyl CoA synthase of the present disclosure has an activity, such as specific activity, of at least 0.0000001 μmol/min/mg (e.g., at least 0.000001 μmol/min/mg, at least 0.00001 μmol/min/mg, at least 0.0001 μmol/min/mg, at least 0.001 μmol/min/mg, at least 0.01 μmol/min/mg, at least 0.1 μmol/min/mg, at least 1 μmol/min/mg, at least 10 μmol/min/mg, or at least 100 μmol/min/mg, including all values in between).

In some embodiments, the activity, such as specific activity, of an acetoacetyl CoA synthase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control acetoacetyl CoA synthase.

In various aspects, the present disclosure pertains to: an acetoacetyl CoA synthase as provided in SEQ ID NO: 6; a polynucleotide encoding an acetoacetyl CoA synthase as provided in SEQ ID NO: 7; a host cell comprising an acetoacetyl CoA synthase as provided in SEQ ID NO: 6; or a host cell comprising a polynucleotide encoding an acetoacetyl CoA synthase as provided in SEQ ID NO: 7. In some aspects, the present disclosure pertains to: a method of making an isoprenoid or isoprenoid precursor, wherein the method comprises the step of: producing the isoprenoid or isoprenoid precursor in a host cell comprising an acetoacetyl CoA synthase as provided in SEQ ID NO: 6, and/or a polynucleotide encoding an acetoacetyl CoA synthase as provided in SEQ ID NO: 7.

In various embodiments, any host cell described herein can further comprise an acetoacetyl CoA synthase described herein; any method described herein can be performed using any host cell described herein that further describes a an acetoacetyl CoA synthase described herein.

### Variants

Aspects of the disclosure relate to polynucleotides encoding any of the recombinant polypeptides described, such as lanosterol synthase, squalene epoxidase, MEV pathway enzyme, MEP pathway enzyme, squalene synthase, prenyltransferase, terpene synthase, and any proteins associated with the disclosure. Variants of polynucleotide or amino acid sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., nucleic acid or amino acid residues). Identity measures the percent of identical matches between the smaller of two or more sequences with gap alignments (if any) addressed by a particular mathematical model, algorithms, or computer program.

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., lanosterol synthase, squalene epoxidase, MEV pathway enzyme, MEP pathway enzyme, squalene synthase, prenyltransferase, terpene synthase, or variant of any protein associated with the disclosure) comprises a domain that shares a secondary structure (e.g., alpha helix, beta sheet) with a reference polypeptide (e.g., a reference lanosterol synthase, MEV pathway enzyme, MEP pathway enzyme, squalene epoxidase, squalene synthase, prenyltransferase, terpene synthase, or any protein associated with the disclosure). In some embodiments, a polypeptide variant (e.g., lanosterol synthase, squalene epoxidase, MEV pathway enzyme, MEP pathway enzyme, squalene synthase, prenyltransferase, terpene synthase, or variant of any protein associated with the disclosure) shares a tertiary structure with a reference polypeptide (e.g., a reference lanosterol synthase, squalene epoxidase, MEV pathway enzyme, MEP pathway enzyme, squalene synthase, prenyltransferase, terpene synthase, or any protein associated with the disclosure). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a protein with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant lanosterol synthases, MEV pathway enzymes, non-mevalonate pathway enzymes, squalene synthases, squalene epoxidases, prenyltransferases, terpene synthases, and any other proteins disclosed in this application are also encompassed by the present disclosure. For example, functional variants may bind one or more of the same substrates (e.g., mogrol, mogroside, or precursors thereof) or produce one or more of the same products (e.g., mogrol, mogroside, or precursors thereof). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain. For example, among oxidosqualene cyclases, additional CDS enzymes may be identified in some instances by searching for polypeptides with a leucine residue corresponding to position 123 of SEQ ID NO: 256. This leucine residue has been implicated in determining the product specificity of the CDS enzyme; mutation of this residue can, for instance, result in cycloartenol or parkeol as a product (Takase et al., Org Biomol Chem. 2015 Jul. 13(26):7331-6).

Homology modeling may also be used to identify amino acid residues that are amenable to mutation without affecting function. A non-limiting example of such a method may include use of position-specific scoring matrix (PSSM) and an energy minimization protocol. See, e.g., Stormo et al., Nucleic Acids Res. 1982 May 11; 10(9):2997-3011.

PSSM may be paired with calculation of a Rosetta energy function, which determines the difference between the wild-type and the single-point mutant. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. doi: 10.1016/j.molcel.2016.06.012.

In some embodiments, a lanosterol synthase, MEV or MEP pathway enzyme, squalene synthase, squalene epoxidase, prenyltransferase, terpene synthase, or coding sequence of any protein associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the lanosterol synthase, MEV pathway enzyme, MEP pathway enzyme, squalene synthase, squalene epoxidase, prenyltransferase, terpene synthase, or coding sequence of any protein associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a recombinant lanosterol synthase, MEV pathway enzyme, MEP pathway enzyme, squalene synthase, squalene epoxidase, prenyltransferase, terpene synthase, or other recombinant protein sequence associated with the disclosure alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

The activity of an enzyme of the present disclosure may be altered using any suitable method or method known in the art. In some embodiments, one or more amino acid changes alters the activity of an enzyme as compared to a control enzyme. In some embodiments, a control enzyme is a wild-type enzyme. In some embodiments, the expression of an enzyme is altered to affect enzyme activity. In some embodiments, a host cell comprises a heterologous polynucleotide that is capable of enzyme activity. In some embodiments, a reduction in enzyme expression in a host cell reduces enzyme activity. In some embodiments, a host cell comprises a heterologous polynucleotide that is capable of increasing enzyme activity. In some embodiments, an increase in enzyme expression in a host cell increases enzyme activity.

In some embodiments, the activity of an enzyme is reduced using: a weak promoter to drive expression of the enzyme, one or more codons that are not optimized for a particular host cell, use of an antisense nucleic acid, genetic modification that alters gene expression and/or introduces one or more alterations, alteration of a promoter driving expression of an enzyme and/or altering the coding sequence of an enzyme.

Reduced enzyme activity can mean decreased enzyme expression, decreased enzyme stability, decreased enzyme specific activity, and/or a decrease in enzyme function due to interference by another protein, a nucleic acid or a small molecule inhibitor as known in the art.

In some embodiments, the activity of an enzyme is increased using: a strong promoter to drive expression of the enzyme, one or more codons that are optimized for a particular host cell, a nucleic acid encoding an enzyme, genetic modification that alters gene expression and/or introduces one or more alterations, alteration of a promoter driving expression of an enzyme and/or altering the coding sequence of an enzyme.

The activity, including specific activity, of any of the recombinant polypeptides described in this application may be measured using methods known in the art. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” or “conservatively substituted” refers to an amino acid substitution that does not alter the relative charge or size characteristics or functional activity of the protein in which the amino acid substitution is made.

In some instances, an amino acid is characterized by its R group (see, e.g., Table 6). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 6.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 6
Non-limiting examples of Conservative Amino Acid Substitutions

| | | Conservative Amino |
|---|---|---|
| Original Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide (e.g., lanosterol synthase, MEV pathway enzyme, MEP pathway enzyme, squalene synthase, squalene epoxidase, prenyltransferase, terpene synthase, or any protein associated with the disclosure).

### Expression of Nucleic Acids in Host Cells

Aspects of the present disclosure relate to the recombinant expression of one or more genes encoding the one or more enzymes in the MEV or MEP pathway for the synthesis of isoprenoid or isoprenoid precursors, functional modifications and variants thereof, as well as uses relating thereto. For example, the methods described in this application may be used to produce isoprenoid precursors and/or isoprenoids.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

A nucleic acid encoding any of the recombinant polypeptides, such as lanosterol synthases, MEV or MEP pathway enzymes, squalene synthases, squalene epoxidase, prenyltransferases, terpene synthases, or any proteins associated with the disclosure, described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell, such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined or linked if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Plslcon, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated.

Regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. Vectors may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a polynucleotide, such as a polynucleotide encoding a recombinant polypeptide, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the recombinant polypeptides described in this application, in its genome.

### Host Cells

Any of the proteins of the disclosure may be expressed in a host cell. As used in this application, the term “host cell” refers to a cell that can be used to express a polynucleotide, such as a polynucleotide that encodes a protein used in production of isoprenoids and precursors thereof.

Any suitable host cell may be used to produce any of the recombinant polypeptides, including lanosterol synthases, MEV or MEP pathway enzymes, squalene synthases, squalene epoxidases, prenyltransferases, terpene synthases, and other proteins disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to: Candida, Hansenula, Saccharomyces (e.g., S. cerevisiae), Schizosaccharomyces, Pichia, Kluyveromyces, and Yarrowia (e.g., Y. lipolytica). In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa, W138, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae or Y. lipolytica) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-CoA) synthase (ERG13), farnesyl-diphosphate farnesyl transferase (squalene synthase) (ERG9), may be modified to overexpress squalene epoxidase, or may be modified to downregulate lanosterol synthase. In some embodiments, a host cell (e.g., S. cerevisiae) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase), 3-hydroxy-3-methylglutaryl-CoA (HMG-CoA) synthase, farnesyl-diphosphate farnesyl transferase (squalene synthase), squalene epoxidase, or lanosterol synthase. In some embodiments, a host cell may be modified to reduce or inactivate the activity of a lanosterol synthase or squalene epoxidase. In some embodiments, the squalene epoxidase is encoded by an ERG1 gene. In some embodiments, the lanosterol synthase is encoded by an ERG7 gene. In some embodiments, a host cell is modified to reduce or eliminate expression of one or more transporter genes, such as PDR1 or PDR3, and/or the glucanase gene EXG1.

In some embodiments, a host cell is modified to reduce or inactivate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes.

In some embodiments, a host cell is modified to reduce or inactivate 1, 2, 3, 4, 5, 6, 7, 8, 9, 10, 11, 12, 13, 14, 15, 16, 17, 18, 19, or 20 genes.

Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014; 1205:45-78) or well-known gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., isoprenoid precursor or isoprenoid) may display some differences from the substrate (e.g., isoprenoid precursor or isoprenoid) in terms of solubility, toxicity, cellular accumulation and secretion and in some embodiments can have different fermentation kinetics.

The methods described in this application encompass production of precursors and isoprenoids using a recombinant cell, cell lysate or isolated recombinant polypeptides (e.g., lanosterol synthase, squalene epoxidase, MEV pathway enzyme, MEP pathway enzyme, squalene synthase, prenyltransferase, terpene synthase, and any proteins associated with the disclosure).

Isoprenoid precursors and isoprenoids produced by any of the recombinant cells disclosed in this application may be identified and extracted using any method known in the art. Mass spectrometry (e.g., LC-MS, GC-MS) is a non-limiting example of a method for identification and may be used to help extract a compound of interest.

In some embodiments, a host cell comprising one or more proteins described herein (e.g., a lanosterol synthase, a MEV pathway enzyme, MEP pathway enzyme, a squalene epoxidase, a squalene synthase, a prenyltransferase, a terpene synthase, and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11 g/L, at least 12 g/L, at least 13 g/L, at least 14 g/L, at least 15 g/L, at least 16 g/L, at least 17 g/L, at least 18 g/L, at least 19 g/L, at least 20 g/L, at least 21 g/L, at least 22 g/L, at least 23 g/L, at least 24 g/L, at least 25 g/L, at least 26 g/L, at least 27 g/L, at least 28 g/L, at least 29 g/L, at least 30 g/L, at least 31 g/L, at least 32 g/L, at least 33 g/L, at least 34 g/L, at least 35 g/L, at least 36 g/L, at least 37 g/L, at least 38 g/L, at least 39 g/L, at least 40 g/L, at least 41 g/L, at least 42 g/L, at least 43 g/L, at least 44 g/L, at least 45 g/L, at least 46 g/L, at least 47 g/L, at least 48 g/L, at least 49 g/L, at least 50 g/L, at least 51 g/L, at least 52 g/L, at least 53 g/L, at least 54 g/L, at least 55 g/L, at least 56 g/L, at least 57 g/L, at least 58 g/L, at least 59 g/L, at least 60 g/L, at least 61 g/L, at least 62 g/L, at least 63 g/L, at least 64 g/L, at least 65 g/L, at least 66 g/L, at least 67 g/L, at least 68 g/L, at least 69 g/L, at least 70 g/L, at least 75 g/L, at least 80 g/L, at least 85 g/L, at least 90 g/L, at least 95 g/L, at least 100 g/L, at least 125 g/L, at least 150 g/L, at least 175 g/L, at least 200 g/L, at least 225 g/L, at least 250 g/L, at least 275 g/L, at least 300 g/L, at least 325 g/L, at least 350 g/L, at least 375 g/L, at least 400 g/L, at least 425 g/L, at least 450 g/L, at least 475 g/L, at least 500 g/L, at least 1,000 g/L, at least 2,000 g/L, at least 3,000 g/L, at least 4,000 g/L, at least 5,000 g/L, at least 6,000 g/L, at least 7,000 g/L, at least 8,000 g/L, at least 9,000 g/L, or at least 10,000 g/L of one or more isoprenoids and/or isoprenoid precursors. In some embodiments, the isoprenoid precursor is mevalonate.

In some embodiments, a host cell comprises one or more enzymes in the yeast mevalonate pathway and a heterologous polynucleotide encoding a lanosterol synthase with reduced activity as compared to a control lanosterol synthase; or a heterologous polynucleotide that reduces lanosterol synthase activity; and/or a heterologous polynucleotide encoding a squalene epoxidase with reduced activity as compared to a control squalene epoxidase; or a heterologous polynucleotide that reduces squalene epoxidase activity. In some embodiments, the one or more enzymes in the yeast mevalonate pathway is selected from the enzymes set forth in Table 1.

In some embodiments, a host cell comprises one or more enzymes in the Archaea I mevalonate pathway and a heterologous polynucleotide encoding a lanosterol synthase with reduced activity as compared to a control lanosterol synthase; or a heterologous polynucleotide that reduces lanosterol synthase activity; and/or a heterologous polynucleotide encoding a squalene epoxidase with reduced activity as compared to a control squalene epoxidase; or a heterologous polynucleotide that reduces squalene epoxidase activity. In some embodiments, the one or more enzymes in the archaea I mevalonate pathway is selected from the enzymes set forth in Table 2.

In some embodiments, a host cell comprises one or more enzymes in the Archaea II mevalonate pathway and a heterologous polynucleotide encoding a lanosterol synthase with reduced activity as compared to a control lanosterol synthase; or a heterologous polynucleotide that reduces lanosterol synthase activity; and/or a heterologous polynucleotide encoding a squalene epoxidase with reduced activity as compared to a control squalene epoxidase; or a heterologous polynucleotide that reduces squalene epoxidase activity. In some embodiments, the one or more enzymes in the Archaea II mevalonate pathway is selected from the enzymes set forth in Table 3.

In some embodiments, a host cell comprises one or more enzymes in the MEP pathway and a heterologous polynucleotide encoding a lanosterol synthase with reduced activity as compared to a control lanosterol synthase; or a heterologous polynucleotide that reduces lanosterol synthase activity; and/or a heterologous polynucleotide encoding a squalene epoxidase with reduced activity as compared to a control squalene epoxidase; or a heterologous polynucleotide that reduces squalene epoxidase activity. In some embodiments, the one or more enzymes in the MEP pathway is selected from the enzymes set forth in Table 4.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification of Lanosterol Synthases with Reduced Activity

This Example describes identification of lanosterol synthases with reduced activity.

Mutagenic PCR was performed on an ERG7 template, and the PCR mixture was cleaved with BsaI and ligated to pERG7.NatR cleaved with HindIII and NcoI, to create a library of mutants, ranging from low (2-4 mutations per gene), to medium (6-9 mutations per gene), to high (12-20 mutations per gene). Cleavage of these plasmids with PacI and SspI and introduction into a Yarrowia strain (genotype pTEF-HMGt erg7Δ13 [GPR1-1 ERG7 HygR]) yielded plates (grown at 22° C. or 30° C.) of nourseothricin resistant (NatR) transformants that were replica-plated to YNBAc (YNB+30 mM glacial acetic acid) at the appropriate temperature. 372 acetate resistant (AcR) clones were identified and picked to YPD medium, grown at the appropriate temperature, and subsequently inoculated to YPD4 medium, grown for three days at 30° C. and the supernatants assayed for mevalonic acid by LC-RIA. AcR cells are able to grow on media containing acetic acid. At the same time, the clones propagated originally at 22° C. were tested for temperature sensitive growth at 32° C., while those grown at 30° C. were tested for cold sensitivity at 18° C.

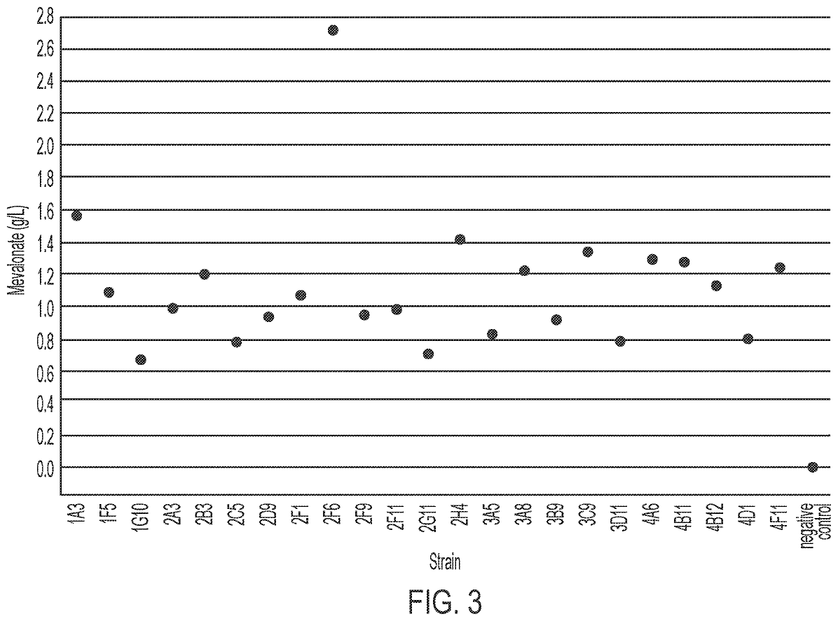

As shown in Table 7 and FIG. 3, nine temperature sensitive (T.s.) and three partially cold sensitive (C.s.) clones were identified that increased mevalonate titer relative to the parent. These strains were 1A3, 2F9, 2F6, 2C5, 2B3, 2A5, 2F1, 3B9, and 3D11. Of the strains tested, 2F6, which harbors the lanosterol synthase set forth in SEQ ID NO: 3, showed the highest mevalonate titer. 4A6 and 4F11 have the same mutations. The strains not labeled as T.s. or C.s. are neither temperature-nor cold-sensitive.

TABLE 7
Lanosterol Synthase Activity as Determined
by Mevalonate Titer in Yarrowia host cells*

| | | Protein | Mevalonate | Type |
|---|---|---|---|---|
| | Mutation(s) relative | SEQ | titer | of |
| Strain | to SEQ ID NO: 1 | ID NO: | (g/L) | Mutant |
| Parent | none | 1 | 0.0 ± 0.0 | |
| 1A3 | R33Q, R193C, D289G, N295I, S296T, | 331 | 1.6 | T.s. |
| | N620S, and Y736F | | | |
| 1F5 | unknown | | 1.1 | |
| 1G10 | unknown | | 0.7 | |
| 2G11 | unknown | | 0.7 | |
| 2F11 | R184W, L235M, L260R, and E710Q | 119 | 1 | |
| 2F9 | K47E, L92I, T360S, S372P, | 325 | 1 | T.s. |
| | T444M, and R578P | | | |
| 2D9 | unknown | | 0.9 | |
| 2F6 | D50G, K66R, N94S, G417S, E617V, and | 3 | 2.7 | T.s. |
| | F726L | | | |
| 2C5 | N14Y, N132S, Y145C, R193H, I286F, | 329 | 0.8 | T.s. |
| | L316R, F432I, E442V, T444S, I479S, | | | |
| | K631R, and T655A | | | |
| 2H4 | F432S, D452G, and I536F | 85 | 1.4 | |
| 2B3 | E287G, K329N, E617V, and F726V | 324 | 1.2 | T.s |
| 2A5 | E231V, A407V, Q423L, A529T, and | 323 | 1 | T.s. |
| | Y564C | | | |
| 2F1 | V248F, D371V, and G702D | 118 | 1.1 | T.s. |
| 3A5 | L197V, K282I, N314S, P370L, A608T, | 326 | 0.8 | |
| | G638D, and F650L | | | |
| 3A8 | L491Q, Y586F, and R660H | 120 | 1.2 | |
| 3B9 | G122C, H249L, and K738M | 316 | 0.9 | C.s. |
| 3C9 | P227L, E474V, V559A, and Y564N | 318 | 1.3 | |
| 3D11 | K85N, G158S, S515L, P526T, Q619L, | 321 | 0.8 | C.s |
| | and Q742* | | | |
| 4D1 | unknown | | 0.8 | |
| 4A6 | G107D and K631E | 319 | 1.3 | |
| 4B11 | T212I, W213L, N544Y, and V552E | 322 | 1.3 | |
| 4F11 | G107D and K631E | 319 | 1.3 | |
| 4B12 | I172N, C414S, L560M, and G679S | 84 | 1.1 | |

*indicates a truncation

Many of the mevalonate-excreting ERG7 alleles also significantly perturbed the steady state levels of other metabolites; 2F6 in particular decreased squalene, and increased oxidosqualene, dioxidosqualene, and ergosterol.

### Example 2. Characterization of an Acetoacetyl CoA Synthase that Increases Squalene Production in Yarrowia Host Cells

This Example describes characterization of the effect of an acetoacetyl CoA synthase on squalene production in a host cell. An acetoacetyl CoA synthase comprising, SEQ ID NO: 6 and encoded by SEQ ID NO: 7 was constructed. Various constructs were constructed, each expressing the acetoacetyl CoA synthase under the control of a different promoter. The constructs were then randomly inserted into a Yarrowia host cell strain that produced about 17.2 mg/L squalene. As shown in Table 8, the acetoacetyl CoA synthase (represented by SEQ ID NO: 6 and 7) increased squalene titers to about 23.8-33 mg/L.

TABLE 8
Expression of an Acetoacetyl COA Synthase (SEQ ID NO:
6) Under the Control of Various Promoters in Yarrowia

| | | Average | promoter |
|---|---|---|---|
| | Squalene | Squalene | expressing |
| | [mg/L] | [mg/L] | NphT7 |
| Control | 18.9 | 17.2 | — |
| Control | 15.1 | | |
| Control | 17.6 | | |
| NphT7-A | 32.4 | 30.1 | tef Yarrowia alimentaria |
| | | | (YAALOS06) |
| NphT7-A | 28.9 | | |
| NphT7-A | 29.1 | | |
| NphT7-B | 27.5 | 28.6 | act1p (YB392) |
| NphT7-B | 28.7 | | |
| NphT7-B | 29.7 | | |
| NphT7-D | 20.9 | 27.8 | pMDH1 (YB392) |
| NphT7-D | 33.5 | | |
| NphT7-D | 29.1 | | |
| NphT7-F | 31.4 | 32.7 | gapDH Y. porcina (YAPOOS01) |
| NphT7-F | 32.3 | | |
| NphT7-F | 34.3 | | |
| NphT7-G | 35.9 | 33.0 | tef Y. deformans (YADEOS01) |
| NphT7-G | 30.0 | | |
| NphT7-G | 33.1 | | |
| NphT7-H | 23.5 | 24.0 | gapDH C. osloensis (YAOSOS01) |
| NphT7-H | 23.2 | | |
| NphT7-H | 25.4 | | |
| NphT7-I | 19.8 | 23.8 | tef Y. sp. (JCM 30694) |
| NphT7-I | 25.2 | | |
| NphT7-I | 26.6 | | |

Several of the nphT7 cassettes also induced very high mevalonate secretion, up to 5 g/L, which represents a significant fraction of the theoretical yield.

### Example 3. Production of cucurbitadienol in ERG7 mutant host cells

This Example describes characterization of cucurbitadienol synthases (CDSs) in different Yarrowia host cells comprising mutants of SEQ ID NO: 1.

Acetate resistant (AcR) cells were generated as in Example 1 using pERG7-NatR plasmids that resulted in clones with high mevalonate titers. AcR cells are able to grow on media containing acetic acid. Constructs encoding a particular CDS were inserted randomly into these cells. All strains except for strains 887779 and 870688 express AquAgaCDS16 (SEQ ID NOs: 226 and 327). Strains 887779 and 870688 express SgCDS1 (SEQ ID NOs: 256 and 332). Strains 950910 and 950917 also express NphT7 (SEQ ID NO: 6). The resulting nourseothricin resistant (NatR) isolates were picked and grown in 96-deepwell plates in 0.5 mL YPD medium for two days at 30° C., subcultured into 0.5 mL YPD10 medium for 4 days at 30° C. and then the cultures were assayed for cucurbitadienol by GC-MS. Nourseothricin resistance allows for the selection of cells comprising a heterologous nucleic acid encoding a CDS. Strain 870688 comprising SEQ ID NO: 1 was used as a control.

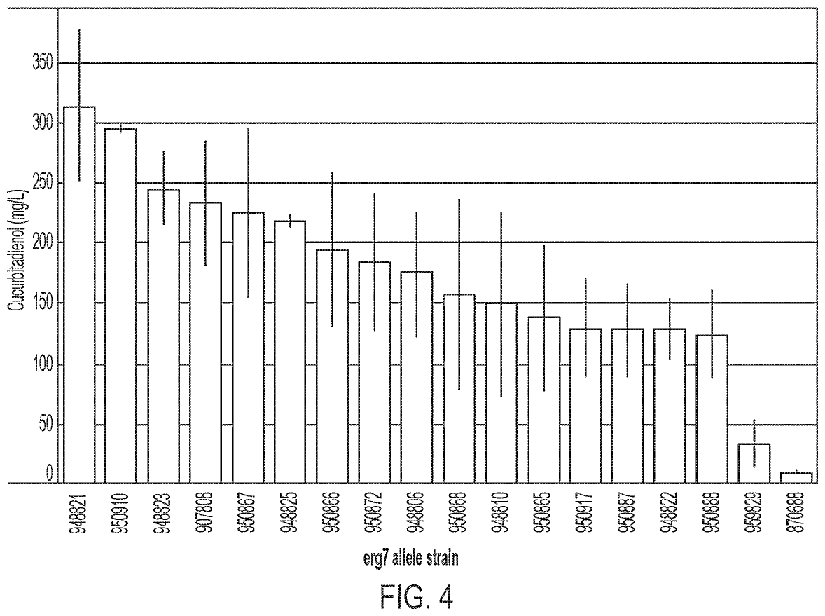

As shown in Table 9 and FIG. 4, cucurbitadienol titers of Yarrowia strains comprising a mutant lanosterol synthase are significantly greater than the strain comprising SEQ ID NO: 1.

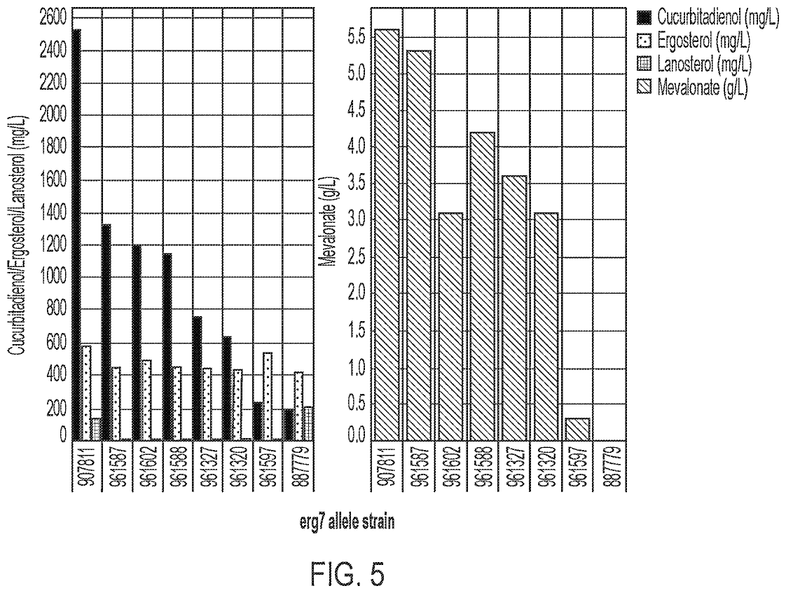

A selection of strains was then run in ambr 250 bioreactors, where cucurbitadienol, ergosterol and lanosterol were assayed by GC-MS and mevalonate by HPLC. Strain 887779 comprising SEQ ID NO: 1 was used as a control. As shown in FIG. 5 and Tables 10A-10B, Yarrowia strains with mutant lanosterol synthase alleles accumulate less lanosterol and more mevalonate and cucurbitadienol relative to a strain comprising the wild-type lanosterol synthase comprising SEQ ID NO: 1.

TABLE 9
Effects of Lanosterol Synthase Mutations
on Cucurbitadienol Production in Yarrowia

| | Lanosterol | | | Average Fold |
|---|---|---|---|---|
| | synthase | | Average | Cucurbitadienol |
| | mutations | Protein | Cucurbitadienol | Titer Increase |
| | relative to | SEQ | Titer | Relative to |
| Strain | SEQ ID NO: 1 | ID NO | (mg/L) | Strain 870688 |
| 948821 | K85N and G158S | 86 | 314.7 | 35.6 |
| 950910 | I172N, C414S, L560M, | 84 | 295.4 | 33.4 |
| | and G679S | | | |
| 948823 | I172N, C414S, L560M, | 84 | 245 | 27.7 |
| | and G679S | | | |
| 907808 | R193C, D289G, N295I, | 83 | 233.7 | 26.4 |
| | S296T, N620S, and Y736F | | | |
| 950867 | D80G, P83L, T170A, | 92 | 225.4 | 25.5 |
| | T198I, and A228T | | | |
| 948825 | I172N, C414S, and L560M | 89 | 218.3 | 24.7 |
| 950866 | D371V, K498N, M610I, | 91 | 194 | 21.9 |
| | and G702D | | | |
| 950872 | T360S, S372P, T444M, | 94 | 184.8 | 20.9 |
| | and R578P | | | |
| 948806 | I172N, C414S, L560M, | 84 | 175 | 19.8 |
| | and G679S | | | |
| 950868 | D50G, K66R, N94S, G417S, | 3 | 157.8 | 17.8 |
| | E617V, and F726L | | | |
| 948810 | F432S, D452G, and I536F | 85 | 149.4 | 16.9 |
| 950865 | D371V, M610I, and G702D | 90 | 137.7 | 15.6 |
| 950917 | D50G, K66R, N94S, | 95 | 129.3 | 14.6 |
| | G417S, and E617V | | | |
| 950887 | D50G, K66R, N94S, G417S, | 95 | 128.1 | 14.5 |
| | and E617V | | | |
| 948822 | L197V, K282I, N314S, and | 87 | 127.6 | 14.4 |
| | P370L | | | |
| 950888 | D80G, P83L, T170A, T198I, | 92 | 124.7 | 14.1 |
| | and A228T | | | |
| 959829 | L309F, V344A, T398I, | 99 | 32.1 | 3.6 |
| | and K686E | | | |
| 870688 | N/A (wild-type | 1 | 8.9 | 1 |
| | ERG7 (WT)) | | | |

TABLE 10A
Effects of Lanosterol Synthase Mutations
on Cucurbitadienol Production in Yarrowia

| | Lanosterol | | | Average Fold |
|---|---|---|---|---|
| | synthase | | Average | Cucurbitadienol |
| | mutations | Protein | Cucurbitadienol | Titer Increase |
| | relative to | SEQ | Titer | Relative to |
| Strain | SEQ ID NO: 1 | ID NO | (mg/L) | Strain 870688 |
| 907811 | D50G, K66R, N94S, | 3 | 2522.1 | 13.3 |
| | G417S, E617V, and | | | |
| | F726L | | | |
| 950865 | D371V, M610I, and | 90 | 1327.7 | 7.0 |
| | G702D | | | |
| 950872 | T360S, S372P, | 94 | 1200.2 | 6.4 |
| | T444M, and R578P | | | |
| 950866 | D371V, K498N, | 91 | 1143.8 | 6.1 |
| | M610I, and G702D | | | |
| 948823 | I172N, C414S, | 84 | 764.5 | 4.0 |
| | L560M, and G679S | | | |
| 948825 | I172N, C414S, | 89 | 638.5 | 3.4 |
| | and L560M | | | |
| 950867 | D80G, P83L, T170A, | 92 | 231.2 | 1.2 |
| | T198I, and A228T | | | |
| 887779 | N/A (wild-type | 1 | 189.0 | 1.0 |
| | ERG7 (WT)) | | | |

TABLE 10B
Effects of Lanosterol Synthase Mutations on Ergosterol,
Lanosterol, and Mevalonate Production in Yarrowia

| | Lanosterol synthase | Protein | | | |
|---|---|---|---|---|---|
| | mutations relative to | SEQ | Ergosterol | Lanosterol | Mevalonate |
| Strain | SEQ ID NO: 1 | ID NO | (mg/L) | (mg/L) | (g/L) |
| 907811 | D50G, K66R, N94S, | 3 | 580.6 | 137.4 | 5.57 |
| | G417S, E617V, and | | | | |
| | F726L | | | | |
| 950865 | D371V, M610I, and | 90 | 452.2 | 8.2 | 5.29 |
| | G702D | | | | |
| 950872 | T360S, S372P, T444M, | 94 | 496.5 | 8.1 | 3.08 |
| | and R578P | | | | |
| 950866 | D371V, K498N, M610I, | 91 | 455.7 | 10.8 | 4.18 |
| | and G702D | | | | |
| 948823 | I172N, C414S, L560M, | 84 | 443.5 | 11.9 | 3.6 |
| | and G679S | | | | |
| 948825 | I172N, C414S, and | 89 | 436.6 | 11.1 | 3.09 |
| | L560M | | | | |
| 950867 | D80G, P83L, T170A, | 92 | 537.9 | 8.2 | 0.293 |
| | T198I, and A228T | | | | |
| 887779 | N/A (wild-type ERG7 | 1 | 422.0 | 207.9 | 0 |
| | (WT)) | | | | |

### Example 4. Production of Oxidosqualene in Saccharomyces cerevisiae Host Cells with Mutants of SEQ ID NO: 313

This Example describes identification of lanosterol synthases with reduced activity using SEQ ID NO: 313 as a template for mutation.

Three different temperature sensitive lanosterol synthase mutants were tested and host cells comprising each of these lanosterol synthase mutants were analyzed for consumption of glucose and production of oxidosqualene, mevalonate, ergosterol, and ethanol. A parent strain with a native lanosterol synthase (SEQ ID NO: 313) was used as the negative control.

Strain 756247 expressed a lanosterol synthase comprising the protein sequence of SEQ ID NO: 100. The nucleotide sequence encoding SEQ ID NO: 100 comprises the following mutations relative to SEQ ID NO: 8 (mutations in SEQ ID NO: 100 relative to SEQ ID NO: 313 are shown in parenthesis): C361T (P121S), C407T (A136V), G474A (silent), A898G (S300G), A909G (silent), T965G (V322G), A1312G (K438E), T1506A (F502L), T1732C (silent), A1882G (K628E), and T2178G (Y726*—truncation mutation). A silent mutation results in no change in the amino acid sequence.

Strain 756248 expressed a lanosterol synthase comprising the protein sequence of SEQ ID NO: 101. The nucleotide sequence encoding SEQ ID NO: 101 comprises the following mutations relative to SEQ ID NO: 8 (mutations in SEQ ID NO: 101 relative to SEQ ID NO: 313 are shown in parenthesis): C333T (silent), A803G/A804T (K268S), A841G (T281A), T1504C (F502L), C1811A (T604N), G1966A (A656T), and A2078G (E693G).

Strain 756249 expressed a lanosterol synthase comprising the protein sequence of SEQ ID NO: 102. The nucleotide sequence encoding SEQ ID NO: 102 comprises the following mutations relative to SEQ ID NO: 8 (mutations in SEQ ID NO: 102 relative to SEQ ID NO: 313 are shown in parenthesis): A190G (R64G), A358G (I120V), G678T (M226I), T823A (F275I), A997G (T333A), and T1855A (C619S).

To measure 2-3-oxidosqualene production, strains were first grown overnight at 30° C., diluted to a starting OD of 0.2 and grown for an additional 16 h either at 30° C. or 35° C. in triplicates in 96-well deep well plates. Cell culture volumes were 500 μL and the media used in this experiment was YPD (10 g/L Yeast Extract, 20 g/L Peptone and 20 g/L Dextrose). 200 μL of the culture and 400 μL of ethyl acetate containing internal standards (100 m tridecane and 100 mg/L pregnenolone) were transferred to a 96-well deep well plate containing 100 μL of silica/zirconia beads (0.5 mm dia., Cat. no. 11079105z Biospec) in each well. The plate containing the samples was heat sealed and agitated at 1750 rpm for 5 minutes using a Genogrinder. The plate was then centrifuged for 10 minutes at 4000 rpm at 4° C. to separate the aqueous and organic layers. The plate was then stored at −30° C. for 2 h to freeze the aqueous layer and 100 μL from the top layer was transferred to a glass vial analyzed by a GC-FID. A gas chromatograph (Thermo Scientific Trace 1310) with a TG-5MS column (15 m×0.25 mm×0.25 m) was used at a flow rate of 1.5 mL/min. The eluents were determined by comparing peak retention times to those of known standard substances, and the amounts were quantified by comparing the peak area of the analyte to the peak area of the standard substance at known concentrations.

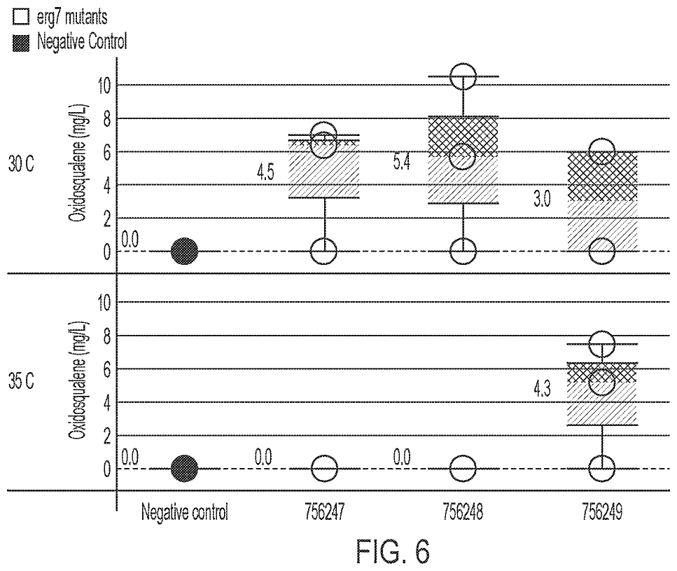

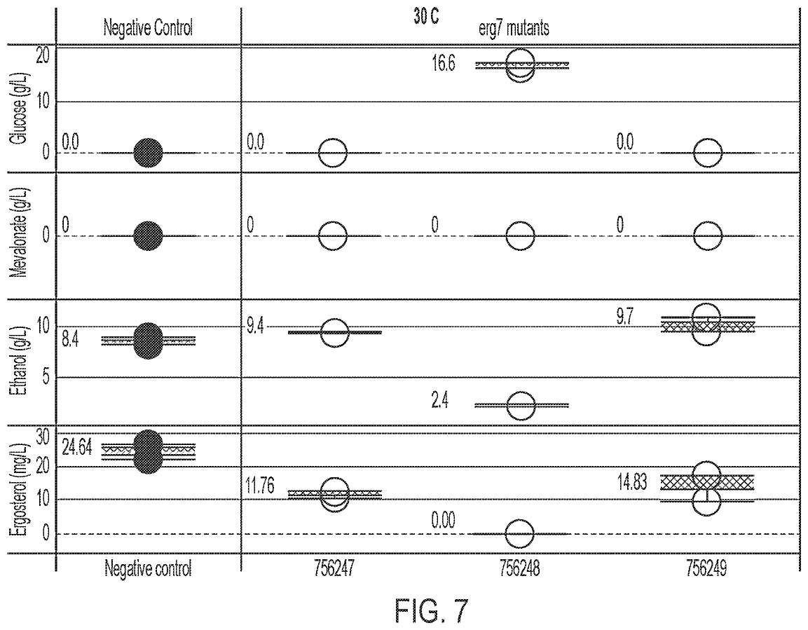

As shown in FIG. 7 and Table 11, at 30° C., Saccharomyces cerevisiae host cells comprising any one of SEQ ID NOs: 100-102 produced less ergosterol than the parent strain (the negative control), indicating that lanosterol synthases comprising any one of SEQ ID NOs: 100-102 were less active and had impaired lanosterol synthase activity compared to a wild-type lanosterol synthase comprising SEQ ID NO: 313 at this temperature. At 30° C., 5-10 mg/L of oxidosqualene was detected in all three lanosterol synthase mutant strains while the control strain did not produce detectable levels of oxidosqualene (FIG. 6 and Table 11). Thus, host cells with decreased lanosterol synthase activity showed increased oxidosqualene production.

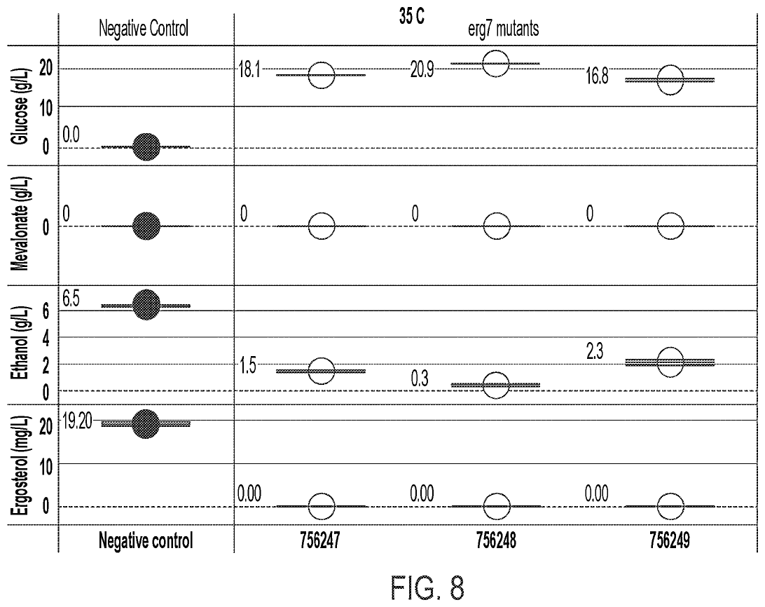

At 35° C., the lanosterol synthase mutant strains were unable to grow or grew minimally compared to the control strain as shown by the residual glucose numbers (FIG. 8 and Table 12). For all strains, the starting glucose concentration was 20 g/L. Without being bound by a particular theory, it is possible that since the lanosterol synthase mutants are temperature sensitive, the cells cannot survive in the absence of a functional lanosterol synthase comprising SEQ ID NO: 313 at higher temperatures. Only strain 756249 accumulated some oxidosqualene at 35° C. The control strain with the native lanosterol synthase gene encoding SEQ ID NO: 313 was able to consume all the glucose at 30° C. and 35° C., but did not produce detectable levels of oxidosqualene. Thus, the results suggest that complete knockout of lanosterol synthase activity is detrimental to these cells.

TABLE 11
Effects of Lanosterol Synthase Mutations Relative to SEQ ID NO: 313 on
Glucose Consumption and Oxidosqualene, Mevalonate, Ergosterol, and Ethanol
Production by Saccharomyces cerevisiae Host Cells at 30° C.

| Saccharomyces | | Oxidosqualene | Glucose | Mevalonate | Ergosterol | Ethanol |
|---|---|---|---|---|---|---|
| cerevisiae Strain | | (mg/L) | [g/L] | [g/L] | [mg/L] | [g/L] |
| Negative control | 1 | 0.00 | 0.04 | 0.00 | 22.29 | 8.98 |
| (parent strain with | 2 | 0.00 | 0.04 | 0.00 | 26.89 | 8.36 |
| a wild-type | 3 | 0.00 | 0.04 | 0.00 | 24.75 | 8.42 |
| lanosterol synthase) | | | | | | |
| 756247 | 1 | 6.38 | 0.04 | 0.00 | 10.49 | 9.35 |
| | 2 | 7.01 | 0.04 | 0.00 | 12.71 | 9.52 |
| | 3 | 0.00 | 0.09 | 0.00 | 12.08 | 9.44 |
| 756248 | 1 | 5.71 | 16.10 | 0.00 | 0.00 | 2.50 |
| | 2 | 0.00 | 17.00 | 0.00 | 0.00 | 2.26 |
| | 3 | 10.53 | 17.00 | 0.00 | 0.00 | 2.36 |
| 756249 | 1 | 6.05 | 0.04 | 0.00 | 9.51 | 10.90 |
| | 2 | 0.00 | 0.03 | 0.00 | 17.32 | 9.52 |
| | 3 | 0.00 | 0.03 | 0.00 | 17.66 | 9.72 |

TABLE 12
Effects of Lanosterol Synthase Mutations Relative to SEQ ID NO: 313 on
Glucose Consumption and Oxidosqualene, Mevalonate, Ergosterol, and Ethanol
Production by Saccharomyces cerevisiae Host Cells at 35° C.

| Saccharomyces | | Oxidosqualene | Glucose | Mevalonate | Ergosterol | Ethanol |
|---|---|---|---|---|---|---|
| cerevisiae Strain | | (mg/L) | [g/L] | [g/L] | [mg/L] | [g/L] |
| Negative control | 1 | 0.00 | 0.04 | 0.00 | 18.78 | 6.37 |
| (parent strain with | 2 | 0.00 | 0.04 | 0.00 | 19.35 | 6.54 |
| a wild-type | 3 | 0.00 | 0.04 | 0.00 | 19.48 | 6.63 |
| lanosterol synthase) | | | | | | |
| 756247 | 1 | 0.00 | 18.00 | 0.00 | 0.00 | 1.54 |
| | 2 | 0.00 | 18.10 | 0.00 | 0.00 | 1.48 |
| | 3 | 0.00 | 18.00 | 0.00 | 0.00 | 1.37 |
| 756248 | 1 | 0.00 | 21.00 | 0.00 | 0.00 | 0.53 |
| | 2 | 0.00 | 21.00 | 0.00 | 0.00 | 0.31 |
| | 3 | 0.00 | 20.70 | 0.00 | 0.00 | 0.28 |
| 756249 | 1 | 5.24 | 17.20 | 0.00 | 0.00 | 1.98 |
| | 2 | 7.54 | 16.40 | 0.00 | 0.00 | 2.29 |
| | 3 | 0.00 | 16.40 | 0.00 | 0.00 | 2.26 |

TABLE 13
Non-limiting Examples of Amino Acid
Changes Relative to SEQ ID NO: 1*

| | |
|---|---|
| | Amino acid change |
| Position | relative to SEQ ID NO: 1 |

| | | |
|---|---|---|
| 14 | N14Y | |
| 33 | R33Q | |
| 47 | K47E | |
| 50 | D50G | |
| 66 | K66R | |
| 80 | D80G | |
| 83 | P83L | |
| 85 | K85N | |
| 92 | L92I | |
| 94 | N94S | |
| 107 | G107D | |
| 122 | G122C | |
| 132 | N132S | |
| 145 | Y145C | |
| 158 | G158S | |
| 170 | T170A | |
| 172 | I172N | |
| 184 | R184W | |
| 193 | R193C | R193H |
| 197 | L197V | |
| 198 | T198I | |
| 212 | T212I | |
| 213 | W213L | |
| 227 | P227L | |
| 228 | A228T | |
| 231 | E231V | |
| 235 | L235M | |
| 248 | V248F | |
| 249 | H249L | |
| 260 | L260R | |
| 282 | K282I | |
| 286 | I286F | |
| 287 | E287G | |
| 289 | D289G | |
| 295 | N295I | |
| 296 | S296T | |
| 309 | L309F | |
| 314 | N314S | |
| 316 | L316R | |
| 329 | K329N | |
| 344 | V344A | |
| 360 | T360S | |
| 370 | P370L | |
| 371 | D371V | |
| 372 | S372P | |
| 398 | T398I | |
| 407 | A407V | |
| 414 | C414S | |
| 417 | G417S | |
| 423 | Q423L | |
| 432 | F432I | F432S |
| 437 | R437L | |
| 442 | E442V | |
| 444 | T444M | T444S |
| 452 | D452G | |
| 474 | E474V | |
| 479 | I479S | |
| 491 | L491Q | |
| 498 | K498N | |
| 515 | S515L | |
| 526 | P526T | |
| 529 | A529T | |
| 536 | I536F | |
| 544 | N544Y | |
| 552 | V552E | |
| 559 | V559A | |
| 560 | L560M | |
| 564 | Y564C | Y564N |
| 578 | R578P | |
| 586 | Y586F | |
| 608 | A608T | |
| 610 | M610I | |
| 617 | E617V | |
| 619 | Q619L | |
| 620 | N620S | |
| 631 | K631E | K631R |
| 638 | G638D | |
| 650 | F650L | |
| 655 | T655A | |
| 660 | R660H | |
| 679 | G679S | |
| 686 | K686E | |
| 702 | G702D | |
| 710 | E710Q | |
| 726 | F726L | F726V |
| 736 | Y736F | |
| 738 | K738M | |
| 742 | Q742* | |

*indicates a truncation

TABLE 14
Non-limiting Examples of Amino Acid
Changes Relative to SEQ ID NO: 313*

| | Amino acid change |
|---|---|
| | relative to SEQ |
| Position | ID NO: 313 |
| 64 | R64G |
| 120 | I120V |
| 121 | P121S |
| 136 | A136V |
| 226 | M226I |
| 268 | K268S |
| 275 | F275I |
| 281 | T281A |
| 300 | S300G |
| 322 | V322G |
| 333 | T333A |
| 438 | K438E |
| 502 | F502L |
| 604 | T604N |
| 619 | C619S |
| 628 | K628E |
| 656 | A656T |
| 693 | E693G |
| 726 | Y726* |

*indicates a truncation that results in deletion of residues 726-731 in SEQ ID NO: 313

TABLE 15
Non-limiting Examples of Lanosterol Synthase Sequences

| | | Nucleic | | |
|---|---|---|---|---|
| | | Acid | | Protein |
| | | SEQ ID | | SEQ ID |
| Strain | Nucleotide Sequence | NO | Protein Sequence | NO |
| 870688 | ATGGGAATCCACGAAAGTGTGTCGA | 61 | MGIHESVSKQFAKNGHSKY | 1 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCGTGGCAG | | PAPGELWLLPYFVPVHPGR | |
| | AGCGAATACGACGGACCGCAGTTCA | | WWVHTRWIYLAMGYLEA | |
| | TGAGCATCGGCTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGCTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGACCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTCCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCGGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TCTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 907808 | ATGGGAATCCACGAAAGTGTGTCGA | 62 | MGIHESVSKQFAKNGHSKY | 83 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGTTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKACKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIGFSKHCITISGVDLY | |
| | GCCGGTCAAAACCGAAATGATCAGA | | YPHTGLLKFGNALLRRYRK | |
| | TACATTGTCAACACAGCCCACCCAGT | | FRPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPDSANYKKIAA | |
| | CCAGCATCAACTACGTGGTCCTGCGA | | RWPEFLSLNPSGMFMNGTN | |
| | CTACTGGGCCTGTCGCGGGATCATCC | | GLQVWDTAFAVQYACVCG | |
| | GGTCTGCGTCAAGGCGTGCAAAACG | | FAELPQYQKTIRAAFDFLDR | |
| | CTGCTCACCAAGTTTGGCGGCGCCAT | | SQINEPTEENSYRDDRVGG | |
| | CAACAACCCCCATTGGGGCAAGACC | | WPFSTKTQGYPVSDCTAEA | |
| | TGGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGNIMVEYPYV | |
| | GGGTCCATACCCGGTGGATCTACCTT | | ECTDSVVLGLSYFRKYHDY | |
| | GCCATGGGCTATCTGGAGGCTGCGG | | RNEDVDRAISAAIGYIIREQ | |
| | AGGCCCAATGCGAACTCACTCCGTTG | | QPDGGFFGSWGVCYCYAH | |
| | CTGGAGGAGCTCCGAGACGAAATCT | | MFAMEALETQSLNYNNCST | |
| | ACAAAAAGCCCTACTCGGAGATTGG | | VQKACDFLAGYQEADGGW | |
| | TTTCTCCAAACATTGCATCACCATCT | | AEDFKSCETQMYVRGPHSL | |
| | CCGGAGTCGACCTCTACTATCCCCAC | | VVPTAMALLSLMSGRYPQE | |
| | ACCGGCCTTTTGAAGTTTGGCAACGC | | DKIHAAARFLMSKQMSNG | |
| | GCTTCTCCGACGATACCGCAAGTTCA | | EWLKEEMEGVFNHTCAIEY | |
| | GACCGCAGTGGATCAAAGAAAAGGT | | PNYRFYFVMKALGLFFKGY | |
| | CAAGGAGGAAATTTATAACTTGTGCC | | CQ | |
| | TTCGAGAGGTTTCCAACACACGACAC | | | |
| | TTGTGTCTCGCTCCCGTCAACAATGC | | | |
| | CATGACCTCCATTGTCATGTATCTCC | | | |
| | ATGAGGGGCCCGATTCGGCGAATTA | | | |
| | CAAAAAGATTGCGGCCCGATGGCCC | | | |
| | GAATTTCTGTCTCTGAATCCGTCGGG | | | |
| | AATGTTTATGAACGGCACCAACGGTC | | | |
| | TGCAGGTCTGGGATACTGCGTTTGCC | | | |
| | GTGCAATACGCGTGTGTTTGTGGCTT | | | |
| | TGCCGAACTTCCCCAGTACCAGAAG | | | |
| | ACGATCCGAGCGGCGTTTGATTTTCT | | | |
| | CGATCGGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAAGCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTGGAATGC | | | |
| | ACTGATTCTGTTGTTCTGGGTCTGTC | | | |
| | CTACTTTCGAAAGTACCACGATTACC | | | |
| | GCAACGAAGACGTGGACCGAGCCAT | | | |
| | CTCTGCTGCCATTGGATACATTATTC | | | |
| | GAGAGCAGCAGCCTGACGGCGGCTT | | | |
| | CTTTGGCTCCTGGGGCGTGTGCTACT | | | |
| | GCTACGCTCACATGTTTGCCATGGAG | | | |
| | GCTCTGGAGACGCAGAGTCTCAACT | | | |
| | ATAACAACTGTTCCACGGTTCAAAAG | | | |
| | GCGTGCGACTTTCTGGCGGGCTACCA | | | |
| | GGAAGCAGATGGAGGCTGGGCCGAG | | | |
| | GACTTTAAGTCGTGCGAGACTCAGAT | | | |
| | GTACGTGCGCGGACCCCATTCGCTGG | | | |
| | TCGTGCCTACTGCCATGGCCCTGTTG | | | |
| | AGTTTGATGAGTGGTCGGTATCCCCA | | | |
| | GGAGGACAAGATTCATGCTGCGGCC | | | |
| | CGGTTTCTCATGAGCAAGCAGATGA | | | |
| | GCAACGGTGAGTGGCTCAAGGAGGA | | | |
| | GATGGAGGGGGTGTTTAACCATACTT | | | |
| | GTGCCATTGAGTATCCCAACTACCGG | | | |
| | TTTTATTTTGTCATGAAGGCTTTGGG | | | |
| | GTTGTTTTTCAAGGGATATTGCCAGT | | | |
| | GA | | | |
| 948806 | ATGGGAATCCACGAAAGTGTGTCGA | 63 | MGIHESVSKQFAKNGHSKY | 84 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSNNYVVLRLLGLSRDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCAACAACTACGTGGTCCTGCG | | IAARWPEFLSLNPSGMFMN | |
| | ACTACTGGGCCTGTCACGGGATCATC | | GTNGLQVWDTAFAVQYAS | |
| | CGGTCTGCGTCAAGGCGCGCAAAAC | | VCGFAELPQYQKTIRAAFD | |
| | GCTGCTCACCAAGTTTGGCGGCGCCA | | FLDRSQINEPTEENSYRDDR | |
| | TCAACAACCCCCATTGGGGCAAGAC | | VGGWPFSTKTQGYPVSDCT | |
| | CTGGCTGTCGATTCTCAATCTCTACA | | AEALKAIIMVQNTPGYEDL | |
| | AATGGGAGGGTGTGAATCCGGCCCC | | KKQVSDKRKHTAIDLLLGM | |
| | TGGCGAGCTCTGGCTGTTGCCCTACT | | QNVGSFEPGSFASYEPIRAS | |
| | TTGTTCCTGTTCATCCGGGCCGATGG | | SMLEKINPAEVFGNIMVEY | |
| | TGGGTCCATACCCGGTGGATCTACCT | | PYVECTDSVVMGLSYFRKY | |
| | TGCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSS | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGAGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTATGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTAGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 948810 | ATGGGAATCCACGAAAGTGTGTCGA | 64 | MGIHESVSKQFAKNGHSKY | 85 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAASD | |
| | CTGCTCACCAAGTTTGGCGGCGCCAT | | FLDRSQINEPTEENSYRDGR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKFNPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTCTGATTTTC | | | |
| | TCGATCGGTCCCAGATCAACGAGCC | | | |
| | GACGGAGGAAAATTCCTATCGAGAC | | | |
| | GGCCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGTT | | | |
| | CAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 948821 | ATGGGAATCCACGAAAGTGTGTCGA | 65 | MGIHESVSKQFAKNGHSKY | 86 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVNNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWSLHKEDKSTCFGT | |
| | GTGACTCTGGACTCCAAGCCCGTGAA | | SINYVVLRLLGLSRDHPVC | |
| | TAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCATGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGAGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTCCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCGGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TCTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 948822 | ATGGGAATCCACGAAAGTGTGTCGA | 66 | MGIHESVSKQFAKNGHSKY | 87 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLVTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYI | |
| | TACTTTGGCGGCAACGAGATCCCCAC | | KPYSEIDFSKHCNSISGVDL | |
| | GCCGGTCAAAACCGAAATGATCAGA | | YYPHTGLLKFGSALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGLDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGGTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACATAAAGCCCTACTCGGAGATTGAT | | NCSTVQKACDFLAGYQEA | |
| | TTCTCCAAACATTGCAACTCCATCTC | | DGGWAEDFKSCETQMYVR | |
| | CGGAGTCGACCTCTACTATCCCCACA | | GPHSLVVPTAMALLSLMSG | |
| | CCGGCCTTTTGAAGTTTGGCAGCGCG | | RYPQEDKIHAAARFLMSKQ | |
| | CTTCTCCGACGATACCGCAAGTTCAG | | MSNGEWLKEEMEGVFNHT | |
| | ACCGCAGTGGATCAAAGAAAAGGTC | | CAIEYPNYRFYFVMKALGL | |
| | AAGGAGGAAATTTACAACTTGTGCCT | | YFKGYCQ | |
| | TCGAGAGGTTTCCAACACACGACACT | | | |
| | TGTGTCTCGCTCCCGTCAACAATGCC | | | |
| | ATGACCTCCATTGTCATGTATCTCCA | | | |
| | TGAGGGGCTCGATTCGGCGAATTAC | | | |
| | AAAAAGATTGCGGCCCGATGGCCCG | | | |
| | AATTTCTGTCTCTGAATCCGTCGGGA | | | |
| | ATGTTTATGAACGGCACCAACGGTCT | | | |
| | GCAGGTCTGGGATACTGCGTTTGCCG | | | |
| | TGCAATACGCGTGTGTTTGTGGCTTT | | | |
| | GCCGAACTTCCCCAGTACCAGAAGA | | | |
| | CGATCCGAGCGGCGTTTGATTTTCTC | | | |
| | GATCGGTCCCAGATCAACGAGCCGA | | | |
| | CGGAGGAAAATTCCTATCGAGACGA | | | |
| | CCGCGTCGGAGGATGGCCCTTTAGTA | | | |
| | CCAAGACCCAGGGGTATCCAGTCTCC | | | |
| | GACTGTACTGCCGAGGCTCTCAAGGC | | | |
| | CATCATCATGGTCCAGAATACGCCTG | | | |
| | GATACGAGGATCTGAAGAAACAAGT | | | |
| | GTCTGACAAGCGGAAACACACTGCC | | | |
| | ATCGATCTACTTTTGGGAATGCAGAA | | | |
| | CGTGGGCTCGTTTGAACCGGGCTCTT | | | |
| | TCGCCTCCTATGAGCCTATCCGGGCG | | | |
| | TCGTCCATGCTGGAGAAGATCAATCC | | | |
| | GGCCGAGGTGTTTGGAAACATCATG | | | |
| | GTGGAGTATCCGTACGTGGAATGCA | | | |
| | CTGATTCTGTTGTTCTGGGTCTGTCCT | | | |
| | ACTTTCGAAAGTACCACGATTACCGC | | | |
| | AACGAAGACGTGGACCGAGCCATCT | | | |
| | CTGCTGCCATTGGATATATTATTCGA | | | |
| | GAGCAGCAGCCTGACGGCGGCTTCTT | | | |
| | TGGCTCCTGGGGCGTGTGCTACTGCT | | | |
| | ACGCTCACATGTTTGCCATGGAGGCT | | | |
| | CTGGAGACGCAGAATCTCAACTATA | | | |
| | ACAACTGTTCCACGGTTCAAAAGGC | | | |
| | GTGCGACTTTCTGGCGGGCTACCAGG | | | |
| | AAGCAGATGGAGGCTGGGCCGAGGA | | | |
| | CTTTAAGTCGTGCGAGACCCAGATGT | | | |
| | ACGTGCGCGGACCCCATTCGCTGGTC | | | |
| | GTGCCTACTGCCATGGCCCTGTTGAG | | | |
| | TTTGATGAGTGGTCGGTATCCCCAGG | | | |
| | AGGACAAGATTCATGCTGCGGCCCG | | | |
| | GTTTCTCATGAGCAAGCAGATGAGC | | | |
| | AACGGTGAGTGGCTCAAGGAGGAGA | | | |
| | TGGAGGGGGTGTTTAACCATACTTGT | | | |
| | GCCATTGAGTATCCCAACTACCGGTT | | | |
| | TTATTTTGTCATGAAGGCTTTGGGGT | | | |
| | TGTATTTCAAGGGATATTGCCAGTGA | | | |
| 948823 | ATGGGAATCCACGAAAGTGTGTCGA | 63 | MGIHESVSKQFAKNGHSKY | 84 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSNNYVVLRLLGLSRDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCAACAACTACGTGGTCCTGCG | | IAARWPEFLSLNPSGMFMN | |
| | ACTACTGGGCCTGTCACGGGATCATC | | GTNGLQVWDTAFAVQYAS | |
| | CGGTCTGCGTCAAGGCGCGCAAAAC | | VCGFAELPQYQKTIRAAFD | |
| | GCTGCTCACCAAGTTTGGCGGCGCCA | | FLDRSQINEPTEENSYRDDR | |
| | TCAACAACCCCCATTGGGGCAAGAC | | VGGWPFSTKTQGYPVSDCT | |
| | CTGGCTGTCGATTCTCAATCTCTACA | | AEALKAIIMVQNTPGYEDL | |
| | AATGGGAGGGTGTGAATCCGGCCCC | | KKQVSDKRKHTAIDLLLGM | |
| | TGGCGAGCTCTGGCTGTTGCCCTACT | | QNVGSFEPGSFASYEPIRAS | |
| | TTGTTCCTGTTCATCCGGGCCGATGG | | SMLEKINPAEVFGNIMVEY | |
| | TGGGTCCATACCCGGTGGATCTACCT | | PYVECTDSVVMGLSYFRKY | |
| | TGCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSS | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGAGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTATGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTAGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 948825 | ATGGGAATCCACGAAAGTGTGTCGA | 68 | MGIHESVSKQFAKNGHSKY | 89 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSNNYVVLRLLGLSRDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCAACAACTACGTGGTCCTGCG | | IAARWPEFLSLNPSGMFMN | |
| | ACTACTGGGCCTGTCACGGGATCATC | | GTNGLQVWDTAFAVQYAS | |
| | CGGTCTGCGTCAAGGCGCGCAAAAC | | VCGFAELPQYQKTIRAAFD | |
| | GCTGCTCACCAAGTTTGGCGGCGCCA | | FLDRSQINEPTEENSYRDDR | |
| | TCAACAACCCCCATTGGGGCAAGAC | | VGGWPFSTKTQGYPVSDCT | |
| | CTGGCTGTCGATTCTCAATCTCTACA | | AEALKAIIMVQNTPGYEDL | |
| | AATGGGAGGGTGTGAATCCGGCCCC | | KKQVSDKRKHTAIDLLLGM | |
| | TGGCGAGCTCTGGCTGTTGCCCTACT | | QNVGSFEPGSFASYEPIRAS | |
| | TTGTTCCTGTTCATCCGGGCCGATGG | | SMLEKINPAEVFGNIMVEY | |
| | TGGGTCCATACCCGGTGGATCTACCT | | PYVECTDSVVMGLSYFRKY | |
| | TGCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGAGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTATGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950865 | ATGGGAATCCACGAAAGTGTGTCGA | 69 | MGIHESVSKQFAKNGHSKY | 90 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCGTGGCAG | | PAPGELWLLPYFVPVHPGR | |
| | AGCGAATACGACGGACCGCAGTTCA | | WWVHTRWIYLAMGYLEA | |
| | TGAGCATCGGCTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGCTTCGGTA | | TSIVMYLHEGPVSANYKKI | |
| | CCAGCATCAACTACGTGGTCCTGCGA | | AARWPEFLSLNPSGMFMN | |
| | CTACTGGGCCTGTCACGGGACCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHIFAMEALETQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNDEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGTTTCGGCGAATTA | | | |
| | CAAAAAGATTGCGGCCCGATGGCCC | | | |
| | GAATTTCTGTCTCTGAATCCGTCGGG | | | |
| | AATGTTTATGAACGGCACCAACGGTC | | | |
| | TGCAGGTCTGGGATACTGCGTTTGCC | | | |
| | GTGCAATACGCGTGTGTTTGTGGCTT | | | |
| | TGCCGAACTTCCCCAGTACCAGAAG | | | |
| | ACGATCCGAGCGGCGTTTGATTTTCT | | | |
| | CGATCGGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAAGCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTGGAATGC | | | |
| | ACTGATTCTGTTGTTCTGGGTCTGTC | | | |
| | CTACTTTCGAAAGTACCACGATTACC | | | |
| | GCAACGAAGACGTGGACCGAGCCAT | | | |
| | CTCTGCTGCCATTGGATACATTATTC | | | |
| | GAGAGCAGCAGCCTGACGGCGGCTT | | | |
| | CTTTGGCTCCTGGGGCGTGTGCTACT | | | |
| | GCTACGCTCACATATTTGCCATGGAG | | | |
| | GCTCTGGAGACGCAGAATCTCAACT | | | |
| | ATAACAACTGTTCCACGGTTCAAAAG | | | |
| | GCGTGCGACTTTCTGGCGGGCTACCA | | | |
| | GGAAGCAGATGGAGGCTGGGCCGAG | | | |
| | GACTTTAAGTCGTGCGAGACTCAGAT | | | |
| | GTACGTGCGCGGACCCCATTCGCTGG | | | |
| | TCGTGCCTACTGCCATGGCCCTGTTG | | | |
| | AGTTTGATGAGTGGTCGGTATCCCCA | | | |
| | GGAGGACAAGATTCATGCTGCGGCC | | | |
| | CGGTTTCTCATGAGCAAGCAGATGA | | | |
| | GCAACGATGAGTGGCTCAAGGAGGA | | | |
| | GATGGAGGGGGTGTTTAACCATACTT | | | |
| | GTGCCATTGAGTATCCCAACTACCGG | | | |
| | TTTTATTTTGTCATGAAGGCTTTGGG | | | |
| | GTTGTATTTCAAGGGATATTGCCAGT | | | |
| | GA | | | |
| 950866 | ATGGGAATCCACGAAAGTGTGTCGA | 70 | MGIHESVSKQFAKNGHSKY | 91 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCGTGGCAG | | PAPGELWLLPYFVPVHPGR | |
| | AGCGAATACGACGGACCGCAGTTCA | | WWVHTRWIYLAMGYLEA | |
| | TGAGCATCGGCTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGCTTCGGTA | | TSIVMYLHEGPVSANYKKI | |
| | CCAGCATCAACTACGTGGTCCTGCGA | | AARWPEFLSLNPSGMFMN | |
| | CTACTGGGCCTGTCACGGGACCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDNRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHIFAMEALETQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNDEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGTTTCGGCGAATTA | | | |
| | CAAAAAGATTGCGGCCCGATGGCCC | | | |
| | GAATTTCTGTCTCTGAATCCGTCGGG | | | |
| | AATGTTTATGAACGGCACCAACGGTC | | | |
| | TGCAGGTCTGGGATACTGCGTTTGCC | | | |
| | GTGCAATACGCGTGTGTTTGTGGCTT | | | |
| | TGCCGAACTTCCCCAGTACCAGAAG | | | |
| | ACGATCCGAGCGGCGTTTGATTTTCT | | | |
| | CGATCGGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAATCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTGGAATGC | | | |
| | ACTGATTCTGTTGTTCTGGGTCTGTC | | | |
| | CTACTTTCGAAAGTACCACGATTACC | | | |
| | GCAACGAAGACGTGGACCGAGCCAT | | | |
| | CTCTGCTGCCATTGGATACATTATTC | | | |
| | GAGAGCAGCAGCCTGACGGCGGCTT | | | |
| | CTTTGGCTCCTGGGGCGTGTGCTACT | | | |
| | GCTACGCTCACATATTTGCCATGGAG | | | |
| | GCTCTGGAGACGCAGAATCTCAACT | | | |
| | ATAACAACTGTTCCACGGTTCAAAAG | | | |
| | GCGTGCGACTTTCTGGCGGGCTACCA | | | |
| | GGAAGCAGATGGAGGCTGGGCCGAG | | | |
| | GACTTTAAGTCGTGCGAGACTCAGAT | | | |
| | GTACGTGCGCGGACCCCATTCGCTGG | | | |
| | TCGTGCCTACTGCCATGGCCCTGTTG | | | |
| | AGTTTGATGAGTGGTCGGTATCCCCA | | | |
| | GGAGGACAAGATTCATGCTGCGGCC | | | |
| | CGGTTTCTCATGAGCAAGCAGATGA | | | |
| | GCAACGATGAGTGGCTCAAGGAGGA | | | |
| | GATGGAGGGGGTGTTTAACCATACTT | | | |
| | GTGCCATTGAGTATCCCAACTACCGG | | | |
| | TTTTATTTTGTCATGAAGGCTTTGGG | | | |
| | GTTGTATTTCAAGGGATATTGCCAGT | | | |
| | GA | | | |
| 950867 | ATGGGAATCCACGAAAGTGTGTCGA | 71 | MGIHESVSKQFAKNGHSKY | 92 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLGSKLVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGGCTCCAAGCTCGTGAA | | ASINYVVLRLLGLSRDHPV | |
| | AAATGCCTACGAAGCGGCTCTCAAA | | CVKARKTLLIKFGGAINNP | |
| | AACTGGCATCTGTTTGCGTCGCTGCA | | HWGKTWLSILNLYKWEGV | |
| | AGACCCCGACTCCGGCGCATGGCAG | | NPTPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTG | | MTSIVMYLHEGPDSANYKK | |
| | CCAGCATCAACTACGTGGTCCTGCGA | | IAARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCATCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGACCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950868 | ATGGGAATCCACGAAAGTGTGTCGA | 4 | MGIHESVSKQFAKNGHSKY | 3 |
| | AACAGTTTGCGAAAAACGGACATTC | | RSDRYGLPKTDLRRWTFHA | |
| | CAAGTACCGCAGCGACCGATACGGC | | SDLGAQWWKYDGTTPLEE | |
| | TTACCTAAGACGGATCTGCGACGATG | | LEKRATDYVRYSLELPGYA | |
| | GACGTTCCACGCGTCCGATCTGGGGG | | PVTLDSKPVKNAYEAALKS | |
| | CGCAATGGTGGAAGTATGACGGTAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAGATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AGCTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCSFAELPQYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGAGCTCTGGCTGTTGCCCTACTT | | NVGSFEPGSFASYEPIRASS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSYFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALVTQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRLYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTAGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATCGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTATATTTTGTCATGAAGGCT | | | |
| | TTGGGGTTGTATTTCAAGGGATATTG | | | |
| | CCAGTGA | | | |
| 950872 | ATGGGAATCCACGAAAGTGTGTCGA | 73 | MGIHESVSKQFAKNGHSKY | 94 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCGTGGCAG | | PAPGELWLLPYFVPVHPGR | |
| | AGCGAATACGACGGACCGCAGTTCA | | WWVHTRWIYLAMGYLEA | |
| | TGAGCATCGGCTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGCTTCGGTA | | SSIVMYLHEGPDPANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPMEENSYRDD | |
| | CAACAACCCCCATTGGGGCAAGACC | | RVGGWPFSTKTQGYPVSDC | |
| | TGGCTGTCGATTCTCAATCTCTACAA | | TAEALKAIIMVQNTPGYED | |
| | ATGGGAGGGTGTGAATCCGGCCCCT | | LKKQVSDKRKHTAIDLLLG | |
| | GGCGAGCTCTGGCTGTTGCCCTACTT | | MQNVGSFEPGSFASYEPIRA | |
| | TGTTCCTGTTCATCCGGGTCGATGGT | | SSMLEKINPAEVFGNIMVE | |
| | GGGTCCATACCCGGTGGATCTACCTT | | YPYVECTDSVVLGLSYFRK | |
| | GCCATGGGCTATCTGGAGGCTGCGG | | YHDYRNEDVDPAISAAIGYI | |
| | AGGCCCAATGCGAACTCACTCCGTTG | | IREQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGTCCTCCATTGTCATGTATCTC | | | |
| | CATGAGGGGCCCGATCCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GATGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | ACCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TCTCCTACTTTCGAAAGTACCACGAT | | | |
| | TACCGCAACGAAGACGTGGACCCAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950887 | ATGGGAATCCACGAAAGTGTGTCGA | 74 | MGIHESVSKQFAKNGHSKY | 95 |
| | AACAGTTTGCGAAAAACGGACATTC | | RSDRYGLPKTDLRRWTFHA | |
| | CAAGTACCGCAGCGACCGATACGGC | | SDLGAQWWKYDGTTPLEE | |
| | TTACCTAAGACGGATCTGCGACGATG | | LEKRATDYVRYSLELPGYA | |
| | GACGTTCCACGCGTCCGATCTGGGGG | | PVTLDSKPVKNAYEAALKS | |
| | CGCAATGGTGGAAGTATGACGGTAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAGATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AGCTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCSFAELPQYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGAGCTCTGGCTGTTGCCCTACTT | | NVGSFEPGSFASYEPIRASS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSYFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALVTQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTAGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATCGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950888 | ATGGGAATCCACGAAAGTGTGTCGA | 71 | MGIHESVSKQFAKNGHSKY | 92 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLGSKLVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGGCTCCAAGCTCGTGAA | | ASINYVVLRLLGLSRDHPV | |
| | AAATGCCTACGAAGCGGCTCTCAAA | | CVKARKTLLIKFGGAINNP | |
| | AACTGGCATCTGTTTGCGTCGCTGCA | | HWGKTWLSILNLYKWEGV | |
| | AGACCCCGACTCCGGCGCATGGCAG | | NPTPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTG | | MTSIVMYLHEGPDSANYKK | |
| | CCAGCATCAACTACGTGGTCCTGCGA | | IAARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCATCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGACCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950910 | ATGGGAATCCACGAAAGTGTGTCGA | 63 | MGIHESVSKQFAKNGHSKY | 84 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSNNYVVLRLLGLSRDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCAACAACTACGTGGTCCTGCG | | IAARWPEFLSLNPSGMFMN | |
| | ACTACTGGGCCTGTCACGGGATCATC | | GTNGLQVWDTAFAVQYAS | |
| | CGGTCTGCGTCAAGGCGCGCAAAAC | | VCGFAELPQYQKTIRAAFD | |
| | GCTGCTCACCAAGTTTGGCGGCGCCA | | FLDRSQINEPTEENSYRDDR | |
| | TCAACAACCCCCATTGGGGCAAGAC | | VGGWPFSTKTQGYPVSDCT | |
| | CTGGCTGTCGATTCTCAATCTCTACA | | AEALKAIIMVQNTPGYEDL | |
| | AATGGGAGGGTGTGAATCCGGCCCC | | KKQVSDKRKHTAIDLLLGM | |
| | TGGCGAGCTCTGGCTGTTGCCCTACT | | QNVGSFEPGSFASYEPIRAS | |
| | TTGTTCCTGTTCATCCGGGCCGATGG | | SMLEKINPAEVFGNIMVEY | |
| | TGGGTCCATACCCGGTGGATCTACCT | | PYVECTDSVVMGLSYFRKY | |
| | TGCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSS | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGAGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTATGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTAGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 950917 | ATGGGAATCCACGAAAGTGTGTCGA | 74 | MGIHESVSKQFAKNGHSKY | 95 |
| | AACAGTTTGCGAAAAACGGACATTC | | RSDRYGLPKTDLRRWTFHA | |
| | CAAGTACCGCAGCGACCGATACGGC | | SDLGAQWWKYDGTTPLEE | |
| | TTACCTAAGACGGATCTGCGACGATG | | LEKRATDYVRYSLELPGYA | |
| | GACGTTCCACGCGTCCGATCTGGGGG | | PVTLDSKPVKNAYEAALKS | |
| | CGCAATGGTGGAAGTATGACGGTAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAGATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AGCTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCSFAELPQYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGAGCTCTGGCTGTTGCCCTACTT | | NVGSFEPGSFASYEPIRASS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSYFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALVTQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTAGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATCGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 959829 | ATGGGAATCCACGAAAGTGTGTCGA | 78 | MGIHESVSKQFAKNGHSKY | 99 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGFLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREASNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GINGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGATGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTACCCCCAC | | GPHSLVVPTAMALLSLMSG | |
| | ACCGGCTTTTTGAAGTTTGGCAACGC | | RYPQEDEIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGCTTCCAACACACGAC | | | |
| | ACTTGTGTCTCGCTCCCGTCAACAAT | | | |
| | GCCATGACCTCCATTGTCATGTATCT | | | |
| | CCATGAGGGGCCCGATTCGGCGAAT | | | |
| | TACAAAAAGATTGCGGCCCGATGGC | | | |
| | CCGAATTTCTGTCTCTGAATCCGTCG | | | |
| | GGAATGTTTATGAACGGCATCAACG | | | |
| | GTCTGCAGGTCTGGGATACTGCGTTT | | | |
| | GCCGTGCAATACGCGTGTGTTTGTGG | | | |
| | CTTTGCCGAACTTCCCCAGTACCAGA | | | |
| | AGACGATCCGAGCGGCGTTTGATTTT | | | |
| | CTCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTATCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGTAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCAGGTT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACGAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| Parent | ATGACAGAATTTTATTCTGACACAAT | 8 | MTEFYSDTIGLPKTDPRLW | 313 |
| strain | CGGTCTACCAAAGACAGATCCACGT | | RLRTDELGRESWEYLTPQQ | |
| from | CTTTGGAGACTGAGAACTGATGAGCT | | AANDPPSTFTQWLLQDPKF | |
| Example | AGGCCGAGAAAGCTGGGAATATTTA | | PQPHPERNKHSPDFSAFDA | |
| 4 | ACCCCTCAGCAAGCCGCAAACGACC | | CHNGASFFKLLQEPDSGIFP | |
| | CACCATCCACTTTCACGCAGTGGCTT | | CQYKGPMFMTIGYVAVNYI | |
| | CTTCAAGATCCCAAATTTCCTCAACC | | AGIEIPEHERIELIRYIVNTA | |
| | TCATCCAGAAAGAAATAAGCATTCA | | HPVDGGWGLHSVDKSTVF | |
| | CCAGATTTTTCAGCCTTCGATGCGTG | | GTVLNYVILRLLGLPKDHP | |
| | TCATAATGGTGCATCTTTTTTCAAAC | | VCAKARSTLLRLGGAIGSP | |
| | TGCTTCAAGAGCCTGACTCAGGTATT | | HWGKIWLSALNLYKWEGV | |
| | TTTCCGTGTCAATATAAAGGACCCAT | | NPAPPETWLLPYSLPMHPG | |
| | GTTCATGACAATCGGTTACGTAGCCG | | RWWVHTRGVYIPVSYLSLV | |
| | TAAACTATATCGCCGGTATTGAAATT | | KFSCPMTPLLEELRNEIYTK | |
| | CCTGAGCATGAGAGAATAGAATTAA | | PFDKINFSKNRNTVCGVDL | |
| | TTAGATACATCGTCAATACAGCACAT | | YYPHSTTLNIANSLVVFYEK | |
| | CCGGTTGATGGTGGCTGGGGTCTACA | | YLRNRFIYSLSKKKVYDLIK | |
| | TTCTGTTGACAAATCCACCGTGTTTG | | TELQNTDSLCIAPVNQAFC | |
| | GTACAGTATTGAACTATGTAATCTTA | | ALVTLIEEGVDSEAFQRLQ | |
| | CGTTTATTGGGTCTACCCAAGGACCA | | YRFKDALFHGPQGMTIMGT | |
| | CCCGGTTTGCGCCAAGGCAAGAAGC | | NGVQTWDCAFAIQYFFVA | |
| | ACATTGTTAAGGTTAGGCGGTGCTAT | | GLAERPEFYNTIVSAYKFLC | |
| | TGGATCCCCTCACTGGGGAAAAATTT | | HAQFDTECVPGSYRDKRKG | |
| | GGCTAAGTGCACTAAACTTGTATAAA | | AWGFSTKTQGYTVADCTA | |
| | TGGGAAGGTGTGAACCCTGCCCCTCC | | EAIKAIIMVKNSPVFSEVHH | |
| | TGAAACTTGGTTACTTCCATATTCAC | | MISSERLFEGIDVLLNLQNI | |
| | TGCCCATGCATCCGGGGAGATGGTG | | GSFEYGSFATYEKIKAPLA | |
| | GGTTCATACTAGAGGTGTTTACATTC | | METLNPAEVFGNIMVEYPY | |
| | CGGTCAGTTACCTGTCATTGGTCAAA | | VECTDSSVLGLTYFHKYFD | |
| | TTTTCTTGCCCAATGACTCCTCTTCTT | | YRKEEIRTRIRIAIEFIKKSQL | |
| | GAAGAACTGAGGAATGAAATTTACA | | PDGSWYGSWGICFTYAGM | |
| | CTAAACCGTTTGACAAGATTAACTTC | | FALEALHTVGETYENSSTV | |
| | TCCAAGAACAGGAATACCGTATGTG | | RKGCDFLVSKQMKDGGWG | |
| | GAGTAGACCTATATTACCCCCATTCT | | ESMKSSELHSYVDSEKSLV | |
| | ACTACTTTGAATATTGCGAACAGCCT | | VQTAWALIALLFAEYPNKE | |
| | TGTAGTATTTTACGAAAAATACCTAA | | VIDRGIDLLKNRQEESGEW | |
| | GAAACCGGTTCATTTACTCTCTATCC | | KFESVEGVFNHSCAIEYPSY | |
| | AAGAAGAAGGTTTATGATCTAATCA | | RFLFPIKALGMYSRAYETH | |
| | AAACGGAGTTACAGAATACTGATTC | | TL | |
| | CTTGTGTATAGCACCTGTTAACCAGG | | | |
| | CGTTTTGCGCACTTGTCACTCTTATTG | | | |
| | AAGAAGGGGTAGACTCGGAAGCGTT | | | |
| | CCAGCGTCTCCAATATAGGTTCAAGG | | | |
| | ATGCATTGTTCCATGGTCCACAGGGT | | | |
| | ATGACCATTATGGGAACAAATGGTG | | | |
| | TGCAAACCTGGGATTGTGCGTTTGCC | | | |
| | ATTCAATACTTTTTCGTCGCAGGCCT | | | |
| | CGCAGAAAGACCTGAATTCTATAAC | | | |
| | ACAATTGTCTCTGCCTATAAATTCTT | | | |
| | GTGTCATGCTCAATTTGACACCGAGT | | | |
| | GCGTTCCAGGTAGTTATAGGGATAA | | | |
| | GAGAAAGGGGGCTTGGGGCTTCTCA | | | |
| | ACAAAAACACAGGGCTATACAGTGG | | | |
| | CAGATTGCACTGCAGAAGCAATTAA | | | |
| | AGCCATCATCATGGTGAAAAACTCTC | | | |
| | CCGTCTTTAGTGAAGTACACCATATG | | | |
| | ATTAGCAGTGAACGTTTATTTGAAGG | | | |
| | CATTGATGTGTTATTGAACCTACAAA | | | |
| | ACATCGGATCTTTTGAATATGGTTCC | | | |
| | TTTGCAACCTATGAAAAAATCAAGG | | | |
| | CCCCACTAGCAATGGAAACCTTGAAT | | | |
| | CCTGCTGAAGTTTTTGGTAACATAAT | | | |
| | GGTAGAATACCCATACGTGGAATGT | | | |
| | ACTGATTCATCCGTTCTGGGGTTGAC | | | |
| | ATATTTTCACAAGTACTTCGACTATA | | | |
| | GGAAAGAGGAAATACGTACACGCAT | | | |
| | CAGAATCGCCATCGAATTCATAAAA | | | |
| | AAATCTCAATTACCAGATGGAAGTTG | | | |
| | GTATGGAAGCTGGGGTATTTGTTTTA | | | |
| | CATATGCCGGTATGTTTGCATTGGAG | | | |
| | GCATTACACACCGTGGGGGAGACCT | | | |
| | ATGAGAATTCCTCAACGGTAAGAAA | | | |
| | AGGTTGCGACTTCTTGGTCAGTAAAC | | | |
| | AGATGAAGGATGGCGGTTGGGGGGA | | | |
| | ATCAATGAAGTCCAGTGAATTACATA | | | |
| | GTTATGTGGATAGTGAAAAATCGCTA | | | |
| | GTCGTTCAAACCGCATGGGCGCTAAT | | | |
| | TGCACTTCTTTTCGCTGAATATCCTA | | | |
| | ATAAAGAAGTCATCGACCGCGGTAT | | | |
| | TGACCTTTTAAAAAATAGACAAGAA | | | |
| | GAATCCGGGGAATGGAAATTTGAAA | | | |
| | GTGTAGAAGGTGTTTTCAACCACTCT | | | |
| | TGTGCAATTGAATACCCAAGTTATCG | | | |
| | ATTCTTATTCCCTATTAAGGCATTAG | | | |
| | GTATGTACAGCAGGGCATATGAAAC | | | |
| | ACATACGCTTTAA | | | |
| 756247 | ATGACAGAATTTTATTCTGACACAAT | 80 | MTEFYSDTIGLPKTDPRLW | 100 |
| | CGGTCTACCAAAGACAGATCCACGT | | RLRTDELGRESWEYLTPQQ | |
| | CTTTGGAGACTGAGAACTGATGAGCT | | AANDPPSTFTQWLLQDPKF | |
| | AGGCCGAGAAAGCTGGGAATATTTA | | PQPHPERNKHSPDFSAFDA | |
| | ACCCCTCAGCAAGCCGCAAACGACC | | CHNGASFFKLLQEPDSGIFP | |
| | CACCATCCACTTTCACGCAGTGGCTT | | CQYKGPMFMTIGYVAVNYI | |
| | CTTCAAGATCCCAAATTTCCTCAACC | | AGIEISEHERIELIRYIVNTV | |
| | TCATCCAGAAAGAAATAAGCATTCA | | HPVDGGWGLHSVDKSTVF | |
| | CCAGATTTTTCAGCCTTCGATGCGTG | | GTVLNYVILRLLGLPKDHP | |
| | TCATAATGGTGCATCTTTTTTCAAAC | | VCAKARSTLLRLGGAIGSP | |
| | TGCTTCAAGAGCCTGACTCAGGTATT | | HWGKIWLSALNLYKWEGV | |
| | TTTCCGTGTCAATATAAAGGACCCAT | | NPAPPETWLLPYSLPMHPG | |
| | GTTCATGACAATCGGTTACGTAGCCG | | RWWVHTRGVYIPVSYLSLV | |
| | TAAACTATATCGCCGGTATTGAAATT | | KFSCPMTPLLEELRNEIYTK | |
| | TCTGAGCATGAGAGAATAGAATTAA | | PFDKINFSKNRNTVCGVDL | |
| | TTAGATACATCGTCAATACAGTACAT | | YYPHSTTLNIANGLVVFYE | |
| | CCGGTTGATGGTGGCTGGGGTCTACA | | KYLRNRFIYSLSKKKGYDLI | |
| | TTCTGTTGACAAATCCACCGTGTTTG | | KTELQNTDSLCIAPVNQAF | |
| | GTACAGTATTAAACTATGTAATCTTA | | CALVTLIEEGVDSEAFQRLQ | |
| | CGTTTATTGGGTCTACCCAAGGACCA | | YRFKDALFHGPQGMTIMGT | |
| | CCCGGTTTGCGCCAAGGCAAGAAGC | | NGVQTWDCAFAIQYFFVA | |
| | ACATTGTTAAGGTTAGGCGGTGCTAT | | GLAERPEFYNTIVSAYKFLC | |
| | TGGATCCCCTCACTGGGGAAAAATTT | | HAQFDTECVPGSYRDERKG | |
| | GGCTAAGTGCACTAAACTTGTATAAA | | AWGFSTKTQGYTVADCTA | |
| | TGGGAAGGTGTGAACCCTGCCCCTCC | | EAIKAIIMVKNSPVFSEVHH | |
| | TGAAACTTGGTTACTTCCATATTCAC | | MISSERLFEGIDVLLNLQNI | |
| | TGCCCATGCATCCGGGGAGATGGTG | | GSLEYGSFATYEKIKAPLA | |
| | GGTTCATACTAGAGGTGTTTACATTC | | METLNPAEVFGNIMVEYPY | |
| | CGGTCAGTTACCTGTCATTGGTCAAA | | VECTDSSVLGLTYFHKYFD | |
| | TTTTCTTGCCCAATGACTCCTCTTCTT | | YRKEEIRTRIRIAIEFIKKSQL | |
| | GAAGAACTGAGGAATGAAATTTACA | | PDGSWYGSWGICFTYAGM | |
| | CTAAACCGTTTGACAAGATTAACTTC | | FALEALHTVGETYENSSTV | |
| | TCCAAGAACAGGAATACCGTATGTG | | RKGCDFLVSKQMEDGGWG | |
| | GAGTAGACCTATATTACCCCCATTCT | | ESMKSSELHSYVDSEKSLV | |
| | ACTACTTTGAATATTGCGAACGGCCT | | VQTAWALIALLFAEYPNKE | |
| | TGTAGTGTTTTACGAAAAATACCTAA | | VIDRGIDLLKNRQEESGEW | |
| | GAAACCGGTTCATTTACTCTCTATCC | | KFESVEGVFNHSCAIEYPSY | |
| | AAGAAGAAGGGTTATGATCTAATCA | | RFLFPIKALGMYSRA | |
| | AAACGGAGTTACAGAATACTGATTC | | | |
| | CTTGTGTATAGCACCTGTTAACCAGG | | | |
| | CGTTTTGCGCACTTGTCACTCTTATTG | | | |
| | AAGAAGGGGTAGACTCGGAAGCGTT | | | |
| | CCAGCGTCTCCAATATAGGTTCAAGG | | | |
| | ATGCATTGTTCCATGGTCCACAGGGT | | | |
| | ATGACCATTATGGGAACAAATGGTG | | | |
| | TGCAAACCTGGGATTGTGCGTTTGCC | | | |
| | ATTCAATACTTTTTCGTCGCAGGCCT | | | |
| | CGCAGAAAGACCTGAATTCTATAAC | | | |
| | ACAATTGTCTCTGCCTATAAATTCTT | | | |
| | GTGTCATGCTCAATTTGACACCGAGT | | | |
| | GCGTTCCAGGTAGTTATAGGGATGA | | | |
| | GAGAAAGGGGGCTTGGGGCTTCTCA | | | |
| | ACAAAAACACAGGGCTATACAGTGG | | | |
| | CAGATTGCACTGCAGAAGCAATTAA | | | |
| | AGCCATCATCATGGTGAAAAACTCTC | | | |
| | CCGTCTTTAGTGAAGTACACCATATG | | | |
| | ATTAGCAGTGAACGTTTATTTGAAGG | | | |
| | CATTGATGTGTTATTGAACCTACAAA | | | |
| | ACATCGGATCTTTAGAATATGGTTCC | | | |
| | TTTGCAACCTATGAAAAAATCAAGG | | | |
| | CCCCACTAGCAATGGAAACCTTGAAT | | | |
| | CCTGCTGAAGTTTTTGGTAACATAAT | | | |
| | GGTAGAATACCCATACGTGGAATGT | | | |
| | ACTGATTCATCCGTTCTGGGGTTGAC | | | |
| | ATATTTTCACAAGTACTTCGACTATA | | | |
| | GGAAAGAGGAAATACGTACACGCAT | | | |
| | CAGAATCGCCATCGAATTCATAAAA | | | |
| | AAATCTCAACTACCAGATGGAAGTT | | | |
| | GGTATGGAAGCTGGGGTATTTGTTTT | | | |
| | ACATATGCCGGTATGTTTGCATTGGA | | | |
| | GGCATTACACACCGTGGGGGAGACC | | | |
| | TATGAGAATTCCTCAACGGTAAGAA | | | |
| | AAGGTTGCGACTTCTTGGTCAGTAAA | | | |
| | CAGATGGAGGATGGCGGTTGGGGGG | | | |
| | AATCAATGAAGTCCAGTGAATTACAT | | | |
| | AGTTATGTGGATAGTGAAAAATCGCT | | | |
| | AGTCGTTCAAACCGCATGGGCGCTA | | | |
| | ATTGCACTTCTTTTCGCTGAATATCCT | | | |
| | AATAAAGAAGTCATCGACCGCGGTA | | | |
| | TTGACCTTTTAAAAAATAGACAAGA | | | |
| | AGAATCCGGGGAATGGAAATTTGAA | | | |
| | AGTGTAGAAGGTGTTTTCAACCACTC | | | |
| | TTGTGCAATTGAATACCCAAGTTATC | | | |
| | GATTCTTATTCCCTATTAAGGCATTA | | | |
| | GGTATGTACAGCAGGGCATAG | | | |
| 756248 | ATGACAGAATTTTATTCTGACACAAT | 81 | MTEFYSDTIGLPKTDPRLW | 101 |
| | CGGTCTACCAAAGACAGATCCACGT | | RLRTDELGRESWEYLTPQQ | |
| | CTTTGGAGACTGAGAACTGATGAGCT | | AANDPPSTFTQWLLQDPKF | |
| | AGGCCGAGAAAGCTGGGAATATTTA | | PQPHPERNKHSPDFSAFDA | |
| | ACCCCTCAGCAAGCCGCAAACGACC | | CHNGASFFKLLQEPDSGIFP | |
| | CACCATCCACTTTCACGCAGTGGCTT | | CQYKGPMFMTIGYVAVNYI | |
| | CTTCAAGATCCCAAATTTCCTCAACC | | AGIEIPEHERIELIRYIVNTA | |
| | TCATCCAGAAAGAAATAAGCATTCA | | HPVDGGWGLHSVDKSTVF | |
| | CCAGATTTTTCAGCCTTCGATGCGTG | | GTVLNYVILRLLGLPKDHP | |
| | TCATAATGGTGCATCTTTTTTCAAAC | | VCAKARSTLLRLGGAIGSP | |
| | TGCTTCAAGAGCCTGACTCAGGTATT | | HWGKIWLSALNLYKWEGV | |
| | TTTCCGTGTCAATATAAAGGACCCAT | | NPAPPETWLLPYSLPMHPG | |
| | GTTCATGACAATCGGTTACGTAGCTG | | RWWVHTRGVYIPVSYLSLV | |
| | TAAACTATATCGCCGGTATTGAAATT | | KFSCPMTPLLEELRNEIYTS | |
| | CCTGAGCATGAGAGAATAGAATTAA | | PFDKINFSKNRNAVCGVDL | |
| | TTAGATACATCGTCAATACAGCACAT | | YYPHSTTLNIANSL VVFYEK | |
| | CCGGTTGATGGTGGCTGGGGTCTACA | | YLRNRFIYSLSKKKVYDLIK | |
| | TTCTGTTGACAAATCCACCGTGTTTG | | TELQNTDSLCIAPVNQAFC | |
| | GTACAGTATTGAACTATGTAATCTTA | | ALVTLIEEGVDSEAFQRLQ | |
| | CGTTTATTGGGTCTACCCAAGGACCA | | YRFKDALFHGPQGMTIMGT | |
| | CCCGGTTTGCGCCAAGGCAAGAAGC | | NGVQTWDCAFAIQYFFVA | |
| | ACATTGTTAAGGTTAGGCGGTGCTAT | | GLAERPEFYNTIVSAYKFLC | |
| | TGGATCCCCTCACTGGGGAAAAATTT | | HAQFDTECVPGSYRDKRKG | |
| | GGCTAAGTGCACTAAACTTGTATAAA | | AWGFSTKTQGYTVADCTA | |
| | TGGGAAGGTGTGAACCCTGCCCCTCC | | EAIKAIIMVKNSPVFSEVHH | |
| | TGAAACTTGGTTACTTCCATATTCAC | | MISSERLFEGIDVLLNLQNI | |
| | TGCCCATGCATCCGGGGAGATGGTG | | GSLEYGSFATYEKIKAPLA | |
| | GGTTCATACTAGAGGTGTTTACATTC | | METLNPAEVFGNIMVEYPY | |
| | CGGTCAGTTACCTGTCATTGGTCAAA | | VECTDSSVLGLTYFHKYFD | |
| | TTTTCTTGCCCAATGACTCCTCTTCTT | | YRKEEIRTRIRIAIEFIKKSQL | |
| | GAAGAACTGAGGAATGAAATTTACA | | PDGSWYGSWGICFTYAGM | |
| | CTAGTCCGTTTGACAAGATTAACTTC | | FALEALHNVGETYENSSTV | |
| | TCCAAGAACAGGAATGCCGTATGTG | | RKGCDFLVSKQMKDGGWG | |
| | GAGTAGACCTATATTACCCCCATTCT | | ESMKSSELHSYVDSEKSLV | |
| | ACTACTTTGAATATTGCGAACAGCCT | | VQTTWALIALLFAEYPNKE | |
| | TGTAGTATTTTACGAAAAATACCTAA | | VIDRGIDLLKNRQEESGEW | |
| | GAAACCGGTTCATTTACTCTCTATCC | | KFGSVEGVFNHSCAIEYPSY | |
| | AAGAAGAAGGTTTATGATCTAATCA | | RFLFPIKALGMYSRAYETH | |
| | AAACGGAGTTACAGAATACTGATTC | | TL | |
| | CTTGTGTATAGCACCTGTTAACCAGG | | | |
| | CGTTTTGCGCACTTGTCACTCTTATTG | | | |
| | AAGAAGGGGTAGACTCGGAAGCGTT | | | |
| | CCAGCGTCTCCAATATAGGTTCAAGG | | | |
| | ATGCATTGTTCCATGGTCCACAGGGT | | | |
| | ATGACCATTATGGGAACAAATGGTG | | | |
| | TGCAAACCTGGGATTGTGCGTTTGCC | | | |
| | ATTCAATACTTTTTCGTCGCAGGCCT | | | |
| | CGCAGAAAGACCTGAATTCTATAAC | | | |
| | ACAATTGTCTCTGCCTATAAATTCTT | | | |
| | GTGTCATGCTCAATTTGACACCGAGT | | | |
| | GCGTTCCAGGTAGTTATAGGGATAA | | | |
| | GAGAAAGGGGGCTTGGGGCTTCTCA | | | |
| | ACAAAAACACAGGGCTATACAGTGG | | | |
| | CAGATTGCACTGCAGAAGCAATTAA | | | |
| | AGCCATCATCATGGTGAAAAACTCTC | | | |
| | CCGTCTTTAGTGAAGTACACCATATG | | | |
| | ATTAGCAGTGAACGTTTATTTGAAGG | | | |
| | CATTGATGTGTTATTGAACCTACAAA | | | |
| | ACATCGGATCTCTTGAATATGGTTCC | | | |
| | TTTGCAACCTATGAAAAAATCAAGG | | | |
| | CCCCACTAGCAATGGAAACCTTGAAT | | | |
| | CCTGCTGAAGTTTTTGGTAACATAAT | | | |
| | GGTAGAATACCCATACGTGGAATGT | | | |
| | ACTGATTCATCCGTTCTGGGGTTGAC | | | |
| | ATATTTTCACAAGTACTTCGACTATA | | | |
| | GGAAAGAGGAAATACGTACACGCAT | | | |
| | CAGAATCGCCATCGAATTCATAAAA | | | |
| | AAATCTCAATTACCAGATGGAAGTTG | | | |
| | GTATGGAAGCTGGGGTATTTGTTTTA | | | |
| | CATATGCCGGTATGTTTGCATTGGAG | | | |
| | GCATTACACAACGTGGGGGAGACCT | | | |
| | ATGAGAATTCCTCAACGGTAAGAAA | | | |
| | AGGTTGCGACTTCTTGGTCAGTAAAC | | | |
| | AGATGAAGGATGGCGGTTGGGGGGA | | | |
| | ATCAATGAAGTCCAGTGAATTACATA | | | |
| | GTTATGTGGATAGTGAAAAATCGCTA | | | |
| | GTCGTTCAAACCACATGGGCGCTAAT | | | |
| | TGCACTTCTTTTCGCTGAATATCCTA | | | |
| | ATAAAGAAGTCATCGACCGCGGTAT | | | |
| | TGACCTTTTAAAAAATAGACAAGAA | | | |
| | GAATCCGGGGAATGGAAATTTGGAA | | | |
| | GTGTAGAAGGTGTTTTCAACCACTCT | | | |
| | TGTGCAATTGAATACCCAAGTTATCG | | | |
| | ATTCTTATTCCCTATTAAGGCATTAG | | | |
| | GTATGTACAGCAGGGCATATGAAAC | | | |
| | ACATACGCTTTAA | | | |
| 756249 | ATGACAGAATTTTATTCTGACACAAT | 82 | MTEFYSDTIGLPKTDPRLW | 102 |
| | CGGTCTACCAAAGACAGATCCACGT | | RLRTDELGRESWEYLTPQQ | |
| | CTTTGGAGACTGAGAACTGATGAGCT | | AANDPPSTFTQWLLQDPKF | |
| | AGGCCGAGAAAGCTGGGAATATTTA | | PQPHPEGNKHSPDFSAFDA | |
| | ACCCCTCAGCAAGCCGCAAACGACC | | CHNGASFFKLLQEPDSGIFP | |
| | CACCATCCACTTTCACGCAGTGGCTT | | CQYKGPMFMTIGYVAVNYI | |
| | CTTCAAGATCCCAAATTTCCTCAACC | | AGIEVPEHERIELIRYIVNTA | |
| | TCATCCAGAAGGAAATAAGCATTCA | | HPVDGGWGLHSVDKSTVF | |
| | CCAGATTTTTCAGCCTTCGATGCGTG | | GTVLNYVILRLLGLPKDHP | |
| | TCATAATGGTGCATCTTTTTTCAAAC | | VCAKARSTLLRLGGAIGSP | |
| | TGCTTCAAGAGCCTGACTCAGGTATT | | HWGKIWLSALNLYKWEGV | |
| | TTTCCGTGTCAATATAAAGGACCCAT | | NPAPPETWLLPYSLPIHPGR | |
| | GTTCATGACAATCGGTTACGTAGCCG | | WWVHTRGVYIPVSYLSLV | |
| | TAAACTATATCGCCGGTATTGAAGTT | | KFSCPMTPLLEELRNEIYTK | |
| | CCTGAGCATGAGAGAATAGAATTAA | | PFDKINISKNRNTVCGVDLY | |
| | TTAGATACATCGTCAATACAGCACAT | | YPHSTTLNIANSLVVFYEKY | |
| | CCGGTTGATGGTGGCTGGGGTCTACA | | LRNRFIYSLSKKKVYDLIKT | |
| | TTCTGTTGACAAATCCACCGTGTTTG | | ELQNADSLCIAPVNQAFCA | |
| | GTACAGTATTGAACTATGTAATCTTA | | LVTLIEEGVDSEAFQRLQYR | |
| | CGTTTATTGGGTCTACCCAAGGACCA | | FKDALFHGPQGMTIMGTNG | |
| | CCCGGTTTGCGCCAAGGCAAGAAGC | | VQTWDCAFAIQYFFVAGLA | |
| | ACATTGTTAAGGTTAGGCGGTGCTAT | | ERPEFYNTIVSAYKFLCHAQ | |
| | TGGATCCCCTCACTGGGGAAAAATTT | | FDTECVPGSYRDKRKGAW | |
| | GGCTAAGTGCACTAAACTTGTATAAA | | GFSTKTQGYTVADCTAEAI | |
| | TGGGAAGGTGTGAACCCTGCCCCTCC | | KAIIMVKNSPVFSEVHHMIS | |
| | TGAAACTTGGTTACTTCCATATTCAC | | SERLFEGIDVLLNLQNIGSF | |
| | TGCCCATTCATCCGGGGAGATGGTGG | | EYGSFATYEKIKAPLAMET | |
| | GTTCATACTAGAGGTGTTTACATTCC | | LNPAEVFGNIMVEYPYVEC | |
| | GGTCAGTTACCTGTCATTGGTCAAAT | | TDSSVLGLTYFHKYFDYRK | |
| | TTTCTTGCCCAATGACTCCTCTTCTTG | | EEIRTRIRIAIEFIKKSQLPDG | |
| | AAGAACTGAGGAATGAAATTTACAC | | SWYGSWGICFTYAGMFAL | |
| | TAAACCGTTTGACAAGATTAACATCT | | EALHTVGETYENSSTVRKG | |
| | CCAAGAACAGGAATACCGTATGTGG | | SDFLVSKQMKDGGWGESM | |
| | AGTAGACCTATATTACCCCCATTCTA | | KSSELHSYVDSEKSLVVQT | |
| | CTACTTTGAATATTGCGAACAGCCTT | | AWALIALLFAEYPNKEVID | |
| | GTAGTATTTTACGAAAAATACCTAAG | | RGIDLLKNRQEESGEWKFE | |
| | AAACCGGTTCATTTACTCTCTATCCA | | SVEGVFNHSCAIEYPSYRFL | |
| | AGAAGAAGGTTTATGATCTAATCAA | | FPIKALGMYSRAYETHTL | |
| | AACGGAGTTACAGAATGCTGATTCCT | | | |
| | TGTGTATAGCACCTGTTAACCAGGCG | | | |
| | TTTTGCGCACTTGTCACTCTTATTGA | | | |
| | AGAAGGGGTAGACTCGGAAGCGTTC | | | |
| | CAGCGTCTCCAATATAGGTTCAAGGA | | | |
| | TGCATTGTTCCATGGTCCACAGGGTA | | | |
| | TGACCATTATGGGAACAAATGGTGT | | | |
| | GCAAACCTGGGATTGTGCGTTTGCCA | | | |
| | TTCAATACTTTTTCGTCGCAGGCCTC | | | |
| | GCAGAAAGACCTGAATTCTATAACA | | | |
| | CAATTGTCTCTGCCTATAAATTCTTG | | | |
| | TGTCATGCTCAATTTGACACCGAGTG | | | |
| | CGTTCCAGGTAGTTATAGGGATAAG | | | |
| | AGAAAGGGGGCTTGGGGCTTCTCAA | | | |
| | CAAAAACACAGGGCTATACAGTGGC | | | |
| | AGATTGCACTGCAGAAGCAATTAAA | | | |
| | GCCATCATCATGGTGAAAAACTCTCC | | | |
| | CGTCTTTAGTGAAGTACACCATATGA | | | |
| | TTAGCAGTGAACGTTTATTTGAAGGC | | | |
| | ATTGATGTGTTATTGAACCTACAAAA | | | |
| | CATCGGATCTTTTGAATATGGTTCCT | | | |
| | TTGCAACCTATGAAAAAATCAAGGC | | | |
| | CCCACTAGCAATGGAAACCTTGAATC | | | |
| | CTGCTGAAGTTTTTGGTAACATAATG | | | |
| | GTAGAATACCCATACGTGGAATGTA | | | |
| | CTGATTCATCCGTTCTGGGGTTGACA | | | |
| | TATTTTCACAAGTACTTCGACTATAG | | | |
| | GAAAGAGGAAATACGTACACGCATC | | | |
| | AGAATCGCCATCGAATTCATAAAAA | | | |
| | AATCTCAATTACCAGATGGAAGTTGG | | | |
| | TATGGAAGCTGGGGTATTTGTTTTAC | | | |
| | ATATGCCGGTATGTTTGCATTGGAGG | | | |
| | CATTACACACCGTGGGGGAGACCTA | | | |
| | TGAGAATTCCTCAACGGTAAGAAAA | | | |
| | GGTAGCGACTTCTTGGTCAGTAAACA | | | |
| | GATGAAGGATGGCGGTTGGGGGGAA | | | |
| | TCAATGAAGTCCAGTGAATTACATAG | | | |
| | TTATGTGGATAGTGAAAAATCGCTAG | | | |
| | TCGTTCAAACCGCATGGGCGCTAATT | | | |
| | GCACTTCTTTTCGCTGAATATCCTAA | | | |
| | TAAAGAAGTCATCGACCGCGGTATT | | | |
| | GACCTTTTAAAAAATAGACAAGAAG | | | |
| | AATCCGGGGAATGGAAATTTGAAAG | | | |
| | TGTAGAAGGTGTTTTCAACCACTCTT | | | |
| | GTGCAATTGAATACCCAAGTTATCGA | | | |
| | TTCTTATTCCCTATTAAGGCATTAGG | | | |
| | TATGTACAGCAGGGCATATGAAACA | | | |
| | CATACGCTTTAA | | | |
| N/A | ATGGGAATCCACGAAAGTGTGTCGA | 2 | MGIHESVSKQFAKNGHSKY | 1 |
| (Wild- | AACAGTTTGCGAAAAACGGACATTC | | RSDRYGLPKTDLRRWTFHA | |
| type | CAAGTACCGCAGCGACCGATACGGC | | SDLGAQWWKYDDTTPLEE | |
| ERG7) | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 2F1 | ATGGGAATCCACGAAAGTGTGTCGA | 103 | MGIHESVSKQFAKNGHSKY | 118 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWFHTRWIYLAMGYLEAA | |
| | TGTCGATCGGTTATGTGACGGCGTGC | | EAQCELTPLLEELRDEIYKK | |
| | TACTTTGGCGGCAACGAGATCCCCAC | | PYSEIDFSKHCNSISGVDLY | |
| | GCCGGTCAAAACCGAAATGATCAGA | | YPHTGLLKFGNALLRRYRK | |
| | TACATTGTCAACACAGCCCACCCAGT | | FRPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPVSANYKKIAA | |
| | CCAGCATCAACTACGTGGTCCTGCGA | | RWPEFLSLNPSGMFMNGTN | |
| | CTACTGGGCCTGTCACGGGATCATCC | | GLQVWDTAFAVQYACVCG | |
| | GGTCTGCGTCAAGGCGCGCAAAACG | | FAELPQYQKTIRAAFDFLDR | |
| | CTGCTCACCAAGTTTGGCGGCGCCAT | | SQINEPTEENSYRDDRVGG | |
| | CAACAACCCCCATTGGGGCAAGACC | | WPFSTKTQGYPVSDCTAEA | |
| | TGGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGNIMVEYPYV | |
| | GGTTCCATACCCGGTGGATCTACCTT | | ECTDSVVLGLSYFRKYHDY | |
| | GCCATGGGCTATCTGGAGGCTGCGG | | RNEDVDRAISAAIGYIIREQ | |
| | AGGCCCAATGCGAACTCACTCCGTTG | | QPDGGFFGSWGVCYCYAH | |
| | CTGGAGGAGCTCCGAGACGAAATCT | | MFAMEALETQNLNYNNCS | |
| | ACAAAAAGCCCTACTCGGAGATTGA | | TVQKACDFLAGYQEADGG | |
| | TTTCTCCAAACATTGCAACTCCATCT | | WAEDFKSCETQMYVRGPH | |
| | CCGGAGTCGACCTCTACTATCCCCAC | | SLVVPTAMALLSLMSGRYP | |
| | ACCGGCCTTTTGAAGTTTGGCAACGC | | QEDKIHAAARFLMSKQMS | |
| | GCTTCTCCGACGATACCGCAAGTTCA | | NDEWLKEEMEGVFNHTCAI | |
| | GACCGCAGTGGATCAAAGAAAAGGT | | EYPNYRFYFVMKALGLYFK | |
| | CAAGGAGGAAATTTACAACTTGTGC | | GYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGTTTCGGCGAATTA | | | |
| | CAAAAAGATTGCGGCCCGATGGCCC | | | |
| | GAATTTCTGTCTCTGAATCCGTCGGG | | | |
| | AATGTTTATGAACGGCACCAACGGTC | | | |
| | TGCAGGTCTGGGATACTGCGTTTGCC | | | |
| | GTGCAATACGCGTGTGTTTGTGGCTT | | | |
| | TGCCGAACTTCCCCAGTACCAGAAG | | | |
| | ACGATCCGAGCGGCGTTTGATTTTCT | | | |
| | CGATCGGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAAGCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTGGAATGC | | | |
| | ACTGATTCTGTTGTTCTGGGTCTGTC | | | |
| | CTACTTTCGAAAGTACCACGATTACC | | | |
| | GCAACGAAGACGTGGACCGAGCCAT | | | |
| | CTCTGCTGCCATTGGATACATTATTC | | | |
| | GAGAGCAGCAGCCTGACGGCGGCTT | | | |
| | CTTTGGCTCCTGGGGCGTGTGCTACT | | | |
| | GCTACGCTCACATGTTTGCCATGGAG | | | |
| | GCTCTGGAGACGCAGAATCTCAACT | | | |
| | ATAACAACTGTTCCACGGTTCAAAAG | | | |
| | GCGTGCGACTTTCTGGCGGGCTACCA | | | |
| | GGAAGCAGATGGAGGCTGGGCCGAG | | | |
| | GACTTTAAGTCGTGCGAGACTCAGAT | | | |
| | GTACGTGCGCGGACCCCATTCGCTGG | | | |
| | TCGTGCCTACTGCCATGGCCCTGTTG | | | |
| | AGTTTGATGAGTGGTCGGTATCCCCA | | | |
| | GGAGGACAAGATTCATGCTGCGGCC | | | |
| | CGGTTTCTCATGAGCAAGCAGATGA | | | |
| | GCAACGATGAGTGGCTCAAGGAGGA | | | |
| | GATGGAGGGGGTGTTTAACCATACTT | | | |
| | GTGCCATTGAGTATCCCAACTACCGG | | | |
| | TTTTATTTTGTCATGAAGGCTTTGGG | | | |
| | GTTGTATTTCAAGGGATATTGCCAGT | | | |
| | GA | | | |
| 2F11 | ATGGGAATCCACGAAAGTGTGTCGA | 104 | MGIHESVSKQFAKNGHSKY | 119 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSWDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLMPYFVPVHPG | |
| | TCGGAATACGACGGACCGCAGTTCA | | RWWVHTRWIYLAMGYRE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCATCAACTACGTGGTCCTGCGA | | IAARWPEFLSLNPSGMFMN | |
| | CTACTGGGCCTGTCATGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGATGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCGGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMQGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGCAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 3A8 | ATGGGAATCCACGAAAGTGTGTCGA | 105 | MGIHESVSKQFAKNGHSKY | 120 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDQ | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCACACCCGGTGGATCTACCTT | | PYVECTDSVVLGLSYFRKY | |
| | GCCATGGGCTATCTGGAGGCTGCGG | | HDYRNEDVDRAISAAIGFII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVH | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCAGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCAGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATTCATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCACGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 3B9 | ATGGGAATCCACGAAAGTGTGTCGA | 106 | MGIHESVSKQFAKNGHSKY | 316 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSICYVTACYFGG | |
| | AGGGCTACTGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | GAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVLTRWIYLAMGYLEAA | |
| | TGTCGATCTGTTATGTGACGGCGTGC | | EAQCELTPLLEELRDEIYKK | |
| | TACTTTGGCGGCAACGAGATCCCCAC | | PYSEIDFSKHCNSISGVDLY | |
| | GCCGGTCAAAACCGAAATGATCAGA | | YPHTGLLKFGNALLRRYRK | |
| | TACATTGTCAACACAGCCCACCCAGT | | FRPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPDSANYKKIAA | |
| | CCAGCATCAACTACGTGGTCCTGCGA | | RWPEFLSLNPSGMFMNGTN | |
| | CTACTGGGCCTGTCACGGGATCATCC | | GLQVWDTAFAVQYACVCG | |
| | GGTCTGCGTCAAGGCGCGCAAAACG | | FAELPQYQKTIRAAFDFLDR | |
| | CTGCTCACCAAGTTTGGCGGCGCCAT | | SQINEPTEENSYRDDRVGG | |
| | CAACAACCCCCATTGGGGCAAGACC | | WPFSTKTQGYPVSDCTAEA | |
| | TGGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGNIMVEYPYV | |
| | GGGTCCTTACCCGGTGGATCTACCTT | | ECTDSVVLGLSYFRKYHDY | |
| | GCCATGGGCTATCTGGAGGCTGCGG | | RNEDVDRAISAAIGYIIREQ | |
| | AGGCCCAATGCGAACTCACTCCGTTG | | QPDGGFFGSWGVCYCYAH | |
| | CTGGAGGAGCTCCGAGACGAAATCT | | MFAMEALETQNLNYNNCS | |
| | ACAAAAAGCCCTACTCGGAGATTGA | | TVQKACDFLAGYQEADGG | |
| | TTTCTCCAAACATTGCAACTCCATCT | | WAEDFKSCETQMYVRGPH | |
| | CCGGAGTCGACCTCTACTATCCCCAC | | SLVVPTAMALLSLMSGRYP | |
| | ACCGGCCTTTTGAAGTTTGGCAACGC | | QEDKIHAAARFLMSKQMS | |
| | GCTTCTCCGACGATACCGCAAGTTCA | | NGEWLKEEMEGVFNHTCAI | |
| | GACCGCAGTGGATCAAAGAAAAGGT | | EYPNYRFYFVMKALGLYF | |
| | CAAGGAGGAAATTTACAACTTGTGC | | MGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCATGGGATATTG | | | |
| | CCAGTGA | | | |
| 3B9b | ATGGGAATCCACGAAAGTGTGTCGA | 107 | MGIHESVSKQFAKNGHSKY | 317 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSICYVTACYFGG | |
| | AGGGCTACTGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | GAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVLTRWIYLAMGYLEAA | |
| | TGTCGATCTGTTATGTGACGGCGTGC | | EAQCELTPLLEELRDEIYKK | |
| | TACTTTGGCGGCAACGAGATCCCCAC | | PYSEIDFSKHCNSISGVDLY | |
| | GCCGGTCAAAACCGAAATGATCAGA | | YPHTGLLKFGNALLRRYRK | |
| | TACATTGTCAACACAGCCCACCCAGT | | FRPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPDSANYKKIAA | |
| | CCAGCATCAACTACGTGGTCCTGCGA | | RWPEFLSLNPSGMFMNGTN | |
| | CTACTGGGCCTGTCACGGGATCATCC | | GLQVWDTAFAVQYACVCG | |
| | GGTCTGCGTCAAGGCGCGCAAAACG | | FAELPQYQKTIRAAFDFLDL | |
| | CTGCTCACCAAGTTTGGCGGCGCCAT | | SQINEPTEENSYRDDRVGG | |
| | CAACAACCCCCATTGGGGCAAGACC | | WPFSTKTQGYPVSDCTAEA | |
| | TGGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGNIMVEYPYV | |
| | GGGTCCTTACCCGGTGGATCTACCTT | | ECTDSVVLGLSYFRKYHDY | |
| | GCCATGGGCTATCTGGAGGCTGCGG | | RNEDVDRAISAAIGYIIREQ | |
| | AGGCCCAATGCGAACTCACTCCGTTG | | QPDGGFFGSWGVCYCYAH | |
| | CTGGAGGAGCTCCGAGACGAAATCT | | MFAMEALETQNLNYNNCS | |
| | ACAAAAAGCCCTACTCGGAGATTGA | | TVQKACDFLAGYQEADGG | |
| | TTTCTCCAAACATTGCAACTCCATCT | | WAEDFKSCETQMYVRGPH | |
| | CCGGAGTCGACCTCTACTATCCCCAC | | SLVVPTAMALLSLMSGRYP | |
| | ACCGGCCTTTTGAAGTTTGGCAACGC | | QEDKIHAAARFLMSKQMS | |
| | GCTTCTCCGACGATACCGCAAGTTCA | | NGEWLKEEMEGVFNHTCAI | |
| | GACCGCAGTGGATCAAAGAAAAGGT | | EYPNYRFYFVMKALGLYF | |
| | CAAGGAGGAAATTTACAACTTGTGC | | MGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCTGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAAGCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTTGAATGCA | | | |
| | CTGATTCTGTTGTTCTGGGTCTGTCCT | | | |
| | ACTTTCGAAAGTACCACGATTACCGC | | | |
| | AACGAAGACGTGGACCGAGCCATCT | | | |
| | CTGCTGCCATTGGATACATTATTCGA | | | |
| | GAGCAGCAGCCTGACGGCGGCTTCTT | | | |
| | TGGCTCCTGGGGCGTGTGCTACTGCT | | | |
| | ACGCTCACATGTTTGCCATGGAGGCT | | | |
| | CTGGAGACGCAGAATCTCAACTATA | | | |
| | ACAACTGTTCCACGGTTCAAAAGGC | | | |
| | GTGCGACTTTCTGGCGGGCTACCAGG | | | |
| | AAGCAGATGGAGGCTGGGCCGAGGA | | | |
| | CTTTAAGTCGTGCGAGACTCAGATGT | | | |
| | ACGTGCGCGGACCCCATTCGCTGGTC | | | |
| | GTGCCTACTGCCATGGCCCTGTTGAG | | | |
| | TTTGATGAGTGGTCGGTATCCCCAGG | | | |
| | AGGACAAGATTCATGCTGCGGCCCG | | | |
| | GTTTCTCATGAGCAAGCAGATGAGC | | | |
| | AACGGTGAGTGGCTCAAGGAGGAGA | | | |
| | TGGAGGGGGTGTTTAACCATACTTGT | | | |
| | GCCATTGAGTATCCCAACTACCGGTT | | | |
| | TTATTTTGTCATGAAGGCTTTGGGGT | | | |
| | TGTATTTCATGGGATATTGCCAGTGA | | | |
| 3C9 | ATGGGAATCCACGAAAGTGTGTCGA | 108 | MGIHESVSKQFAKNGHSKY | 318 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | LAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCTAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AVALKAIIMVQNTPGYEDL | |
| | ATGGGAGGGTGTGAATCTGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVALGLSNFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAAGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGTGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGCTCTGGGTC | | | |
| | TGTCCAACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 4A6 | ATGGGAATCCACGAAAGTGTGTCGA | 109 | MGIHESVSKQFAKNGHSKY | 319 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSDAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGACGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQEACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AGAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 4F11 | ATGGGAATCCACGAAAGTGTGTCGA | 109 | MGIHESVSKQFAKNGHSKY | 319 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSDAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGACGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQEACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AGAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 3D11 | ATGGGAATCCACGAAAGTGTGTCGA | 111 | MGIHESVSKQFAKNGHSKY | 321 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVNNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWSLHKEDKSTCFGT | |
| | GTGACTCTGGACTCCAAGCCCGTGAA | | SINYVVLRLLGLSRDHPVC | |
| | TAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCATGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGAGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGLFEPGSFASYETIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETLNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYC | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTTGTTTGAACCGG | | | |
| | GCTCTTTCGCCTCCTATGAGACTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCTGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CTAG | | | |
| 4B11 | ATGGGAATCCACGAAAGTGTGTCGA | 112 | MGIHESVSKQFAKNGHSKY | 322 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKILLSILNLYKWEGVNP | |
| | AGACCCCGACTCCGGCGCATGGCAG | | APGELWLLPYFVPVHPGRW | |
| | TCGGAATACGACGGACCGCAGTTCA | | WVHTRWIYLAMGYLEAAE | |
| | TGTCGATCGGTTATGTGACGGCATGC | | AQCELTPLLEELRDEIYKKP | |
| | TACTTTGGCGGCAACGAGATCCCCAC | | YSEIDFSKHCNSISGVDLYY | |
| | GCCGGTCAAAACTGAAATGATCAGA | | PHTGLLKFGNALLRRYRKF | |
| | TACATTGTCAACACAGCCCACCCAGT | | RPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPDSANYKKIAA | |
| | CCAGCATCAACTACGTGGTCCTGCGA | | RWPEFLSLNPSGMFMNGTN | |
| | CTACTGGGCCTGTCACGGGATCATCC | | GLQVWDTAFAVQYACVCG | |
| | GGTCTGCGTCAAGGCGCGCAAAACG | | FAELPQYQKTIRAAFDFLDR | |
| | CTGCTCACCAAGTTTGGCGGCGCCAT | | SQINEPTEENSYRDDRVGG | |
| | CAACAACCCCCATTGGGGCAAGATC | | WPFSTKTQGYPVSDCTAEA | |
| | TTGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGYIMVEYPYEE | |
| | GGGTCCATACCCGGTGGATCTACCTT | | CTDSVVLGLSYFRKYHDYR | |
| | GCCATGGGCTATCTGGAGGCTGCGG | | NEDVDRAISAAIGYIIREQQ | |
| | AGGCCCAATGCGAACTCACTCCGTTG | | PDGGFFGSWGVCYCYAHM | |
| | CTGGAGGAGCTCCGAGACGAAATCT | | FAMEALETQNLNYNNCSTV | |
| | ACAAAAAGCCCTACTCGGAGATTGA | | QKACDFLAGYQEADGGWA | |
| | TTTCTCCAAACATTGCAACTCCATCT | | EDFKSCETQMYVRGPHSLV | |
| | CCGGAGTCGACCTCTACTATCCCCAC | | VPTAMALLSLMSGRYPQED | |
| | ACCGGCCTTTTGAAGTTTGGCAACGC | | KIHAAARFLMSKQMSNGE | |
| | GCTTCTCCGACGATACCGCAAGTTCA | | WLKEEMEGVFNHTCAIEYP | |
| | GACCGCAGTGGATCAAAGAAAAGGT | | NYRFYFVMKALGLYFKGY | |
| | CAAGGAGGAAATTTACAACTTGTGC | | CQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGATAC | | | |
| | ATCATGGTGGAGTATCCGTACGAGG | | | |
| | AATGCACTGATTCTGTTGTTCTGGGT | | | |
| | CTGTCCTACTTTCGAAAGTACCACGA | | | |
| | TTACCGCAACGAAGACGTGGACCGA | | | |
| | GCCATCTCTGCTGCCATTGGATACAT | | | |
| | TATTCGAGAGCAGCAGCCTGACGGC | | | |
| | GGCTTCTTTGGCTCCTGGGGCGTGTG | | | |
| | CTACTGCTACGCTCACATGTTTGCCA | | | |
| | TGGAGGCTCTGGAGACGCAGAATCT | | | |
| | CAACTATAACAACTGTTCCACAGTTC | | | |
| | AAAAGGCGTGCGACTTTCTGGCGGG | | | |
| | CTACCAGGAAGCAGATGGAGGCTGG | | | |
| | GCCGAGGACTTTAAGTCGTGCGAGA | | | |
| | CTCAGATGTACGTGCGCGGACCCCAT | | | |
| | TCGCTGGTCGTGCCTACTGCCATGGC | | | |
| | CCTGTTGAGTTTGATGAGTGGTCGGT | | | |
| | ATCCCCAGGAGGACAAGATTCATGC | | | |
| | TGCGGCCCGGTTTCTCATGAGCAAGC | | | |
| | AGATGAGCAACGGTGAGTGGCTCAA | | | |
| | GGAGGAGATGGAGGGGGTGTTTAAC | | | |
| | CATACTTGTGCCATTGAGTATCCCAA | | | |
| | CTACCGGTTTTATTTTGTCATGAAGG | | | |
| | CTTTGGGGTTGTATTTCAAGGGATAT | | | |
| | TGCCAGTGA | | | |
| 4B12 | ATGGGAATCCACGAAAGTGTGTCGA | 63 | MGIHESVSKQFAKNGHSKY | 84 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSNNYVVLRLLGLSRDHPV | |
| | AAATGCCTACGAAGCGGCTCTCAAA | | CVKARKTLLTKFGGAINNP | |
| | AACTGGCATCTGTTTGCGTCGCTGCA | | HWGKTWLSILNLYKWEGV | |
| | AGACCCCGACTCCGGCGCATGGCAG | | NPAPGELWLLPYFVPVHPG | |
| | TCGGAATACGACGGACCGCAGTTCA | | RWWVHTRWIYLAMGYLE | |
| | TGTCGATCGGTTATGTGACGGCGTGC | | AAEAQCELTPLLEELRDEIY | |
| | TACTTTGGCGGCAACGAGATCCCCAC | | KKPYSEIDFSKHCNSISGVD | |
| | GCCGGTCAAAACCGAAATGATCAGA | | LYYPHTGLLKFGNALLRRY | |
| | TACATTGTCAACACAGCCCACCCAGT | | RKFRPQWIKEKVKEEIYNL | |
| | TGACGGAGGCTGGGGCCTTCACAAA | | CLREVSNTRHLCLAPVNNA | |
| | GAAGACAAGAGCACCTGTTTCGGTA | | MTSIVMYLHEGPDSANYKK | |
| | CCAGCAACAACTACGTGGTCCTGCG | | IAARWPEFLSLNPSGMFMN | |
| | ACTACTGGGCCTGTCACGGGATCATC | | GTNGLQVWDTAFAVQYAS | |
| | CGGTCTGCGTCAAGGCGCGCAAAAC | | VCGFAELPQYQKTIRAAFD | |
| | GCTGCTCACCAAGTTTGGCGGCGCCA | | FLDRSQINEPTEENSYRDDR | |
| | TCAACAACCCCCATTGGGGCAAGAC | | VGGWPFSTKTQGYPVSDCT | |
| | CTGGCTGTCGATTCTCAATCTCTACA | | AEALKAIIMVQNTPGYEDL | |
| | AATGGGAGGGTGTGAATCCGGCCCC | | KKQVSDKRKHTAIDLLLGM | |
| | TGGCGAGCTCTGGCTGTTGCCCTACT | | QNVGSFEPGSFASYEPIRAS | |
| | TTGTTCCTGTTCATCCGGGCCGATGG | | SMLEKINPAEVFGNIMVEY | |
| | TGGGTCCATACCCGGTGGATCTACCT | | PYVECTDSVVMGLSYFRKY | |
| | TGCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSS | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGAGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTATGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTAGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 2A5 | ATGGGAATCCACGAAAGTGTGTCGA | 113 | MGIHESVSKQFAKNGHSKY | 323 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGVLWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTVFAVQYAC | |
| | GGTCTGCGTCAAGGCGCGCAAAACG | | VCGFAELPLYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGTGCTCTGGCTGTTGCCCTACTT | | NVGSFEPGSFASYEPIRTSS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSCFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALETQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGTGTTTG | | | |
| | CCGTGCAATACGCGTGTGTTTGTGGC | | | |
| | TTTGCCGAACTTCCCCTGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGACGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTGCTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 2B3 | ATGGGAATCCACGAAAGTGTGTCGA | 114 | MGIHESVSKQFAKNGHSKY | 324 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCAGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSGIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWINEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALVTQNLNYN | |
| | ACAAAAAGCCCTACTCGGGGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAATGAAAAGGT | | CAIEYPNYRVYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGGTTTATTTTGTCATGAAGGCT | | | |
| | TTGGGGTTGTATTTCAAGGGATATTG | | | |
| | CCAGTGA | | | |
| 2F9 | ATGGGAATCCACGAAAGTGTGTCGA | 115 | MGIHESVSKQFAKNGHSKY | 325 |
| | AACAGTTTGCGAAAAACGGACATTC | | RSDRYGLPKTDLRRWTFHA | |
| | CAAGTACCGCAGCGACCGATACGGC | | SDLGAQWWEYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAAIKN | |
| | CGCAATGGTGGGAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTATCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | SSIVMYLHEGPDPANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPMEENSYRDD | |
| | CAACAACCCCCATTGGGGCAAGACC | | RVGGWPFSTKTQGYPVSDC | |
| | TGGCTGTCGATTCTCAATCTCTACAA | | TAEALKAIIMVQNTPGYED | |
| | ATGGGAGGGTGTGAATCCGGCCCCT | | LKKQVSDKRKHTAIDLLLG | |
| | GGCGAGCTCTGGCTGTTGCCCTACTT | | MQNVGSFEPGSFASYEPIRA | |
| | TGTTCCTGTTCATCCGGGTCGATGGT | | SSMLEKINPAEVFGNIMVE | |
| | GGGTCCATACCCGGTGGATCTACCTT | | YPYVECTDSVVLGLSYFRK | |
| | GCCATGGGCTATCTGGAGGCTGCGG | | YHDYRNEDVDPAISAAIGYI | |
| | AGGCCCAATGCGAACTCACTCCGTTG | | IREQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGTCCTCCATTGTCATGTATCTC | | | |
| | CATGAGGGGCCCGATCCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GATGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | ACCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCCAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 1A3 | ATGGGAATCCACGAAAGTGTGTCGA | 330 | MGIHESVSKQFAKNGHSKY | 331 |
| | AACAGTTTGCGAAAAACGGACATTC | | RSDRYGLPKTDLRQWTFHA | |
| | CAAGTACCGCAGCGACCGATACGGC | | SDLGAQWWKYDDTTPLEE | |
| | TTACCTAAGACGGATCTGCGACAATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGTTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKACKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIGFSKHCITISGVDLY | |
| | GCCGGTCAAAACCGAAATGATCAGA | | YPHTGLLKFGNALLRRYRK | |
| | TACATTGTCAACACAGCCCACCCAGT | | FRPQWIKEKVKEEIYNLCLR | |
| | TGACGGAGGCTGGGGCCTTCACAAA | | EVSNTRHLCLAPVNNAMTS | |
| | GAAGACAAGAGCACCTGTTTCGGTA | | IVMYLHEGPDSANYKKIAA | |
| | CCAGCATCAACTACGTGGTCCTGCGA | | RWPEFLSLNPSGMFMNGTN | |
| | CTACTGGGCCTGTCGCGGGATCATCC | | GLQVWDTAFAVQYACVCG | |
| | GGTCTGCGTCAAGGCGTGCAAAACG | | FAELPQYQKTIRAAFDFLDR | |
| | CTGCTCACCAAGTTTGGCGGCGCCAT | | SQINEPTEENSYRDDRVGG | |
| | CAACAACCCCCATTGGGGCAAGACC | | WPFSTKTQGYPVSDCTAEA | |
| | TGGCTGTCGATTCTCAATCTCTACAA | | LKAIIMVQNTPGYEDLKKQ | |
| | ATGGGAGGGTGTGAATCCGGCCCCT | | VSDKRKHTAIDLLLGMQNV | |
| | GGCGAGCTCTGGCTGTTGCCCTACTT | | GSFEPGSFASYEPIRASSML | |
| | TGTTCCTGTTCATCCGGGCCGATGGT | | EKINPAEVFGNIMVEYPYV | |
| | GGGTCCATACCCGGTGGATCTACCTT | | ECTDSVVLGLSYFRKYHDY | |
| | GCCATGGGCTATCTGGAGGCTGCGG | | RNEDVDRAISAAIGYIIREQ | |
| | AGGCCCAATGCGAACTCACTCCGTTG | | QPDGGFFGSWGVCYCYAH | |
| | CTGGAGGAGCTCCGAGACGAAATCT | | MFAMEALETQSLNYNNCST | |
| | ACAAAAAGCCCTACTCGGAGATTGG | | VQKACDFLAGYQEADGGW | |
| | TTTCTCCAAACATTGCATCACCATCT | | AEDFKSCETQMYVRGPHSL | |
| | CCGGAGTCGACCTCTACTATCCCCAC | | VVPTAMALLSLMSGRYPQE | |
| | ACCGGCCTTTTGAAGTTTGGCAACGC | | DKIHAAARFLMSKQMSNG | |
| | GCTTCTCCGACGATACCGCAAGTTCA | | EWLKEEMEGVFNHTCAIEY | |
| | GACCGCAGTGGATCAAAGAAAAGGT | | PNYRFYFVMKALGLFFKGY | |
| | CAAGGAGGAAATTTATAACTTGTGCC | | CQ | |
| | TTCGAGAGGTTTCCAACACACGACAC | | | |
| | TTGTGTCTCGCTCCCGTCAACAATGC | | | |
| | CATGACCTCCATTGTCATGTATCTCC | | | |
| | ATGAGGGGCCCGATTCGGCGAATTA | | | |
| | CAAAAAGATTGCGGCCCGATGGCCC | | | |
| | GAATTTCTGTCTCTGAATCCGTCGGG | | | |
| | AATGTTTATGAACGGCACCAACGGTC | | | |
| | TGCAGGTCTGGGATACTGCGTTTGCC | | | |
| | GTGCAATACGCGTGTGTTTGTGGCTT | | | |
| | TGCCGAACTTCCCCAGTACCAGAAG | | | |
| | ACGATCCGAGCGGCGTTTGATTTTCT | | | |
| | CGATCGGTCCCAGATCAACGAGCCG | | | |
| | ACGGAGGAAAATTCCTATCGAGACG | | | |
| | ACCGCGTCGGAGGATGGCCCTTTAGT | | | |
| | ACCAAGACCCAGGGGTATCCAGTCT | | | |
| | CCGACTGTACTGCCGAGGCTCTCAAG | | | |
| | GCCATCATCATGGTCCAGAATACGCC | | | |
| | TGGATACGAGGATCTGAAGAAACAA | | | |
| | GTGTCTGACAAGCGGAAACACACTG | | | |
| | CCATCGATCTACTTTTGGGAATGCAG | | | |
| | AACGTGGGCTCGTTTGAACCGGGCTC | | | |
| | TTTCGCCTCCTATGAGCCTATCCGGG | | | |
| | CGTCGTCCATGCTGGAGAAGATCAAT | | | |
| | CCGGCCGAGGTGTTTGGAAACATCAT | | | |
| | GGTGGAGTATCCGTACGTGGAATGC | | | |
| | ACTGATTCTGTTGTTCTGGGTCTGTC | | | |
| | CTACTTTCGAAAGTACCACGATTACC | | | |
| | GCAACGAAGACGTGGACCGAGCCAT | | | |
| | CTCTGCTGCCATTGGATACATTATTC | | | |
| | GAGAGCAGCAGCCTGACGGCGGCTT | | | |
| | CTTTGGCTCCTGGGGCGTGTGCTACT | | | |
| | GCTACGCTCACATGTTTGCCATGGAG | | | |
| | GCTCTGGAGACGCAGAGTCTCAACT | | | |
| | ATAACAACTGTTCCACGGTTCAAAAG | | | |
| | GCGTGCGACTTTCTGGCGGGCTACCA | | | |
| | GGAAGCAGATGGAGGCTGGGCCGAG | | | |
| | GACTTTAAGTCGTGCGAGACTCAGAT | | | |
| | GTACGTGCGCGGACCCCATTCGCTGG | | | |
| | TCGTGCCTACTGCCATGGCCCTGTTG | | | |
| | AGTTTGATGAGTGGTCGGTATCCCCA | | | |
| | GGAGGACAAGATTCATGCTGCGGCC | | | |
| | CGGTTTCTCATGAGCAAGCAGATGA | | | |
| | GCAACGGTGAGTGGCTCAAGGAGGA | | | |
| | GATGGAGGGGGTGTTTAACCATACTT | | | |
| | GTGCCATTGAGTATCCCAACTACCGG | | | |
| | TTTTATTTTGTCATGAAGGCTTTGGG | | | |
| | GTTGTTTTTCAAGGGATATTGCCAGT | | | |
| | GA | | | |
| 2H4 | ATGGGAATCCACGAAAGTGTGTCGA | 116 | MGIHESVSKQFAKNGHSKY | 85 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAASD | |
| | CTGCTCACCAAGTTTGGCGGCGCCAT | | FLDRSQINEPTEENSYRDGR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKFNPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTCTGATTTTC | | | |
| | TCGATCGGTCCCAGATCAACGAGCC | | | |
| | GACGGAGGAAAATTCCTATCGAGAC | | | |
| | GGCCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGTT | | | |
| | CAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 2F6 | ATGGGAATCCACGAAAGTGTGTCGA | 4 | MGIHESVSKQFAKNGHSKY | 3 |
| | AACAGTTTGCGAAAAACGGACATTC | | RSDRYGLPKTDLRRWTFHA | |
| | CAAGTACCGCAGCGACCGATACGGC | | SDLGAQWWKYDGTTPLEE | |
| | TTACCTAAGACGGATCTGCGACGATG | | LEKRATDYVRYSLELPGYA | |
| | GACGTTCCACGCGTCCGATCTGGGGG | | PVTLDSKPVKNAYEAALKS | |
| | CGCAATGGTGGAAGTATGACGGTAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAGATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AGCTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCSFAELPQYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGAGCTCTGGCTGTTGCCCTACTT | | NVGSFEPGSFASYEPIRASS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSYFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALVTQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRLYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTAGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATCGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTATATTTTGTCATGAAGGCT | | | |
| | TTGGGGTTGTATTTCAAGGGATATTG | | | |
| | CCAGTGA | | | |
| 3A5 | ATGGGAATCCACGAAAGTGTGTCGA | 117 | MGIHESVSKQFAKNGHSKY | 326 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLVTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYI | |
| | TACTTTGGCGGCAACGAGATCCCCAC | | KPYSEIDFSKHCNSISGVDL | |
| | GCCGGTCAAAACCGAAATGATCAGA | | YYPHTGLLKFGSALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGLDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCGFAELPQYQKTIRAAFD | |
| | CTGGTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YTHMFAMEALETQNLNYN | |
| | ACATAAAGCCCTACTCGGAGATTGAT | | NCSTVQKACDFLADYQEA | |
| | TTCTCCAAACATTGCAACTCCATCTC | | DGGWAEDLKSCETQMYVR | |
| | CGGAGTCGACCTCTACTATCCCCACA | | GPHSLVVPTAMALLSLMSG | |
| | CCGGCCTTTTGAAGTTTGGCAGCGCG | | RYPQEDKIHAAARFLMSKQ | |
| | CTTCTCCGACGATACCGCAAGTTCAG | | MSNGEWLKEEMEGVFNHT | |
| | ACCGCAGTGGATCAAAGAAAAGGTC | | CAIEYPNYRFYFVMKALGL | |
| | AAGGAGGAAATTTACAACTTGTGCCT | | YFKGYCQ | |
| | TCGAGAGGTTTCCAACACACGACACT | | | |
| | TGTGTCTCGCTCCCGTCAACAATGCC | | | |
| | ATGACCTCCATTGTCATGTATCTCCA | | | |
| | TGAGGGGCTCGATTCGGCGAATTAC | | | |
| | AAAAAGATTGCGGCCCGATGGCCCG | | | |
| | AATTTCTGTCTCTGAATCCGTCGGGA | | | |
| | ATGTTTATGAACGGCACCAACGGTCT | | | |
| | GCAGGTCTGGGATACTGCGTTTGCCG | | | |
| | TGCAATACGCGTGTGTTTGTGGCTTT | | | |
| | GCCGAACTTCCCCAGTACCAGAAGA | | | |
| | CGATCCGAGCGGCGTTTGATTTTCTC | | | |
| | GATCGGTCCCAGATCAACGAGCCGA | | | |
| | CGGAGGAAAATTCCTATCGAGACGA | | | |
| | CCGCGTCGGAGGATGGCCCTTTAGTA | | | |
| | CCAAGACCCAGGGGTATCCAGTCTCC | | | |
| | GACTGTACTGCCGAGGCTCTCAAGGC | | | |
| | CATCATCATGGTCCAGAATACGCCTG | | | |
| | GATACGAGGATCTGAAGAAACAAGT | | | |
| | GTCTGACAAGCGGAAACACACTGCC | | | |
| | ATCGATCTACTTTTGGGAATGCAGAA | | | |
| | CGTGGGCTCGTTTGAACCGGGCTCTT | | | |
| | TCGCCTCCTATGAGCCTATCCGGGCG | | | |
| | TCGTCCATGCTGGAGAAGATCAATCC | | | |
| | GGCCGAGGTGTTTGGAAACATCATG | | | |
| | GTGGAGTATCCGTACGTGGAATGCA | | | |
| | CTGATTCTGTTGTTCTGGGTCTGTCCT | | | |
| | ACTTTCGAAAGTACCACGATTACCGC | | | |
| | AACGAAGACGTGGACCGAGCCATCT | | | |
| | CTGCTGCCATTGGATATATTATTCGA | | | |
| | GAGCAGCAGCCTGACGGCGGCTTCTT | | | |
| | TGGCTCCTGGGGCGTGTGCTACTGCT | | | |
| | ACACTCACATGTTTGCCATGGAGGCT | | | |
| | CTGGAGACGCAGAATCTCAACTATA | | | |
| | ACAACTGTTCCACGGTTCAAAAGGC | | | |
| | GTGCGACTTTCTGGCGGACTACCAGG | | | |
| | AAGCAGATGGAGGCTGGGCCGAGGA | | | |
| | CCTTAAGTCGTGCGAGACTCAGATGT | | | |
| | ACGTGCGCGGACCCCATTCGCTGGTC | | | |
| | GTGCCTACTGCCATGGCCCTGTTGAG | | | |
| | TTTGATGAGTGGTCGGTATCCCCAGG | | | |
| | AGGACAAGATTCATGCTGCGGCCCG | | | |
| | GTTTCTCATGAGCAAGCAGATGAGC | | | |
| | AACGGTGAGTGGCTCAAGGAGGAGA | | | |
| | TGGAGGGGGTGTTTAACCATACTTGT | | | |
| | GCCATTGAGTATCCCAACTACCGGTT | | | |
| | TTATTTTGTCATGAAGGCTTTGGGGT | | | |
| | TGTATTTCAAGGGATATTGCCAGTGA | | | |
| 2C5 | ATGGGAATCCACGAAAGTGTGTCGA | 328 | MGIHESVSKQFAKYGHSKY | 329 |
| | AACAGTTTGCGAAATACGGACATTCC | | RSDRYGLPKTDLRRWTFHA | |
| | AAGTACCGCAGCGACCGATACGGCT | | SDLGAQWWKYDDTTPLEE | |
| | TACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | SEIPTPVKTEMIRCIVNTAHP | |
| | GCTGGAGCTGCCGGGATACGCGCCC | | VDGGWGLHKEDKSTCFGT | |
| | GTGACTCTGGACTCCAAGCCCGTGAA | | SINYVVLRLLGLSRDHPVC | |
| | AAATGCCTACGAAGCGGCTCTCAAA | | VKAHKTLLTKFGGAINNPH | |
| | AACTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAGCGAGATCCCCAC | | KPYSEFDFSKHCNSISGVDL | |
| | GCCGGTCAAAACCGAAATGATCAGA | | YYPHTGLLKFGNARLRRYR | |
| | TGCATTGTCAACACAGCCCACCCAGT | | KFRPQWIKEKVKEEIYNLC | |
| | TGACGGAGGCTGGGGCCTTCATAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCACAAAACG | | VCGFAELPQYQKTIRAAIDF | |
| | CTGCTCACCAAGTTTGGCGGCGCCAT | | LDRSQINVPSEENSYRDDR | |
| | CAACAACCCCCATTGGGGCAAGACC | | VGGWPFSTKTQGYPVSDCT | |
| | TGGCTGTCGATTCTCAATCTCTACAA | | AEALKASIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGTTTGAT | | NCSTVQRACDFLAGYQEA | |
| | TTCTCCAAACATTGCAACTCCATCTC | | DGGWAEDFKSCEAQMYVR | |
| | CGGAGTCGACCTCTACTATCCCCACA | | GPHSLVVPTAMALLSLMSG | |
| | CCGGCCTTTTGAAGTTTGGCAACGCG | | RYPQEDKIHAAARFLMSKQ | |
| | CGTCTCCGACGATACCGCAAGTTCAG | | MSNGEWLKEEMEGVFNHT | |
| | ACCGCAGTGGATCAAAGAAAAGGTC | | CAIEYPNYRFYFVMKALGL | |
| | AAGGAGGAAATTTACAACTTGTGCCT | | YFKGYCQ | |
| | TCGAGAGGTTTCCAACACACGACACT | | | |
| | TGTGTCTCGCTCCCGTCAACAATGCC | | | |
| | ATGACCTCCATTGTCATGTATCTCCA | | | |
| | TGAGGGGCCCGATTCGGCGAATTAC | | | |
| | AAAAAGATTGCGGCCCGATGGCCCG | | | |
| | AATTTCTGTCTCTGAATCCGTCGGGA | | | |
| | ATGTTTATGAACGGCACCAACGGTCT | | | |
| | GCAGGTCTGGGATACTGCGTTTGCCG | | | |
| | TGCAATACGCGTGTGTTTGTGGCTTT | | | |
| | GCCGAACTTCCCCAGTACCAGAAGA | | | |
| | CGATCCGAGCGGCGATTGATTTTCTC | | | |
| | GATCGGTCCCAGATCAACGTGCCGTC | | | |
| | GGAGGAAAATTCCTATCGAGACGAC | | | |
| | CGCGTCGGAGGATGGCCCTTTAGTAC | | | |
| | CAAGACCCAGGGGTATCCAGTCTCC | | | |
| | GACTGTACTGCCGAGGCTCTCAAGGC | | | |
| | CAGCATCATGGTCCAGAATACGCCTG | | | |
| | GATACGAGGATCTGAAGAAACAAGT | | | |
| | GTCTGACAAGCGGAAACACACTGCC | | | |
| | ATCGATCTACTTTTGGGAATGCAGAA | | | |
| | CGTGGGCTCGTTTGAACCGGGCTCTT | | | |
| | TCGCCTCCTATGAGCCTATCCGGGCG | | | |
| | TCGTCCATGCTGGAGAAGATCAATCC | | | |
| | GGCCGAGGTGTTTGGAAACATCATG | | | |
| | GTGGAGTATCCGTACGTGGAATGCA | | | |
| | CTGATTCTGTTGTTCTGGGTCTGTCCT | | | |
| | ACTTTCGAAAGTACCACGATTACCGC | | | |
| | AACGAAGACGTGGACCGAGCCATCT | | | |
| | CTGCTGCCATTGGATACATTATTCGA | | | |
| | GAGCAGCAGCCTGACGGCGGCTTCTT | | | |
| | TGGCTCCTGGGGCGTGTGCTACTGCT | | | |
| | ACGCTCACATGTTTGCCATGGAGGCT | | | |
| | CTGGAGACGCAGAATCTCAACTATA | | | |
| | ACAACTGTTCCACGGTTCAAAGGGC | | | |
| | GTGCGACTTTCTGGCGGGCTACCAGG | | | |
| | AAGCAGATGGAGGCTGGGCCGAGGA | | | |
| | CTTTAAGTCGTGCGAGGCTCAGATGT | | | |
| | ACGTGCGCGGACCCCATTCGCTGGTC | | | |
| | GTGCCTACTGCCATGGCCCTGTTGAG | | | |
| | TTTGATGAGTGGTCGGTATCCCCAGG | | | |
| | AGGACAAGATTCATGCTGCGGCCCG | | | |
| | GTTTCTCATGAGCAAGCAGATGAGC | | | |
| | AATGGTGAGTGGCTCAAGGAGGAGA | | | |
| | TGGAGGGGGTGTTTAACCATACTTGT | | | |
| | GCCATTGAGTATCCCAACTACCGGTT | | | |
| | TTATTTTGTCATGAAGGCTTTGGGGT | | | |
| | TGTATTTCAAGGGATATTGCCAGTGA | | | |
| 887779 | ATGGGAATCCACGAAAGTGTGTCGA | 61 | MGIHESVSKQFAKNGHSKY | 1 |
| | AACAGTTTGCGAAAAACGGACATTC | | RSDRYGLPKTDLRRWTFHA | |
| | CAAGTACCGCAGCGACCGATACGGC | | SDLGAQWWKYDDTTPLEE | |
| | TTACCTAAGACGGATCTGCGACGATG | | LEKRATDYVKYSLELPGYA | |
| | GACGTTCCACGCGTCCGATCTGGGGG | | PVTLDSKPVKNAYEAALKN | |
| | CGCAATGGTGGAAGTATGACGATAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAAATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AACTGGCATCTGTTTGCGTCGCTGCA | | WGKTWLSILNLYKWEGVN | |
| | AGACCCCGACTCCGGCGCGTGGCAG | | PAPGELWLLPYFVPVHPGR | |
| | AGCGAATACGACGGACCGCAGTTCA | | WWVHTRWIYLAMGYLEA | |
| | TGAGCATCGGCTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGCTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGACCATCC | | GTNGLQVWDTAFAVQYAC | |
| | GGTCTGCGTCAAGGCGCGCAAAACG | | VCGFAELPQYQKTIRAAFD | |
| | CTGCTCACCAAGTTTGGCGGCGCCAT | | FLDRSQINEPTEENSYRDDR | |
| | CAACAACCCCCATTGGGGCAAGACC | | VGGWPFSTKTQGYPVSDCT | |
| | TGGCTGTCGATTCTCAATCTCTACAA | | AEALKAIIMVQNTPGYEDL | |
| | ATGGGAGGGTGTGAATCCGGCCCCT | | KKQVSDKRKHTAIDLLLGM | |
| | GGCGAGCTCTGGCTGTTGCCCTACTT | | QNVGSFEPGSFASYEPIRAS | |
| | TGTTCCTGTTCATCCGGGCCGATGGT | | SMLEKINPAEVFGNIMVEY | |
| | GGGTCCATACCCGGTGGATCTACCTT | | PYVECTDSVVLGLSYFRKY | |
| | GCCATGGGCTATCTGGAGGCTGCGG | | HDYRNEDVDRAISAAIGYII | |
| | AGGCCCAATGCGAACTCACTCCGTTG | | REQQPDGGFFGSWGVCYC | |
| | CTGGAGGAGCTCCGAGACGAAATCT | | YAHMFAMEALETQNLNYN | |
| | ACAAAAAGCCCTACTCGGAGATTGA | | NCSTVQKACDFLAGYQEA | |
| | TTTCTCCAAACATTGCAACTCCATCT | | DGGWAEDFKSCETQMYVR | |
| | CCGGAGTCGACCTCTACTATCCCCAC | | GPHSLVVPTAMALLSLMSG | |
| | ACCGGCCTTTTGAAGTTTGGCAACGC | | RYPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRFYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTCCAGGTCTGGGATACTGCGTTTG | | | |
| | CCGTGCAATACGCGTGTGTTTGTGGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCGGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TCTCCTACTTTCGAAAGTACCACGAT | | | |
| | TACCGCAACGAAGACGTGGACCGAG | | | |
| | CCATCTCTGCTGCCATTGGATACATT | | | |
| | ATTCGAGAGCAGCAGCCTGACGGCG | | | |
| | GCTTCTTTGGCTCCTGGGGCGTGTGC | | | |
| | TACTGCTACGCTCACATGTTTGCCAT | | | |
| | GGAGGCTCTGGAGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACC | | | |
| | CAGATGTACGTGCGCGGACCCCATTC | | | |
| | GCTGGTCGTGCCTACTGCCATGGCCC | | | |
| | TGTTGAGTTTGATGAGTGGTCGGTAT | | | |
| | CCCCAGGAGGACAAGATTCATGCTG | | | |
| | CGGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTTTATTTTGTCATGAAGGCT | | | |
| | TTGGGGTTGTATTTCAAGGGATATTG | | | |
| | CCAGTGA | | | |
| 907811 | ATGGGAATCCACGAAAGTGTGTCGA | 4 | MGIHESVSKQFAKNGHSKY | 3 |
| | AACAGTTTGCGAAAAACGGACATTC | | RSDRYGLPKTDLRRWTFHA | |
| | CAAGTACCGCAGCGACCGATACGGC | | SDLGAQWWKYDGTTPLEE | |
| | TTACCTAAGACGGATCTGCGACGATG | | LEKRATDYVRYSLELPGYA | |
| | GACGTTCCACGCGTCCGATCTGGGGG | | PVTLDSKPVKNAYEAALKS | |
| | CGCAATGGTGGAAGTATGACGGTAC | | WHLFASLQDPDSGAWQSE | |
| | CACACCGCTGGAAGAGCTGGAAAAG | | YDGPQFMSIGYVTACYFGG | |
| | AGGGCTACCGACTACGTCAGATACTC | | NEIPTPVKTEMIRYIVNTAH | |
| | GCTGGAGCTGCCGGGATACGCGCCC | | PVDGGWGLHKEDKSTCFG | |
| | GTGACTCTGGACTCCAAGCCCGTGAA | | TSINYVVLRLLGLSRDHPVC | |
| | AAATGCCTACGAAGCGGCTCTCAAA | | VKARKTLLTKFGGAINNPH | |
| | AGCTGGCATCTGTTTGCGTCGCTGCA | | WGKTWLSILNLYKWEGVN | |
| | AGACCCCGACTCCGGCGCATGGCAG | | PAPGELWLLPYFVPVHPGR | |
| | TCGGAATACGACGGACCGCAGTTCA | | WWVHTRWIYLAMGYLEA | |
| | TGTCGATCGGTTATGTGACGGCGTGC | | AEAQCELTPLLEELRDEIYK | |
| | TACTTTGGCGGCAACGAGATCCCCAC | | KPYSEIDFSKHCNSISGVDL | |
| | GCCGGTCAAAACCGAAATGATCAGA | | YYPHTGLLKFGNALLRRYR | |
| | TACATTGTCAACACAGCCCACCCAGT | | KFRPQWIKEKVKEEIYNLC | |
| | TGACGGAGGCTGGGGCCTTCACAAA | | LREVSNTRHLCLAPVNNAM | |
| | GAAGACAAGAGCACCTGTTTCGGTA | | TSIVMYLHEGPDSANYKKI | |
| | CCAGCATCAACTACGTGGTCCTGCGA | | AARWPEFLSLNPSGMFMN | |
| | CTACTGGGCCTGTCACGGGATCATCC | | GTNGLQVWDTAFAVQYAC | |
| | GGTCTGCGTCAAGGCGCGCAAAACG | | VCSFAELPQYQKTIRAAFDF | |
| | CTGCTCACCAAGTTTGGCGGCGCCAT | | LDRSQINEPTEENSYRDDRV | |
| | CAACAACCCCCATTGGGGCAAGACC | | GGWPFSTKTQGYPVSDCTA | |
| | TGGCTGTCGATTCTCAATCTCTACAA | | EALKAIIMVQNTPGYEDLK | |
| | ATGGGAGGGTGTGAATCCGGCCCCT | | KQVSDKRKHTAIDLLLGMQ | |
| | GGCGAGCTCTGGCTGTTGCCCTACTT | | NVGSFEPGSFASYEPIRASS | |
| | TGTTCCTGTTCATCCGGGCCGATGGT | | MLEKINPAEVFGNIMVEYP | |
| | GGGTCCATACCCGGTGGATCTACCTT | | YVECTDSVVLGLSYFRKYH | |
| | GCCATGGGCTATCTGGAGGCTGCGG | | DYRNEDVDRAISAAIGYIIR | |
| | AGGCCCAATGCGAACTCACTCCGTTG | | EQQPDGGFFGSWGVCYCY | |
| | CTGGAGGAGCTCCGAGACGAAATCT | | AHMFAMEALVTQNLNYNN | |
| | ACAAAAAGCCCTACTCGGAGATTGA | | CSTVQKACDFLAGYQEAD | |
| | TTTCTCCAAACATTGCAACTCCATCT | | GGWAEDFKSCETQMYVRG | |
| | CCGGAGTCGACCTCTACTATCCCCAC | | PHSLVVPTAMALLSLMSGR | |
| | ACCGGCCTTTTGAAGTTTGGCAACGC | | YPQEDKIHAAARFLMSKQ | |
| | GCTTCTCCGACGATACCGCAAGTTCA | | MSNGEWLKEEMEGVFNHT | |
| | GACCGCAGTGGATCAAAGAAAAGGT | | CAIEYPNYRLYFVMKALGL | |
| | CAAGGAGGAAATTTACAACTTGTGC | | YFKGYCQ | |
| | CTTCGAGAGGTTTCCAACACACGACA | | | |
| | CTTGTGTCTCGCTCCCGTCAACAATG | | | |
| | CCATGACCTCCATTGTCATGTATCTC | | | |
| | CATGAGGGGCCCGATTCGGCGAATT | | | |
| | ACAAAAAGATTGCGGCCCGATGGCC | | | |
| | CGAATTTCTGTCTCTGAATCCGTCGG | | | |
| | GAATGTTTATGAACGGCACCAACGG | | | |
| | TCTGCAGGTCTGGGATACTGCGTTTG | | | |
| | CCGTGCAATACGCGTGTGTTTGTAGC | | | |
| | TTTGCCGAACTTCCCCAGTACCAGAA | | | |
| | GACGATCCGAGCGGCGTTTGATTTTC | | | |
| | TCGATCGGTCCCAGATCAACGAGCC | | | |
| | GACGGAGGAAAATTCCTATCGAGAC | | | |
| | GACCGCGTCGGAGGATGGCCCTTTA | | | |
| | GTACCAAGACCCAGGGGTATCCAGT | | | |
| | CTCCGACTGTACTGCCGAGGCTCTCA | | | |
| | AGGCCATCATCATGGTCCAGAATAC | | | |
| | GCCTGGATACGAGGATCTGAAGAAA | | | |
| | CAAGTGTCTGACAAGCGGAAACACA | | | |
| | CTGCCATCGATCTACTTTTGGGAATG | | | |
| | CAGAACGTGGGCTCGTTTGAACCGG | | | |
| | GCTCTTTCGCCTCCTATGAGCCTATC | | | |
| | CGGGCGTCGTCCATGCTGGAGAAGA | | | |
| | TCAATCCGGCCGAGGTGTTTGGAAAC | | | |
| | ATCATGGTGGAGTATCCGTACGTGGA | | | |
| | ATGCACTGATTCTGTTGTTCTGGGTC | | | |
| | TGTCCTACTTTCGAAAGTACCACGAT | | | |
| | TACCGCAACGAAGACGTGGACCGAG | | | |
| | CCATCTCTGCTGCCATCGGATACATT | | | |
| | ATTCGAGAGCAGCAGCCTGACGGTG | | | |
| | GCTTCTTTGGCTCCTGGGGCGTGTGC | | | |
| | TACTGCTACGCTCACATGTTTGCCAT | | | |
| | GGAGGCTCTGGTGACGCAGAATCTC | | | |
| | AACTATAACAACTGTTCCACGGTTCA | | | |
| | AAAGGCGTGCGACTTTCTGGCGGGCT | | | |
| | ACCAGGAAGCAGATGGAGGCTGGGC | | | |
| | CGAGGACTTTAAGTCGTGCGAGACTC | | | |
| | AGATGTACGTGCGCGGACCCCATTCG | | | |
| | CTGGTCGTGCCTACTGCCATGGCCCT | | | |
| | GTTGAGTTTGATGAGTGGTCGGTATC | | | |
| | CCCAGGAGGACAAGATTCATGCTGC | | | |
| | GGCCCGGTTTCTCATGAGCAAGCAG | | | |
| | ATGAGCAACGGTGAGTGGCTCAAGG | | | |
| | AGGAGATGGAGGGGGTGTTTAACCA | | | |
| | TACTTGTGCCATTGAGTATCCCAACT | | | |
| | ACCGGTTATATTTTGTCATGAAGGCT | | | |
| | TTGGGGTTGTATTTCAAGGGATATTG | | | |
| | CCAGTGA | | | |

TABLE 16
Sequences of Additional Enzymes Associated with the Disclosure

| | | SEQ | | SEQ |
|---|---|---|---|---|
| | | ID | | ID |
| Enzyme | Nucleotide Sequence | NO | Amino Acid Sequence | NO |
| Squalene | ATGGTGGACCAATGTGCACT | 277 | MVDQCALGWILASALGLVIA | 293 |
| epoxidase | TGGGTGGATCCTAGCGTCAG | | LCFFVAPRRNHRGVDSKERD | |
| | CTCTGGGCTTAGTAATAGCC | | ECVQSAATTKGECRFNDRDV | |
| | CTCTGCTTCTTTGTCGCTCC | | DVIVVGAGVAGSALAHTLGK | |
| | TCGAAGAAACCACCGCGGAG | | DGRRVHVIERDLTEPDRIVG | |
| | TTGATTCGAAAGAGAGAGAT | | ELLQPGGYLKLIELGLQDCV | |
| | GAATGTGTTCAGTCTGCGGC | | EEIDAQRVYGYALFKDGKNT | |
| | AACCACTAAGGGTGAATGTA | | RLSYPLENFHSDVSGRSFHN | |
| | GGTTTAATGACCGTGATGTA | | GRFIQRMREKAASLPNVRLE | |
| | GATGTTATTGTCGTGGGTGC | | QGTVTSLLEEKGTIKGVQYK | |
| | TGGTGTTGCCGGATCCGCAT | | SKNGEEKTAYAPLTIVCDGC | |
| | TGGCACATACGTTGGGCAAA | | FSNLRRSLCNPMVDVPSYFV | |
| | GACGGTAGAAGGGTGCATGT | | GLVLENCELPFANHGHVILG | |
| | AATTGAAAGAGACCTCACAG | | D | |
| | AACCAGATCGGATAGTTGGG | | PSPILFYQISRTEIRCLVDV | |
| | GAGTTACTTCAACCGGGTGG | | PGQKVPSIANGEMEKYLKTV | |
| | TTACTTAAAGCTAATCGAGT | | VAPQVPPQIYDSFIAAIDKG | |
| | TAGGATTGCAAGATTGCGTG | | NIRTMPNRSMPAAPHPTPGA | |
| | GAAGAAATTGATGCTCAGAG | | LLMGDAFNMRHPLTGGGMTV | |
| | AGTATATGGCTATGCCTTGT | | ALSDIVVLRNLLKPLKDLSD | |
| | TCAAAGATGGAAAAAATACA | | ASTLCKYLESFYTLRKPVAS | |
| | CGTTTGAGCTACCCATTAGA | | TINTLAGALYKVFCASPDQA | |
| | GAACTTTCACAGTGACGTTT | | RKEMRQACFDYLSLGGIFSN | |
| | CTGGTCGATCATTCCATAAT | | GPVSLLSGLNPRPLSLVLHF | |
| | GGTAGATTTATTCAACGTAT | | FAVAIYGVGRLLLPFPSVKG | |
| | GAGAGAAAAGGCTGCGTCCC | | IWIGARLIYSASGIIFPIIR | |
| | TACCCAACGTCAGGCTGGAA | | AEGVRQMFFPATVPAYYRSP | |
| | CAAGGAACTGTTACCTCGCT | | PVFKPIV | |
| | CTTGGAGGAAAAAGGCACTA | | | |
| | TCAAGGGTGTCCAATATAAA | | | |
| | TCAAAGAATGGGGAAGAAAA | | | |
| | AACAGCATACGCTCCGCTCA | | | |
| | CTATAGTGTGTGACGGTTGT | | | |
| | TTCTCTAACTTACGCCGAAG | | | |
| | TCTGTGCAATCCTATGGTCG | | | |
| | ATGTTCCAAGCTATTTTGTA | | | |
| | GGCTTGGTGTTGGAAAATTG | | | |
| | CGAGCTGCCATTCGCTAACC | | | |
| | ACGGACATGTAATTTTAGGC | | | |
| | GATCCTTCTCCCATTCTTTT | | | |
| | TTACCAGATTTCCAGGACCG | | | |
| | AAATAAGATGTTTGGTTGAT | | | |
| | GTCCCTGGTCAAAAAGTTCC | | | |
| | ATCAATAGCAAATGGCGAGA | | | |
| | TGGAAAAGTATCTGAAAACA | | | |
| | GTGGTAGCTCCTCAGGTTCC | | | |
| | TCCACAAATCTATGATAGTT | | | |
| | TTATTGCGGCCATAGACAAG | | | |
| | GGTAACATCAGGACGATGCC | | | |
| | CAATAGATCTATGCCAGCTG | | | |
| | CCCCACATCCTACGCCGGGT | | | |
| | GCCCTTCTAATGGGGGATGC | | | |
| | ATTTAACATGAGACATCCCC | | | |
| | TGACAGGAGGTGGTATGACC | | | |
| | GTGGCATTGAGCGATATTGT | | | |
| | AGTTTTACGTAATCTTTTAA | | | |
| | AACCTCTCAAGGACCTGTCA | | | |
| | GATGCAAGTACTCTGTGCAA | | | |
| | GTATTTAGAAAGTTTCTACA | | | |
| | CCCTTAGAAAACCAGTTGCT | | | |
| | TCAACTATTAACACGTTGGC | | | |
| | CGGGGCTCTATATAAAGTAT | | | |
| | TTTGTGCCTCTCCGGACCAG | | | |
| | GCTAGGAAAGAAATGCGTCA | | | |
| | AGCTTGTTTCGATTATTTAT | | | |
| | CCTTGGGAGGCATATTTTCA | | | |
| | AATGGCCCTGTATCGCTATT | | | |
| | AAGCGGACTAAACCCAAGAC | | | |
| | CACTATCTCTAGTCCTCCAC | | | |
| | TTCTTTGCTGTGGCAATATA | | | |
| | CGGTGTTGGTCGCTTGCTAC | | | |
| | TTCCATTTCCTTCTGTCAAG | | | |
| | GGGATCTGGATTGGAGCGCG | | | |
| | TTTAATCTATAGCGCGAGTG | | | |
| | GTATTATTTTTCCCATTATA | | | |
| | AGAGCTGAGGGTGTTAGACA | | | |
| | GATGTTTTTCCCTGCAACAG | | | |
| | TTCCTGCCTACTATAGGTCC | | | |
| | CCACCCGTGTTCAAACCCAT | | | |
| | AGTTTAA | | | |
| Squalene | ATGGTGGACCAATGTGCACT | 278 | MVDQCALGWILASVLGAAAL | 294 |
| epoxidase | TGGGTGGATCCTAGCGTCAG | | YFLFGRKNGGVSNERRHESI | |
| | TTCTGGGCGCTGCCGCACTC | | KNIATTNGEYKSSNSDGDII | |
| | TACTTTTTGTTCGGACGAAA | | IVGAGVAGSALAYTLGKDGR | |
| | AAATGGTGGTGTATCGAACG | | RVHVIERDLTEPDRIVGELL | |
| | AAAGGCGCCATGAGTCCATA | | QPGGYLKLTELGLEDCVDDI | |
| | AAGAATATTGCTACTACCAA | | DAQRVYGYALFKDGKDTRLS | |
| | CGGTGAATATAAAAGCTCTA | | YPLEKFHSDVAGRSFHNGRF | |
| | ATTCCGATGGGGATATTATA | | IQRMREKAASLPKVSLEQGT | |
| | ATCGTCGGCGCAGGAGTTGC | | VTSLLEENGIIKGVQYKTKT | |
| | TGGTAGTGCCTTAGCTTATA | | GQEMTAYAPLTIVCDGCFSN | |
| | CGTTGGGTAAGGACGGAAGA | | LRRSLCNPKVDVPSCFVGLV | |
| | AGAGTGCACGTCATTGAGCG | | LENCDLPYANHGHVILADPS | |
| | TGATTTGACAGAACCGGATA | | PILFYRISSTEIRCLVDVPG | |
| | GAATCGTGGGGGAATTATTG | | QKVPSISNGEMANYLKNVVA | |
| | CAGCCCGGCGGTTACTTAAA | | PQIPSQLYDSFVAAIDKGNI | |
| | ACTAACTGAGTTAGGTCTTG | | RTMPNRSMPADPYPTPGALL | |
| | AAGACTGCGTTGATGATATT | | MGDAFNMRHPLTGGGMTVAL | |
| | GACGCACAAAGGGTATATGG | | SDVVVLRDLLKPLRDLNDAP | |
| | TTACGCTTTATTTAAGGATG | | TLSKYLEAFYTLRKPVASTI | |
| | GTAAAGATACACGGCTATCT | | NTLAGALYKVFCASPDQARK | |
| | TATCCATTAGAAAAGTTCCA | | EMRQACFDYLSLGGIFSNGP | |
| | TTCAGACGTAGCCGGAAGGT | | VSLLSGLNPRPISLVLHFFA | |
| | CTTTTCACAACGGCAGATTC | | VAIYGVGRLLIPFPSPKRVW | |
| | ATCCAAAGAATGCGGGAAAA | | IGARIISGASAIIFPIIKAE | |
| | AGCGGCTAGTTTGCCAAAAG | | GVRQMFFPATVAAYYRAPRV | |
| | TTTCACTTGAGCAGGGTACC | | VKGR | |
| | GTAACTTCGCTGCTTGAAGA | | | |
| | AAATGGCATTATAAAGGGCG | | | |
| | TCCAATACAAAACAAAAACA | | | |
| | GGTCAAGAAATGACCGCCTA | | | |
| | TGCACCTCTCACTATTGTTT | | | |
| | GTGACGGATGCTTTTCTAAC | | | |
| | CTGCGTAGATCCTTGTGTAA | | | |
| | TCCTAAGGTTGATGTACCAT | | | |
| | CATGCTTTGTGGGCTTAGTT | | | |
| | CTTGAGAACTGTGATCTACC | | | |
| | CTACGCTAATCATGGGCATG | | | |
| | TGATACTGGCTGACCCTAGT | | | |
| | CCAATATTGTTCTATCGAAT | | | |
| | TTCTTCAACGGAGATCAGAT | | | |
| | GTTTAGTCGACGTTCCCGGA | | | |
| | CAAAAAGTTCCTTCGATCTC | | | |
| | TAATGGTGAAATGGCGAATT | | | |
| | ACCTGAAGAACGTAGTCGCC | | | |
| | CCACAGATACCAAGTCAGTT | | | |
| | GTATGATAGCTTTGTTGCAG | | | |
| | CAATTGATAAAGGAAATATT | | | |
| | AGGACTATGCCGAACCGTAG | | | |
| | CATGCCGGCCGATCCTTATC | | | |
| | CCACTCCGGGTGCTTTATTG | | | |
| | ATGGGTGACGCGTTTAATAT | | | |
| | GAGACACCCATTGACAGGCG | | | |
| | GAGGTATGACCGTCGCGCTG | | | |
| | AGTGATGTTGTCGTGCTAAG | | | |
| | AGACCTATTAAAACCATTAC | | | |
| | GCGATTTAAACGATGCTCCT | | | |
| | ACACTGTCAAAGTACCTTGA | | | |
| | AGCATTCTACACGCTACGAA | | | |
| | AACCAGTAGCCAGTACCATC | | | |
| | AACACATTGGCTGGTGCATT | | | |
| | GTATAAGGTGTTTTGCGCAT | | | |
| | CTCCAGATCAAGCACGTAAA | | | |
| | GAAATGAGACAGGCATGTTT | | | |
| | CGATTATTTGTCTCTTGGTG | | | |
| | GTATTTTTTCCAATGGACCT | | | |
| | GTATCACTACTCTCAGGGTT | | | |
| | GAATCCAAGGCCAATTAGCC | | | |
| | TAGTACTACATTTCTTTGCC | | | |
| | GTTGCCATCTACGGCGTAGG | | | |
| | TCGTCTATTAATACCGTTTC | | | |
| | CTTCTCCTAAGAGAGTCTGG | | | |
| | ATCGGCGCTAGAATTATAAG | | | |
| | CGGCGCTTCGGCAATTATCT | | | |
| | TCCCTATAATAAAGGCTGAA | | | |
| | GGAGTTAGACAAATGTTCTT | | | |
| | TCCGGCTACTGTGGCGGCTT | | | |
| | ATTATCGTGCACCAAGAGTT | | | |
| | GTCAAGGGTAGGTAA | | | |
| Squalene | ATGGACGGGGTAATTGATAT | 279 | MDGVIDMQTIPLRTAIAIGG | 295 |
| epoxidase | GCAGACCATACCCCTACGTA | | TAVALVVALYFWFLRSYASP | |
| | CGGCCATCGCAATTGGAGGC | | SHHSNHLPPVPEVPGVPVLG | |
| | ACTGCGGTTGCTCTTGTCGT | | NLLQLKEKKPYMTFTKWAEM | |
| | GGCTTTGTACTTCTGGTTTC | | YGPIYSIRTGATSMVVVSSN | |
| | TCCGTAGCTATGCATCTCCT | | EIAKEVVVTRFPSISTRKLS | |
| | TCCCACCATTCAAATCATTT | | YALKVLTEDKSMVAMSDYHD | |
| | ACCGCCTGTTCCAGAAGTAC | | YHKTVKRHILTAVLGPNAQK | |
| | CAGGTGTGCCAGTTTTAGGT | | KFRAHRDTMMENVSNELHAF | |
| | AACCTGTTGCAATTGAAAGA | | FEKNPNQEVNLRKIFQSQLF | |
| | GAAGAAACCCTATATGACAT | | GLAMKQALGKDVESIYVKDL | |
| | TTACTAAGTGGGCCGAAATG | | ETTMKREEIFEVLVVDPMMG | |
| | TACGGTCCTATCTATTCGAT | | AIEVDWRDFFPYLKWVPNKS | |
| | TCGCACAGGAGCTACATCAA | | FENIIHRMYTRREAVMKALI | |
| | TGGTCGTTGTAAGTAGTAAT | | QEHKKRIASGENLNSYIDYL | |
| | GAGATAGCGAAAGAAGTGGT | | LSEAQTLTDKQLLMSLWEPI | |
| | TGTGACTAGGTTCCCATCTA | | IESSDTTMVTTEWAMYELAK | |
| | TCTCAACTAGAAAACTGTCC | | NPNMQDRLYEEIQSVCGSEK | |
| | TATGCCTTGAAGGTCTTAAC | | ITEENLSQLPYLYAVFQETL | |
| | CGAAGATAAATCTATGGTCG | | RKHCPVPIMPLRYVHENTVL | |
| | CAATGAGCGATTACCATGAC | | GGYHVPAGTEVAINIYGCNM | |
| | TACCACAAAACAGTTAAGCG | | DKKVWENPEEWNPERFLSEK | |
| | ACATATTTTAACGGCTGTTC | | ESMDLYKTMAFGGGKRVCAG | |
| | TAGGCCCAAACGCACAAAAG | | SLQAMVISCIGIGRLVQDFE | |
| | AAATTTAGAGCTCACAGAGA | | WKLKDDAEEDVNTLGLTTQK | |
| | TACCATGATGGAGAATGTAA | | LHPLLALINPRK | |
| | GCAACGAATTGCATGCTTTC | | | |
| | TTTGAAAAGAATCCTAATCA | | | |
| | GGAAGTCAACCTTAGAAAAA | | | |
| | TATTTCAATCCCAACTTTTC | | | |
| | GGTTTAGCTATGAAACAGGC | | | |
| | ATTAGGGAAGGATGTGGAAT | | | |
| | CTATTTATGTAAAGGACCTG | | | |
| | GAAACTACCATGAAAAGGGA | | | |
| | AGAGATATTCGAAGTTCTTG | | | |
| | TTGTTGACCCCATGATGGGA | | | |
| | GCCATTGAAGTCGATTGGCG | | | |
| | AGATTTTTTCCCGTATCTCA | | | |
| | AATGGGTGCCAAATAAATCT | | | |
| | TTTGAAAATATAATCCATAG | | | |
| | GATGTACACGAGAAGAGAGG | | | |
| | CCGTTATGAAAGCGTTGATT | | | |
| | CAAGAGCACAAGAAAAGGAT | | | |
| | CGCTTCAGGTGAGAACCTAA | | | |
| | ATTCCTACATAGATTATCTC | | | |
| | TTGAGTGAAGCTCAAACATT | | | |
| | AACTGACAAACAGTTATTAA | | | |
| | TGTCGTTGTGGGAACCTATT | | | |
| | ATAGAATCGTCTGATACTAC | | | |
| | CATGGTAACAACAGAATGGG | | | |
| | CAATGTATGAACTTGCGAAG | | | |
| | AACCCTAATATGCAAGATCG | | | |
| | TCTGTACGAAGAGATTCAAT | | | |
| | CGGTTTGTGGAAGCGAGAAG | | | |
| | ATTACAGAAGAAAACTTATC | | | |
| | ACAACTCCCATATCTATATG | | | |
| | CCGTTTTCCAGGAAACTCTG | | | |
| | AGAAAGCATTGCCCTGTGCC | | | |
| | GATCATGCCACTTAGATACG | | | |
| | TACATGAGAACACTGTTTTG | | | |
| | GGTGGTTACCACGTACCAGC | | | |
| | AGGCACCGAAGTGGCAATCA | | | |
| | ATATTTATGGCTGTAATATG | | | |
| | GACAAGAAAGTGTGGGAAAA | | | |
| | TCCCGAAGAGTGGAATCCAG | | | |
| | AACGTTTTCTGTCTGAAAAA | | | |
| | GAGAGTATGGATTTGTATAA | | | |
| | AACGATGGCATTTGGTGGAG | | | |
| | GTAAAAGAGTTTGTGCGGGA | | | |
| | TCTCTACAAGCTATGGTAAT | | | |
| | TAGTTGCATCGGCATCGGGA | | | |
| | GGTTAGTTCAAGATTTTGAA | | | |
| | TGGAAACTAAAAGATGACGC | | | |
| | TGAAGAAGATGTGAATACAT | | | |
| | TAGGTTTGACTACGCAAAAG | | | |
| | CTACACCCTTTGTTAGCACT | | | |
| | AATTAACCCGCGGAAA | | | |
| ERG9 | ATGGGAAAGCTATTACAATT | 302 | MGKLLQLALHPVEMKAALKL | 311 |
| | GGCATTGCATCCGGTCGAGA | | KFCRTPLFSIYDQSTSPYLL | |
| | TGAAGGCAGCTTTGAAGCTG | | HCFELLNLTSRSFAAVIREL | |
| | AAGTTTTGCAGAACACCGCT | | HPELRNCVTLFYLILRALDT | |
| | ATTCTCCATCTATGATCAGT | | IEDDMSIEHDLKIDLLRHFH | |
| | CCACGTCTCCATATCTCTTG | | EKLLLTKWSFDGNAPDVKDR | |
| | CACTGTTTCGAACTGTTGAA | | AVLTDFESILIEFHKLKPEY | |
| | CTTGACCTCCAGATCGTTTG | | QEVIKEITEKMGNGMADYIL | |
| | CTGCTGTGATCAGAGAGCTG | | DENYNLNGLQTVHDYDVYCH | |
| | CATCCAGAATTGAGAAACTG | | YVAGLVGDGLTRLIVIAKFA | |
| | TGTTACTCTCTTTTATTTGA | | NESLYSNEQLYESMGLFLQK | |
| | TTTTAAGGGCTTTGGATACC | | TNIIRDYNEDLVDGRSFWPK | |
| | ATCGAAGACGATATGTCCAT | | EIWSQYAPQLKDFMKPENEQ | |
| | CGAACACGATTTGAAAATTG | | LGLDCINHLVLNALSHVIDV | |
| | ACTTGTTGCGTCACTTCCAC | | LTYLASIHEQSTFQFCAIPQ | |
| | GAGAAATTGTTGTTAACTAA | | VMAIATLALVENNREVLHGN | |
| | ATGGAGTTTCGACGGAAATG | | VKIRKGTTCYLILKSRTLRG | |
| | CCCCCGATGTGAAGGACAGA | | CVEIFDYYLRDIKSKLAVQD | |
| | GCCGTTTTGACAGATTTCGA | | PNFLKLNIQISKIEQFMEEM | |
| | ATCGATTCTTATTGAATTCC | | YQDKLPPNVKPNETPIFLKV | |
| | ACAAATTGAAACCAGAATAT | | KERSRYDDELVPTQQEEEYK | |
| | CAAGAAGTCATCAAGGAGAT | | FNMVLSIILSVLLGFYYIYT | |
| | CACCGAGAAAATGGGTAATG | | LHRA | |
| | GTATGGCCGACTACATCTTG | | | |
| | GATGAAAATTACAACTTGAA | | | |
| | TGGGTTGCAAACCGTCCACG | | | |
| | ACTACGACGTGTACTGTCAC | | | |
| | TACGTAGCTGGTTTGGTCGG | | | |
| | TGATGGTTTGACCCGTTTGA | | | |
| | TTGTCATTGCCAAGTTTGCC | | | |
| | AACGAATCTTTGTATTCTAA | | | |
| | TGAGCAATTGTATGAAAGCA | | | |
| | TGGGTCTTTTCCTACAAAAA | | | |
| | ACCAACATCATCAGAGACTA | | | |
| | CAATGAAGATTTGGTCGATG | | | |
| | GTAGATCCTTCTGGCCCAAG | | | |
| | GAAATCTGGTCACAATACGC | | | |
| | TCCTCAGTTGAAGGACTTCA | | | |
| | TGAAACCTGAAAACGAACAA | | | |
| | CTGGGGTTGGACTGTATAAA | | | |
| | CCACCTCGTCTTAAACGCAT | | | |
| | TGAGTCATGTTATCGATGTG | | | |
| | TTGACTTATTTGGCCAGTAT | | | |
| | CCACGAGCAATCCACTTTCC | | | |
| | AATTTTGTGCCATTCCCCAA | | | |
| | GTTATGGCCATTGCAACCTT | | | |
| | GGCTTTGGTATTCAACAACC | | | |
| | GTGAAGTGCTACATGGCAAT | | | |
| | GTAAAGATTCGTAAGGGTAC | | | |
| | TACCTGCTATTTAATTTTGA | | | |
| | AATCAAGGACTTTGCGTGGC | | | |
| | TGTGTCGAGATTTTTGACTA | | | |
| | TTACTTACGTGATATCAAAT | | | |
| | CTAAATTGGCTGTGCAAGAT | | | |
| | CCAAATTTCTTAAAATTGAA | | | |
| | CATTCAAATCTCCAAGATCG | | | |
| | AACAATTCATGGAAGAAATG | | | |
| | TACCAGGATAAATTACCTCC | | | |
| | TAACGTGAAGCCAAATGAAA | | | |
| | CTCCAATTTTCTTGAAAGTT | | | |
| | AAAGAAAGATCCAGATACGA | | | |
| | TGATGAATTGGTCCCAACCC | | | |
| | AACAAGAAGAAGAGTACAAG | | | |
| | TTCAATATGGTTTTATCTAT | | | |
| | CATCTTGTCCGTTCTTCTTG | | | |
| | GGTTTTATTATATATACACT | | | |
| | TTACACAGAGCGTGA | | | |
| ERG1 | ATGTCTGCTGTTAACGTTGC | 303 | MSAVNVAPELINADNTITYD | 312 |
| | ACCTGAATTGATTAATGCCG | | AIVIGAGVIGPCVATGLARK | |
| | ACAACACAATTACCTACGAT | | GKKVLIVERDWAMPDRIVGE | |
| | GCGATTGTCATCGGTGCTGG | | LMQPGGVRALRSLGMIQSIN | |
| | TGTTATCGGTCCATGTGTTG | | NIEAYPVTGYTVFFNGEQVD | |
| | CTACTGGTCTAGCAAGAAAG | | IPYPYKADIPKVEKLKDLVK | |
| | GGTAAGAAAGTTCTTATCGT | | DGNDKVLEDSTIHIKDYEDD | |
| | AGAACGTGACTGGGCTATGC | | ERERGVAFVHGRFLNNLRNI | |
| | CTGATAGAATTGTTGGTGAA | | TAQEPNVTRVQGNCIEILKD | |
| | TTGATGCAACCAGGTGGTGT | | EKNEVVGAKVDIDGRGKVEF | |
| | TAGAGCATTGAGAAGTCTGG | | KAHLTFICDGIFSRFRKELH | |
| | GTATGATTCAATCTATCAAC | | PDHVPTVGSSFVGMSLFNAK | |
| | AACATCGAAGCATATCCTGT | | NPAPMHGHVILGSDHMPILV | |
| | TACCGGTTATACCGTCTTTT | | YQISPEETRILCAYNSPKVP | |
| | TCAACGGCGAACAAGTTGAT | | ADIKSWMIKDVQPFIPKSLR | |
| | ATTCCATACCCTTACAAGGC | | PSFDEAVSQGKFRAMPNSYL | |
| | CGATATCCCTAAAGTTGAAA | | PARQNDVTGMCVIGDALNMR | |
| | AATTGAAGGACTTGGTCAAA | | HPLTGGGMTVGLHDVVLLIK | |
| | GATGGTAATGACAAGGTCTT | | KIGDLDFSDREKVLDELLDY | |
| | GGAAGACAGCACTATTCACA | | HFERKSYDSVINVLSVALYS | |
| | TCAAGGATTACGAAGATGAT | | LFAADSDNLKALQKGCFKYF | |
| | GAAAGAGAAAGGGGTGTTGC | | QRGGDCVNKPVEFLSGVLPK | |
| | TTTTGTTCATGGTAGATTCT | | PLQLTRVFFAVAFYTIYLNM | |
| | TGAACAACTTGAGAAACATT | | EERGFLGLPMALLEGIMILI | |
| | ACTGCTCAAGAGCCAAATGT | | TAIRVFTPFLFGELIG | |
| | TACTAGAGTGCAAGGTAACT | | | |
| | GTATTGAGATATTGAAGGAT | | | |
| | GAAAAGAATGAGGTTGTTGG | | | |
| | TGCCAAGGTTGACATTGATG | | | |
| | GCCGTGGCAAGGTGGAATTC | | | |
| | AAAGCCCACTTGACATTTAT | | | |
| | CTGTGACGGTATCTTTTCAC | | | |
| | GTTTCAGAAAGGAATTGCAC | | | |
| | CCAGACCATGTTCCAACTGT | | | |
| | CGGTTCTTCGTTTGTCGGTA | | | |
| | TGTCTTTGTTCAATGCTAAG | | | |
| | AATCCTGCTCCTATGCACGG | | | |
| | TCACGTTATTCTTGGTAGTG | | | |
| | ATCATATGCCAATCTTGGTT | | | |
| | TACCAAATCAGTCCAGAAGA | | | |
| | AACAAGAATCCTTTGTGCTT | | | |
| | ACAACTCTCCAAAGGTCCCA | | | |
| | GCTGATATCAAGAGTTGGAT | | | |
| | GATTAAGGATGTCCAACCTT | | | |
| | TCATTCCAAAGAGTCTACGT | | | |
| | CCTTCATTTGATGAAGCCGT | | | |
| | CAGCCAAGGTAAATTTAGAG | | | |
| | CTATGCCAAACTCCTACTTG | | | |
| | CCAGCTAGACAAAACGACGT | | | |
| | CACTGGTATGTGTGTTATCG | | | |
| | GTGACGCTCTAAATATGAGA | | | |
| | CATCCATTGACTGGTGGTGG | | | |
| | TATGACTGTCGGTTTGCATG | | | |
| | ATGTTGTCTTGTTGATTAAG | | | |
| | AAAATAGGTGACCTAGACTT | | | |
| | CAGCGACCGTGAAAAGGTTT | | | |
| | TGGATGAATTACTAGACTAC | | | |
| | CATTTCGAAAGAAAGAGTTA | | | |
| | CGATTCCGTTATTAACGTTT | | | |
| | TGTCAGTGGCTTTGTATTCT | | | |
| | TTGTTCGCTGCTGACAGCGA | | | |
| | TAACTTGAAGGCATTACAAA | | | |
| | AAGGTTGTTTCAAATATTTC | | | |
| | CAAAGAGGTGGCGATTGTGT | | | |
| | CAACAAACCCGTTGAATTTC | | | |
| | TGTCTGGTGTCTTGCCAAAG | | | |
| | CCTTTGCAATTGACCAGGGT | | | |
| | TTTCTTCGCTGTCGCTTTTT | | | |
| | ACACCATTTACTTGAACATG | | | |
| | GAAGAACGTGGTTTCTTGGG | | | |
| | ATTACCAATGGCTTTATTGG | | | |
| | AAGGTATTATGATTTTGATC | | | |
| | ACAGCTATTAGAGTATTCAC | | | |
| | CCCATTTTTGTTTGGTGAGT | | | |
| | TGATTGGTTAA | | | |
| ERG7 | ATGACAGAATTTTATTCTGA | 304 | MTEFYSDTIGLPKTDPRLWR | 313 |
| | CACAATCGGTCTACCAAAGA | | LRTDELGRESWEYLTPQQAA | |
| | CAGATCCACGTCTTTGGAGA | | NDPPSTFTQWLLQDPKFPQP | |
| | CTGAGAACTGATGAGCTAGG | | HPERNKHSPDFSAFDACHNG | |
| | CCGAGAAAGCTGGGAATATT | | ASFFKLLQEPDSGIFPCQYK | |
| | TAACCCCTCAGCAAGCCGCA | | GPMFMTIGYVAVNYIAGIEI | |
| | AACGACCCACCATCTACCTT | | PEHERIELIRYIVNTAHPVD | |
| | TACACAATGGCTACTGCAAG | | GGWGLHSVDKSTVFGTVLNY | |
| | ATCCCAAATTTCCTCAACCT | | VILRLLGLPKDHPVCAKARS | |
| | CATCCAGAAAGAAATAAGCA | | TLLRLGGAIGSPHWGKIWLS | |
| | TTCACCAGATTTTTCAGCCT | | ALNLYKWEGVNPAPPETWLL | |
| | TCGATGCGTGTCATAATGGT | | PYSLPMHPGRWWVHTRGVYI | |
| | GCATCTTTTTTCAAACTGCT | | PVSYLSLVKFSCPMTPLLEE | |
| | TCAAGAGCCTGACTCAGGTA | | LRNEIYTKPFDKINFSKNRN | |
| | TTTTTCCGTGTCAATATAAA | | TVCGVDLYYPHSTTLNIANS | |
| | GGACCCATGTTCATGACAAT | | LVVFYEKYLRNRFIYSLSKK | |
| | CGGTTACGTAGCCGTAAACT | | KVYDLIKTELQNTDSLCIAP | |
| | ATATCGCCGGTATTGAAATT | | VNQAFCALVTLIEEGVDSEA | |
| | CCTGAGCATGAGAGAATAGA | | FQRLQYRFKDALFHGPQGMT | |
| | ATTAATTAGATACATCGTCA | | IMGTNGVQTWDCAFAIQYFF | |
| | ATACTGCTCACCCTGTCGAC | | VAGLAERPEFYNTIVSAYKF | |
| | GGAGGTTGGGGTCTACATTC | | LCHAQFDTECVPGSYRDKRK | |
| | TGTTGACAAATCCACCGTGT | | GAWGFSTKTQGYTVADCTAE | |
| | TTGGTACAGTATTGAACTAT | | AIKAIIMVKNSPVFSEVHHM | |
| | GTAATCTTACGTTTATTGGG | | ISSERLFEGIDVLLNLQNIG | |
| | TCTACCCAAGGACCACCCGG | | SFEYGSFATYEKIKAPLAME | |
| | TTTGCGCCAAGGCAAGAAGC | | TLNPAEVFGNIMVEYPYVEC | |
| | ACATTGTTAAGGTTAGGCGG | | TDSSVLGLTYFHKYFDYRKE | |
| | TGCTATTGGATCCCCTCACT | | EIRTRIRIAIEFIKKSQLPD | |
| | GGGGAAAAATTTGGCTAAGT | | GSWYGSWGICFTYAGMFALE | |
| | GCACTAAACTTGTATAAATG | | ALHTVGETYENSSTVRKGCD | |
| | GGAAGGTGTGAACCCTGCCC | | FLVSKQMKDGGWGESMKSSE | |
| | CTCCTGAAACTTGGTTACTT | | LHSYVDSEKSLVVQTAWALI | |
| | CCATATTCACTGCCCATGCA | | ALLFAEYPNKEVIDRGIDLL | |
| | TCCGGGGAGATGGTGGGTTC | | KNRQEESGEWKFESVEGVFN | |
| | ATACTAGAGGTGTTTACATT | | HSCAIEYPSYRFLFPIKALG | |
| | CCGGTCAGTTACCTGTCATT | | MYSRAYETHTL | |
| | GGTCAAATTTTCTTGCCCAA | | | |
| | TGACTCCTCTTCTTGAAGAA | | | |
| | CTGAGGAATGAAATTTACAC | | | |
| | TAAACCGTTTGACAAGATTA | | | |
| | ACTTCTCCAAGAACAGGAAT | | | |
| | ACCGTATGTGGAGTAGACCT | | | |
| | ATATTACCCCCATTCTACTA | | | |
| | CTTTGAATATTGCGAACAGC | | | |
| | CTTGTAGTATTTTACGAAAA | | | |
| | ATACCTAAGAAACCGGTTCA | | | |
| | TTTACTCTCTATCCAAGAAG | | | |
| | AAGGTTTATGATCTAATCAA | | | |
| | AACGGAGTTACAGAATACTG | | | |
| | ATTCCTTGTGTATAGCACCT | | | |
| | GTTAACCAGGCGTTTTGCGC | | | |
| | ACTTGTCACTCTTATTGAAG | | | |
| | AAGGGGTAGACTCGGAAGCG | | | |
| | TTCCAGCGTCTCCAATATAG | | | |
| | GTTCAAGGATGCATTGTTCC | | | |
| | ATGGTCCACAGGGTATGACC | | | |
| | ATTATGGGAACAAATGGTGT | | | |
| | GCAAACCTGGGATTGTGCGT | | | |
| | TTGCCATTCAATACTTTTTC | | | |
| | GTCGCAGGCCTCGCAGAAAG | | | |
| | ACCTGAATTCTATAACACAA | | | |
| | TTGTCTCTGCCTATAAATTC | | | |
| | TTGTGTCATGCTCAATTTGA | | | |
| | CACCGAGTGCGTTCCAGGTA | | | |
| | GTTATAGGGATAAGAGAAAG | | | |
| | GGGGCTTGGGGCTTCTCAAC | | | |
| | AAAAACACAGGGCTATACAG | | | |
| | TGGCAGATTGCACTGCAGAA | | | |
| | GCAATTAAAGCCATCATCAT | | | |
| | GGTGAAAAACTCTCCCGTCT | | | |
| | TTAGTGAAGTACACCATATG | | | |
| | ATTAGCAGTGAACGTTTATT | | | |
| | TGAAGGCATTGATGTGTTAT | | | |
| | TGAACCTACAAAACATCGGA | | | |
| | TCTTTTGAATATGGTTCCTT | | | |
| | TGCAACCTATGAAAAAATCA | | | |
| | AGGCCCCACTAGCAATGGAA | | | |
| | ACCTTGAATCCTGCTGAAGT | | | |
| | TTTTGGTAACATAATGGTAG | | | |
| | AATACCCATACGTGGAATGT | | | |
| | ACTGATTCATCCGTTCTGGG | | | |
| | GTTGACATATTTTCACAAGT | | | |
| | ACTTCGACTATAGGAAAGAG | | | |
| | GAAATACGTACACGCATCAG | | | |
| | AATCGCCATCGAATTCATAA | | | |
| | AAAAATCTCAATTACCAGAT | | | |
| | GGAAGTTGGTATGGAAGCTG | | | |
| | GGGTATTTGTTTTACATATG | | | |
| | CCGGTATGTTTGCATTGGAG | | | |
| | GCATTACACACCGTGGGGGA | | | |
| | GACCTATGAGAATTCCTCAA | | | |
| | CGGTAAGAAAAGGTTGCGAC | | | |
| | TTCTTGGTCAGTAAACAGAT | | | |
| | GAAGGATGGCGGTTGGGGGG | | | |
| | AATCAATGAAGTCCAGTGAA | | | |
| | TTACATAGTTATGTGGATAG | | | |
| | TGAAAAATCGCTAGTCGTTC | | | |
| | AAACCGCATGGGCGCTAATT | | | |
| | GCACTTCTTTTCGCTGAATA | | | |
| | TCCTAATAAAGAAGTCATCG | | | |
| | ACCGCGGTATTGACCTTTTA | | | |
| | AAAAATAGACAAGAAGAATC | | | |
| | CGGGGAATGGAAATTTGAAA | | | |
| | GTGTAGAAGGTGTTTTCAAC | | | |
| | CACTCTTGTGCAATTGAATA | | | |
| | CCCAAGTTATCGATTCTTAT | | | |
| | TCCCTATTAAGGCATTAGGT | | | |
| | ATGTACAGCAGGGCATATGA | | | |
| | AACACATACGCTTTAA | | | |
| AquAga | ATGTGGAGACTTAAGACCGG | 327 | MWRLKTGSETVGDNGRWLRS | 226 |
| CDS16 | TTCCGAGACTGTCGGCGACA | | TNNHVGRQVWEFFPEMGSPE | |
| | ACGGCCGATGGCTTCGAAGC | | ELVAIEAAHREFHLNRFHKQ | |
| | ACCAACAACCATGTGGGTCG | | HSSDLLMRLQYEREKPCVQK | |
| | ACAGGTCTGGGAGTTCTTCC | | EGAVRLDATETPTEAAVETT | |
| | CCGAGATGGGCTCCCCCGAG | | LRRALTFYSTMQSDDGHWAN | |
| | GAGCTGGTCGCCATTGAGGC | | DLGGPMFLLPGLVITLTITG | |
| | CGCCCACCGAGAGTTCCACC | | TINVVLSKEHQREIRRYLYN | |
| | TCAATCGATTCCACAAGCAG | | HQNQDGGWGLHIEGPSTMFG | |
| | CATTCTTCCGACCTGCTGAT | | SALNYVTLRLLGEGPDDGEG | |
| | GCGACTTCAGTACGAGCGAG | | AMERARQWILSRGGAVAVTS | |
| | AGAAGCCATGTGTTCAGAAG | | WGKLWLSVLGVYEWDGNNPL | |
| | GAGGGCGCCGTTCGACTTGA | | PPELWLLPYSLPLHPGRMWC | |
| | CGCAACCGAAACCCCTACCG | | HCRMVYLPMSYLYGKRFVGP | |
| | AGGCGGCCGTTGAGACTACC | | ITPTVLSLREELYPIPYHHV | |
| | CTGCGAAGGGCCCTTACATT | | DWNKARNTCAQDDLYYPHPF | |
| | CTACTCTACTATGCAGTCCG | | VQDLLWGSLYHVYEPLVMRW | |
| | ACGACGGGCATTGGGCCAAC | | PGKRLRERALQHVMKHIHYE | |
| | GACCTGGGTGGACCCATGTT | | DENTEYICLGPVNKALNMLC | |
| | CCTCCTACCCGGACTGGTTA | | CWVEDPHSEAFKMHIPRIYD | |
| | TTACTCTGACCATCACCGGC | | YLWIAEDGMKMQGYNGSQLW | |
| | ACCATCAACGTTGTTCTGAG | | DTAFAVQAIVATKLTDEFSE | |
| | CAAGGAGCATCAGCGAGAGA | | TLAKANKYILDAQILKNCPG | |
| | TTCGACGATACCTCTACAAC | | DPNVWYRHITKGAWSFSTAD | |
| | CATCAGAATCAGGATGGCGG | | QGWLVSDCTAEGLKALLLYS | |
| | ATGGGGCCTGCATATTGAGG | | MLPHQKAPSSIEKNRLYDAV | |
| | GCCCTAGCACTATGTTCGGC | | NVLLSMQNADGGFASFELTR | |
| | TCTGCCCTTAACTACGTCAC | | SYPWLEMINPAETFGDIVID | |
| | GCTACGACTTCTTGGGGAGG | | YTYVECTSAVIQALALFKRL | |
| | GCCCCGACGACGGCGAAGGT | | HPGHRKKEIERCMANAAKFL | |
| | GCCATGGAGCGTGCACGACA | | EMRQEADGSWYGCWGVCYTY | |
| | GTGGATTTTGAGCCGAGGTG | | AGWFGIKGLTSCGRTYNNCA | |
| | GCGCGGTTGCAGTCACTTCT | | NIRRACDFLLSKQLPNGGWG | |
| | TGGGGCAAGCTCTGGCTTTC | | ESYLSCQNKLYTNLNNDRMH | |
| | GGTCCTGGGCGTTTACGAGT | | TVNTAWAMMALIEAGQAKTD | |
| | GGGATGGCAACAACCCCCTG | | PMPLHHAARTLINAQMETGD | |
| | CCTCCCGAACTCTGGTTATT | | FPQQEIMGVFNKNCMISYAG | |
| | GCCCTACTCCCTTCCTCTCC | | YRNVFPVWALGEYHHRVLNG | |
| | ACCCCGGCCGGATGTGGTGC | | C | |
| | CACTGCCGAATGGTGTACCT | | | |
| | TCCCATGTCGTACTTGTACG | | | |
| | GTAAGCGATTCGTGGGTCCC | | | |
| | ATCACACCCACGGTACTCAG | | | |
| | TCTTCGAGAGGAGCTCTACC | | | |
| | CCATCCCCTACCATCATGTG | | | |
| | GACTGGAACAAGGCCCGAAA | | | |
| | CACTTGTGCCCAGGACGATT | | | |
| | TGTACTACCCTCATCCGTTC | | | |
| | GTTCAGGACCTGCTTTGGGG | | | |
| | TTCCCTCTACCACGTCTACG | | | |
| | AGCCCCTTGTTATGCGATGG | | | |
| | CCCGGAAAGCGATTGCGAGA | | | |
| | GAGAGCGCTTCAGCACGTCA | | | |
| | TGAAGCACATACACTATGAG | | | |
| | GATGAGAACACTGAGTACAT | | | |
| | CTGCCTCGGCCCCGTGAACA | | | |
| | AGGCCCTCAACATGCTGTGT | | | |
| | TGTTGGGTCGAGGACCCCCA | | | |
| | CTCAGAGGCCTTCAAGATGC | | | |
| | ACATCCCACGCATTTACGAC | | | |
| | TACCTCTGGATTGCAGAGGA | | | |
| | TGGGATGAAGATGCAGGGTT | | | |
| | ACAACGGCAGCCAGCTCTGG | | | |
| | GACACCGCCTTTGCCGTTCA | | | |
| | AGCCATTGTCGCCACCAAGC | | | |
| | TCACTGATGAGTTTTCCGAA | | | |
| | ACCCTCGCCAAGGCGAATAA | | | |
| | GTACATTCTCGACGCTCAAA | | | |
| | TCCTGAAGAACTGTCCAGGC | | | |
| | GACCCCAACGTTTGGTACCG | | | |
| | ACACATCACAAAGGGCGCCT | | | |
| | GGTCCTTCTCCACTGCTGAC | | | |
| | CAGGGCTGGCTGGTTTCTGA | | | |
| | CTGTACTGCTGAGGGTCTGA | | | |
| | AAGCCCTTCTGCTGTACTCC | | | |
| | ATGCTGCCTCACCAGAAGGC | | | |
| | CCCCTCCTCTATCGAAAAGA | | | |
| | ACCGACTGTACGACGCTGTG | | | |
| | AACGTCCTTCTGTCTATGCA | | | |
| | GAACGCGGACGGTGGCTTCG | | | |
| | CTTCTTTCGAGTTGACCCGT | | | |
| | AGCTACCCCTGGCTGGAGAT | | | |
| | GATCAACCCCGCTGAGACAT | | | |
| | TTGGCGATATCGTTATCGAT | | | |
| | TACACCTACGTCGAGTGTAC | | | |
| | ATCCGCCGTTATCCAGGCCC | | | |
| | TCGCCCTCTTCAAGCGACTC | | | |
| | CATCCCGGTCACCGAAAGAA | | | |
| | GGAGATCGAGCGCTGCATGG | | | |
| | CCAACGCGGCTAAGTTTCTT | | | |
| | GAGATGCGACAGGAGGCTGA | | | |
| | CGGCTCTTGGTACGGTTGCT | | | |
| | GGGGCGTGTGCTACACCTAC | | | |
| | GCAGGTTGGTTCGGCATCAA | | | |
| | GGGCCTCACATCCTGTGGCC | | | |
| | GAACATACAACAACTGTGCC | | | |
| | AACATCAGACGAGCATGCGA | | | |
| | TTTCCTCCTCTCTAAGCAGC | | | |
| | TGCCTAACGGAGGCTGGGGC | | | |
| | GAATCATACTTATCCTGCCA | | | |
| | AAACAAGCTGTACACAAACC | | | |
| | TCAATAACGACCGAATGCAC | | | |
| | ACTGTCAACACCGCTTGGGC | | | |
| | AATGATGGCTCTGATCGAGG | | | |
| | CTGGCCAGGCTAAGACCGAC | | | |
| | CCTATGCCCTTGCATCACGC | | | |
| | CGCGCGAACCCTCATTAACG | | | |
| | CCCAGATGGAAACAGGAGAC | | | |
| | TTCCCCCAGCAGGAGATCAT | | | |
| | GGGCGTTTTCAATAAGAACT | | | |
| | GCATGATTTCTTACGCGGGC | | | |
| | TACCGAAACGTTTTCCCTGT | | | |
| | GTGGGCTTTGGGTGAGTACC | | | |
| | ACCACCGAGTTCTTAACGGT | | | |
| | TGCTAA | | | |
| SgCDS1 | ATGTGGCGACTGAAGGTTGG | 332 | MWRLKVGAESVGENDEKWLK | 256 |
| | TGCTGAGTCCGTAGGCGAAA | | SISNHLGRQVWEFCPDAGTQ | |
| | ATGACGAGAAGTGGCTCAAA | | QQLLQVHKARKAFHDDRFHR | |
| | TCTATCAGTAACCATCTTGG | | KQSSDLFITIQYGKEVENGG | |
| | AAGACAAGTGTGGGAGTTTT | | KTAGVKLKEGEEVRKEAVES | |
| | GCCCTGATGCCGGGACTCAG | | SLERALSFYSSIQTSDGNWA | |
| | CAGCAGCTGTTGCAGGTCCA | | SDLGGPMFLLPGLVIALYVT | |
| | CAAGGCCCGTAAGGCATTCC | | GVLNSVLSKHHRQEMCRYVY | |
| | ACGACGACCGATTCCACCGA | | NHQNEDGGWGLHIEGPSTMF | |
| | AAGCAGTCGTCTGACCTTTT | | GSALNYVALRLLGEDANAGA | |
| | CATTACCATCCAGTATGGTA | | MPKARAWILDHGGATGITSW | |
| | AGGAGGTTGAGAACGGTGGC | | GKLWLSVLGVYEWSGNNPLP | |
| | AAGACCGCTGGTGTCAAGCT | | PEFWLFPYFLPFHPGRMWCH | |
| | GAAGGAGGGTGAGGAGGTCC | | CRMVYLPMSYLYGKRFVGPI | |
| | GCAAGGAGGCCGTTGAGTCC | | TPIVLSLRKELYAVPYHEID | |
| | TCTCTCGAACGAGCGCTCTC | | WNKSRNTCAKEDLYYPHPKM | |
| | CTTTTACTCCTCTATTCAGA | | QDILWGSLHHVYEPLFTRWP | |
| | CCTCCGATGGCAACTGGGCC | | AKRLREKALQTAMQHIHYED | |
| | AGCGATCTGGGTGGTCCCAT | | ENTRYICLGPVNKVLNLLCC | |
| | GTTCTTACTGCCCGGATTGG | | WVEDPYSDAFKLHLQRVHDY | |
| | TCATCGCCCTCTACGTTACG | | LWVAEDGMKMQGYNGSQLWD | |
| | GGTGTGCTAAACTCTGTTCT | | TAFSIQAIVSTKLVDNYGPT | |
| | GTCCAAGCACCATCGACAGG | | LRKAHDFVKSSQIQQDCPGD | |
| | AGATGTGTCGGTACGTCTAC | | PNVWYRHIHKGA WPFSTRD | |
| | AACCACCAAAACGAGGACGG | | HGWLISDCTAEGLKAALMLS | |
| | TGGCTGGGGTCTTCACATTG | | KLPSETVGESLERNRLCDAV | |
| | AGGGACCATCTACCATGTTC | | NVLLSLQNDNGGFASYELTR | |
| | GGTTCAGCTCTAAACTACGT | | SYPWLELINPAETFGDIVID | |
| | CGCCCTCCGACTGCTTGGGG | | YPYVECTSATMEALTLFKKL | |
| | AGGACGCTAACGCCGGTGCA | | HPGHRTKEIDTAIVRAANFL | |
| | ATGCCCAAGGCTCGAGCCTG | | ENMQRTDGSWYGCWGVCFTY | |
| | GATCCTCGACCACGGCGGTG | | AGWFGIKGLVAAGRTYNNCL | |
| | CTACTGGTATCACCTCCTGG | | AIRKACDFLLSKELPGGGWG | |
| | GGTAAGCTCTGGCTGAGTGT | | ESYLSCQNKVYTNLEGNRPH | |
| | GCTTGGCGTCTACGAGTGGT | | LVNTAWVLMALIEAGQAERD | |
| | CCGGCAACAACCCCCTCCCT | | PTPLHRAARLLINSQLENGD | |
| | CCCGAGTTCTGGCTGTTTCC | | FPQQEIMGVFNKNCMITYAA | |
| | CTACTTCCTGCCTTTCCATC | | YRNIFPIWALGEYCHRVLTE | |
| | CCGGAAGGATGTGGTGTCAC | | | |
| | TGCCGAATGGTCTACTTGCC | | | |
| | CATGTCTTATCTCTACGGTA | | | |
| | AGCGATTCGTTGGTCCCATC | | | |
| | ACCCCTATCGTCCTGTCCCT | | | |
| | TCGAAAGGAGCTTTACGCCG | | | |
| | TCCCGTACCACGAGATTGAT | | | |
| | TGGAACAAGTCCCGAAACAC | | | |
| | TTGTGCCAAGGAGGACCTCT | | | |
| | ACTACCCTCACCCCAAGATG | | | |
| | CAGGACATTCTGTGGGGCTC | | | |
| | CCTTCATCACGTGTACGAGC | | | |
| | CCCTGTTCACCCGATGGCCC | | | |
| | GCTAAGCGACTTCGAGAGAA | | | |
| | GGCCTTGCAGACAGCCATGC | | | |
| | AGCACATCCACTACGAAGAC | | | |
| | GAAAATACCCGATACATCTG | | | |
| | CCTGGGTCCCGTCAACAAGG | | | |
| | TTCTGAACCTCTTGTGTTGT | | | |
| | TGGGTCGAGGATCCCTACTC | | | |
| | TGATGCTTTCAAACTCCACC | | | |
| | TCCAGCGAGTTCACGACTAC | | | |
| | CTGTGGGTTGCCGAGGACGG | | | |
| | AATGAAGATGCAGGGATACA | | | |
| | ACGGTTCTCAGCTCTGGGAT | | | |
| | ACTGCATTTTCGATTCAGGC | | | |
| | CATCGTCAGCACCAAGCTGG | | | |
| | TAGACAACTACGGACCGACA | | | |
| | CTCCGAAAGGCTCACGACTT | | | |
| | TGTTAAGTCTTCCCAGATCC | | | |
| | AACAGGACTGCCCCGGTGAT | | | |
| | CCCAACGTCTGGTACAGACA | | | |
| | CATTCATAAAGGTGCCTGGC | | | |
| | CTTTCTCCACCCGTGACCAC | | | |
| | GGCTGGCTCATTTCTGATTG | | | |
| | TACCGCTGAGGGCCTTAAGG | | | |
| | CCGCCCTGATGCTGTCCAAG | | | |
| | CTCCCCTCTGAGACTGTGGG | | | |
| | TGAGTCGCTCGAGCGAAACC | | | |
| | GACTTTGCGACGCCGTGAAC | | | |
| | GTTCTCCTTAGTCTCCAGAA | | | |
| | CGACAACGGTGGTTTCGCTT | | | |
| | CCTATGAGCTGACCCGTTCC | | | |
| | TACCCCTGGCTTGAACTGAT | | | |
| | TAACCCTGCGGAGACATTCG | | | |
| | GTGATATCGTCATCGACTAC | | | |
| | CCCTACGTTGAGTGTACGTC | | | |
| | TGCCACCATGGAGGCTCTTA | | | |
| | CCCTGTTTAAGAAGCTCCAT | | | |
| | CCTGGTCACCGAACCAAGGA | | | |
| | GATTGACACCGCCATCGTCC | | | |
| | GAGCCGCTAATTTCCTGGAG | | | |
| | AACATGCAGCGAACCGACGG | | | |
| | GTCATGGTACGGTTGCTGGG | | | |
| | GAGTCTGTTTTACCTACGCC | | | |
| | GGATGGTTCGGTATTAAGGG | | | |
| | TCTTGTCGCCGCTGGCCGAA | | | |
| | CTTACAACAACTGTTTGGCC | | | |
| | ATCAGAAAGGCCTGCGACTT | | | |
| | CCTCCTGTCTAAGGAGCTGC | | | |
| | CCGGAGGTGGCTGGGGCGAA | | | |
| | TCCTATCTCTCCTGTCAGAA | | | |
| | TAAGGTTTACACCAACTTAG | | | |
| | AGGGTAACAGGCCCCACTTG | | | |
| | GTGAATACTGCTTGGGTTCT | | | |
| | TATGGCGCTGATCGAGGCCG | | | |
| | GTCAGGCGGAGCGAGATCCC | | | |
| | ACCCCCCTACACCGAGCTGC | | | |
| | CCGACTGCTTATCAACTCCC | | | |
| | AGCTCGAGAACGGTGACTTC | | | |
| | CCTCAACAGGAGATTATGGG | | | |
| | TGTTTTTAACAAGAACTGCA | | | |
| | TGATCACGTACGCCGCCTAC | | | |
| | CGAAACATCTTCCCTATCTG | | | |
| | GGCTCTTGGTGAATACTGTC | | | |
| | ACCGAGTCCTGACCGAGTAA | | | |

### EQUIVALENTS

Those skilled in the art will recognize or be able to ascertain using no more than routine experimentation many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

1. A host cell for producing an isoprenoid precursor or isoprenoid, wherein the host cell comprises a heterologous polynucleotide encoding a lanosterol synthase with reduced activity as compared to a wild-type lanosterol synthase, wherein the host cell is capable of producing more of an isoprenoid or isoprenoid precursor as compared to a control host cell that does not comprise the heterologous polynucleotide.

2. The host cell of claim 1, wherein the wild-type lanosterol synthase comprises SEQ ID NO: 1 or SEQ ID NO: 313.

3. The host cell of claim 1 or 2, wherein the lanosterol synthase comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

4. The host cell of any one of claims 1-3, wherein the lanosterol synthase comprises 2, 3, 4, 5, 6, 7, 8, 9, 10, 11, or 12 amino acid substitutions and/or deletions relative to SEQ ID NO: 1.

5. The host cell of any one of claims 1-4, wherein the lanosterol synthase comprises:
a) the amino acid Y at the residue corresponding to position 14 in SEQ ID NO:1;
b) the amino acid Q at the residue corresponding to position 33 in SEQ ID NO:1;
c) the amino acid E at the residue corresponding to position 47 in SEQ ID NO:1;
d) the amino acid G at the residue corresponding to position 50 in SEQ ID NO:1;
e) the amino acid R at the residue corresponding to position 66 in SEQ ID NO:1;
f) the amino acid G at the residue corresponding to position 80 in SEQ ID NO: 1;
g) the amino acid L at the residue corresponding to position 83 in SEQ ID NO: 1;
h) the amino acid N at the residue corresponding to position 85 in SEQ ID NO:1;
i) the amino acid I at the residue corresponding to position 92 in SEQ ID NO:1;
j) the amino acid S at the residue corresponding to position 94 in SEQ ID NO:1;
k) the amino acid D at the residue corresponding to position 107 in SEQ ID NO:1;
l) the amino acid C at the residue corresponding to position 122 in SEQ ID NO:1;
m) the amino acid S at the residue corresponding to position 132 in SEQ ID NO:1;
n) the amino acid C at the residue corresponding to position 145 in SEQ ID NO:1;
o) the amino acid S at the residue corresponding to position 158 in SEQ ID NO:1;
p) the amino acid A at the residue corresponding to position 170 in SEQ ID NO: 1;
q) the amino acid N at the residue corresponding to position 172 in SEQ ID NO:1;
r) the amino acid W at the residue corresponding to position 184 in SEQ ID NO:1;
s) the amino acid C or H at the residue corresponding to position 193 in SEQ ID NO:1;
t) the amino acid V at the residue corresponding to position 197 in SEQ ID NO:1;
u) the amino acid I at the residue corresponding to position 198 in SEQ ID NO: 1;
v) the amino acid I at the residue corresponding to position 212 in SEQ ID NO:1;
w) the amino acid L at the residue corresponding to position 213 in SEQ ID NO:1;
x) the amino acid L at the residue corresponding to position 227 in SEQ ID NO:1;
y) the amino acid T at the residue corresponding to position 228 in SEQ ID NO: 1;
z) the amino acid V at the residue corresponding to position 231 in SEQ ID NO:1;
aa) the amino acid M at the residue corresponding to position 235 in SEQ ID NO:1;
bb) the amino acid F at the residue corresponding to position 248 in SEQ ID NO:1;
cc) the amino acid L at the residue corresponding to position 249 in SEQ ID NO:1;
dd) the amino acid R at the residue corresponding to position 260 in SEQ ID NO:1;
ee) the amino acid I at the residue corresponding to position 282 in SEQ ID NO:1;
ff) the amino acid F at the residue corresponding to position 286 in SEQ ID NO: 1;
gg) the amino acid G at the residue corresponding to position 287 in SEQ ID NO:1;
hh) the amino acid G at the residue corresponding to position 289 in SEQ ID NO: 1;
ii) the amino acid I at the residue corresponding to position 295 in SEQ ID NO: 1;
jj) the amino acid T at the residue corresponding to position 296 in SEQ ID NO: 1;
kk) the amino acid F at the residue corresponding to position 309 in SEQ ID NO: 1;
ll) the amino acid S at the residue corresponding to position 314 in SEQ ID NO:1;
mm) the amino acid R at the residue corresponding to position 316 in SEQ ID NO:1;
nn) the amino acid N at the residue corresponding to position 329 in SEQ ID NO:1;
oo) the amino acid A at the residue corresponding to position 344 in SEQ ID NO: 1;
pp) the amino acid S at the residue corresponding to position 360 in SEQ ID NO:1;
qq) the amino acid L at the residue corresponding to position 370 in SEQ ID NO:1;
rr) the amino acid V at the residue corresponding to position 371 in SEQ ID NO:1;
ss) the amino acid P at the residue corresponding to position 372 in SEQ ID NO:1;
tt) the amino acid I at the residue corresponding to position 398 in SEQ ID NO: 1;
uu) the amino acid V at the residue corresponding to position 407 in SEQ ID NO:1;
vv) the amino acid S at the residue corresponding to position 414 in SEQ ID NO:1;
ww) the amino acid S at the residue corresponding to position 417 in SEQ ID NO:1;
xx) the amino acid L at the residue corresponding to position 423 in SEQ ID NO:1;
yy) the amino acid I or S at the residue corresponding to position 432 in SEQ ID NO:1;
zz) the amino acid L at the residue corresponding to position 437 in SEQ ID NO:1;
aaa) the amino acid V at the residue corresponding to position 442 in SEQ ID NO:1;
bbb) the amino acid M or S at the residue corresponding to position 444 in SEQ ID NO:1;
ccc) the amino acid G at the residue corresponding to position 452 in SEQ ID NO:1;
ddd) the amino acid V at the residue corresponding to position 474 in SEQ ID NO:1;
eee) the amino acid S at the residue corresponding to position 479 in SEQ ID NO:1;
fff) the amino acid Q at the residue corresponding to position 491 in SEQ ID NO:1;
ggg) the amino acid N at the residue corresponding to position 498 in SEQ ID NO: 1;
hhh) the amino acid L at the residue corresponding to position 515 in SEQ ID NO:1;
iii) the amino acid T at the residue corresponding to position 526 in SEQ ID NO:1;
jjj) the amino acid T at the residue corresponding to position 529 in SEQ ID NO:1;
kkk) the amino acid F at the residue corresponding to position 536 in SEQ ID NO:1;
lll) the amino acid Y at the residue corresponding to position 544 in SEQ ID NO:1;
mmm) the amino acid E at the residue corresponding to position 552 in SEQ ID NO:1;
nnn) the amino acid A at the residue corresponding to position 559 in SEQ ID NO:1;
ooo) the amino acid M at the residue corresponding to position 560 in SEQ ID NO:1;
ppp) the amino acid C or N at the residue corresponding to position 564 in SEQ ID NO:1;
qqq) the amino acid P at the residue corresponding to position 578 in SEQ ID NO:1;
rrr) the amino acid F at the residue corresponding to position 586 in SEQ ID NO:1;
sss) the amino acid T at the residue corresponding to position 608 in SEQ ID NO:1;
ttt) the amino acid I at the residue corresponding to position 610 in SEQ ID NO: 1;
uuu) the amino acid V at the residue corresponding to position 617 in SEQ ID NO:1;
vvv) the amino acid L at the residue corresponding to position 619 in SEQ ID NO:1;
www) the amino acid S at the residue corresponding to position 620 in SEQ ID NO:1;
xxx) the amino acid E or R at the residue corresponding to position 631 in SEQ ID NO:1;
yyy) the amino acid D at the residue corresponding to position 638 in SEQ ID NO:1;
zzz) the amino acid L at the residue corresponding to position 650 in SEQ ID NO:1;
aaaa) the amino acid A at the residue corresponding to position 655 in SEQ ID NO:1;
bbbb) the amino acid H at the residue corresponding to position 660 in SEQ ID NO:1;
cccc) the amino acid S at the residue corresponding to position 679 in SEQ ID NO:1;
dddd) the amino acid E at the residue corresponding to position 686 in SEQ ID NO: 1;
eeee) the amino acid D at the residue corresponding to position 702 in SEQ ID NO:1;
ffff) the amino acid Q at the residue corresponding to position 710 in SEQ ID NO:1;
gggg) the amino acid L or V at the residue corresponding to position 726 in SEQ ID NO:1;
hhhh) the amino acid F at the residue corresponding to position 736 in SEQ ID NO:1;
iiii) the amino acid M at the residue corresponding to position 738 in SEQ ID NO:1; and/or
jjjj) a truncation that results in deletion of the residue corresponding to position 742 in SEQ ID NO: 1.

6. The host cell of any one of claims 1-5, wherein the lanosterol synthase comprises the amino acid substitution E617V, G107D, and/or K631E relative to SEQ ID NO: 1.

7. The host cell of any one of claims 1-5, wherein relative to SEQ ID NO: 1, the lanosterol synthase comprises:
a) R33Q, R193C, D289G, N295I, S296T, N620S, and Y736F;
b) R184W, L235M, L260R, and E710Q;
c) K47E, L92I, T360S, S372P, T444M, and R578P;
d) D50G, K66R, N94S, G417S, E617V, and F726L;
e) N14Y, N132S, Y145C, R193H, I286F, L316R, F432I, E442V, T444S, I479S, K631R, and T655A;
f) F432S, D452G, and I536F;
g) E287G, K329N, E617V, and F726V;
h) E231V, A407V, Q423L, A529T, and Y564C;
i) V248F, D371V, and G702D;
j) L197V, K282I, N314S, P370L, A608T, G638D, and F650L;
k) L491Q, Y586F, and R660H;
l) G122C, H249L, and K738M;
m) P227L, E474V, V559A, and Y564N;
n) K85N, G158S, S515L, P526T, Q619L, and a truncation resulting in a deletion of the residue corresponding to Q742 in SEQ ID NO: 1;
o) G107D and K631E;
p) T212I, W213L, N544Y, and V552E;
q) I172N, C414S, L560M, and G679S;
r) R193C, D289G, N295I, S296T, N620S, and Y736F;
s) K85N and G158S;
t) L197V, K282I, N314S, and P370L;
u) I172N, C414S, and L560M;
v) D371V, M610I, and G702D;
w) D371V, K498N, M610I, and G702D;
x) D80G, P83L, T170A, T198I, and A228T;
y) T360S, S372P, T444M, and R578P;
z) D50G, K66R, N94S, G417S, and E617V; or
aa) L309F, V344A, T398I, and K686E.

8. The host cell of any one of claims 1-5, wherein relative to SEQ ID NO: 1, the lanosterol synthase comprises the following amino acid substitutions:
(a) R193C, D289G, N295I, S296T, N620S, and Y736F;
(b) F432S, D452G, and I536F;
(c) K85N and G158S;
(d) L197V, K282I, N314S, and P370L;
(e) I172N, C414S, L560M, and G679S;
(f) I172N, C414S, and L560M;
(g) D371V, M610I, and G702D;
(h) D371V, K498N, M610I, and G702D;
(i) D80G, P83L, T170A, T198I, and A228T;
(j) D50G, K66R, N94S, G417S, E617V, and F726L;
(k) T360S, S372P, T444M, and R578P;
(l) D50G, K66R, N94S, G417S, and E617V; and
(m) L309F, V344A, T398I, and K686E.

9. The host cell of any one of claims 1-5, wherein relative to SEQ ID NO: 1, the lanosterol synthase comprises the following amino acid substitutions:
(a) D50G, K66R, N94S, G417S, E617V, and F726L;
(b) K85N and G158S;
(c) K47E, L92I, T360S, S372P, T444M, and R578P;
(d) F432S, D452G, and I536F;
(e) T360S, S372P, T444M, and R578P;
(f) L491Q, Y586F, and R660H;
(g) K85N, G158S, S515L, P526T, Q619L, and a truncation that results in deletion of the residue corresponding to position 742 in SEQ ID NO: 1; or
(h) I172N, C414S, L560M, and G679S.

10. The host cell of any one of claims 1-5, wherein the lanosterol synthase comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

11. The host cell of any one of claims 1-5 and 10, wherein the lanosterol synthase comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

12. The host cell of any one of claims 1-11, wherein the lanosterol synthase comprises a sequence that is at least 90% identical to SEQ ID NO: 3, 83-87, 89-92, 94-95, 99, 118-120, 316-319, 321-326, 329, or 331.

13. The host cell of claim 12, wherein the lanosterol synthase comprises SEQ ID NO: 3, 83-87, 89-92, 94-95, 99, 118-120, 316-319, 321-326, 329, or 331.

14. The host cell of any one of claims 1-13, wherein the heterologous polynucleotide comprises a sequence that is at least 90% identical to SEQ ID NO: 4, 62-66, 68-71, 73-74, 78, 103-109, 111-117, 328, or 330.

15. The host cell of claim 14, wherein the heterologous polynucleotide comprises the sequence of SEQ ID NO: 4, 62-66, 68-71, 73-74, 78, 103-109, 111-117, 328, or 330.

16. A host cell that comprises a heterologous polynucleotide encoding a lanosterol synthase, wherein the lanosterol synthase comprises a sequence that is at least 90% identical to SEQ ID NO: 3, 83-87, 89-92, 94-95, 99, 100-102, 118-120, 316-319, 321-326, 329, or 331.

17. The host cell of claim 16, wherein the lanosterol synthase comprises SEQ ID NO: 3, 83-87, 89-92, 94-95, 99, 100-102, 118-120, 316-319, 321-326, 329, or 331.

18. A host cell that comprises a heterologous polynucleotide encoding a lanosterol synthase, wherein the lanosterol synthase comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

19. A host cell that comprises a heterologous polynucleotide encoding a lanosterol synthase, wherein the heterologous polynucleotide comprises a sequence that is at least 90% identical to SEQ ID NO: 4, 62-66, 68-71, 73-74, 78, 80-82, 103-109, 111-117, 328, or 330.

20. The host cell of claim 19, wherein the heterologous polynucleotide comprises SEQ ID NO: 4, 62-66, 68-71, 73-74, 78, 80-82, 103-109, 111-117, 328, or 330.

21. The host cell of claim 1 or 2, wherein the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

22. The host cell of claim 21, wherein the lanosterol synthase comprises:
(a) the amino acid G at the residue corresponding to position 64 in SEQ ID NO: 313;
(b) the amino acid V at the residue corresponding to position 120 in SEQ ID NO: 313;
(c) the amino acid S at the residue corresponding to position 121 in SEQ ID NO: 313;
(d) the amino acid V at the residue corresponding to position 136 in SEQ ID NO: 313;
(e) the amino acid I at the residue corresponding to position 226 in SEQ ID NO: 313;
(f) the amino acid S at the residue corresponding to position 268 in SEQ ID NO: 313;
(g) the amino acid I at the residue corresponding to position 275 in SEQ ID NO: 313;
(h) the amino acid A at the residue corresponding to position 281 in SEQ ID NO: 313;
(i) the amino acid G at the residue corresponding to position 300 in SEQ ID NO: 313;
(j) the amino acid G at the residue corresponding to position 322 in SEQ ID NO: 313;
(k) the amino acid A at the residue corresponding to position 333 in SEQ ID NO: 313;
(l) the amino acid E at the residue corresponding to position 438 in SEQ ID NO: 313;
(m) the amino acid L at the residue corresponding to position 502 in SEQ ID NO: 313;
(n) the amino acid N at the residue corresponding to position 604 in SEQ ID NO: 313;
(o) the amino acid S at the residue corresponding to position 619 in SEQ ID NO: 313;
(p) the amino acid E at the residue corresponding to position 628 in SEQ ID NO: 313;
(q) the amino acid T at the residue corresponding to position 656 in SEQ ID NO: 313;
(r) the amino acid G at the residue corresponding to position 693 in SEQ ID NO: 313; and/or
(s) deletion of residues corresponding to positions 726-731 in SEQ ID NO: 313.

23. The host cell of any one of claims 1-2 and 21-22, wherein the lanosterol synthase comprises relative to SEQ ID NO: 313:
(a) P121S, A136V, S300G, V322G, K438E, F502L, K628E, and deletion of residues corresponding to positions 726-731 in SEQ ID NO: 313;
(b) K268S, T281A, F502L, T604N, A656T, and E693G; or
(c) C619S, F275I, I120V, M226I, R64G, and T333A.

24. The host cell of any one of claims 1-2 and 21-23, wherein the lanosterol synthase comprises a sequence that is at least 90% identical to any one of SEQ ID NOs: 100-102.

25. The host cell of claim 24, wherein the lanosterol synthase comprises a sequence selected from SEQ ID NOs: 100-102.

26. The host of any one of claims 1-2 and 21-25, wherein the heterologous polynucleotide encoding the lanosterol synthase comprises a sequence that is at least 90% identical to a sequence selected from SEQ ID NOs: 80-82.

27. The host cell of claim 26, wherein the heterologous polynucleotide encoding the lanosterol synthase comprises a sequence selected from SEQ ID NOs: 80-82.

28. The host cell of any one of claims 1-27, wherein the host cell is capable of producing mevalonate.

29. The host cell of any one of claims 1-28, wherein the host cell is capable of producing at least 0.2 g/L mevalonate.

30. The host cell of any one of claims 1-29, wherein the host cell is capable of producing at least 0.7 g/L mevalonate.

31. The host cell of any one of claims 1-30, wherein the host cell is capable of producing at least 9 mg/L of an isoprenoid.

32. The host cell of any one of claims 1-31, wherein the host cell is capable of producing at least 1.1 fold more of an isoprenoid than a control host cell comprising SEQ ID NO: 1 and/or a control host cell comprising SEQ ID NO: 313.

33. The host cell of any one of claims 1-32, wherein the host cell is capable of producing at least 3 fold more of an isoprenoid than a control host cell comprising SEQ ID NO: 1 and/or a control host cell comprising SEQ ID NO: 313.

34. The host cell of any one of claims 1-33, wherein the host cell is capable of producing at most 200 mg/L lanosterol.

35. The host cell of any one of claims 1-34, wherein the host cell is capable of producing at least 5 mg/L oxidosqualene.

36. The host cell of any one of claims 1-35, wherein the host cell is capable of producing more mevalonate than a control host cell that does not comprise the heterologous polynucleotide encoding the lanosterol synthase with reduced activity as compared to the wild-type lanosterol synthase.

37. The host cell of any one of claims 1-36, wherein the host cell is capable of producing more 2-3-oxidosqualene as compared to a host cell that does not comprise the heterologous polynucleotide encoding the lanosterol synthase with reduced activity as compared to the wild-type lanosterol synthase.

38. The host cell of any one of claims 1-37, wherein the host cell further comprises:
(a) a heterologous polynucleotide encoding a squalene epoxidase with reduced activity as compared to a wild-type squalene epoxidase; or
(b) a heterologous polynucleotide that reduces squalene epoxidase activity, wherein the host cell is capable of producing more of an isoprenoid or isoprenoid precursor as compared to a control host cell that does not comprise the heterologous polynucleotide of (a) and/or (b).

39. The host cell of claim 38, wherein the wild-type squalene epoxidase comprises SEQ ID NO: 9 or 312.

40. A host cell for producing an isoprenoid precursor or isoprenoid, wherein the host cell comprises:
(a) a heterologous polynucleotide encoding a squalene epoxidase with reduced activity as compared to a wild-type squalene epoxidase; or
(b) a heterologous polynucleotide that reduces squalene epoxidase activity, wherein the host cell is capable of producing more of an isoprenoid or isoprenoid precursor as compared to a control host cell that does not comprise the heterologous polynucleotide of (a) and/or (b).

41. The host cell of claim 40, wherein the wild-type squalene epoxidase comprises SEQ ID NO: 9 or 312.

42. The host cell of claim 40 or 41, wherein the heterologous polynucleotide encodes a squalene epoxidase that comprises 1, 2, 3, 4, 5, 6, 7, 8, 9, or 10 amino acid substitutions and/or deletions relative to SEQ ID NO: 9 or 312.

43. The host cell of any one of claims 40-42, wherein the host cell is capable of producing mevalonate.

44. The host cell of any one of claims 40-43, wherein the host cell is capable of producing at least 0.2 g/L mevalonate.

45. The host cell of any one of claims 40-44, wherein the host cell is capable of producing at least 0.7 g/L mevalonate.

46. The host cell of any one of claims 40-45, wherein the host cell is capable of producing more mevalonate than a control host cell that does not comprise (a) the heterologous polynucleotide encoding the squalene epoxidase with reduced activity as compared to the wild-type squalene epoxidase; and/or (b) the heterologous polynucleotide that reduces squalene epoxidase activity.

47. The host cell of any one of claims 40-46, wherein the host cell is capable of producing more 2-3-oxidosqualene as compared to a host cell that does not comprise (a) the heterologous polynucleotide encoding the squalene epoxidase with reduced activity as compared to the wild-type squalene epoxidase; and/or (b) the heterologous polynucleotide that reduces squalene epoxidase activity.

48. The host cell of any one of claims 40-47, wherein the host cell further comprises:
(a) a heterologous polynucleotide encoding a lanosterol synthase with reduced activity as compared to a lanosterol synthase; or
(b) a heterologous polynucleotide that reduces lanosterol synthase activity, wherein the host cell is capable of producing more of an isoprenoid or isoprenoid precursor as compared to a control host cell that does not comprise the heterologous polynucleotide of (a) and/or (b).

49. The host cell of claim 48, wherein the wild-type lanosterol synthase comprises SEQ ID NO: 1 or 313.

50. A host cell comprising:
(a) one or more enzymes in the yeast mevalonate pathway; and
(b) a heterologous polynucleotide encoding a lanosterol synthase with reduced activity as compared to a wild-type lanosterol synthase; and/or
(c) a heterologous polynucleotide encoding a squalene epoxidase with reduced activity as compared to a wild-type squalene epoxidase; or
(d) a heterologous polynucleotide that reduces squalene epoxidase activity.

51. The host cell of claim 50, wherein the one or more enzymes in the yeast mevalonate pathway is selected from an enzyme with one of the following enzyme classification numbers: EC 2.3.1.9, EC 2.3.3.10, EC 1.1.1.88, EC 1.1.1.34, EC 2.7.1.36, EC 2.7.4.2, EC 4.1.1.33, and/or EC 5.3.3.2,

52. A host cell comprising:
(a) one or more enzymes in the Archaea I mevalonate pathway; and
(b) a heterologous polynucleotide encoding a lanosterol synthase with reduced activity as compared to a wild-type lanosterol synthase; or
(c) a heterologous polynucleotide that reduces lanosterol synthase activity; and/or
(d) a heterologous polynucleotide encoding a squalene epoxidase with reduced activity as compared to a wild-type squalene epoxidase; or
(e) a heterologous polynucleotide that reduces squalene epoxidase activity.

53. The host cell of claim 36, wherein the one or more enzymes in the archaea I mevalonate pathway is selected from an enzyme with one of the following enzyme classification numbers: EC 4.1.1.99, EC 2.7.4.26, EC 2.3.1.9, EC 2.3.3.10, EC 1.1.1.88, EC 1.1.1.34, EC 2.7.1.36, and/or EC 5.3.3.2.

54. A host cell comprising:
(a) one or more enzymes in the Archaea II mevalonate pathway; and
(b) a heterologous polynucleotide encoding a lanosterol synthase with reduced activity as compared to a wild-type lanosterol synthase; or
(c) a heterologous polynucleotide that reduces lanosterol synthase activity; and/or
(d) a heterologous polynucleotide encoding a squalene epoxidase with reduced activity as compared to a wild-type squalene epoxidase; or
(e) a heterologous polynucleotide that reduces squalene epoxidase activity.

55. The host cell of claim 54, wherein the one or more enzymes in the Archaea II mevalonate pathway is selected from an enzyme with one of the following enzyme classification numbers: EC 2.7.1.185, EC 2.7.1.186, EC 2.7.4.26, EC 4.1.1.99, EC 2.3.1.9, EC 2.3.3.10, EC 1.1.1.88, EC 1.1.1.34, EC 2.7.1.36, and/or EC 5.3.3.2.

56. A host cell comprising:
(a) one or more enzymes in the MEP pathway; and
(b) a heterologous polynucleotide encoding a lanosterol synthase with reduced activity as compared to a wild-type lanosterol synthase; or
(c) a heterologous polynucleotide that reduces lanosterol synthase activity; and/or
(d) a heterologous polynucleotide encoding a squalene epoxidase with reduced activity as compared to a wild-type squalene epoxidase; or
(e) a heterologous polynucleotide that reduces squalene epoxidase activity.

57. The host cell of claim 56, wherein the one or more enzymes in the MEP pathway is selected from an enzyme with one of the following enzyme classification numbers: EC 2.2.1.7, EC 1.1.1.267, EC 2.7.7.60, EC 2.7.1.148, EC 4.6.1.12, EC 1.17.7.1, and/or EC 1.17.1.2.

58. The host cell of any one of claims 1-57, wherein the host cell is a yeast cell, a plant cell, or a bacterial cell.

59. The host cell of claim 58, wherein the host cell is a yeast cell.

60. The host cell of claim 59, wherein the yeast cell is a Saccharomyces cerevisiae cell.

61. The host cell of claim 59, wherein the yeast cell is a Yarrowia lipolytica cell.

62. The host cell of claim 58, wherein the host cell is a bacterial cell.

63. The host cell of claim 62, wherein the bacterial cell is an E. coli cell.

64. A method of producing mevalonate comprising culturing the host cell of any one of claims 1-63.

65. A method of producing an isoprenoid precursor or isoprenoid comprising culturing the host cell of any one of claims 1-63.

66. A method of producing 2-C-Methyl-d-erythritol-2,4-cyclopyrophosphate (MEcPP) comprising culturing the host cell of any one of claims 1-63.

67. A method of producing an isoprenoid precursor or isoprenoid comprising culturing a host that comprises:
(a) a heterologous polynucleotide encoding a lanosterol synthase with reduced activity as compared to a wild-type lanosterol synthase; and/or
(b) a heterologous polynucleotide encoding a squalene epoxidase with reduced activity as compared to a wild-type squalene epoxidase; or
(c) a heterologous polynucleotide that reduces squalene epoxidase activity, wherein the host cell is capable of producing more of an isoprenoid or isoprenoid precursor as compared to a control host cell that does not comprise one or more of (a)-(c).

68. The method of claim 67, wherein the wild-type lanosterol synthase comprises SEQ ID NO: 1 or 313.

69. The method of claim 67 or 68, wherein the heterologous polynucleotide in (a) encodes a lanosterol synthase that comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

70. The method of any one of claims 67-69, wherein the heterologous polynucleotide in (a) encodes a lanosterol synthase that comprises 2, 3, 4, 5, 6, 7, 8, 9, 10, 11, or 12 amino acid substitutions and/or deletions relative to SEQ ID NO: 1.

71. The method of any one of claims 67-70, wherein the heterologous polynucleotide encoding the lanosterol synthase with reduced activity encodes a lanosterol synthase that comprises:
a) the amino acid Y at the residue corresponding to position 14 in SEQ ID NO:1;
b) the amino acid Q at the residue corresponding to position 33 in SEQ ID NO:1;
c) the amino acid E at the residue corresponding to position 47 in SEQ ID NO:1;
d) the amino acid G at the residue corresponding to position 50 in SEQ ID NO:1;
e) the amino acid R at the residue corresponding to position 66 in SEQ ID NO:1;
f) the amino acid G at the residue corresponding to position 80 in SEQ ID NO: 1;
g) the amino acid L at the residue corresponding to position 83 in SEQ ID NO: 1;
h) the amino acid N at the residue corresponding to position 85 in SEQ ID NO:1;
i) the amino acid I at the residue corresponding to position 92 in SEQ ID NO:1;
j) the amino acid S at the residue corresponding to position 94 in SEQ ID NO:1;
k) the amino acid D at the residue corresponding to position 107 in SEQ ID NO:1;
l) the amino acid C at the residue corresponding to position 122 in SEQ ID NO:1;
m) the amino acid S at the residue corresponding to position 132 in SEQ ID NO:1;
n) the amino acid C at the residue corresponding to position 145 in SEQ ID NO:1;
o) the amino acid S at the residue corresponding to position 158 in SEQ ID NO:1;
p) the amino acid A at the residue corresponding to position 170 in SEQ ID NO: 1;
q) the amino acid N at the residue corresponding to position 172 in SEQ ID NO:1;
r) the amino acid W at the residue corresponding to position 184 in SEQ ID NO:1;
s) the amino acid C or H at the residue corresponding to position 193 in SEQ ID NO:1;
t) the amino acid V at the residue corresponding to position 197 in SEQ ID NO:1;
u) the amino acid I at the residue corresponding to position 198 in SEQ ID NO: 1;
v) the amino acid I at the residue corresponding to position 212 in SEQ ID NO:1;
w) the amino acid L at the residue corresponding to position 213 in SEQ ID NO:1;
x) the amino acid L at the residue corresponding to position 227 in SEQ ID NO:1;
y) the amino acid T at the residue corresponding to position 228 in SEQ ID NO: 1;
z) the amino acid V at the residue corresponding to position 231 in SEQ ID NO:1;
aa) the amino acid M at the residue corresponding to position 235 in SEQ ID NO:1;
bb) the amino acid F at the residue corresponding to position 248 in SEQ ID NO:1;
cc) the amino acid L at the residue corresponding to position 249 in SEQ ID NO:1;
dd) the amino acid R at the residue corresponding to position 260 in SEQ ID NO:1;
ee) the amino acid I at the residue corresponding to position 282 in SEQ ID NO:1;
ff) the amino acid F at the residue corresponding to position 286 in SEQ ID NO: 1;
gg) the amino acid G at the residue corresponding to position 287 in SEQ ID NO:1;
hh) the amino acid G at the residue corresponding to position 289 in SEQ ID NO: 1;
ii) the amino acid I at the residue corresponding to position 295 in SEQ ID NO: 1;
jj) the amino acid T at the residue corresponding to position 296 in SEQ ID NO: 1;
kk) the amino acid F at the residue corresponding to position 309 in SEQ ID NO: 1;
ll) the amino acid S at the residue corresponding to position 314 in SEQ ID NO:1;
mm) the amino acid R at the residue corresponding to position 316 in SEQ ID NO:1;
nn) the amino acid N at the residue corresponding to position 329 in SEQ ID NO:1;
oo) the amino acid A at the residue corresponding to position 344 in SEQ ID NO: 1;
pp) the amino acid S at the residue corresponding to position 360 in SEQ ID NO:1;
qq) the amino acid L at the residue corresponding to position 370 in SEQ ID NO:1;
rr) the amino acid V at the residue corresponding to position 371 in SEQ ID NO:1;
ss) the amino acid P at the residue corresponding to position 372 in SEQ ID NO:1;
tt) the amino acid I at the residue corresponding to position 398 in SEQ ID NO: 1;
uu) the amino acid V at the residue corresponding to position 407 in SEQ ID NO:1;
vv) the amino acid S at the residue corresponding to position 414 in SEQ ID NO:1;
ww) the amino acid S at the residue corresponding to position 417 in SEQ ID NO:1;
xx) the amino acid L at the residue corresponding to position 423 in SEQ ID NO:1;
yy) the amino acid I or S at the residue corresponding to position 432 in SEQ ID NO:1;
zz) the amino acid L at the residue corresponding to position 437 in SEQ ID NO:1;
aaa) the amino acid V at the residue corresponding to position 442 in SEQ ID NO:1;
bbb) the amino acid M or S at the residue corresponding to position 444 in SEQ ID NO:1;
ccc) the amino acid G at the residue corresponding to position 452 in SEQ ID NO:1;
ddd) the amino acid V at the residue corresponding to position 474 in SEQ ID NO:1;
eee) the amino acid S at the residue corresponding to position 479 in SEQ ID NO:1;
fff) the amino acid Q at the residue corresponding to position 491 in SEQ ID NO:1;
ggg) the amino acid N at the residue corresponding to position 498 in SEQ ID NO: 1;
hhh) the amino acid L at the residue corresponding to position 515 in SEQ ID NO:1;
iii) the amino acid T at the residue corresponding to position 526 in SEQ ID NO:1;
jjj) the amino acid T at the residue corresponding to position 529 in SEQ ID NO:1;
kkk) the amino acid F at the residue corresponding to position 536 in SEQ ID NO:1;
lll) the amino acid Y at the residue corresponding to position 544 in SEQ ID NO:1;
mmm) the amino acid E at the residue corresponding to position 552 in SEQ ID NO:1;
nnn) the amino acid A at the residue corresponding to position 559 in SEQ ID NO:1;
ooo) the amino acid M at the residue corresponding to position 560 in SEQ ID NO:1;
ppp) the amino acid C or N at the residue corresponding to position 564 in SEQ ID NO:1;
qqq) the amino acid P at the residue corresponding to position 578 in SEQ ID NO:1;
rrr) the amino acid F at the residue corresponding to position 586 in SEQ ID NO:1;
sss) the amino acid T at the residue corresponding to position 608 in SEQ ID NO:1;
ttt) the amino acid I at the residue corresponding to position 610 in SEQ ID NO: 1;
uuu) the amino acid V at the residue corresponding to position 617 in SEQ ID NO:1;
vvv) the amino acid L at the residue corresponding to position 619 in SEQ ID NO:1;
www) the amino acid S at the residue corresponding to position 620 in SEQ ID NO:1;
xxx) the amino acid E or R at the residue corresponding to position 631 in SEQ ID NO:1;
yyy) the amino acid D at the residue corresponding to position 638 in SEQ ID NO:1;
zzz) the amino acid L at the residue corresponding to position 650 in SEQ ID NO:1;
aaaa) the amino acid A at the residue corresponding to position 655 in SEQ ID NO:1;
bbbb) the amino acid H at the residue corresponding to position 660 in SEQ ID NO:1;
cccc) the amino acid S at the residue corresponding to position 679 in SEQ ID NO:1;
dddd) the amino acid E at the residue corresponding to position 686 in SEQ ID NO: 1;
eeee) the amino acid D at the residue corresponding to position 702 in SEQ ID NO:1;
ffff) the amino acid Q at the residue corresponding to position 710 in SEQ ID NO:1;
gggg) the amino acid L or V at the residue corresponding to position 726 in SEQ ID NO:1;
hhhh) the amino acid F at the residue corresponding to position 736 in SEQ ID NO:1;
iiii) the amino acid M at the residue corresponding to position 738 in SEQ ID NO:1; and/or
jjjj) a truncation that results in deletion of the residue corresponding to position 742 in SEQ ID NO: 1.

72. The method of any one of claims 67-71, wherein the heterologous polynucleotide encoding the lanosterol synthase with reduced activity encodes a lanosterol synthase that comprises the amino acid substitution E617V, G107D, and/or K631E relative to SEQ ID NO: 1.

73. The method of any one of claims 67-71, wherein the heterologous polynucleotide encoding the lanosterol synthase with reduced activity encodes a lanosterol synthase that comprises relative to SEQ ID NO: 1, the lanosterol synthase comprises:
a) R33Q, R193C, D289G, N295I, S296T, N620S, and Y736F;
b) R184W, L235M, L260R, and E710Q;
c) K47E, L92I, T360S, S372P, T444M, and R578P;
d) D50G, K66R, N94S, G417S, E617V, and F726L;
e) N14Y, N132S, Y145C, R193H, I286F, L316R, F432I, E442V, T444S, I479S, K631R, and T655A;
f) F432S, D452G, and I536F;
g) E287G, K329N, E617V, and F726V;
h) E231V, A407V, Q423L, A529T, and Y564C;
i) V248F, D371V, and G702D;
j) L197V, K282I, N314S, P370L, A608T, G638D, and F650L;
k) L491Q, Y586F, and R660H;
l) G122C, H249L, and K738M;
m) P227L, E474V, V559A, and Y564N;
n) K85N, G158S, S515L, P526T, Q619L, and a truncation resulting in a deletion of the residue corresponding to Q742 in SEQ ID NO: 1;
o) G107D and K631E;
p) T212I, W213L, N544Y, and V552E;
q) I172N, C414S, L560M, and G679S;
r) R193C, D289G, N295I, S296T, N620S, and Y736F;
s) K85N and G158S;
t) L197V, K282I, N314S, and P370L;
u) I172N, C414S, and L560M;
v) D371V, M610I, and G702D;
w) D371V, K498N, M610I, and G702D;
x) D80G, P83L, T170A, T198I, and A228T;
y) T360S, S372P, T444M, and R578P;
z) D50G, K66R, N94S, G417S, and E617V; or
aa) L309F, V344A, T398I, and K686E.

74. The method of any one of claims 67-71 and 73, wherein relative to SEQ ID NO: 1, the lanosterol synthase comprises the following amino acid substitutions:
a) R193C, D289G, N295I, S296T, N620S, and Y736F;
b) F432S, D452G, and I536F;
c) K85N and G158S;
d) L197V, K282I, N314S, and P370L;
e) I172N, C414S, L560M, and G679S;
f) I172N, C414S, and L560M;
g) D371V, M610I, and G702D;
h) D371V, K498N, M610I, and G702D;
i) D80G, P83L, T170A, T198I, and A228T;
j) D50G, K66R, N94S, G417S, E617V, and F726L;
k) T360S, S372P, T444M, and R578P;
l) D50G, K66R, N94S, G417S, and E617V; and
m) L309F, V344A, T398I, and K686E.

75. The method of any one of claims 67-71 and 73, wherein relative to SEQ ID NO: 1, the lanosterol synthase comprises the following amino acid substitutions:
a) D50G, K66R, N94S, G417S, E617V, and F726L;
b) K85N and G158S;
c) K47E, L92I, T360S, S372P, T444M, and R578P;
d) F432S, D452G, and I536F;
e) T360S, S372P, T444M, and R578P;
f) L491Q, Y586F, and R660H;
g) K85N, G158S, S515L, P526T, Q619L, and a truncation that results in deletion of the residue corresponding to position 742 in SEQ ID NO: 1; or
h) I172N, C414S, L560M, and G679S.

76. The method of any one of claims 67-71 and 73, wherein the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

77. The method of any one of claims 67-71, 73, and 76, wherein the heterologous polynucleotide encodes a lanosterol synthase that comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

78. The method of any one of claims 67-77, wherein the heterologous polynucleotide encodes a lanosterol synthase that comprises a sequence that is at least 90% identical to SEQ ID NO: 3, 83-87, 89-92, 94-95, 99, 118-120, 316-319, 321-326, 329, or 331.

79. The method of claim 78, wherein the lanosterol synthase comprises SEQ ID NO: 3, 83-87, 89-92, 94-95, 99, 118-120, 316-319, 321-326, 329, or 331.

80. The method of any one of claims 67-79, wherein the heterologous polynucleotide encoding the lanosterol synthase comprises a sequence that is at least 90% identical to SEQ ID NO: 4, 62-66, 68-71, 73-74, 78, 103-109, 111-117, 328, or 330.

81. The method of claim 80, wherein the heterologous polynucleotide comprises the sequence of SEQ ID NO: 4, 62-66, 68-71, 73-74, 78, 103-109, 111-117, 328, or 330.

82. The method of claim 67 or claim 68, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

83. The method of claim 82, wherein the lanosterol synthase comprises:
(a) the amino acid G at the residue corresponding to position 64 in SEQ ID NO: 313;
(b) the amino acid V at the residue corresponding to position 120 in SEQ ID NO: 313;
(c) the amino acid S at the residue corresponding to position 121 in SEQ ID NO: 313;
(d) the amino acid V at the residue corresponding to position 136 in SEQ ID NO: 313;
(e) the amino acid I at the residue corresponding to position 226 in SEQ ID NO: 313;
(f) the amino acid S at the residue corresponding to position 268 in SEQ ID NO: 313;
(g) the amino acid I at the residue corresponding to position 275 in SEQ ID NO: 313;
(h) the amino acid A at the residue corresponding to position 281 in SEQ ID NO: 313;
(i) the amino acid G at the residue corresponding to position 300 in SEQ ID NO: 313;
(j) the amino acid G at the residue corresponding to position 322 in SEQ ID NO: 313;
(k) the amino acid A at the residue corresponding to position 333 in SEQ ID NO: 313;
(l) the amino acid E at the residue corresponding to position 438 in SEQ ID NO: 313;
(m) the amino acid L at the residue corresponding to position 502 in SEQ ID NO: 313;
(n) the amino acid N at the residue corresponding to position 604 in SEQ ID NO: 313;
(o) the amino acid S at the residue corresponding to position 619 in SEQ ID NO: 313;
(p) the amino acid E at the residue corresponding to position 628 in SEQ ID NO: 313;
(q) the amino acid T at the residue corresponding to position 656 in SEQ ID NO: 313;
(r) the amino acid G at the residue corresponding to position 693 in SEQ ID NO: 313; and/or
(s) deletion of residues corresponding to positions 726-731 in SEQ ID NO: 313.

84. The method of claim 82 or 83, wherein the lanosterol synthase comprises relative to SEQ ID NO: 313:
(a) P121S, A136V, S300G, V322G, K438E, F502L, K628E, and deletion of residues corresponding to positions 726-731 in SEQ ID NO: 313;
(b) K268S, T281A, F502L, T604N, A656T, and E693G; or
(c) C619S, F275I, I120V, M226I, R64G, and T333A.

85. The method of any one of claims 82-84, wherein the lanosterol synthase comprises a sequence that is at least 90% identical to any one of SEQ ID NOs: 100-102.

86. The method of claim 85, wherein the lanosterol synthase comprises a sequence selected from SEQ ID NOs: 100-102.

87. The method of any one of claims 82-86, wherein the heterologous polynucleotide encoding the lanosterol synthase comprises a sequence that is at least 90% identical to a sequence selected from SEQ ID NOs: 80-82.

88. The method of claim 87, wherein the heterologous polynucleotide encoding the lanosterol synthase comprises a sequence selected from SEQ ID NOs: 80-82.

89. The method of any one of claims 67-88, wherein the host cell is capable of producing mevalonate.

90. The method of any one of claims 67-89, wherein the host cell is capable of producing at least 0.2 g/L mevalonate.

91. The method of any one of claims 67-90, wherein the host cell is capable of producing at least 0.7 g/L mevalonate.

92. The method of any one of claims 67-91, wherein the host cell is capable of producing at least 9 mg/L of an isoprenoid.

93. The method of any one of claims 67-92, wherein the host cell is capable of producing at least 1.1 fold more of an isoprenoid than a control host cell comprising SEQ ID NO: 1 and/or a control host cell comprising SEQ ID NO: 313.

94. The method of any one of claims 67-93, wherein the host cell is capable of producing at least 3 fold more of an isoprenoid than a control host cell comprising SEQ ID NO: 1 and/or a control host cell comprising SEQ ID NO: 313.

95. The method of any one of claims 67-94, wherein the host cell is capable of producing at most 200 mg/L lanosterol.

96. The method of any one of claims 67-95, wherein the host cell is capable of producing at least 5 mg/L oxidosqualene.

97. The method of any one of claims 67-96, wherein the host cell is capable of producing more mevalonate than a control host cell that does not comprise: (a) the heterologous polynucleotide encoding the lanosterol synthase with reduced activity as compared to a wild-type lanosterol synthase; and/or (b) the heterologous polynucleotide encoding the squalene epoxidase with reduced activity as compared to a wild-type squalene epoxidase; or (c) the heterologous polynucleotide that reduces squalene epoxidase activity.

98. The method of any one of claims 67-97, wherein the host cell is capable of producing more 2-3-oxidosqualene as compared to a host cell that does not comprise:
(a) the heterologous polynucleotide encoding the lanosterol synthase with reduced activity as compared to a wild-type lanosterol synthase; and/or
(b) the heterologous polynucleotide encoding the squalene epoxidase with reduced activity as compared to a wild-type squalene epoxidase; or
(c) the heterologous polynucleotide that reduces squalene epoxidase activity.

99. The method of any one of claims 67-98, wherein the wild-type squalene epoxidase comprises SEQ ID NO: 9 or 312.

100. The method of any one of claims 67-99, wherein the heterologous polynucleotide encodes a squalene epoxidase comprises 1, 2, 3, 4, 5, 6, 7, 8, 9, or 10 amino acid substitutions and/or deletions relative to SEQ ID NO: 9 or 312.

101. The method of any one of claims 67-100, wherein the host cell is a yeast cell, a plant cell, or a bacterial cell.

102. The method of claim 101, wherein the host cell is a yeast cell.

103. The method of claim 102, wherein the yeast cell is a Saccharomyces cerevisiae cell.

104. The method of claim 102, wherein the yeast cell is a Yarrowia lipolytica cell.

105. The method of claim 101, wherein the host cell is a bacterial cell.

106. The method of claim 105, wherein the bacterial cell is an E. coli cell.

107. The method of any one of claims 67-106, wherein the isoprenoid precursor is mevalonate, 2-C-Methyl-d-erythritol-2,4-cyclopyrophosphate (MEcPP), and/or 2-3-oxidosqualene.

108. The host cell of any one of claims 50-51, wherein the host cell is capable of producing more of an isoprenoid or isoprenoid precursor as compared to a control host cell that does not comprise:
a) the heterologous polynucleotide encoding the lanosterol synthase with reduced activity as compared to the control wild-type lanosterol synthase; and/or
b) the heterologous polynucleotide encoding the squalene epoxidase with reduced activity as compared to the control wild-type squalene epoxidase; or
c) the heterologous polynucleotide that reduces squalene epoxidase activity.

109. The host cell of any one of claims 48-49 and 52-57, wherein the host cell is capable of producing more of an isoprenoid or isoprenoid precursor as compared to a control host cell that does not comprise:
a) the heterologous polynucleotide encoding the lanosterol synthase with reduced activity as compared to the control wild-type lanosterol synthase;
b) the heterologous polynucleotide that reduces lanosterol synthase activity; and/or
c) the heterologous polynucleotide encoding the squalene epoxidase with reduced activity as compared to the control wild-type squalene epoxidase; or
d) the heterologous polynucleotide that reduces squalene epoxidase activity.

110. The host cell of claim 108 or 109, wherein the wild-type lanosterol synthase comprises SEQ ID NO: 1 or 313.

111. The host cell of any one of claims 108-110, wherein the wild-type squalene epoxidase comprises SEQ ID NO: 9 or 312.

112. The host cell of any one of claims 48-57 or 108-111, wherein the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 80, 83, 85, 92, 94, 107, 122, 132, 145, 158, 170, 172, 184, 193, 197, 198, 212, 213, 227, 228, 231, 235, 248, 249, 260, 282, 286, 287, 289, 295, 296, 309, 314, 316, 329, 344, 360, 370, 371, 372, 398, 407, 414, 417, 423, 432, 437, 442, 444, 452, 474, 479, 491, 498, 515, 526, 529, 536, 544, 552, 559, 560, 564, 578, 586, 608, 610, 617, 619, 620, 631, 638, 650, 655, 660, 679, 686, 702, 710, 726, 736, 738, and/or 742 in SEQ ID NO: 1.

113. The host cell of any one of claims 48-57 and 108-112, wherein the heterologous polynucleotide encoding the lanosterol synthase with reduced activity encodes a lanosterol synthase that comprises 2, 3, 4, 5, 6, 7, 8, 9, 10, 11, or 12 amino acid substitutions and/or deletions relative to SEQ ID NO: 1.

114. The host cell of any one of claims 48-57 and 108-113, wherein the heterologous polynucleotide encoding the lanosterol synthase with reduced activity encodes a lanosterol synthase that comprises:
a) the amino acid Y at the residue corresponding to position 14 in SEQ ID NO:1;
b) the amino acid Q at the residue corresponding to position 33 in SEQ ID NO:1;
c) the amino acid E at the residue corresponding to position 47 in SEQ ID NO:1;
d) the amino acid G at the residue corresponding to position 50 in SEQ ID NO:1;
e) the amino acid R at the residue corresponding to position 66 in SEQ ID NO:1;
f) the amino acid G at the residue corresponding to position 80 in SEQ ID NO: 1;
g) the amino acid L at the residue corresponding to position 83 in SEQ ID NO: 1;
h) the amino acid N at the residue corresponding to position 85 in SEQ ID NO:1;
i) the amino acid I at the residue corresponding to position 92 in SEQ ID NO:1;
j) the amino acid S at the residue corresponding to position 94 in SEQ ID NO:1;
k) the amino acid D at the residue corresponding to position 107 in SEQ ID NO:1;
l) the amino acid C at the residue corresponding to position 122 in SEQ ID NO:1;
m) the amino acid S at the residue corresponding to position 132 in SEQ ID NO:1;
n) the amino acid C at the residue corresponding to position 145 in SEQ ID NO:1;
o) the amino acid S at the residue corresponding to position 158 in SEQ ID NO:1;
p) the amino acid A at the residue corresponding to position 170 in SEQ ID NO: 1;
q) the amino acid N at the residue corresponding to position 172 in SEQ ID NO:1;
r) the amino acid W at the residue corresponding to position 184 in SEQ ID NO:1;
s) the amino acid C or H at the residue corresponding to position 193 in SEQ ID NO:1;
t) the amino acid V at the residue corresponding to position 197 in SEQ ID NO:1;
u) the amino acid I at the residue corresponding to position 198 in SEQ ID NO: 1;
v) the amino acid I at the residue corresponding to position 212 in SEQ ID NO:1;
w) the amino acid L at the residue corresponding to position 213 in SEQ ID NO:1;
x) the amino acid L at the residue corresponding to position 227 in SEQ ID NO:1;
y) the amino acid T at the residue corresponding to position 228 in SEQ ID NO: 1;
z) the amino acid V at the residue corresponding to position 231 in SEQ ID NO:1;
aa) the amino acid M at the residue corresponding to position 235 in SEQ ID NO:1;
bb) the amino acid F at the residue corresponding to position 248 in SEQ ID NO:1;
cc) the amino acid L at the residue corresponding to position 249 in SEQ ID NO:1;
dd) the amino acid R at the residue corresponding to position 260 in SEQ ID NO:1;
ee) the amino acid I at the residue corresponding to position 282 in SEQ ID NO:1;
ff) the amino acid F at the residue corresponding to position 286 in SEQ ID NO: 1;
gg) the amino acid G at the residue corresponding to position 287 in SEQ ID NO:1;
hh) the amino acid G at the residue corresponding to position 289 in SEQ ID NO: 1;
ii) the amino acid I at the residue corresponding to position 295 in SEQ ID NO: 1;
jj) the amino acid T at the residue corresponding to position 296 in SEQ ID NO: 1;
kk) the amino acid F at the residue corresponding to position 309 in SEQ ID NO: 1;
ll) the amino acid S at the residue corresponding to position 314 in SEQ ID NO:1;
mm) the amino acid R at the residue corresponding to position 316 in SEQ ID NO:1;
nn) the amino acid N at the residue corresponding to position 329 in SEQ ID NO:1;
oo) the amino acid A at the residue corresponding to position 344 in SEQ ID NO: 1;
pp) the amino acid S at the residue corresponding to position 360 in SEQ ID NO:1;
qq) the amino acid L at the residue corresponding to position 370 in SEQ ID NO:1;
rr) the amino acid V at the residue corresponding to position 371 in SEQ ID NO:1;
ss) the amino acid P at the residue corresponding to position 372 in SEQ ID NO:1;
tt) the amino acid I at the residue corresponding to position 398 in SEQ ID NO: 1;
uu) the amino acid V at the residue corresponding to position 407 in SEQ ID NO:1;
vv) the amino acid S at the residue corresponding to position 414 in SEQ ID NO:1;
ww) the amino acid S at the residue corresponding to position 417 in SEQ ID NO:1;
xx) the amino acid L at the residue corresponding to position 423 in SEQ ID NO:1;
yy) the amino acid I or S at the residue corresponding to position 432 in SEQ ID NO:1;
zz) the amino acid L at the residue corresponding to position 437 in SEQ ID NO:1;
aaa) the amino acid V at the residue corresponding to position 442 in SEQ ID NO:1;
bbb) the amino acid M or S at the residue corresponding to position 444 in SEQ ID NO:1;
ccc) the amino acid G at the residue corresponding to position 452 in SEQ ID NO:1;
ddd) the amino acid V at the residue corresponding to position 474 in SEQ ID NO:1;
eee) the amino acid S at the residue corresponding to position 479 in SEQ ID NO:1;
fff) the amino acid Q at the residue corresponding to position 491 in SEQ ID NO:1;
ggg) the amino acid N at the residue corresponding to position 498 in SEQ ID NO: 1;
hhh) the amino acid L at the residue corresponding to position 515 in SEQ ID NO:1;
iii) the amino acid T at the residue corresponding to position 526 in SEQ ID NO:1;
jjj) the amino acid T at the residue corresponding to position 529 in SEQ ID NO:1;
kkk) the amino acid F at the residue corresponding to position 536 in SEQ ID NO:1;
lll) the amino acid Y at the residue corresponding to position 544 in SEQ ID NO:1;
mmm) the amino acid E at the residue corresponding to position 552 in SEQ ID NO:1;
nnn) the amino acid A at the residue corresponding to position 559 in SEQ ID NO:1;
ooo) the amino acid M at the residue corresponding to position 560 in SEQ ID NO:1;
ppp) the amino acid C or N at the residue corresponding to position 564 in SEQ ID NO:1;
qqq) the amino acid P at the residue corresponding to position 578 in SEQ ID NO:1;
rrr) the amino acid F at the residue corresponding to position 586 in SEQ ID NO:1;
sss) the amino acid T at the residue corresponding to position 608 in SEQ ID NO:1;
ttt) the amino acid I at the residue corresponding to position 610 in SEQ ID NO: 1;
uuu) the amino acid V at the residue corresponding to position 617 in SEQ ID NO:1;
vvv) the amino acid L at the residue corresponding to position 619 in SEQ ID NO:1;
www) the amino acid S at the residue corresponding to position 620 in SEQ ID NO:1;
xxx) the amino acid E or R at the residue corresponding to position 631 in SEQ ID NO:1;
yyy) the amino acid D at the residue corresponding to position 638 in SEQ ID NO:1;
zzz) the amino acid L at the residue corresponding to position 650 in SEQ ID NO:1;
aaaa) the amino acid A at the residue corresponding to position 655 in SEQ ID NO:1;
bbbb) the amino acid H at the residue corresponding to position 660 in SEQ ID NO:1;
cccc) the amino acid S at the residue corresponding to position 679 in SEQ ID NO:1;
dddd) the amino acid E at the residue corresponding to position 686 in SEQ ID NO: 1;
eeee) the amino acid D at the residue corresponding to position 702 in SEQ ID NO:1;
ffff) the amino acid Q at the residue corresponding to position 710 in SEQ ID NO:1;
gggg) the amino acid L or V at the residue corresponding to position 726 in SEQ ID NO:1;
hhhh) the amino acid F at the residue corresponding to position 736 in SEQ ID NO:1;
iiii) the amino acid M at the residue corresponding to position 738 in SEQ ID NO:1; and/or
jjjj) a truncation that results in deletion of the residue corresponding to position 742 in SEQ ID NO: 1.

115. The host cell of any one of claims 48-57 and 108-114, wherein the heterologous polynucleotide encoding the lanosterol synthase with reduced activity encodes a lanosterol synthase that comprises the amino acid substitution E617V, G107D, and/or K631E relative to SEQ ID NO: 1.

116. The host cell of any one of claims 48-57 and 108-114, wherein the heterologous polynucleotide encoding the lanosterol synthase with reduced activity encodes a lanosterol synthase that comprises relative to SEQ ID NO: 1, the lanosterol synthase comprises:
a) R33Q, R193C, D289G, N295I, S296T, N620S, and Y736F;
b) R184W, L235M, L260R, and E710Q;
c) K47E, L92I, T360S, S372P, T444M, and R578P;
d) D50G, K66R, N94S, G417S, E617V, and F726L;
e) N14Y, N132S, Y145C, R193H, I286F, L316R, F432I, E442V, T444S, I479S, K631R, and T655A;
f) F432S, D452G, and I536F;
g) E287G, K329N, E617V, and F726V;
h) E231V, A407V, Q423L, A529T, and Y564C;
i) V248F, D371V, and G702D;
j) L197V, K282I, N314S, P370L, A608T, G638D, and F650L;
k) L491Q, Y586F, and R660H;
l) G122C, H249L, and K738M;
m) P227L, E474V, V559A, and Y564N;
n) K85N, G158S, S515L, P526T, Q619L, and a truncation resulting in a deletion of the residue corresponding to Q742 in SEQ ID NO: 1;
o) G107D and K631E;
p) T212I, W213L, N544Y, and V552E;
q) I172N, C414S, L560M, and G679S;
r) R193C, D289G, N295I, S296T, N620S, and Y736F;
s) K85N and G158S;
t) L197V, K282I, N314S, and P370L;
u) I172N, C414S, and L560M;
v) D371V, M610I, and G702D;
w) D371V, K498N, M610I, and G702D;
x) D80G, P83L, T170A, T198I, and A228T;
y) T360S, S372P, T444M, and R578P;
z) D50G, K66R, N94S, G417S, and E617V; or
aa) L309F, V344A, T398I, and K686E.

117. The host cell of any one of claims 48-57, 108-114, and 116, wherein relative to SEQ ID NO: 1, the lanosterol synthase comprises the following amino acid substitutions:
(a) R193C, D289G, N295I, S296T, N620S, and Y736F;
(b) F432S, D452G, and I536F;
(c) K85N and G158S;
(d) L197V, K282I, N314S, and P370L;
(e) I172N, C414S, L560M, and G679S;
(f) I172N, C414S, and L560M;
(g) D371V, M610I, and G702D;
(h) D371V, K498N, M610I, and G702D;
(i) D80G, P83L, T170A, T198I, and A228T;
(j) D50G, K66R, N94S, G417S, E617V, and F726L;
(k) T360S, S372P, T444M, and R578P;
(l) D50G, K66R, N94S, G417S, and E617V; and
(m) L309F, V344A, T398I, and K686E.

118. The host cell of any one of claims 48-57, 108-114, and 116, wherein relative to SEQ ID NO: 1, the lanosterol synthase comprises the following amino acid substitutions:
(a) D50G, K66R, N94S, G417S, E617V, and F726L;
(b) K85N and G158S;
(c) K47E, L92I, T360S, S372P, T444M, and R578P;
(d) F432S, D452G, and I536F;
(e) T360S, S372P, T444M, and R578P;
(f) L491Q, Y586F, and R660H;
(g) K85N, G158S, S515L, P526T, Q619L, and a truncation that results in deletion of the residue corresponding to position 742 in SEQ ID NO: 1; or
(h) I172N, C414S, L560M, and G679S.

119. The host cell of any one of claims 48-57, 108-114, and 116, wherein the heterologous polynucleotide encoding the lanosterol synthase with reduced activity encodes a lanosterol synthase that comprises an amino acid substitution or deletion relative to SEQ ID NO: 1 at one or more residues corresponding to position 14, 33, 47, 50, 66, 85, 92, 94, 122, 132, 145, 158, 193, 231, 248, 249, 286, 287, 289, 295, 296, 316, 329, 360, 371, 372, 407, 417, 423, 432, 442, 444, 479, 515, 526, 529, 564, 578, 617, 619, 620, 631, 655, 702, 726, 736, 738, and/or 742 in SEQ ID NO: 1.

120. The host cell of any one of claims 48-57, 108-114, 116, and 119, wherein the heterologous polynucleotide encodes a lanosterol synthase that comprises relative to SEQ ID NO: 1:
a) R33Q, R193C, D289G, N295I, S296T, N620S, and Y736F;
b) K47E, L92I, T360S, S372P, T444M, and R578P;
c) D50G, K66R, N94S, G417S, E617V, and F726L;
d) N14Y, N132S, Y145C, R193H, I286F, L316R, F432I, E442V, T444S, I479S, K631R, and T655A;
e) E287G, K329N, E617V, and F726V;
f) E231V, A407V, Q423L, A529T, and Y564C;
g) V248F, D371V, and G702D;
h) G122C, H249L, and K738M; or
i) K85N, G158S, S515L, P526T, and Q619L, and a truncation resulting in a deletion of the residue corresponding to Q742 in SEQ ID NO: 1.

121. The host cell of any one of claims 48-57 and 108-120, wherein the heterologous polynucleotide encodes a lanosterol synthase that comprises a sequence that is at least 90% identical to SEQ ID NO: 33, 83-87, 89-92, 94-95, 99, 118-120, 316-319, 321-326, 329, or 331.

122. The host cell of claim 121, wherein the lanosterol synthase comprises SEQ ID NO: 33, 83-87, 89-92, 94-95, 99, 118-120, 316-319, 321-326, 329, or 331.

123. The host cell of any one of claims 48-57 and 108-122, wherein the heterologous polynucleotide encoding the lanosterol synthase comprises a sequence that is at least 90% identical to SEQ ID NO: 4, 62-66, 68-71, 73-74, 78, 103-109, 111-117, 328, or 330.

124. The host cell of claim 123, wherein the heterologous polynucleotide comprises the sequence of SEQ ID NO: 4, 62-66, 68-71, 73-74, 78, 103-109, 111-117, 328, or 330.

125. The host cell of any one of claims claim 48-57 and 108-111, wherein the host cell comprises a heterologous polynucleotide encoding a lanosterol synthase, wherein the lanosterol synthase comprises an amino acid substitution or deletion relative to SEQ ID NO: 313 at one or more residues corresponding to position 64, 120, 121, 136, 226, 268, 275, 281, 300, 322, 333, 438, 502, 604, 619, 628, 656, 693, 726, 727, 728, 729, 730, and/or 731.

126. The host cell of claim 125, wherein the lanosterol synthase comprises:
(a) the amino acid G at the residue corresponding to position 64 in SEQ ID NO: 313;
(b) the amino acid V at the residue corresponding to position 120 in SEQ ID NO: 313;
(c) the amino acid S at the residue corresponding to position 121 in SEQ ID NO: 313;
(d) the amino acid V at the residue corresponding to position 136 in SEQ ID NO: 313;
(e) the amino acid I at the residue corresponding to position 226 in SEQ ID NO: 313;
(f) the amino acid S at the residue corresponding to position 268 in SEQ ID NO: 313;
(g) the amino acid I at the residue corresponding to position 275 in SEQ ID NO: 313;
(h) the amino acid A at the residue corresponding to position 281 in SEQ ID NO: 313;
(i) the amino acid G at the residue corresponding to position 300 in SEQ ID NO: 313;
(j) the amino acid G at the residue corresponding to position 322 in SEQ ID NO: 313;
(k) the amino acid A at the residue corresponding to position 333 in SEQ ID NO: 313;
(l) the amino acid E at the residue corresponding to position 438 in SEQ ID NO: 313;
(m) the amino acid L at the residue corresponding to position 502 in SEQ ID NO: 313;
(n) the amino acid N at the residue corresponding to position 604 in SEQ ID NO: 313;
(o) the amino acid S at the residue corresponding to position 619 in SEQ ID NO: 313;
(p) the amino acid E at the residue corresponding to position 628 in SEQ ID NO: 313;
(q) the amino acid T at the residue corresponding to position 656 in SEQ ID NO: 313;
(r) the amino acid G at the residue corresponding to position 693 in SEQ ID NO: 313; and/or
(s) deletion of residues corresponding to positions 726-731 in SEQ ID NO: 313.

127. The host cell of any one of claims 48-57, 108-111, and 125-126, wherein the lanosterol synthase comprises relative to SEQ ID NO: 313:
(a) P121S, A136V, S300G, V322G, K438E, F502L, K628E, and deletion of residues corresponding to positions 726-731 in SEQ ID NO: 313;
(b) K268S, T281A, F502L, T604N, A656T, and E693G; or
(c) C619S, F275I, I120V, M226I, R64G, and T333A.

128. The host cell of any one of claims 48-57, 108-111, and 125-127, wherein the lanosterol synthase comprises a sequence that is at least 90% identical to any one of SEQ ID NOs: 100-102.

129. The host cell of claim 128, wherein the lanosterol synthase comprises a sequence selected from SEQ ID NOs: 100-102.

130. The host of any one of claims 48-57, 108-111, and 125-129, wherein the heterologous polynucleotide encoding the lanosterol synthase comprises a sequence that is at least 90% identical to a sequence selected from SEQ ID NOs: 80-82.

131. The host cell of claim 130, wherein the heterologous polynucleotide encoding the lanosterol synthase comprises a sequence selected from SEQ ID NOs: 80-82.

132. The host cell of any one of claims 50-57 and 108-131, wherein the host cell is capable of producing mevalonate.

133. The host cell of any one of claims 50-57 and 108-132, wherein the host cell is capable of producing at least 0.2 g/L mevalonate.

134. The host cell of any one of claims 50-57 and 108-133, wherein the host cell is capable of producing at least 0.7 g/L mevalonate.

135. The host cell of any one of claims 50-51, 108, and 110-134, wherein the host cell is capable of producing more mevalonate than a control host cell that does not comprise: (a) the heterologous polynucleotide encoding the lanosterol synthase with reduced activity as compared to a wild-type lanosterol synthase; and/or (b) the heterologous polynucleotide encoding the squalene epoxidase with reduced activity as compared to a wild-type squalene epoxidase; or (c) the heterologous polynucleotide that reduces squalene epoxidase activity.

136. The host cell of any one of claims 52-57 or 109-134, wherein the host cell is capable of producing more mevalonate than a control host cell that does not comprise: (a) the heterologous polynucleotide encoding the lanosterol synthase with reduced activity as compared to a wild-type lanosterol synthase; or (b) the heterologous polynucleotide that reduces lanosterol synthase activity; and/or (c) the heterologous polynucleotide encoding the squalene epoxidase with reduced activity as compared to a wild-type squalene epoxidase; or (d) the heterologous polynucleotide that reduces squalene epoxidase activity.

137. The host cell of any one of claims 50-51, 108, and 110-135, wherein the host cell is capable of producing more 2-3-oxidosqualene as compared to a host cell that does not comprise:
(a) the heterologous polynucleotide encoding the lanosterol synthase with reduced activity as compared to a wild-type lanosterol synthase; and/or
(b) the heterologous polynucleotide encoding the squalene epoxidase with reduced activity as compared to a wild-type squalene epoxidase; or
(c) the heterologous polynucleotide that reduces squalene epoxidase activity.

138. The host cell of any one of claims 52-57, 109-134 and 136, wherein the host cell is capable of producing more 2-3-oxidosqualene as compared to a host cell that does not comprise:
(a) the heterologous polynucleotide encoding the lanosterol synthase with reduced activity as compared to a wild-type lanosterol synthase; or
(b) the heterologous polynucleotide that reduces lanosterol synthase activity; and/or
(c) the heterologous polynucleotide encoding the squalene epoxidase with reduced activity as compared to a wild-type squalene epoxidase; or
(d) the heterologous polynucleotide that reduces squalene epoxidase activity.

139. The host cell of any one of claims 50-57 and 108-138, wherein the heterologous polynucleotide encoding the squalene epoxidase with reduced activity encodes a squalene epoxidase comprises 1, 2, 3, 4, 5, 6, 7, 8, 9, or 10 amino acid substitutions and/or deletions relative to SEQ ID NO: 9 or 312.

140. The host cell of any one of claims 108-139, wherein the host cell is a yeast cell, a plant cell, or a bacterial cell.

141. The host cell of claim 140, wherein the host cell is a yeast cell.

142. The host cell of claim 141, wherein the yeast cell is a Saccharomyces cerevisiae cell.

143. The host cell of claim 141, wherein the yeast cell is a Yarrowia lipolytica cell.

144. The host cell of claim 140, wherein the host cell is a bacterial cell.

145. The host cell of claim 144, wherein the bacterial cell is an E. coli cell.
